SEQUENCE LISTING (1492 sequences)

SEQ ID NO: 1 - RNA, 19 nt, Artificial Sequence
  Description of Artificial Sequence Synthetic oligonucleotide
ggacacggacuucauuguu

SEQ ID NO: 2 - RNA, 19 nt, Artificial Sequence
  Description of Artificial Sequence Synthetic oligonucleotide
gcacucauguggaaguucg

SEQ ID NO: 3 - RNA, 19 nt, Artificial Sequence
  Description of Artificial Sequence Synthetic oligonucleotide
agaaagggcagcucuauaa

SEQ ID NO: 4 - RNA, 19 nt, Artificial Sequence
  Description of Artificial Sequence Synthetic oligonucleotide
ugaaagggcuuccgggugg

SEQ ID NO: 5 - RNA, 19 nt, Artificial Sequence
  Description of Artificial Sequence Synthetic oligonucleotide
cccaaagucucucacauua

SEQ ID NO: 6 - RNA, 19 nt, Artificial Sequence
  Description of Artificial Sequence Synthetic oligonucleotide
gcaagggaaggaauaugua

SEQ ID NO: 7 - RNA, 19 nt, Artificial Sequence
  Description of Artificial Sequence Synthetic oligonucleotide
ggacaaacuuaacaacaau

SEQ ID NO: 8 - RNA, 19 nt, Artificial Sequence
  Description of Artificial Sequence Synthetic oligonucleotide
gcuaauggcuggauuuauu

SEQ ID NO: 9 - RNA, 19 nt, Artificial Sequence
  Description of Artificial Sequence Synthetic oligonucleotide
cuacaaagccucagggaua

SEQ ID NO: 10 - RNA, 19 nt, Artificial Sequence
  Description of Artificial Sequence Synthetic oligonucleotide
gcacagcagccuuacuaau

SEQ ID NO: 11 - RNA, 19 nt, Artificial Sequence
  Description of Artificial Sequence Synthetic oligonucleotide
gaaugugggugucaacaag

SEQ ID NO: 12 - RNA, 19 nt, Artificial Sequence
  Description of Artificial Sequence Synthetic oligonucleotide
gcugaagccuggaaacaaa

SEQ ID NO: 13 - RNA, 19 nt, Artificial Sequence
  Description of Artificial Sequence Synthetic oligonucleotide
gggcgagauuccugcauua

SEQ ID NO: 14 - RNA, 19 nt, Artificial Sequence
  Description of Artificial Sequence Synthetic oligonucleotide
gaaauucacccgauacaua

SEQ ID NO: 15 - RNA, 19 nt, Artificial Sequence
  Description of Artificial Sequence Synthetic oligonucleotide
gaaagucucauccuaauuu

SEQ ID NO: 16 - RNA, 19 nt, Artificial Sequence
  Description of Artificial Sequence Synthetic oligonucleotide
ugaagaagauuaucgaaga

SEQ ID NO: 17 - RNA, 19 nt, Artificial Sequence
  Description of Artificial Sequence Synthetic oligonucleotide
gaccgccucugcuuuguca

SEQ ID NO: 18 - RNA, 19 nt, Artificial Sequence
  Description of Artificial Sequence Synthetic oligonucleotide
ggacaaggacgggcacauu

SEQ ID NO: 19 - RNA, 19 nt, Artificial Sequence
  Description of Artificial Sequence Synthetic oligonucleotide
gacaaggacgggcacauua

SEQ ID NO: 20 - RNA, 19 nt, Artificial Sequence
  Description of Artificial Sequence Synthetic oligonucleotide
gcuacuuccuccucaagaa

SEQ ID NO: 21 - RNA, 19 nt, Artificial Sequence
  Description of Artificial Sequence Synthetic oligonucleotide
gagccugguuguugccaau

SEQ ID NO: 22 - RNA, 19 nt, Artificial Sequence
  Description of Artificial Sequence Synthetic oligonucleotide
gcauugagacaguauccua

SEQ ID NO: 23 - RNA, 19 nt, Artificial Sequence
  Description of Artificial Sequence Synthetic oligonucleotide
cguacucauuugcaagaua

SEQ ID NO: 24 - RNA, 19 nt, Artificial Sequence
  Description of Artificial Sequence Synthetic oligonucleotide
cagcaaccaucaaaguaau

SEQ ID NO: 25 - RNA, 19 nt, Artificial Sequence
  Description of Artificial Sequence Synthetic oligonucleotide
ucagaaugcuuacggcuau

SEQ ID NO: 26 - RNA, 19 nt, Artificial Sequence
  Description of Artificial Sequence Synthetic oligonucleotide
guucgagcacuaauauuua

SEQ ID NO: 27 - RNA, 19 nt, Artificial Sequence
  Description of Artificial Sequence Synthetic oligonucleotide
guaaucgacugugaggaua

SEQ ID NO: 28 - RNA, 19 nt, Artificial Sequence
  Description of Artificial Sequence Synthetic oligonucleotide
cuagaggugccaauauuaa

SEQ ID NO: 29 - RNA, 19 nt, Artificial Sequence
  Description of Artificial Sequence Synthetic oligonucleotide
gcaagaguaacaucuaaua

SEQ ID NO: 30 - RNA, 19 nt, Artificial Sequence
  Description of Artificial Sequence Synthetic oligonucleotide
ugaaggacaugcaaacuuu

SEQ ID NO: 31 - RNA, 19 nt, Artificial Sequence
  Description of Artificial Sequence Synthetic oligonucleotide
gcagauauauguggaguaa

SEQ ID NO: 32 - RNA, 19 nt, Artificial Sequence
  Description of Artificial Sequence Synthetic oligonucleotide
cgaaugcaguuaauaagua

SEQ ID NO: 33 - RNA, 19 nt, Artificial Sequence
  Description of Artificial Sequence Synthetic oligonucleotide
gcgcaccagucgacgugaa

SEQ ID NO: 34 - RNA, 19 nt, Artificial Sequence
  Description of Artificial Sequence Synthetic oligonucleotide
gcccauggcuccacguaaa

SEQ ID NO: 35 - RNA, 19 nt, Artificial Sequence
  Description of Artificial Sequence Synthetic oligonucleotide
gccuuuagcuggucuagug

SEQ ID NO: 36 - RNA, 19 nt, Artificial Sequence
  Description of Artificial Sequence Synthetic oligonucleotide
gcccgaggcuugaagaagu

SEQ ID NO: 37 - RNA, 19 nt, Artificial Sequence
  Description of Artificial Sequence Synthetic oligonucleotide
caagauaaggcuacauuga

SEQ ID NO: 38 - RNA, 19 nt, Artificial Sequence
  Description of Artificial Sequence Synthetic oligonucleotide
uaucagcucuacacauugu

SEQ ID NO: 39 - RNA, 19 nt, Artificial Sequence
  Description of Artificial Sequence Synthetic oligonucleotide
cagaggcacucaaacuaag

SEQ ID NO: 40 - RNA, 19 nt, Artificial Sequence
  Description of Artificial Sequence Synthetic oligonucleotide
gcagauacgaccauuguua

SEQ ID NO: 41 - RNA, 19 nt, Artificial Sequence
  Description of Artificial Sequence Synthetic oligonucleotide
accaagcguacgcgcauua

SEQ ID NO: 42 - RNA, 19 nt, Artificial Sequence
  Description of Artificial Sequence Synthetic oligonucleotide
gcaagucgucguucgccuu

SEQ ID NO: 43 - RNA, 19 nt, Artificial Sequence
  Description of Artificial Sequence Synthetic oligonucleotide
cguuggaccucacuggcaa

SEQ ID NO: 44 - RNA, 19 nt, Artificial Sequence
  Description of Artificial Sequence Synthetic oligonucleotide
gcuacaaccgcgugggcau

SEQ ID NO: 45 - RNA, 19 nt, Artificial Sequence
  Description of Artificial Sequence Synthetic oligonucleotide
gaagaugccuugauugaaa

SEQ ID NO: 46 - RNA, 19 nt, Artificial Sequence
  Description of Artificial Sequence Synthetic oligonucleotide
ggccauaguuaauugugug

SEQ ID NO: 47 - RNA, 19 nt, Artificial Sequence
  Description of Artificial Sequence Synthetic oligonucleotide
ucaagccuauuauacagua

SEQ ID NO: 48 - RNA, 19 nt, Artificial Sequence
  Description of Artificial Sequence Synthetic oligonucleotide
aggaauaucaagcagcaua

SEQ ID NO: 49 - RNA, 19 nt, Artificial Sequence
  Description of Artificial Sequence Synthetic oligonucleotide
gaagagcagucacagauca

SEQ ID NO: 50 - RNA, 19 nt, Artificial Sequence
  Description of Artificial Sequence Synthetic oligonucleotide
uauaugagcugcuggauga

SEQ ID NO: 51 - RNA, 19 nt, Artificial Sequence
  Description of Artificial Sequence Synthetic oligonucleotide
ggaggcuuauucaucuaua

SEQ ID NO: 52 - RNA, 19 nt, Artificial Sequence
  Description of Artificial Sequence Synthetic oligonucleotide
cgugauggcugccuacuuu

SEQ ID NO: 53 - RNA, 19 nt, Artificial Sequence
  Description of Artificial Sequence Synthetic oligonucleotide
gaucaaagguuuucacuaa

SEQ ID NO: 54 - RNA, 19 nt, Artificial Sequence
  Description of Artificial Sequence Synthetic oligonucleotide
gaagauaacaauuggugua

SEQ ID NO: 55 - RNA, 19 nt, Artificial Sequence
  Description of Artificial Sequence Synthetic oligonucleotide
caacaucccugguuacaag

SEQ ID NO: 56 - RNA, 19 nt, Artificial Sequence
  Description of Artificial Sequence Synthetic oligonucleotide
ccaaaguucuugaucaaua

SEQ ID NO: 57 - RNA, 19 nt, Artificial Sequence
  Description of Artificial Sequence Synthetic oligonucleotide
acguggaaaccguggagua

SEQ ID NO: 58 - RNA, 19 nt, Artificial Sequence
  Description of Artificial Sequence Synthetic oligonucleotide
gaccaccauucccaccaua

SEQ ID NO: 59 - RNA, 19 nt, Artificial Sequence
  Description of Artificial Sequence Synthetic oligonucleotide
acagagagcgugugaacga

SEQ ID NO: 60 - RNA, 19 nt, Artificial Sequence
  Description of Artificial Sequence Synthetic oligonucleotide
cggccgagaucacagacaa

SEQ ID NO: 61 - RNA, 19 nt, Artificial Sequence
  Description of Artificial Sequence Synthetic oligonucleotide
gagaagaccugagcucauu

SEQ ID NO: 62 - RNA, 19 nt, Artificial Sequence
  Description of Artificial Sequence Synthetic oligonucleotide
ggaggaaugugaaguagaa

SEQ ID NO: 63 - RNA, 19 nt, Artificial Sequence
  Description of Artificial Sequence Synthetic oligonucleotide
agacagagauuugggauua

SEQ ID NO: 64 - RNA, 19 nt, Artificial Sequence
  Description of Artificial Sequence Synthetic oligonucleotide
gacaggaagugauuaauga

SEQ ID NO: 65 - RNA, 19 nt, Artificial Sequence
  Description of Artificial Sequence Synthetic oligonucleotide
aauggaggcucgaagugua

SEQ ID NO: 66 - RNA, 19 nt, Artificial Sequence
  Description of Artificial Sequence Synthetic oligonucleotide
ggacaagcaguggcuguuu

SEQ ID NO: 67 - RNA, 19 nt, Artificial Sequence
  Description of Artificial Sequence Synthetic oligonucleotide
gagucuaccugcccuaugu

SEQ ID NO: 68 - RNA, 19 nt, Artificial Sequence
  Description of Artificial Sequence Synthetic oligonucleotide
ggagugcaaugcuagugua

SEQ ID NO: 69 - RNA, 19 nt, Artificial Sequence
  Description of Artificial Sequence Synthetic oligonucleotide
gcaagauugucgcaaauuu

SEQ ID NO: 70 - RNA, 19 nt, Artificial Sequence
  Description of Artificial Sequence Synthetic oligonucleotide
gaaaaugacugguggguga

SEQ ID NO: 71 - RNA, 19 nt, Artificial Sequence
  Description of Artificial Sequence Synthetic oligonucleotide
acgaagugcacaucuauaa

SEQ ID NO: 72 - RNA, 19 nt, Artificial Sequence
  Description of Artificial Sequence Synthetic oligonucleotide
gaauuaaucgcgcagcuac

SEQ ID NO: 73 - RNA, 19 nt, Artificial Sequence
  Description of Artificial Sequence Synthetic oligonucleotide
gcucagcccuuaacaauga

SEQ ID NO: 74 - RNA, 19 nt, Artificial Sequence
  Description of Artificial Sequence Synthetic oligonucleotide
gagaauuaccuguucacua

SEQ ID NO: 75 - RNA, 19 nt, Artificial Sequence
  Description of Artificial Sequence Synthetic oligonucleotide
gcacgccuucgaaauguua

SEQ ID NO: 76 - RNA, 19 nt, Artificial Sequence
  Description of Artificial Sequence Synthetic oligonucleotide
ccacagcuucuaauuauaa

SEQ ID NO: 77 - RNA, 19 nt, Artificial Sequence
  Description of Artificial Sequence Synthetic oligonucleotide
gggaaggacuuacggaugu

SEQ ID NO: 78 - RNA, 19 nt, Artificial Sequence
  Description of Artificial Sequence Synthetic oligonucleotide
gaacaccaaguuucacugu

SEQ ID NO: 79 - RNA, 19 nt, Artificial Sequence
  Description of Artificial Sequence Synthetic oligonucleotide
gcagcuuccuacuucaauu

SEQ ID NO: 80 - RNA, 19 nt, Artificial Sequence
  Description of Artificial Sequence Synthetic oligonucleotide
gcaguagagcgaacacgaa

SEQ ID NO: 81 - RNA, 19 nt, Artificial Sequence
  Description of Artificial Sequence Synthetic oligonucleotide
guaaugacguggugugugg

SEQ ID NO: 82 - RNA, 19 nt, Artificial Sequence
  Description of Artificial Sequence Synthetic oligonucleotide
gaggagaggucacucaauu

SEQ ID NO: 83 - RNA, 19 nt, Artificial Sequence
  Description of Artificial Sequence Synthetic oligonucleotide
ggacacaaggauaagguga

SEQ ID NO: 84 - RNA, 19 nt, Artificial Sequence
  Description of Artificial Sequence Synthetic oligonucleotide
gagcagcgauaccagauca

SEQ ID NO: 85 - RNA, 21 nt, Artificial Sequence
  Description of Artificial Sequence Synthetic oligonucleotide
ccaaaguucuugaucaauauu

SEQ ID NO: 86 - RNA, 21 nt, Artificial Sequence
  Description of Artificial Sequence Synthetic oligonucleotide
gaucaaagguuuucacuaauu

SEQ ID NO: 87 - RNA, 21 nt, Artificial Sequence
  Description of Artificial Sequence Synthetic oligonucleotide
gaagauaacaauugguguauu

SEQ ID NO: 88 - RNA, 21 nt, Artificial Sequence
  Description of Artificial Sequence Synthetic oligonucleotide
caacaucccugguuacaaguu

SEQ ID NO: 89 - RNA, 19 nt, Artificial Sequence
  Description of Artificial Sequence Synthetic oligonucleotide
ccaauaaacugacguucuu

SEQ ID NO: 90 - RNA, 19 nt, Artificial Sequence
  Description of Artificial Sequence Synthetic oligonucleotide
gaagaugucuguuauccuu

SEQ ID NO: 91 - RNA, 19 nt, Artificial Sequence
  Description of Artificial Sequence Synthetic oligonucleotide
gaacuuaccgagagauaaa

SEQ ID NO: 92 - RNA, 19 nt, Artificial Sequence
  Description of Artificial Sequence Synthetic oligonucleotide
ggaagaggcacuccuuuaa

SEQ ID NO: 93 - RNA, 19 nt, Artificial Sequence
  Description of Artificial Sequence Synthetic oligonucleotide
gaaaggacgccucgugcaa

SEQ ID NO: 94 - RNA, 19 nt, Artificial Sequence
  Description of Artificial Sequence Synthetic oligonucleotide
gaaugcagcuggaggugug

SEQ ID NO: 95 - RNA, 19 nt, Artificial Sequence
  Description of Artificial Sequence Synthetic oligonucleotide
ccgaguacaugacaauuuc

SEQ ID NO: 96 - RNA, 19 nt, Artificial Sequence
  Description of Artificial Sequence Synthetic oligonucleotide
aggaagaguuuaacauuga

SEQ ID NO: 97 - RNA, 19 nt, Artificial Sequence
  Description of Artificial Sequence Synthetic oligonucleotide
gaaagucccuucccaugcu

SEQ ID NO: 98 - RNA, 19 nt, Artificial Sequence
  Description of Artificial Sequence Synthetic oligonucleotide
uuacagaccugagcacuua

SEQ ID NO: 99 - RNA, 19 nt, Artificial Sequence
  Description of Artificial Sequence Synthetic oligonucleotide
gaacgcagcugcuuuccua

SEQ ID NO: 100 - RNA, 19 nt, Artificial Sequence
  Description of Artificial Sequence Synthetic oligonucleotide
ccgcacuaauggcugggua

SEQ ID NO: 101 - RNA, 19 nt, Artificial Sequence
  Description of Artificial Sequence Synthetic oligonucleotide
gccagacgcugcaauauuu

SEQ ID NO: 102 - RNA, 19 nt, Artificial Sequence
  Description of Artificial Sequence Synthetic oligonucleotide
caacucaacucccaacaau

SEQ ID NO: 103 - RNA, 19 nt, Artificial Sequence
  Description of Artificial Sequence Synthetic oligonucleotide
gaacauuucuaacaacaag

SEQ ID NO: 104 - RNA, 19 nt, Artificial Sequence
  Description of Artificial Sequence Synthetic oligonucleotide
cuacugagauggccaauaa

SEQ ID NO: 105 - RNA, 19 nt, Artificial Sequence
  Description of Artificial Sequence Synthetic oligonucleotide
acaagcaccucucgcauua

SEQ ID NO: 106 - RNA, 19 nt, Artificial Sequence
  Description of Artificial Sequence Synthetic oligonucleotide
ggucaaagaucacuuagua

SEQ ID NO: 107 - RNA, 19 nt, Artificial Sequence
  Description of Artificial Sequence Synthetic oligonucleotide
gcaacgaugugccauguua

SEQ ID NO: 108 - RNA, 19 nt, Artificial Sequence
  Description of Artificial Sequence Synthetic oligonucleotide
ccaaagauacgguuacaua

SEQ ID NO: 109 - RNA, 19 nt, Artificial Sequence
  Description of Artificial Sequence Synthetic oligonucleotide
ggucagagcuacacauuga

SEQ ID NO: 110 - RNA, 19 nt, Artificial Sequence
  Description of Artificial Sequence Synthetic oligonucleotide
guacaagaucacugacucu

SEQ ID NO: 111 - RNA, 19 nt, Artificial Sequence
  Description of Artificial Sequence Synthetic oligonucleotide
ggacaaggcccucaugaac

SEQ ID NO: 112 - RNA, 19 nt, Artificial Sequence
  Description of Artificial Sequence Synthetic oligonucleotide
gaagucgucagaacacauc

SEQ ID NO: 113 - RNA, 19 nt, Artificial Sequence
  Description of Artificial Sequence Synthetic oligonucleotide
gaaaugcuguuaguuguua

SEQ ID NO: 114 - RNA, 19 nt, Artificial Sequence
  Description of Artificial Sequence Synthetic oligonucleotide
ggagacagaugagagauua

SEQ ID NO: 115 - RNA, 19 nt, Artificial Sequence
  Description of Artificial Sequence Synthetic oligonucleotide
cagaagcgcuguauugaua

SEQ ID NO: 116 - RNA, 19 nt, Artificial Sequence
  Description of Artificial Sequence Synthetic oligonucleotide
gaaccuacccuuucucuua

SEQ ID NO: 117 - RNA, 19 nt, Artificial Sequence
  Description of Artificial Sequence Synthetic oligonucleotide
ggaauacuauacccaugag

SEQ ID NO: 118 - RNA, 19 nt, Artificial Sequence
  Description of Artificial Sequence Synthetic oligonucleotide
uaacagccgucauuuaugc

SEQ ID NO: 119 - RNA, 19 nt, Artificial Sequence
  Description of Artificial Sequence Synthetic oligonucleotide
gccaaccguuauuuaugua

SEQ ID NO: 120 - RNA, 19 nt, Artificial Sequence
  Description of Artificial Sequence Synthetic oligonucleotide
gaagaaaccauucgagaug

SEQ ID NO: 121 - RNA, 19 nt, Artificial Sequence
  Description of Artificial Sequence Synthetic oligonucleotide
gcaauacggagagcacuuu

SEQ ID NO: 122 - RNA, 19 nt, Artificial Sequence
  Description of Artificial Sequence Synthetic oligonucleotide
caacuucacucuccauuaa

SEQ ID NO: 123 - RNA, 19 nt, Artificial Sequence
  Description of Artificial Sequence Synthetic oligonucleotide
caaagugcggaaucuuaaa

SEQ ID NO: 124 - RNA, 19 nt, Artificial Sequence
  Description of Artificial Sequence Synthetic oligonucleotide
agguggagcuuaugaaauu

SEQ ID NO: 125 - RNA, 19 nt, Artificial Sequence
  Description of Artificial Sequence Synthetic oligonucleotide
gaugaagaccgauuaguua

SEQ ID NO: 126 - RNA, 19 nt, Artificial Sequence
  Description of Artificial Sequence Synthetic oligonucleotide
guacuuggcggugcacuuu

SEQ ID NO: 127 - RNA, 19 nt, Artificial Sequence
  Description of Artificial Sequence Synthetic oligonucleotide
cgucagguaucuggauuaa

SEQ ID NO: 128 - RNA, 19 nt, Artificial Sequence
  Description of Artificial Sequence Synthetic oligonucleotide
ugacugaacccacuaagca

SEQ ID NO: 129 - RNA, 19 nt, Artificial Sequence
  Description of Artificial Sequence Synthetic oligonucleotide
cauuuaggauucucuguug

SEQ ID NO: 130 - RNA, 19 nt, Artificial Sequence
  Description of Artificial Sequence Synthetic oligonucleotide
ccagauauaugcucucgaa

SEQ ID NO: 131 - RNA, 19 nt, Artificial Sequence
  Description of Artificial Sequence Synthetic oligonucleotide
guagggaguaucuucacua

SEQ ID NO: 132 - RNA, 19 nt, Artificial Sequence
  Description of Artificial Sequence Synthetic oligonucleotide
guagagugugcaauacaaa

SEQ ID NO: 133 - RNA, 19 nt, Artificial Sequence
  Description of Artificial Sequence Synthetic oligonucleotide
gcaaagcuuaucucccuug

SEQ ID NO: 134 - RNA, 19 nt, Artificial Sequence
  Description of Artificial Sequence Synthetic oligonucleotide
ucaaugaacugugggauua

SEQ ID NO: 135 - RNA, 19 nt, Artificial Sequence
  Description of Artificial Sequence Synthetic oligonucleotide
aaucagaccuguggcuaau

SEQ ID NO: 136 - RNA, 19 nt, Artificial Sequence
  Description of Artificial Sequence Synthetic oligonucleotide
gagugaugauggacauuag

SEQ ID NO: 137 - RNA, 19 nt, Artificial Sequence
  Description of Artificial Sequence Synthetic oligonucleotide
ccaaggugguacauggcug

SEQ ID NO: 138 - RNA, 19 nt, Artificial Sequence
  Description of Artificial Sequence Synthetic oligonucleotide
ggugcugggucuccauuuc

SEQ ID NO: 139 - RNA, 19 nt, Artificial Sequence
  Description of Artificial Sequence Synthetic oligonucleotide
cuuuguggguguugagcuc

SEQ ID NO: 140 - RNA, 19 nt, Artificial Sequence
  Description of Artificial Sequence Synthetic oligonucleotide
ggaugccacugcuggaugu

SEQ ID NO: 141 - RNA, 19 nt, Artificial Sequence
  Description of Artificial Sequence Synthetic oligonucleotide
acaacaaccuuauuucgug

SEQ ID NO: 142 - RNA, 19 nt, Artificial Sequence
  Description of Artificial Sequence Synthetic oligonucleotide
auccagagcucuucauuaa

SEQ ID NO: 143 - RNA, 19 nt, Artificial Sequence
  Description of Artificial Sequence Synthetic oligonucleotide
ggcauauauugguuaucua

SEQ ID NO: 144 - RNA, 19 nt, Artificial Sequence
  Description of Artificial Sequence Synthetic oligonucleotide
ggaccucuaugaagcuauu

SEQ ID NO: 145 - RNA, 19 nt, Artificial Sequence
  Description of Artificial Sequence Synthetic oligonucleotide
ccacaaagcacuuauauag

SEQ ID NO: 146 - RNA, 19 nt, Artificial Sequence
  Description of Artificial Sequence Synthetic oligonucleotide
caugucaccucucuacucc

SEQ ID NO: 147 - RNA, 19 nt, Artificial Sequence
  Description of Artificial Sequence Synthetic oligonucleotide
cgagaucgguucacuuugu

SEQ ID NO: 148 - RNA, 19 nt, Artificial Sequence
  Description of Artificial Sequence Synthetic oligonucleotide
ucucagaucuacuagcaca

SEQ ID NO: 149 - RNA, 19 nt, Artificial Sequence
  Description of Artificial Sequence Synthetic oligonucleotide
ggaaggauacugagcauuc

SEQ ID NO: 150 - RNA, 19 nt, Artificial Sequence
  Description of Artificial Sequence Synthetic oligonucleotide
gcacccagcuuguaacuua

SEQ ID NO: 151 - RNA, 19 nt, Artificial Sequence
  Description of Artificial Sequence Synthetic oligonucleotide
gaaccacacagaaucaauc

SEQ ID NO: 152 - RNA, 19 nt, Artificial Sequence
  Description of Artificial Sequence Synthetic oligonucleotide
ccucaaggcuauagggaua

SEQ ID NO: 153 - RNA, 19 nt, Artificial Sequence
  Description of Artificial Sequence Synthetic oligonucleotide
ccgucuggaucgaguacua

SEQ ID NO: 154 - RNA, 19 nt, Artificial Sequence
  Description of Artificial Sequence Synthetic oligonucleotide
ugacagccgugguaaccga

SEQ ID NO: 155 - RNA, 19 nt, Artificial Sequence
  Description of Artificial Sequence Synthetic oligonucleotide
ggaagaagagggacuaucu

SEQ ID NO: 156 - RNA, 19 nt, Artificial Sequence
  Description of Artificial Sequence Synthetic oligonucleotide
gcucggagaucuucgaauu

SEQ ID NO: 157 - RNA, 19 nt, Artificial Sequence
  Description of Artificial Sequence Synthetic oligonucleotide
gaauacaguucaugccauu

SEQ ID NO: 158 - RNA, 19 nt, Artificial Sequence
  Description of Artificial Sequence Synthetic oligonucleotide
ggacugaaguggugauuga

SEQ ID NO: 159 - RNA, 19 nt, Artificial Sequence
  Description of Artificial Sequence Synthetic oligonucleotide
ggccauaccuauaccauga

SEQ ID NO: 160 - RNA, 19 nt, Artificial Sequence
  Description of Artificial Sequence Synthetic oligonucleotide
agaaggaccugcgcacuua

SEQ ID NO: 161 - RNA, 19 nt, Artificial Sequence
  Description of Artificial Sequence Synthetic oligonucleotide
uaucauugcuccauugugu

SEQ ID NO: 162 - RNA, 19 nt, Artificial Sequence
  Description of Artificial Sequence Synthetic oligonucleotide
guaaagaccugccuaaugg

SEQ ID NO: 163 - RNA, 19 nt, Artificial Sequence
  Description of Artificial Sequence Synthetic oligonucleotide
cggcucaacucgcaucuca

SEQ ID NO: 164 - RNA, 19 nt, Artificial Sequence
  Description of Artificial Sequence Synthetic oligonucleotide
ggacguacagcucuucaug

SEQ ID NO: 165 - RNA, 19 nt, Artificial Sequence
  Description of Artificial Sequence Synthetic oligonucleotide
cgaggauugugcacuauua

SEQ ID NO: 166 - RNA, 19 nt, Artificial Sequence
  Description of Artificial Sequence Synthetic oligonucleotide
agagaaacgccgaaagaaa

SEQ ID NO: 167 - RNA, 19 nt, Artificial Sequence
  Description of Artificial Sequence Synthetic oligonucleotide
gcucaccgcugcugaugug

SEQ ID NO: 168 - RNA, 19 nt, Artificial Sequence
  Description of Artificial Sequence Synthetic oligonucleotide
gcacagaguucauucccag

SEQ ID NO: 169 - RNA, 19 nt, Artificial Sequence
  Description of Artificial Sequence Synthetic oligonucleotide
gguuaccuuccuucaauua

SEQ ID NO: 170 - RNA, 19 nt, Artificial Sequence
  Description of Artificial Sequence Synthetic oligonucleotide
ggacagauugcccuaguac

SEQ ID NO: 171 - RNA, 19 nt, Artificial Sequence
  Description of Artificial Sequence Synthetic oligonucleotide
gcacucaaaugggaagaaa

SEQ ID NO: 172 - RNA, 19 nt, Artificial Sequence
  Description of Artificial Sequence Synthetic oligonucleotide
gaccaccugucucuagugu

SEQ ID NO: 173 - RNA, 19 nt, Artificial Sequence
  Description of Artificial Sequence Synthetic oligonucleotide
gcgcaucacugacugcuau

SEQ ID NO: 174 - RNA, 19 nt, Artificial Sequence
  Description of Artificial Sequence Synthetic oligonucleotide
ccaccagccuccugugauc

SEQ ID NO: 175 - RNA, 19 nt, Artificial Sequence
  Description of Artificial Sequence Synthetic oligonucleotide
ggaccucggagaugaguug

SEQ ID NO: 176 - RNA, 19 nt, Artificial Sequence
  Description of Artificial Sequence Synthetic oligonucleotide
gcagccaacuauccagauc

SEQ ID NO: 177 - RNA, 19 nt, Artificial Sequence
  Description of Artificial Sequence Synthetic oligonucleotide
aaucagggcuccuucuuaa

SEQ ID NO: 178 - RNA, 19 nt, Artificial Sequence
  Description of Artificial Sequence Synthetic oligonucleotide
ucaagagacuccucaguga

SEQ ID NO: 179 - RNA, 19 nt, Artificial Sequence
  Description of Artificial Sequence Synthetic oligonucleotide
gaacugaccuguacagcuu

SEQ ID NO: 180 - RNA, 19 nt, Artificial Sequence
  Description of Artificial Sequence Synthetic oligonucleotide
gaagaagagcauacaauuc

SEQ ID NO: 181 - RNA, 19 nt, Artificial Sequence
  Description of Artificial Sequence Synthetic oligonucleotide
gagaaacgaagcuuggaua

SEQ ID NO: 182 - RNA, 19 nt, Artificial Sequence
  Description of Artificial Sequence Synthetic oligonucleotide
aaacagagcuuccuacuaa

SEQ ID NO: 183 - RNA, 19 nt, Artificial Sequence
  Description of Artificial Sequence Synthetic oligonucleotide
uuaacgggcucgucaagga

SEQ ID NO: 184 - RNA, 19 nt, Artificial Sequence
  Description of Artificial Sequence Synthetic oligonucleotide
gagcgcgagucguggauuc

SEQ ID NO: 185 - RNA, 19 nt, Artificial Sequence
  Description of Artificial Sequence Synthetic oligonucleotide
agacccagcuagacaugaa

SEQ ID NO: 186 - RNA, 19 nt, Artificial Sequence
  Description of Artificial Sequence Synthetic oligonucleotide
gauaagugggaccaguaua

SEQ ID NO: 187 - RNA, 19 nt, Artificial Sequence
  Description of Artificial Sequence Synthetic oligonucleotide
gaggcgaauucuacaguua

SEQ ID NO: 188 - RNA, 19 nt, Artificial Sequence
  Description of Artificial Sequence Synthetic oligonucleotide
gcuggaagaucacgaguac

SEQ ID NO: 189 - RNA, 19 nt, Artificial Sequence
  Description of Artificial Sequence Synthetic oligonucleotide
caauguauccggugaauuu

SEQ ID NO: 190 - RNA, 19 nt, Artificial Sequence
  Description of Artificial Sequence Synthetic oligonucleotide
aaaccacaauagcgggauu

SEQ ID NO: 191 - RNA, 19 nt, Artificial Sequence
  Description of Artificial Sequence Synthetic oligonucleotide
ggaccuaucuuuggcguug

SEQ ID NO: 192 - RNA, 19 nt, Artificial Sequence
  Description of Artificial Sequence Synthetic oligonucleotide
ccggaaagaguacaccuua

SEQ ID NO: 193 - RNA, 19 nt, Artificial Sequence
  Description of Artificial Sequence Synthetic oligonucleotide
gcaacaacuucucuuaugu

SEQ ID NO: 194 - RNA, 19 nt, Artificial Sequence
  Description of Artificial Sequence Synthetic oligonucleotide
ggucuucgcuagcaucuca

SEQ ID NO: 195 - RNA, 19 nt, Artificial Sequence
  Description of Artificial Sequence Synthetic oligonucleotide
cgggaaagguggacaguau

SEQ ID NO: 196 - RNA, 19 nt, Artificial Sequence
  Description of Artificial Sequence Synthetic oligonucleotide
ucaagggucugcuguggua

SEQ ID NO: 197 - RNA, 19 nt, Artificial Sequence
  Description of Artificial Sequence Synthetic oligonucleotide
gugagcagccaguauaaua

SEQ ID NO: 198 - RNA, 19 nt, Artificial Sequence
  Description of Artificial Sequence Synthetic oligonucleotide
aaucagcguuggaggcaau

SEQ ID NO: 199 - RNA, 19 nt, Artificial Sequence
  Description of Artificial Sequence Synthetic oligonucleotide
uuagauuugugccuaguga

SEQ ID NO: 200 - RNA, 19 nt, Artificial Sequence
  Description of Artificial Sequence Synthetic oligonucleotide
ggaccgaagugacugucua

SEQ ID NO: 201 - RNA, 19 nt, Artificial Sequence
  Description of Artificial Sequence Synthetic oligonucleotide
ggacuuacgccacgauuga

SEQ ID NO: 202 - RNA, 19 nt, Artificial Sequence
  Description of Artificial Sequence Synthetic oligonucleotide
aaaguaagaccgcguauag

SEQ ID NO: 203 - RNA, 19 nt, Artificial Sequence
  Description of Artificial Sequence Synthetic oligonucleotide
gauacucuguguuugucaa

SEQ ID NO: 204 - RNA, 19 nt, Artificial Sequence
  Description of Artificial Sequence Synthetic oligonucleotide
guuauucggucaguugaga

SEQ ID NO: 205 - RNA, 19 nt, Artificial Sequence
  Description of Artificial Sequence Synthetic oligonucleotide
ccgaguucaucuuguuuaa

SEQ ID NO: 206 - RNA, 19 nt, Artificial Sequence
  Description of Artificial Sequence Synthetic oligonucleotide
gacaaagguuucagcguua

SEQ ID NO: 207 - RNA, 19 nt, Artificial Sequence
  Description of Artificial Sequence Synthetic oligonucleotide
cauugucggugaauaguga

SEQ ID NO: 208 - RNA, 19 nt, Artificial Sequence
  Description of Artificial Sequence Synthetic oligonucleotide
ccauugaucauucauguua

SEQ ID NO: 209 - RNA, 19 nt, Artificial Sequence
  Description of Artificial Sequence Synthetic oligonucleotide
gcaaaguucgucagcuuga

SEQ ID NO: 210 - RNA, 19 nt, Artificial Sequence
  Description of Artificial Sequence Synthetic oligonucleotide
cagcacauauucaucgcua

SEQ ID NO: 211 - RNA, 19 nt, Artificial Sequence
  Description of Artificial Sequence Synthetic oligonucleotide
ucuauacccugaucaaaua

SEQ ID NO: 212 - RNA, 19 nt, Artificial Sequence
  Description of Artificial Sequence Synthetic oligonucleotide
gcuggaggcuguauacgaa

SEQ ID NO: 213 - RNA, 19 nt, Artificial Sequence
  Description of Artificial Sequence Synthetic oligonucleotide
ccaaucaacuucauaggau

SEQ ID NO: 214 - RNA, 19 nt, Artificial Sequence
  Description of Artificial Sequence Synthetic oligonucleotide
gcauauuuggugaccuuaa

SEQ ID NO: 215 - RNA, 19 nt, Artificial Sequence
  Description of Artificial Sequence Synthetic oligonucleotide
gcacggaauaucagaauaa

SEQ ID NO: 216 - RNA, 19 nt, Artificial Sequence
  Description of Artificial Sequence Synthetic oligonucleotide
gcacuuaucuagaagacug

SEQ ID NO: 217 - RNA, 19 nt, Artificial Sequence
  Description of Artificial Sequence Synthetic oligonucleotide
gaaagcuguuuauccauuc

SEQ ID NO: 218 - RNA, 19 nt, Artificial Sequence
  Description of Artificial Sequence Synthetic oligonucleotide
uccgauaccuggaaauuag

SEQ ID NO: 219 - RNA, 19 nt, Artificial Sequence
  Description of Artificial Sequence Synthetic oligonucleotide
gcacaggcaugccaaauuu

SEQ ID NO: 220 - RNA, 19 nt, Artificial Sequence
  Description of Artificial Sequence Synthetic oligonucleotide
gauaagacccgagcuuuga

SEQ ID NO: 221 - RNA, 19 nt, Artificial Sequence
  Description of Artificial Sequence Synthetic oligonucleotide
gcauggugguucuguaggu

SEQ ID NO: 222 - RNA, 19 nt, Artificial Sequence
  Description of Artificial Sequence Synthetic oligonucleotide
gucagacgcuguuaccgua

SEQ ID NO: 223 - RNA, 19 nt, Artificial Sequence
  Description of Artificial Sequence Synthetic oligonucleotide
gugccgauguggagugcca

SEQ ID NO: 224 - RNA, 19 nt, Artificial Sequence
  Description of Artificial Sequence Synthetic oligonucleotide
ucacacaugucgaugcgga

SEQ ID NO: 225 - RNA, 19 nt, Artificial Sequence
  Description of Artificial Sequence Synthetic oligonucleotide
ccaugcaauucgcuuauua

SEQ ID NO: 226 - RNA, 19 nt, Artificial Sequence
  Description of Artificial Sequence Synthetic oligonucleotide
gaaguuucguguugaagga

SEQ ID NO: 227 - RNA, 19 nt, Artificial Sequence
  Description of Artificial Sequence Synthetic oligonucleotide
ggaccaccauuuaaagcua

SEQ ID NO: 228 - RNA, 19 nt, Artificial Sequence
  Description of Artificial Sequence Synthetic oligonucleotide
gaauagcauugcacgauua

SEQ ID NO: 229 - RNA, 19 nt, Artificial Sequence
  Description of Artificial Sequence Synthetic oligonucleotide
gaagauggcaagcuugaua

SEQ ID NO: 230 - RNA, 19 nt, Artificial Sequence
  Description of Artificial Sequence Synthetic oligonucleotide
ggaaagguguugaaaguga

SEQ ID NO: 231 - RNA, 19 nt, Artificial Sequence
  Description of Artificial Sequence Synthetic oligonucleotide
guaaagggauauaacgaga

SEQ ID NO: 232 - RNA, 19 nt, Artificial Sequence
  Description of Artificial Sequence Synthetic oligonucleotide
ggacaaaacuccucucuau

SEQ ID NO: 233 - RNA, 19 nt, Artificial Sequence
  Description of Artificial Sequence Synthetic oligonucleotide
gagaucagauuauagagaa

SEQ ID NO: 234 - RNA, 19 nt, Artificial Sequence
  Description of Artificial Sequence Synthetic oligonucleotide
ucgaacggcuucauccuau

SEQ ID NO: 235 - RNA, 19 nt, Artificial Sequence
  Description of Artificial Sequence Synthetic oligonucleotide
ggcaaucaguuccaaugug

SEQ ID NO: 236 - RNA, 19 nt, Artificial Sequence
  Description of Artificial Sequence Synthetic oligonucleotide
gcaccuggaacgaguaauu

SEQ ID NO: 237 - RNA, 19 nt, Artificial Sequence
  Description of Artificial Sequence Synthetic oligonucleotide
caucccaagugcuaaagua

SEQ ID NO: 238 - RNA, 19 nt, Artificial Sequence
  Description of Artificial Sequence Synthetic oligonucleotide
ggaaaggacuagacauuga

SEQ ID NO: 239 - RNA, 19 nt, Artificial Sequence
  Description of Artificial Sequence Synthetic oligonucleotide
agacauauccugccauuga

SEQ ID NO: 240 - RNA, 19 nt, Artificial Sequence
  Description of Artificial Sequence Synthetic oligonucleotide
gaacgaaugcgaagacuga

SEQ ID NO: 241 - RNA, 19 nt, Artificial Sequence
  Description of Artificial Sequence Synthetic oligonucleotide
cgggaaaccuuagaaaucu

SEQ ID NO: 242 - RNA, 19 nt, Artificial Sequence
  Description of Artificial Sequence Synthetic oligonucleotide
aaagaugucuggaaguuug

SEQ ID NO: 243 - RNA, 19 nt, Artificial Sequence
  Description of Artificial Sequence Synthetic oligonucleotide
gggcacgggugauaugaau

SEQ ID NO: 244 - RNA, 19 nt, Artificial Sequence
  Description of Artificial Sequence Synthetic oligonucleotide
gaacagcccugcggccuuu

SEQ ID NO: 245 - RNA, 19 nt, Artificial Sequence
  Description of Artificial Sequence Synthetic oligonucleotide
guuccugguuggccaauau

SEQ ID NO: 246 - RNA, 19 nt, Artificial Sequence
  Description of Artificial Sequence Synthetic oligonucleotide
ccagauuguuuagagguua

SEQ ID NO: 247 - RNA, 19 nt, Artificial Sequence
  Description of Artificial Sequence Synthetic oligonucleotide
gcaaaaugcuauugugaau

SEQ ID NO: 248 - RNA, 19 nt, Artificial Sequence
  Description of Artificial Sequence Synthetic oligonucleotide
ccacauacguuacuggguc

SEQ ID NO: 249 - RNA, 19 nt, Artificial Sequence
  Description of Artificial Sequence Synthetic oligonucleotide
uaacuacaccgaggaaaug

SEQ ID NO: 250 - RNA, 19 nt, Artificial Sequence
  Description of Artificial Sequence Synthetic oligonucleotide
gaagcaugacggacaagua

SEQ ID NO: 251 - RNA, 19 nt, Artificial Sequence
  Description of Artificial Sequence Synthetic oligonucleotide
caagcaagggugugaguuu

SEQ ID NO: 252 - RNA, 19 nt, Artificial Sequence
  Description of Artificial Sequence Synthetic oligonucleotide
gagucugagucuucaaguu

SEQ ID NO: 253 - RNA, 19 nt, Artificial Sequence
  Description of Artificial Sequence Synthetic oligonucleotide
ggaggcacauuucgucuaa

SEQ ID NO: 254 - RNA, 19 nt, Artificial Sequence
  Description of Artificial Sequence Synthetic oligonucleotide
ccgaguuagucuuuggguu

SEQ ID NO: 255 - RNA, 19 nt, Artificial Sequence
  Description of Artificial Sequence Synthetic oligonucleotide
gcacuccuaugucuugauc

SEQ ID NO: 256 - RNA, 19 nt, Artificial Sequence
  Description of Artificial Sequence Synthetic oligonucleotide
gaccagggcacaccaaaga

SEQ ID NO: 257 - RNA, 19 nt, Artificial Sequence
  Description of Artificial Sequence Synthetic oligonucleotide
gaucgggaguugagaaaau

SEQ ID NO: 258 - RNA, 19 nt, Artificial Sequence
  Description of Artificial Sequence Synthetic oligonucleotide
gaaugcaacgucuugauua

SEQ ID NO: 259 - RNA, 19 nt, Artificial Sequence
  Description of Artificial Sequence Synthetic oligonucleotide
gaagaaugaguacguagaa

SEQ ID NO: 260 - RNA, 19 nt, Artificial Sequence
  Description of Artificial Sequence Synthetic oligonucleotide
gcuuaaggaaugucacaaa

SEQ ID NO: 261 - RNA, 19 nt, Artificial Sequence
  Description of Artificial Sequence Synthetic oligonucleotide
cgacguguaugguguauuc

SEQ ID NO: 262 - RNA, 19 nt, Artificial Sequence
  Description of Artificial Sequence Synthetic oligonucleotide
ggaauuccucuaugacaau

SEQ ID NO: 263 - RNA, 19 nt, Artificial Sequence
  Description of Artificial Sequence Synthetic oligonucleotide
gaccaucagugccuuuauu

SEQ ID NO: 264 - RNA, 19 nt, Artificial Sequence
  Description of Artificial Sequence Synthetic oligonucleotide
gacaggcaacuaugaacua

SEQ ID NO: 265 - RNA, 19 nt, Artificial Sequence
  Description of Artificial Sequence Synthetic oligonucleotide
gaggaugccaacacauaua

SEQ ID NO: 266 - RNA, 19 nt, Artificial Sequence
  Description of Artificial Sequence Synthetic oligonucleotide
gagccaagccgauaaagua

SEQ ID NO: 267 - RNA, 19 nt, Artificial Sequence
  Description of Artificial Sequence Synthetic oligonucleotide
gaauggaagucuauaauga

SEQ ID NO: 268 - RNA, 19 nt, Artificial Sequence
  Description of Artificial Sequence Synthetic oligonucleotide
cacaggugguaucaacuua

SEQ ID NO: 269 - RNA, 19 nt, Artificial Sequence
  Description of Artificial Sequence Synthetic oligonucleotide
ucacacaggucaacguggu

SEQ ID NO: 270 - RNA, 19 nt, Artificial Sequence
  Description of Artificial Sequence Synthetic oligonucleotide
gugcaagaccugccagauu

SEQ ID NO: 271 - RNA, 19 nt, Artificial Sequence
  Description of Artificial Sequence Synthetic oligonucleotide
caucguggcucguggaaca

SEQ ID NO: 272 - RNA, 19 nt, Artificial Sequence
  Description of Artificial Sequence Synthetic oligonucleotide
gcgaagagagugugagauc

SEQ ID NO: 273 - RNA, 19 nt, Artificial Sequence
  Description of Artificial Sequence Synthetic oligonucleotide
cauaauaaggguagggauu

SEQ ID NO: 274 - RNA, 19 nt, Artificial Sequence
  Description of Artificial Sequence Synthetic oligonucleotide
ggucauugguuacagugga

SEQ ID NO: 275 - RNA, 19 nt, Artificial Sequence
  Description of Artificial Sequence Synthetic oligonucleotide
ggaagaucuuguaguaaug

SEQ ID NO: 276 - RNA, 19 nt, Artificial Sequence
  Description of Artificial Sequence Synthetic oligonucleotide
guacgucaacuagcacuuu

SEQ ID NO: 277 - RNA, 19 nt, Artificial Sequence
  Description of Artificial Sequence Synthetic oligonucleotide
gugaagggcgugaagauua

SEQ ID NO: 278 - RNA, 19 nt, Artificial Sequence
  Description of Artificial Sequence Synthetic oligonucleotide
cagcggaccuucugccaaa

SEQ ID NO: 279 - RNA, 19 nt, Artificial Sequence
  Description of Artificial Sequence Synthetic oligonucleotide
ggaagaggguucugauauu

SEQ ID NO: 280 - RNA, 19 nt, Artificial Sequence
  Description of Artificial Sequence Synthetic oligonucleotide
ugucauauguccaagcuua

SEQ ID NO: 281 - RNA, 19 nt, Artificial Sequence
  Description of Artificial Sequence Synthetic oligonucleotide
cuacgucggcuaugguuua

SEQ ID NO: 282 - RNA, 19 nt, Artificial Sequence
  Description of Artificial Sequence Synthetic oligonucleotide
ggaguuagcauaucaugaa

SEQ ID NO: 283 - RNA, 19 nt, Artificial Sequence
  Description of Artificial Sequence Synthetic oligonucleotide
gcgagcaccuguuugauag

SEQ ID NO: 284 - RNA, 19 nt, Artificial Sequence
  Description of Artificial Sequence Synthetic oligonucleotide
gcacacagguuuggguuug

SEQ ID NO: 285 - RNA, 19 nt, Artificial Sequence
  Description of Artificial Sequence Synthetic oligonucleotide
acaguggucuugagguguu

SEQ ID NO: 286 - RNA, 19 nt, Artificial Sequence
  Description of Artificial Sequence Synthetic oligonucleotide
cuacuagagucucagauga

SEQ ID NO: 287 - RNA, 19 nt, Artificial Sequence
  Description of Artificial Sequence Synthetic oligonucleotide
ccaaagggcuaucgcaaag

SEQ ID NO: 288 - RNA, 19 nt, Artificial Sequence
  Description of Artificial Sequence Synthetic oligonucleotide
guuaggugcucuugaacau

SEQ ID NO: 289 - RNA, 19 nt, Artificial Sequence
  Description of Artificial Sequence Synthetic oligonucleotide
gcagaucaggccucuauaa

SEQ ID NO: 290 - RNA, 19 nt, Artificial Sequence
  Description of Artificial Sequence Synthetic oligonucleotide
uagaagagcuccgucagaa

SEQ ID NO: 291 - RNA, 19 nt, Artificial Sequence
  Description of Artificial Sequence Synthetic oligonucleotide
ccgacaauauacaaguuua

SEQ ID NO: 292 - RNA, 19 nt, Artificial Sequence
  Description of Artificial Sequence Synthetic oligonucleotide
ggaaaccacuuggaugacu

SEQ ID NO: 293 - RNA, 19 nt, Artificial Sequence
  Description of Artificial Sequence Synthetic oligonucleotide
cgagacaacccagaucuuu

SEQ ID NO: 294 - RNA, 19 nt, Artificial Sequence
  Description of Artificial Sequence Synthetic oligonucleotide
gcaaguccauagaugauaa

SEQ ID NO: 295 - RNA, 19 nt, Artificial Sequence
  Description of Artificial Sequence Synthetic oligonucleotide
gggcaaaugguccgagguu

SEQ ID NO: 296 - RNA, 19 nt, Artificial Sequence
  Description of Artificial Sequence Synthetic oligonucleotide
ggaagagucugacguuuga

SEQ ID NO: 297 - RNA, 19 nt, Artificial Sequence
  Description of Artificial Sequence Synthetic oligonucleotide
gagcuugcccggauuaauu

SEQ ID NO: 298 - RNA, 19 nt, Artificial Sequence
  Description of Artificial Sequence Synthetic oligonucleotide
ugauuuagcuugccacuca

SEQ ID NO: 299 - RNA, 19 nt, Artificial Sequence
  Description of Artificial Sequence Synthetic oligonucleotide
agacaaccguucacuguua

SEQ ID NO: 300 - RNA, 19 nt, Artificial Sequence
  Description of Artificial Sequence Synthetic oligonucleotide
gaaguuagcugugcacaua

SEQ ID NO: 301 - RNA, 19 nt, Artificial Sequence
  Description of Artificial Sequence Synthetic oligonucleotide
caacaacauuccagcugau

SEQ ID NO: 302 - RNA, 19 nt, Artificial Sequence
  Description of Artificial Sequence Synthetic oligonucleotide
gcucugaugucauuuaucc

SEQ ID NO: 303 - RNA, 19 nt, Artificial Sequence
  Description of Artificial Sequence Synthetic oligonucleotide
gaaagagcauucgaaacga

SEQ ID NO: 304 - RNA, 19 nt, Artificial Sequence
  Description of Artificial Sequence Synthetic oligonucleotide
gagcagguucuuccaauau

SEQ ID NO: 305 - RNA, 19 nt, Artificial Sequence
  Description of Artificial Sequence Synthetic oligonucleotide
gaacugccaugccucgaag

SEQ ID NO: 306 - RNA, 19 nt, Artificial Sequence
  Description of Artificial Sequence Synthetic oligonucleotide
gcuaauugcgagaagcuuu

SEQ ID NO: 307 - RNA, 19 nt, Artificial Sequence
  Description of Artificial Sequence Synthetic oligonucleotide
gaacuuaaauggacuggag

SEQ ID NO: 308 - RNA, 19 nt, Artificial Sequence
  Description of Artificial Sequence Synthetic oligonucleotide
guugaaagggauccacaua

SEQ ID NO: 309 - RNA, 19 nt, Artificial Sequence
  Description of Artificial Sequence Synthetic oligonucleotide
ucagcgagcguaaauauga

SEQ ID NO: 310 - RNA, 19 nt, Artificial Sequence
  Description of Artificial Sequence Synthetic oligonucleotide
aaucauccacucacaauaa

SEQ ID NO: 311 - RNA, 19 nt, Artificial Sequence
  Description of Artificial Sequence Synthetic oligonucleotide
aaagaggcccaaauauuga

SEQ ID NO: 312 - RNA, 19 nt, Artificial Sequence
  Description of Artificial Sequence Synthetic oligonucleotide
gauaucgcgucaugauuaa

SEQ ID NO: 313 - RNA, 19 nt, Artificial Sequence
  Description of Artificial Sequence Synthetic oligonucleotide
caaugcagaucacucauga

SEQ ID NO: 314 - RNA, 19 nt, Artificial Sequence
  Description of Artificial Sequence Synthetic oligonucleotide
ucacaucacucgucagaac

SEQ ID NO: 315 - RNA, 19 nt, Artificial Sequence
  Description of Artificial Sequence Synthetic oligonucleotide
gagauacggucgggacuca

SEQ ID NO: 316 - RNA, 19 nt, Artificial Sequence
  Description of Artificial Sequence Synthetic oligonucleotide
caugaaagauuaaugcuag

SEQ ID NO: 317 - RNA, 19 nt, Artificial Sequence
  Description of Artificial Sequence Synthetic oligonucleotide
guacaugugugcagacaaa

SEQ ID NO: 318 - RNA, 19 nt, Artificial Sequence
  Description of Artificial Sequence Synthetic oligonucleotide
gacacaagcuccugaaaug

SEQ ID NO: 319 - RNA, 19 nt, Artificial Sequence
  Description of Artificial Sequence Synthetic oligonucleotide
guacaugggauacaaauuu

SEQ ID NO: 320 - RNA, 19 nt, Artificial Sequence
  Description of Artificial Sequence Synthetic oligonucleotide
ccaugaagauguuugaaua

SEQ ID NO: 321 - RNA, 19 nt, Artificial Sequence
  Description of Artificial Sequence Synthetic oligonucleotide
guauauggcugggauuuga

SEQ ID NO: 322 - RNA, 19 nt, Artificial Sequence
  Description of Artificial Sequence Synthetic oligonucleotide
gaacaaauauucggugcuu

SEQ ID NO: 323 - RNA, 19 nt, Artificial Sequence
  Description of Artificial Sequence Synthetic oligonucleotide
cuucuaagaccacgagaga

SEQ ID NO: 324 - RNA, 19 nt, Artificial Sequence
  Description of Artificial Sequence Synthetic oligonucleotide
ggagaugauuguucgggca

SEQ ID NO: 325 - RNA, 19 nt, Artificial Sequence
  Description of Artificial Sequence Synthetic oligonucleotide
gacagaccguguaauguuu

SEQ ID NO: 326 - RNA, 19 nt, Artificial Sequence
  Description of Artificial Sequence Synthetic oligonucleotide
ccuaugagaacgagacuaa

SEQ ID NO: 327 - RNA, 19 nt, Artificial Sequence
  Description of Artificial Sequence Synthetic oligonucleotide
gaagugcgccuacaucuag

SEQ ID NO: 328 - RNA, 19 nt, Artificial Sequence
  Description of Artificial Sequence Synthetic oligonucleotide
ggacagaccguguaauguu

SEQ ID NO: 329 - RNA, 19 nt, Artificial Sequence
  Description of Artificial Sequence Synthetic oligonucleotide
ccagacagauugcugauaa

SEQ ID NO: 330 - RNA, 19 nt, Artificial Sequence
  Description of Artificial Sequence Synthetic oligonucleotide
gaaaccagaaggauauuua

SEQ ID NO: 331 - RNA, 19 nt, Artificial Sequence
  Description of Artificial Sequence Synthetic oligonucleotide
gaacugaccacccuuagaa

SEQ ID NO: 332 - RNA, 19 nt, Artificial Sequence
  Description of Artificial Sequence Synthetic oligonucleotide
gaaccgaugugaauuacuu

SEQ ID NO: 333 - RNA, 19 nt, Artificial Sequence
  Description of Artificial Sequence Synthetic oligonucleotide
gcacagagguugucuucua

SEQ ID NO: 334 - RNA, 19 nt, Artificial Sequence
  Description of Artificial Sequence Synthetic oligonucleotide
ggacuucuauccggcguca

SEQ ID NO: 335 - RNA, 19 nt, Artificial Sequence
  Description of Artificial Sequence Synthetic oligonucleotide
gaaugugaaugguuaccua

SEQ ID NO: 336 - RNA, 19 nt, Artificial Sequence
  Description of Artificial Sequence Synthetic oligonucleotide
gcccuuacgucccuacgaa

SEQ ID NO: 337 - RNA, 19 nt, Artificial Sequence
  Description of Artificial Sequence Synthetic oligonucleotide
gacagaaaccgagucagua

SEQ ID NO: 338 - RNA, 19 nt, Artificial Sequence
  Description of Artificial Sequence Synthetic oligonucleotide
ugugagagcuaccuaguca

SEQ ID NO: 339 - RNA, 19 nt, Artificial Sequence
  Description of Artificial Sequence Synthetic oligonucleotide
gaaaccgagucaguagugu

SEQ ID NO: 340 - RNA, 19 nt, Artificial Sequence
  Description of Artificial Sequence Synthetic oligonucleotide
cgcuaaacauggagaagua

SEQ ID NO: 341 - RNA, 19 nt, Artificial Sequence
  Description of Artificial Sequence Synthetic oligonucleotide
gagagaguauguaauauag

SEQ ID NO: 342 - RNA, 19 nt, Artificial Sequence
  Description of Artificial Sequence Synthetic oligonucleotide
gaccaccacuauuccguaa

SEQ ID NO: 343 - RNA, 19 nt, Artificial Sequence
  Description of Artificial Sequence Synthetic oligonucleotide
gcuaugccaucaguaauaa

SEQ ID NO: 344 - RNA, 19 nt, Artificial Sequence
  Description of Artificial Sequence Synthetic oligonucleotide
ucaaaacgccgaagacuua

SEQ ID NO: 345 - RNA, 19 nt, Artificial Sequence
  Description of Artificial Sequence Synthetic oligonucleotide
guaacaagcucacgcaguu

SEQ ID NO: 346 - RNA, 19 nt, Artificial Sequence
  Description of Artificial Sequence Synthetic oligonucleotide
ggaaauauguacuacgaaa

SEQ ID NO: 347 - RNA, 19 nt, Artificial Sequence
  Description of Artificial Sequence Synthetic oligonucleotide
ccacaaagcagugaauuua

SEQ ID NO: 348 - RNA, 19 nt, Artificial Sequence
  Description of Artificial Sequence Synthetic oligonucleotide
gaaggaaacugaauucaaa

SEQ ID NO: 349 - RNA, 19 nt, Artificial Sequence
  Description of Artificial Sequence Synthetic oligonucleotide
guaagaagugcaaauuaug

SEQ ID NO: 350 - RNA, 19 nt, Artificial Sequence
  Description of Artificial Sequence Synthetic oligonucleotide
ucggagggaucaauaauau

SEQ ID NO: 351 - RNA, 19 nt, Artificial Sequence
  Description of Artificial Sequence Synthetic oligonucleotide
gaagugacucauuguggaa

SEQ ID NO: 352 - RNA, 19 nt, Artificial Sequence
  Description of Artificial Sequence Synthetic oligonucleotide
gaacaguagcgcaguauuu

SEQ ID NO: 353 - RNA, 19 nt, Artificial Sequence
  Description of Artificial Sequence Synthetic oligonucleotide
uacuauggcucauucguua

SEQ ID NO: 354 - RNA, 19 nt, Artificial Sequence
  Description of Artificial Sequence Synthetic oligonucleotide
aaggaucucucucacuaaa

SEQ ID NO: 355 - RNA, 19 nt, Artificial Sequence
  Description of Artificial Sequence Synthetic oligonucleotide
gaagaucggcuugaaauua

SEQ ID NO: 356 - RNA, 19 nt, Artificial Sequence
  Description of Artificial Sequence Synthetic oligonucleotide
gaagugccgauuguaauua

SEQ ID NO: 357 - RNA, 19 nt, Artificial Sequence
  Description of Artificial Sequence Synthetic oligonucleotide
gugaacaucuuaaugacua

SEQ ID NO: 358 - RNA, 19 nt, Artificial Sequence
  Description of Artificial Sequence Synthetic oligonucleotide
gcaguuagcuaaauuacaa

SEQ ID NO: 359 - RNA, 19 nt, Artificial Sequence
  Description of Artificial Sequence Synthetic oligonucleotide
gaacgagccaaguccuuaa

SEQ ID NO: 360 - RNA, 19 nt, Artificial Sequence
  Description of Artificial Sequence Synthetic oligonucleotide
agucuaaacucauccuuaa

SEQ ID NO: 361 - RNA, 19 nt, Artificial Sequence
  Description of Artificial Sequence Synthetic oligonucleotide
gcuaagcagugaaagugaa

SEQ ID NO: 362 - RNA, 19 nt, Artificial Sequence
  Description of Artificial Sequence Synthetic oligonucleotide
gaauuugcucgcagacaua

SEQ ID NO: 363 - RNA, 19 nt, Artificial Sequence
  Description of Artificial Sequence Synthetic oligonucleotide
gcucaaaggcuuacaugua

SEQ ID NO: 364 - RNA, 19 nt, Artificial Sequence
  Description of Artificial Sequence Synthetic oligonucleotide
ggaaauggccagugcaguu

SEQ ID NO: 365 - RNA, 19 nt, Artificial Sequence
  Description of Artificial Sequence Synthetic oligonucleotide
gcgagagucuauagccaaa

SEQ ID NO: 366 - RNA, 19 nt, Artificial Sequence
  Description of Artificial Sequence Synthetic oligonucleotide
ucggaaagaugcuaugauc

SEQ ID NO: 367 - RNA, 19 nt, Artificial Sequence
  Description of Artificial Sequence Synthetic oligonucleotide
ugucaauagucuuggagua

SEQ ID NO: 368 - RNA, 19 nt, Artificial Sequence
  Description of Artificial Sequence Synthetic oligonucleotide
aaacacggauuuaaccuuc

SEQ ID NO: 369 - RNA, 19 nt, Artificial Sequence
  Description of Artificial Sequence Synthetic oligonucleotide
gggaauauguggcaaucaa

SEQ ID NO: 370 - RNA, 19 nt, Artificial Sequence
  Description of Artificial Sequence Synthetic oligonucleotide
gaauaugacuuccggaaug

SEQ ID NO: 371 - RNA, 19 nt, Artificial Sequence
  Description of Artificial Sequence Synthetic oligonucleotide
gagaaugccgcuuaagaaa

SEQ ID NO: 372 - RNA, 19 nt, Artificial Sequence
  Description of Artificial Sequence Synthetic oligonucleotide
gaacaaacccuauaucuac

SEQ ID NO: 373 - RNA, 19 nt, Artificial Sequence
  Description of Artificial Sequence Synthetic oligonucleotide
ggacagugguaugaaagaa

SEQ ID NO: 374 - RNA, 19 nt, Artificial Sequence
  Description of Artificial Sequence Synthetic oligonucleotide
caacagcguaauucagauu

SEQ ID NO: 375 - RNA, 19 nt, Artificial Sequence
  Description of Artificial Sequence Synthetic oligonucleotide
ccacguggcucacagaugu

SEQ ID NO: 376 - RNA, 19 nt, Artificial Sequence
  Description of Artificial Sequence Synthetic oligonucleotide
cgaagacauugaaagcauu

SEQ ID NO: 377 - RNA, 19 nt, Artificial Sequence
  Description of Artificial Sequence Synthetic oligonucleotide
gaucagagguuacaaucaa

SEQ ID NO: 378 - RNA, 19 nt, Artificial Sequence
  Description of Artificial Sequence Synthetic oligonucleotide
gaaacacgguagaggaggu

SEQ ID NO: 379 - RNA, 19 nt, Artificial Sequence
  Description of Artificial Sequence Synthetic oligonucleotide
aauguuagcggaugaguuu

SEQ ID NO: 380 - RNA, 19 nt, Artificial Sequence
  Description of Artificial Sequence Synthetic oligonucleotide
uaacuauguucggaaagua

SEQ ID NO: 381 - RNA, 19 nt, Artificial Sequence
  Description of Artificial Sequence Synthetic oligonucleotide
gcagauagacuuugcuguu

SEQ ID NO: 382 - RNA, 19 nt, Artificial Sequence
  Description of Artificial Sequence Synthetic oligonucleotide
gaucuacccugcucacgau

SEQ ID NO: 383 - RNA, 19 nt, Artificial Sequence
  Description of Artificial Sequence Synthetic oligonucleotide
caggcugacuuacacauug

SEQ ID NO: 384 - RNA, 19 nt, Artificial Sequence
  Description of Artificial Sequence Synthetic oligonucleotide
ucugucaucucccgccuua

SEQ ID NO: 385 - RNA, 19 nt, Artificial Sequence
  Description of Artificial Sequence Synthetic oligonucleotide
cggcagcuuggauuaauua

SEQ ID NO: 386 - RNA, 19 nt, Artificial Sequence
  Description of Artificial Sequence Synthetic oligonucleotide
uacaaugacugcauguuaa

SEQ ID NO: 387 - RNA, 19 nt, Artificial Sequence
  Description of Artificial Sequence Synthetic oligonucleotide
ccaagcaguuguuugacua

SEQ ID NO: 388 - RNA, 19 nt, Artificial Sequence
  Description of Artificial Sequence Synthetic oligonucleotide
gaucagagaugguugugua

SEQ ID NO: 389 - RNA, 19 nt, Artificial Sequence
  Description of Artificial Sequence Synthetic oligonucleotide
ggagugagccagcuucuuu

SEQ ID NO: 390 - RNA, 19 nt, Artificial Sequence
  Description of Artificial Sequence Synthetic oligonucleotide
acucugggcuuaauuagaa

SEQ ID NO: 391 - RNA, 19 nt, Artificial Sequence
  Description of Artificial Sequence Synthetic oligonucleotide
ggagauaguauuuggaaug

SEQ ID NO: 392 - RNA, 19 nt, Artificial Sequence
  Description of Artificial Sequence Synthetic oligonucleotide
gccaguuugugguugcuaa

SEQ ID NO: 393 - RNA, 19 nt, Artificial Sequence
  Description of Artificial Sequence Synthetic oligonucleotide
caacaaggccacacucgaa

SEQ ID NO: 394 - RNA, 19 nt, Artificial Sequence
  Description of Artificial Sequence Synthetic oligonucleotide
caaaauccgugcugaaaau

SEQ ID NO: 395 - RNA, 19 nt, Artificial Sequence
  Description of Artificial Sequence Synthetic oligonucleotide
caacacgucuuccguuucu

SEQ ID NO: 396 - RNA, 19 nt, Artificial Sequence
  Description of Artificial Sequence Synthetic oligonucleotide
caugcgggcucucuucugu

SEQ ID NO: 397 - RNA, 19 nt, Artificial Sequence
  Description of Artificial Sequence Synthetic oligonucleotide
ggauagccgcauugaggua

SEQ ID NO: 398 - RNA, 19 nt, Artificial Sequence
  Description of Artificial Sequence Synthetic oligonucleotide
guacaggauuuauagggag

SEQ ID NO: 399 - RNA, 19 nt, Artificial Sequence
  Description of Artificial Sequence Synthetic oligonucleotide
ccaccgcgcucaggaguau

SEQ ID NO: 400 - RNA, 19 nt, Artificial Sequence
  Description of Artificial Sequence Synthetic oligonucleotide
gagacaaucuacuaugagc

SEQ ID NO: 401 - RNA, 19 nt, Artificial Sequence
  Description of Artificial Sequence Synthetic oligonucleotide
gaacagugcaaacagcaau

SEQ ID NO: 402 - RNA, 19 nt, Artificial Sequence
  Description of Artificial Sequence Synthetic oligonucleotide
uaaauccucugcaacaauu

SEQ ID NO: 403 - RNA, 19 nt, Artificial Sequence
  Description of Artificial Sequence Synthetic oligonucleotide
gauggaaaguuauuaugcu

SEQ ID NO: 404 - RNA, 19 nt, Artificial Sequence
  Description of Artificial Sequence Synthetic oligonucleotide
gauugcacauccuauugua

SEQ ID NO: 405 - RNA, 19 nt, Artificial Sequence
  Description of Artificial Sequence Synthetic oligonucleotide
ucacgugccuauuuggugu

SEQ ID NO: 406 - RNA, 19 nt, Artificial Sequence
  Description of Artificial Sequence Synthetic oligonucleotide
gagccaagcuuguguguaa

SEQ ID NO: 407 - RNA, 19 nt, Artificial Sequence
  Description of Artificial Sequence Synthetic oligonucleotide
ggaaauagcuuauguggga

SEQ ID NO: 408 - RNA, 19 nt, Artificial Sequence
  Description of Artificial Sequence Synthetic oligonucleotide
gcacuucagaguugaguca

SEQ ID NO: 409 - RNA, 19 nt, Artificial Sequence
  Description of Artificial Sequence Synthetic oligonucleotide
gaaacggugcagaauauuu

SEQ ID NO: 410 - RNA, 19 nt, Artificial Sequence
  Description of Artificial Sequence Synthetic oligonucleotide
gaacugguguguauccuaa

SEQ ID NO: 411 - RNA, 19 nt, Artificial Sequence
  Description of Artificial Sequence Synthetic oligonucleotide
ugaaggugcuguauauauu

SEQ ID NO: 412 - RNA, 19 nt, Artificial Sequence
  Description of Artificial Sequence Synthetic oligonucleotide
gaacaggaaucccaaucag

SEQ ID NO: 413 - RNA, 19 nt, Artificial Sequence
  Description of Artificial Sequence Synthetic oligonucleotide
guaaaggugucuacuguuu

SEQ ID NO: 414 - RNA, 19 nt, Artificial Sequence
  Description of Artificial Sequence Synthetic oligonucleotide
gagcaaggcuuagaucaca

SEQ ID NO: 415 - RNA, 19 nt, Artificial Sequence
  Description of Artificial Sequence Synthetic oligonucleotide
guuaguggaucaucagaua

SEQ ID NO: 416 - RNA, 19 nt, Artificial Sequence
  Description of Artificial Sequence Synthetic oligonucleotide
gcacauugguggaacauuc

SEQ ID NO: 417 - RNA, 19 nt, Artificial Sequence
  Description of Artificial Sequence Synthetic oligonucleotide
gaagggaccgguuuuauaa

SEQ ID NO: 418 - RNA, 19 nt, Artificial Sequence
  Description of Artificial Sequence Synthetic oligonucleotide
gcucaaagaugccuuaaua

SEQ ID NO: 419 - RNA, 19 nt, Artificial Sequence
  Description of Artificial Sequence Synthetic oligonucleotide
gaauuccgagucuaaagua

SEQ ID NO: 420 - RNA, 19 nt, Artificial Sequence
  Description of Artificial Sequence Synthetic oligonucleotide
gcucgucuguuacgccaaa

SEQ ID NO: 421 - RNA, 19 nt, Artificial Sequence
  Description of Artificial Sequence Synthetic oligonucleotide
gaagaucccuacugugugg

SEQ ID NO: 422 - RNA, 19 nt, Artificial Sequence
  Description of Artificial Sequence Synthetic oligonucleotide
cgagggagcuguucuauga

SEQ ID NO: 423 - RNA, 19 nt, Artificial Sequence
  Description of Artificial Sequence Synthetic oligonucleotide
gcaaguauguguccuuuga

SEQ ID NO: 424 - RNA, 19 nt, Artificial Sequence
  Description of Artificial Sequence Synthetic oligonucleotide
cgacaagggugcucacuac

SEQ ID NO: 425 - RNA, 19 nt, Artificial Sequence
  Description of Artificial Sequence Synthetic oligonucleotide
guuacugaaagcacgauaa

SEQ ID NO: 426 - RNA, 19 nt, Artificial Sequence
  Description of Artificial Sequence Synthetic oligonucleotide
gaaagcaucuucacaaaag

SEQ ID NO: 427 - RNA, 19 nt, Artificial Sequence
  Description of Artificial Sequence Synthetic oligonucleotide
guaaggauggucaguauga

SEQ ID NO: 428 - RNA, 19 nt, Artificial Sequence
  Description of Artificial Sequence Synthetic oligonucleotide
caacagggaacacugauga

SEQ ID NO: 429 - RNA, 19 nt, Artificial Sequence
  Description of Artificial Sequence Synthetic oligonucleotide
cgugaagccuccaauaugg

SEQ ID NO: 430 - RNA, 19 nt, Artificial Sequence
  Description of Artificial Sequence Synthetic oligonucleotide
gaauugggacgauguguug

SEQ ID NO: 431 - RNA, 19 nt, Artificial Sequence
  Description of Artificial Sequence Synthetic oligonucleotide
caggauguaucguguguau

SEQ ID NO: 432 - RNA, 19 nt, Artificial Sequence
  Description of Artificial Sequence Synthetic oligonucleotide
ugccacagaucaacuacaa

SEQ ID NO: 433 - RNA, 19 nt, Artificial Sequence
  Description of Artificial Sequence Synthetic oligonucleotide
aagaguagaagaauuggua

SEQ ID NO: 434 - RNA, 19 nt, Artificial Sequence
  Description of Artificial Sequence Synthetic oligonucleotide
ccaaaucucgggaaaguaa

SEQ ID NO: 435 - RNA, 19 nt, Artificial Sequence
  Description of Artificial Sequence Synthetic oligonucleotide
cgucuaaacuuaacauugc

SEQ ID NO: 436 - RNA, 19 nt, Artificial Sequence
  Description of Artificial Sequence Synthetic oligonucleotide
ggugcugacugauaacuua

SEQ ID NO: 437 - RNA, 19 nt, Artificial Sequence
  Description of Artificial Sequence Synthetic oligonucleotide
caacaucacucggauaguc

SEQ ID NO: 438 - RNA, 19 nt, Artificial Sequence
  Description of Artificial Sequence Synthetic oligonucleotide
ggaagggucguucgcauug

SEQ ID NO: 439 - RNA, 19 nt, Artificial Sequence
  Description of Artificial Sequence Synthetic oligonucleotide
ggaauaugguggacuauca

SEQ ID NO: 440 - RNA, 19 nt, Artificial Sequence
  Description of Artificial Sequence Synthetic oligonucleotide
uaagaagugucucgucauu

SEQ ID NO: 441 - RNA, 19 nt, Artificial Sequence
  Description of Artificial Sequence Synthetic oligonucleotide
agacaaagcuauacccucg

SEQ ID NO: 442 - RNA, 19 nt, Artificial Sequence
  Description of Artificial Sequence Synthetic oligonucleotide
caaagaaccacccucaaua

SEQ ID NO: 443 - RNA, 19 nt, Artificial Sequence
  Description of Artificial Sequence Synthetic oligonucleotide
ggaauaagucauuacaagu

SEQ ID NO: 444 - RNA, 19 nt, Artificial Sequence
  Description of Artificial Sequence Synthetic oligonucleotide
uaacugcgcugguuuguug

SEQ ID NO: 445 - RNA, 19 nt, Artificial Sequence
  Description of Artificial Sequence Synthetic oligonucleotide
ugacaaacccauaagcuua

SEQ ID NO: 446 - RNA, 19 nt, Artificial Sequence
  Description of Artificial Sequence Synthetic oligonucleotide
cagcauacaccuagcuaga

SEQ ID NO: 447 - RNA, 19 nt, Artificial Sequence
  Description of Artificial Sequence Synthetic oligonucleotide
gaagaugcuugggcaauua

SEQ ID NO: 448 - RNA, 19 nt, Artificial Sequence
  Description of Artificial Sequence Synthetic oligonucleotide
guacugagauuguagccuu

SEQ ID NO: 449 - RNA, 19 nt, Artificial Sequence
  Description of Artificial Sequence Synthetic oligonucleotide
gaacagagcgucagcauau

SEQ ID NO: 450 - RNA, 19 nt, Artificial Sequence
  Description of Artificial Sequence Synthetic oligonucleotide
gcaaucgaauuacaacuac

SEQ ID NO: 451 - RNA, 19 nt, Artificial Sequence
  Description of Artificial Sequence Synthetic oligonucleotide
caaagacgauaguacccau

SEQ ID NO: 452 - RNA, 19 nt, Artificial Sequence
  Description of Artificial Sequence Synthetic oligonucleotide
gcaguugccuugaauagaa

SEQ ID NO: 453 - RNA, 19 nt, Artificial Sequence
  Description of Artificial Sequence Synthetic oligonucleotide
ggaaauaauccaagugcca

SEQ ID NO: 454 - RNA, 19 nt, Artificial Sequence
  Description of Artificial Sequence Synthetic oligonucleotide
gagaaccgcugugcuauca

SEQ ID NO: 455 - RNA, 19 nt, Artificial Sequence
  Description of Artificial Sequence Synthetic oligonucleotide
ggccaucucugaaggguau

SEQ ID NO: 456 - RNA, 19 nt, Artificial Sequence
  Description of Artificial Sequence Synthetic oligonucleotide
gaaacugccuacaagauac

SEQ ID NO: 457 - RNA, 19 nt, Artificial Sequence
  Description of Artificial Sequence Synthetic oligonucleotide
gacagccgauucuuuacaa

SEQ ID NO: 458 - RNA, 19 nt, Artificial Sequence
  Description of Artificial Sequence Synthetic oligonucleotide
gcuauuugaaccacuuaua

SEQ ID NO: 459 - RNA, 19 nt, Artificial Sequence
  Description of Artificial Sequence Synthetic oligonucleotide
cgaagaacauuguggcuga

SEQ ID NO: 460 - RNA, 19 nt, Artificial Sequence
  Description of Artificial Sequence Synthetic oligonucleotide
gcucugagaaguccaauac

SEQ ID NO: 461 - RNA, 19 nt, Artificial Sequence
  Description of Artificial Sequence Synthetic oligonucleotide
cugcaaagggcgagugcuu

SEQ ID NO: 462 - RNA, 19 nt, Artificial Sequence
  Description of Artificial Sequence Synthetic oligonucleotide
gcgaugcuguuaagaaagg

SEQ ID NO: 463 - RNA, 19 nt, Artificial Sequence
  Description of Artificial Sequence Synthetic oligonucleotide
cugcacagcuacacagcga

SEQ ID NO: 464 - RNA, 19 nt, Artificial Sequence
  Description of Artificial Sequence Synthetic oligonucleotide
cguccuugccugaaacaaa

SEQ ID NO: 465 - RNA, 19 nt, Artificial Sequence
  Description of Artificial Sequence Synthetic oligonucleotide
cagguaggcuuauuuauca

SEQ ID NO: 466 - RNA, 19 nt, Artificial Sequence
  Description of Artificial Sequence Synthetic oligonucleotide
gaagccaagcaacuuaaug

SEQ ID NO: 467 - RNA, 19 nt, Artificial Sequence
  Description of Artificial Sequence Synthetic oligonucleotide
gaaagaacccaaucucaca

SEQ ID NO: 468 - RNA, 19 nt, Artificial Sequence
  Description of Artificial Sequence Synthetic oligonucleotide
guaugagaaggcaacauuu

SEQ ID NO: 469 - RNA, 19 nt, Artificial Sequence
  Description of Artificial Sequence Synthetic oligonucleotide
ggaaugaauugcuggacgu

SEQ ID NO: 470 - RNA, 19 nt, Artificial Sequence
  Description of Artificial Sequence Synthetic oligonucleotide
ccaaagcuccacacggaua

SEQ ID NO: 471 - RNA, 19 nt, Artificial Sequence
  Description of Artificial Sequence Synthetic oligonucleotide
ggacuugaguuucaugcuu

SEQ ID NO: 472 - RNA, 19 nt, Artificial Sequence
  Description of Artificial Sequence Synthetic oligonucleotide
ccaacaagcuaacccauug

SEQ ID NO: 473 - RNA, 19 nt, Artificial Sequence
  Description of Artificial Sequence Synthetic oligonucleotide
gggcagagcucgaugugga

SEQ ID NO: 474 - RNA, 19 nt, Artificial Sequence
  Description of Artificial Sequence Synthetic oligonucleotide
gaacaagcauucucuguga

SEQ ID NO: 475 - RNA, 19 nt, Artificial Sequence
  Description of Artificial Sequence Synthetic oligonucleotide
ugacaaggaucgcaccuau

SEQ ID NO: 476 - RNA, 19 nt, Artificial Sequence
  Description of Artificial Sequence Synthetic oligonucleotide
gaaccaugacgguaccuuu

SEQ ID NO: 477 - RNA, 19 nt, Artificial Sequence
  Description of Artificial Sequence Synthetic oligonucleotide
ccaaagauacuucguuauu

SEQ ID NO: 478 - RNA, 19 nt, Artificial Sequence
  Description of Artificial Sequence Synthetic oligonucleotide
gaaagugcauggaacuauu

SEQ ID NO: 479 - RNA, 19 nt, Artificial Sequence
  Description of Artificial Sequence Synthetic oligonucleotide
gagcagaaugggcugauau

SEQ ID NO: 480 - RNA, 19 nt, Artificial Sequence
  Description of Artificial Sequence Synthetic oligonucleotide
gaaaaugacucaccaacaa

SEQ ID NO: 481 - RNA, 19 nt, Artificial Sequence
  Description of Artificial Sequence Synthetic oligonucleotide
ucauguggaucaucaggaa

SEQ ID NO: 482 - RNA, 19 nt, Artificial Sequence
  Description of Artificial Sequence Synthetic oligonucleotide
guacuugguuccaucugau

SEQ ID NO: 483 - RNA, 19 nt, Artificial Sequence
  Description of Artificial Sequence Synthetic oligonucleotide
uaacuaugggacagcuuua

SEQ ID NO: 484 - RNA, 19 nt, Artificial Sequence
  Description of Artificial Sequence Synthetic oligonucleotide
ggucucaggcucucagauu

SEQ ID NO: 485 - RNA, 19 nt, Artificial Sequence
  Description of Artificial Sequence Synthetic oligonucleotide
gcuaacagauggacugaua

SEQ ID NO: 486 - RNA, 19 nt, Artificial Sequence
  Description of Artificial Sequence Synthetic oligonucleotide
aaagagaacucgcaugaug

SEQ ID NO: 487 - RNA, 19 nt, Artificial Sequence
  Description of Artificial Sequence Synthetic oligonucleotide
gagaaaggcauuacugcua

SEQ ID NO: 488 - RNA, 19 nt, Artificial Sequence
  Description of Artificial Sequence Synthetic oligonucleotide
gaucggaacuucuaauuau

SEQ ID NO: 489 - RNA, 19 nt, Artificial Sequence
  Description of Artificial Sequence Synthetic oligonucleotide
gaaaguuuaucacccuaua

SEQ ID NO: 490 - RNA, 19 nt, Artificial Sequence
  Description of Artificial Sequence Synthetic oligonucleotide
ggaguacaaucagcgcaua

SEQ ID NO: 491 - RNA, 19 nt, Artificial Sequence
  Description of Artificial Sequence Synthetic oligonucleotide
ggacacagcaaauucuugg

SEQ ID NO: 492 - RNA, 19 nt, Artificial Sequence
  Description of Artificial Sequence Synthetic oligonucleotide
gcaccucggcccauuccua

SEQ ID NO: 493 - RNA, 19 nt, Artificial Sequence
  Description of Artificial Sequence Synthetic oligonucleotide
gucaagagguguugccaaa

SEQ ID NO: 494 - RNA, 19 nt, Artificial Sequence
  Description of Artificial Sequence Synthetic oligonucleotide
gauccggaccuaauauaua

SEQ ID NO: 495 - RNA, 19 nt, Artificial Sequence
  Description of Artificial Sequence Synthetic oligonucleotide
ccggaaaguugauccaaga

SEQ ID NO: 496 - RNA, 19 nt, Artificial Sequence
  Description of Artificial Sequence Synthetic oligonucleotide
gaaacaagcaaucuauaca

SEQ ID NO: 497 - RNA, 19 nt, Artificial Sequence
  Description of Artificial Sequence Synthetic oligonucleotide
gcaagcagaucuacaacau

SEQ ID NO: 498 - RNA, 19 nt, Artificial Sequence
  Description of Artificial Sequence Synthetic oligonucleotide
gcucauagguggcaaguac

SEQ ID NO: 499 - RNA, 19 nt, Artificial Sequence
  Description of Artificial Sequence Synthetic oligonucleotide
gcacgaagacaucgauaag

SEQ ID NO: 500 - RNA, 19 nt, Artificial Sequence
  Description of Artificial Sequence Synthetic oligonucleotide
ccacgaugaucuccugcua

SEQ ID NO: 501 - RNA, 19 nt, Artificial Sequence
  Description of Artificial Sequence Synthetic oligonucleotide
gcacaacauucucuacuuc

SEQ ID NO: 502 - RNA, 19 nt, Artificial Sequence
  Description of Artificial Sequence Synthetic oligonucleotide
agaaagagaucaucaagaa

SEQ ID NO: 503 - RNA, 19 nt, Artificial Sequence
  Description of Artificial Sequence Synthetic oligonucleotide
ccauggagcuggucaauga

SEQ ID NO: 504 - RNA, 19 nt, Artificial Sequence
  Description of Artificial Sequence Synthetic oligonucleotide
ccaagcaggucuauuucua

SEQ ID NO: 505 - RNA, 19 nt, Artificial Sequence
  Description of Artificial Sequence Synthetic oligonucleotide
gcacacagaaucuauggua

SEQ ID NO: 506 - RNA, 19 nt, Artificial Sequence
  Description of Artificial Sequence Synthetic oligonucleotide
gauucacccucauggcuua

SEQ ID NO: 507 - RNA, 19 nt, Artificial Sequence
  Description of Artificial Sequence Synthetic oligonucleotide
gaacaacugaagcgguuug

SEQ ID NO: 508 - RNA, 19 nt, Artificial Sequence
  Description of Artificial Sequence Synthetic oligonucleotide
aauacaaacuggcagagaa

SEQ ID NO: 509 - RNA, 19 nt, Artificial Sequence
  Description of Artificial Sequence Synthetic oligonucleotide
gacaaggucugcuccaaca

SEQ ID NO: 510 - RNA, 19 nt, Artificial Sequence
  Description of Artificial Sequence Synthetic oligonucleotide
gcucaaggaagccguguua

SEQ ID NO: 511 - RNA, 19 nt, Artificial Sequence
  Description of Artificial Sequence Synthetic oligonucleotide
guacgacgagcauguaauc

SEQ ID NO: 512 - RNA, 19 nt, Artificial Sequence
  Description of Artificial Sequence Synthetic oligonucleotide
gcaucaaugugaaaugugu

SEQ ID NO: 513 - RNA, 19 nt, Artificial Sequence
  Description of Artificial Sequence Synthetic oligonucleotide
guaauagcaugguuugcaa

SEQ ID NO: 514 - RNA, 19 nt, Artificial Sequence
  Description of Artificial Sequence Synthetic oligonucleotide
cauuagaguauguccguau

SEQ ID NO: 515 - RNA, 19 nt, Artificial Sequence
  Description of Artificial Sequence Synthetic oligonucleotide
ggacauguuacccgaugaa

SEQ ID NO: 516 - RNA, 19 nt, Artificial Sequence
  Description of Artificial Sequence Synthetic oligonucleotide
ucaguggacuuacaguuug

SEQ ID NO: 517 - RNA, 19 nt, Artificial Sequence
  Description of Artificial Sequence Synthetic oligonucleotide
ggagaaacccagcacauuu

SEQ ID NO: 518 - RNA, 19 nt, Artificial Sequence
  Description of Artificial Sequence Synthetic oligonucleotide
cgggaugucuuccagcgua

SEQ ID NO: 519 - RNA, 19 nt, Artificial Sequence
  Description of Artificial Sequence Synthetic oligonucleotide
gaucaaguccgcagguuca

SEQ ID NO: 520 - RNA, 19 nt, Artificial Sequence
  Description of Artificial Sequence Synthetic oligonucleotide
ggacgcaaucccucaacua

SEQ ID NO: 521 - RNA, 19 nt, Artificial Sequence
  Description of Artificial Sequence Synthetic oligonucleotide
aaacagaacuuccuacuuu

SEQ ID NO: 522 - RNA, 19 nt, Artificial Sequence
  Description of Artificial Sequence Synthetic oligonucleotide
gagaggaagguuacaacua

SEQ ID NO: 523 - RNA, 19 nt, Artificial Sequence
  Description of Artificial Sequence Synthetic oligonucleotide
uuacguggcuguauguuaa

SEQ ID NO: 524 - RNA, 19 nt, Artificial Sequence
  Description of Artificial Sequence Synthetic oligonucleotide
ucaaggaccgcgaggguua

SEQ ID NO: 525 - RNA, 19 nt, Artificial Sequence
  Description of Artificial Sequence Synthetic oligonucleotide
gauccagucuugcauggga

SEQ ID NO: 526 - RNA, 19 nt, Artificial Sequence
  Description of Artificial Sequence Synthetic oligonucleotide
cgaaauccaagcaagcaua

SEQ ID NO: 527 - RNA, 19 nt, Artificial Sequence
  Description of Artificial Sequence Synthetic oligonucleotide
gaagaagauccuugacauu

SEQ ID NO: 528 - RNA, 19 nt, Artificial Sequence
  Description of Artificial Sequence Synthetic oligonucleotide
acgaaucacugcaguuuaa

SEQ ID NO: 529 - RNA, 19 nt, Artificial Sequence
  Description of Artificial Sequence Synthetic oligonucleotide
gcgcugcaccggcacauua

SEQ ID NO: 530 - RNA, 19 nt, Artificial Sequence
  Description of Artificial Sequence Synthetic oligonucleotide
uggauaagaugauggaaca

SEQ ID NO: 531 - RNA, 19 nt, Artificial Sequence
  Description of Artificial Sequence Synthetic oligonucleotide
gcgauucuaccacaaguga

SEQ ID NO: 532 - RNA, 19 nt, Artificial Sequence
  Description of Artificial Sequence Synthetic oligonucleotide
ugacagagccaaacaaaug

SEQ ID NO: 533 - RNA, 19 nt, Artificial Sequence
  Description of Artificial Sequence Synthetic oligonucleotide
acucaacugcccugaacua

SEQ ID NO: 534 - RNA, 19 nt, Artificial Sequence
  Description of Artificial Sequence Synthetic oligonucleotide
gaagcagugucguugcaua

SEQ ID NO: 535 - RNA, 19 nt, Artificial Sequence
  Description of Artificial Sequence Synthetic oligonucleotide
gcaaauaccugaagcgaga

SEQ ID NO: 536 - RNA, 19 nt, Artificial Sequence
  Description of Artificial Sequence Synthetic oligonucleotide
gccggcugcugaagggaaa

SEQ ID NO: 537 - RNA, 19 nt, Artificial Sequence
  Description of Artificial Sequence Synthetic oligonucleotide
gcgauaaguuuaagcagau

SEQ ID NO: 538 - RNA, 19 nt, Artificial Sequence
  Description of Artificial Sequence Synthetic oligonucleotide
gaagaauacuaccagauua

SEQ ID NO: 539 - RNA, 19 nt, Artificial Sequence
  Description of Artificial Sequence Synthetic oligonucleotide
gcuucgugauggaguguca

SEQ ID NO: 540 - RNA, 19 nt, Artificial Sequence
  Description of Artificial Sequence Synthetic oligonucleotide
aacgaaggcucgaagauua

SEQ ID NO: 541 - RNA, 19 nt, Artificial Sequence
  Description of Artificial Sequence Synthetic oligonucleotide
gcaccucaaugaaugugua

SEQ ID NO: 542 - RNA, 19 nt, Artificial Sequence
  Description of Artificial Sequence Synthetic oligonucleotide
gugucaaccugucuaucaa

SEQ ID NO: 543 - RNA, 19 nt, Artificial Sequence
  Description of Artificial Sequence Synthetic oligonucleotide
ccuacugccuggugguuag

SEQ ID NO: 544 - RNA, 19 nt, Artificial Sequence
  Description of Artificial Sequence Synthetic oligonucleotide
gaaggaagcugcagggguc

SEQ ID NO: 545 - RNA, 19 nt, Artificial Sequence
  Description of Artificial Sequence Synthetic oligonucleotide
caaaucgaccggugguaaa

SEQ ID NO: 546 - RNA, 19 nt, Artificial Sequence
  Description of Artificial Sequence Synthetic oligonucleotide
gcgcagcuaucggugcuuu

SEQ ID NO: 547 - RNA, 19 nt, Artificial Sequence
  Description of Artificial Sequence Synthetic oligonucleotide
cuacaaaagccgcucgcaa

SEQ ID NO: 548 - RNA, 19 nt, Artificial Sequence
  Description of Artificial Sequence Synthetic oligonucleotide
gcaagugaggccuaucugg

SEQ ID NO: 549 - RNA, 19 nt, Artificial Sequence
  Description of Artificial Sequence Synthetic oligonucleotide
gaagcuagcggaggugauu

SEQ ID NO: 550 - RNA, 19 nt, Artificial Sequence
  Description of Artificial Sequence Synthetic oligonucleotide
agaauccacugcuccgaaa

SEQ ID NO: 551 - RNA, 19 nt, Artificial Sequence
  Description of Artificial Sequence Synthetic oligonucleotide
ggaagaaccuaugaaucuc

SEQ ID NO: 552 - RNA, 19 nt, Artificial Sequence
  Description of Artificial Sequence Synthetic oligonucleotide
gacaagagcaagcgaagug

SEQ ID NO: 553 - RNA, 19 nt, Artificial Sequence
  Description of Artificial Sequence Synthetic oligonucleotide
uaaagaagcuugaaagugu

SEQ ID NO: 554 - RNA, 19 nt, Artificial Sequence
  Description of Artificial Sequence Synthetic oligonucleotide
uggguuaaguugcuugaua

SEQ ID NO: 555 - RNA, 19 nt, Artificial Sequence
  Description of Artificial Sequence Synthetic oligonucleotide
gcucagaaguccucagaua

SEQ ID NO: 556 - RNA, 19 nt, Artificial Sequence
  Description of Artificial Sequence Synthetic oligonucleotide
ccgcugacauauuaauuaa

SEQ ID NO: 557 - RNA, 19 nt, Artificial Sequence
  Description of Artificial Sequence Synthetic oligonucleotide
gagcugaucgcuuuaaaua

SEQ ID NO: 558 - RNA, 19 nt, Artificial Sequence
  Description of Artificial Sequence Synthetic oligonucleotide
gaacucaacuaggguguua

SEQ ID NO: 559 - RNA, 19 nt, Artificial Sequence
  Description of Artificial Sequence Synthetic oligonucleotide
gcaaaguacuagauaacug

SEQ ID NO: 560 - RNA, 19 nt, Artificial Sequence
  Description of Artificial Sequence Synthetic oligonucleotide
cgagaaagcuauggaguga

SEQ ID NO: 561 - RNA, 19 nt, Artificial Sequence
  Description of Artificial Sequence Synthetic oligonucleotide
ugaaagagaucacccaaca

SEQ ID NO: 562 - RNA, 19 nt, Artificial Sequence
  Description of Artificial Sequence Synthetic oligonucleotide
ucacccagauuuauacuua

SEQ ID NO: 563 - RNA, 19 nt, Artificial Sequence
  Description of Artificial Sequence Synthetic oligonucleotide
gaagucgccugguuaaaga

SEQ ID NO: 564 - RNA, 19 nt, Artificial Sequence
  Description of Artificial Sequence Synthetic oligonucleotide
agacagcucuuucgggaua

SEQ ID NO: 565 - RNA, 19 nt, Artificial Sequence
  Description of Artificial Sequence Synthetic oligonucleotide
gcacugacucuuaauauga

SEQ ID NO: 566 - RNA, 19 nt, Artificial Sequence
  Description of Artificial Sequence Synthetic oligonucleotide
gaagauagcuccaguuuuc

SEQ ID NO: 567 - RNA, 19 nt, Artificial Sequence
  Description of Artificial Sequence Synthetic oligonucleotide
gaaaccagcugaucuaaaa

SEQ ID NO: 568 - RNA, 19 nt, Artificial Sequence
  Description of Artificial Sequence Synthetic oligonucleotide
cagaagaggugcuauugga

SEQ ID NO: 569 - RNA, 19 nt, Artificial Sequence
  Description of Artificial Sequence Synthetic oligonucleotide
ccgacaugcuguacuucaa

SEQ ID NO: 570 - RNA, 19 nt, Artificial Sequence
  Description of Artificial Sequence Synthetic oligonucleotide
cagcucaacucgugaacua

SEQ ID NO: 571 - RNA, 19 nt, Artificial Sequence
  Description of Artificial Sequence Synthetic oligonucleotide
ccucuucgaucagacguuu

SEQ ID NO: 572 - RNA, 19 nt, Artificial Sequence
  Description of Artificial Sequence Synthetic oligonucleotide
cuguggagauguuugauua

SEQ ID NO: 573 - RNA, 19 nt, Artificial Sequence
  Description of Artificial Sequence Synthetic oligonucleotide
gaacagcauggguggcuau

SEQ ID NO: 574 - RNA, 19 nt, Artificial Sequence
  Description of Artificial Sequence Synthetic oligonucleotide
cgaccgagaacgacauuua

SEQ ID NO: 575 - RNA, 19 nt, Artificial Sequence
  Description of Artificial Sequence Synthetic oligonucleotide
ccaauaugcagcacagaua

SEQ ID NO: 576 - RNA, 19 nt, Artificial Sequence
  Description of Artificial Sequence Synthetic oligonucleotide
ggauguaugaccacagaua

SEQ ID NO: 577 - RNA, 19 nt, Artificial Sequence
  Description of Artificial Sequence Synthetic oligonucleotide
caccaaggaugcugucuau

SEQ ID NO: 578 - RNA, 19 nt, Artificial Sequence
  Description of Artificial Sequence Synthetic oligonucleotide
gcacggaugucacugagua

SEQ ID NO: 579 - RNA, 19 nt, Artificial Sequence
  Description of Artificial Sequence Synthetic oligonucleotide
ggaugucgaucuuggacga

SEQ ID NO: 580 - RNA, 19 nt, Artificial Sequence
  Description of Artificial Sequence Synthetic oligonucleotide
ggaucgaggcucugagaaa

SEQ ID NO: 581 - RNA, 19 nt, Artificial Sequence
  Description of Artificial Sequence Synthetic oligonucleotide
gacacucucugcugcauuu

SEQ ID NO: 582 - RNA, 19 nt, Artificial Sequence
  Description of Artificial Sequence Synthetic oligonucleotide
ugcagacucucaauuaaua

SEQ ID NO: 583 - RNA, 19 nt, Artificial Sequence
  Description of Artificial Sequence Synthetic oligonucleotide
gauuugccauucuuugaua

SEQ ID NO: 584 - RNA, 19 nt, Artificial Sequence
  Description of Artificial Sequence Synthetic oligonucleotide
ggaaugggauggucuagua

SEQ ID NO: 585 - RNA, 19 nt, Artificial Sequence
  Description of Artificial Sequence Synthetic oligonucleotide
caggaugguuucauguuga

SEQ ID NO: 586 - RNA, 19 nt, Artificial Sequence
  Description of Artificial Sequence Synthetic oligonucleotide
gaugaagacugcucugaaa

SEQ ID NO: 587 - RNA, 19 nt, Artificial Sequence
  Description of Artificial Sequence Synthetic oligonucleotide
gaaauuagaggcuacaaau

SEQ ID NO: 588 - RNA, 19 nt, Artificial Sequence
  Description of Artificial Sequence Synthetic oligonucleotide
cuacauaucaggccaauua

SEQ ID NO: 589 - RNA, 19 nt, Artificial Sequence
  Description of Artificial Sequence Synthetic oligonucleotide
ggaagaggcuaaaugucua

SEQ ID NO: 590 - RNA, 19 nt, Artificial Sequence
  Description of Artificial Sequence Synthetic oligonucleotide
gcaaagaaauggauaucaa

SEQ ID NO: 591 - RNA, 19 nt, Artificial Sequence
  Description of Artificial Sequence Synthetic oligonucleotide
uaacaucaauuguccacuu

SEQ ID NO: 592 - RNA, 19 nt, Artificial Sequence
  Description of Artificial Sequence Synthetic oligonucleotide
gaaauggauaucaaacgua

SEQ ID NO: 593 - RNA, 19 nt, Artificial Sequence
  Description of Artificial Sequence Synthetic oligonucleotide
gagcaaagcuugauaacaa

SEQ ID NO: 594 - RNA, 19 nt, Artificial Sequence
  Description of Artificial Sequence Synthetic oligonucleotide
guacauaacuuugaagaag

SEQ ID NO: 595 - RNA, 19 nt, Artificial Sequence
  Description of Artificial Sequence Synthetic oligonucleotide
caagauaagucaauugaag

SEQ ID NO: 596 - RNA, 19 nt, Artificial Sequence
  Description of Artificial Sequence Synthetic oligonucleotide
ggacuuggcugcuugcauu

SEQ ID NO: 597 - RNA, 19 nt, Artificial Sequence
  Description of Artificial Sequence Synthetic oligonucleotide
gaaauacacccgcugacau

SEQ ID NO: 598 - RNA, 19 nt, Artificial Sequence
  Description of Artificial Sequence Synthetic oligonucleotide
gaaguccgccucgucaguu

SEQ ID NO: 599 - RNA, 19 nt, Artificial Sequence
  Description of Artificial Sequence Synthetic oligonucleotide
ugauaacaccugcggcuuu

SEQ ID NO: 600 - RNA, 19 nt, Artificial Sequence
  Description of Artificial Sequence Synthetic oligonucleotide
guacgaugcugcuaaugag

SEQ ID NO: 601 - RNA, 19 nt, Artificial Sequence
  Description of Artificial Sequence Synthetic oligonucleotide
aaacaggaggugaucgaua

SEQ ID NO: 602 - RNA, 19 nt, Artificial Sequence
  Description of Artificial Sequence Synthetic oligonucleotide
aacaggaggugaucgauaa

SEQ ID NO: 603 - RNA, 19 nt, Artificial Sequence
  Description of Artificial Sequence Synthetic oligonucleotide
ggaagagccaacuguguga

SEQ ID NO: 604 - RNA, 19 nt, Artificial Sequence
  Description of Artificial Sequence Synthetic oligonucleotide
uggagaagcucgaucugaa

SEQ ID NO: 605 - RNA, 19 nt, Artificial Sequence
  Description of Artificial Sequence Synthetic oligonucleotide
gcuguuuacugauaucuca

SEQ ID NO: 606 - RNA, 19 nt, Artificial Sequence
  Description of Artificial Sequence Synthetic oligonucleotide
ucaagacgcugcccggauu

SEQ ID NO: 607 - RNA, 19 nt, Artificial Sequence
  Description of Artificial Sequence Synthetic oligonucleotide
caacuuaucuguacuugua

SEQ ID NO: 608 - RNA, 19 nt, Artificial Sequence
  Description of Artificial Sequence Synthetic oligonucleotide
gaagaaugaugucuguaua

SEQ ID NO: 609 - RNA, 19 nt, Artificial Sequence
  Description of Artificial Sequence Synthetic oligonucleotide
ccaagacaaguuucauaua

SEQ ID NO: 610 - RNA, 19 nt, Artificial Sequence
  Description of Artificial Sequence Synthetic oligonucleotide
ggaguagaguaucacaaug

SEQ ID NO: 611 - RNA, 19 nt, Artificial Sequence
  Description of Artificial Sequence Synthetic oligonucleotide
acacaacaccuaagauuau

SEQ ID NO: 612 - RNA, 19 nt, Artificial Sequence
  Description of Artificial Sequence Synthetic oligonucleotide
gcauauaccggugucguaa

SEQ ID NO: 613 - RNA, 19 nt, Artificial Sequence
  Description of Artificial Sequence Synthetic oligonucleotide
ggaauucacugcgggauuu

SEQ ID NO: 614 - RNA, 19 nt, Artificial Sequence
  Description of Artificial Sequence Synthetic oligonucleotide
ggaaaguguuggacugaca

SEQ ID NO: 615 - RNA, 19 nt, Artificial Sequence
  Description of Artificial Sequence Synthetic oligonucleotide
cagaugaggcuaauacuua

SEQ ID NO: 616 - RNA, 19 nt, Artificial Sequence
  Description of Artificial Sequence Synthetic oligonucleotide
ggaaaucaaagacgaaugu

SEQ ID NO: 617 - RNA, 19 nt, Artificial Sequence
  Description of Artificial Sequence Synthetic oligonucleotide
ggucauaagcccuuucaag

SEQ ID NO: 618 - RNA, 19 nt, Artificial Sequence
  Description of Artificial Sequence Synthetic oligonucleotide
cgacggaccuugugugcuc

SEQ ID NO: 619 - RNA, 19 nt, Artificial Sequence
  Description of Artificial Sequence Synthetic oligonucleotide
cgucaggacuuaccuagag

SEQ ID NO: 620 - RNA, 19 nt, Artificial Sequence
  Description of Artificial Sequence Synthetic oligonucleotide
gcaccgacuucaagacuac

SEQ ID NO: 621 - RNA, 19 nt, Artificial Sequence
  Description of Artificial Sequence Synthetic oligonucleotide
uaaggaaccucuaucauga

SEQ ID NO: 622 - RNA, 19 nt, Artificial Sequence
  Description of Artificial Sequence Synthetic oligonucleotide
ugaaaucacucacauugua

SEQ ID NO: 623 - RNA, 19 nt, Artificial Sequence
  Description of Artificial Sequence Synthetic oligonucleotide
ccacauagcugaucugaaa

SEQ ID NO: 624 - RNA, 19 nt, Artificial Sequence
  Description of Artificial Sequence Synthetic oligonucleotide
gcagguggcuguuaaaucu

SEQ ID NO: 625 - RNA, 19 nt, Artificial Sequence
  Description of Artificial Sequence Synthetic oligonucleotide
gcacagacaugacuacaca

SEQ ID NO: 626 - RNA, 19 nt, Artificial Sequence
  Description of Artificial Sequence Synthetic oligonucleotide
ggaaacuucgagaucauaa

SEQ ID NO: 627 - RNA, 19 nt, Artificial Sequence
  Description of Artificial Sequence Synthetic oligonucleotide
ggacauaccuuauuuguuc

SEQ ID NO: 628 - RNA, 19 nt, Artificial Sequence
  Description of Artificial Sequence Synthetic oligonucleotide
gauaccaugucaagacuga

SEQ ID NO: 629 - RNA, 19 nt, Artificial Sequence
  Description of Artificial Sequence Synthetic oligonucleotide
cccagaauccaaauuguaa

SEQ ID NO: 630 - RNA, 19 nt, Artificial Sequence
  Description of Artificial Sequence Synthetic oligonucleotide
ggaagaaccgcaucuauaa

SEQ ID NO: 631 - RNA, 19 nt, Artificial Sequence
  Description of Artificial Sequence Synthetic oligonucleotide
ugucauagaaggcgucaau

SEQ ID NO: 632 - RNA, 19 nt, Artificial Sequence
  Description of Artificial Sequence Synthetic oligonucleotide
agagagaccuaugauaaua

SEQ ID NO: 633 - RNA, 19 nt, Artificial Sequence
  Description of Artificial Sequence Synthetic oligonucleotide
gagagugagcggacuguau

SEQ ID NO: 634 - RNA, 19 nt, Artificial Sequence
  Description of Artificial Sequence Synthetic oligonucleotide
gcacggagugagucuagua

SEQ ID NO: 635 - RNA, 19 nt, Artificial Sequence
  Description of Artificial Sequence Synthetic oligonucleotide
gaauggaggcggauuagua

SEQ ID NO: 636 - RNA, 19 nt, Artificial Sequence
  Description of Artificial Sequence Synthetic oligonucleotide
gaagaucaguggauuaaua

SEQ ID NO: 637 - RNA, 19 nt, Artificial Sequence
  Description of Artificial Sequence Synthetic oligonucleotide
gaagaucaguggauuaaua

SEQ ID NO: 638 - RNA, 19 nt, Artificial Sequence
  Description of Artificial Sequence Synthetic oligonucleotide
gaauggaggcggauuagua

SEQ ID NO: 639 - RNA, 19 nt, Artificial Sequence
  Description of Artificial Sequence Synthetic oligonucleotide
gcacggagugagucuagua

SEQ ID NO: 640 - RNA, 19 nt, Artificial Sequence
  Description of Artificial Sequence Synthetic oligonucleotide
gagagugagcggacuguau

SEQ ID NO: 641 - RNA, 19 nt, Artificial Sequence
  Description of Artificial Sequence Synthetic oligonucleotide
ccacagggcucagauagca

SEQ ID NO: 642 - RNA, 19 nt, Artificial Sequence
  Description of Artificial Sequence Synthetic oligonucleotide
gcacacaccacuuagcaag

SEQ ID NO: 643 - RNA, 19 nt, Artificial Sequence
  Description of Artificial Sequence Synthetic oligonucleotide
cggagcacguggagagguu

SEQ ID NO: 644 - RNA, 19 nt, Artificial Sequence
  Description of Artificial Sequence Synthetic oligonucleotide
ccuuuaaucucuacgacau

SEQ ID NO: 645 - RNA, 19 nt, Artificial Sequence
  Description of Artificial Sequence Synthetic oligonucleotide
gaggagaucucaccaagca

SEQ ID NO: 646 - RNA, 19 nt, Artificial Sequence
  Description of Artificial Sequence Synthetic oligonucleotide
gaucucaccaagcacauua

SEQ ID NO: 647 - RNA, 19 nt, Artificial Sequence
  Description of Artificial Sequence Synthetic oligonucleotide
gcgaggaggagaaacucaa

SEQ ID NO: 648 - RNA, 19 nt, Artificial Sequence
  Description of Artificial Sequence Synthetic oligonucleotide
gcaacagcauggucauuca

SEQ ID NO: 649 - RNA, 19 nt, Artificial Sequence
  Description of Artificial Sequence Synthetic oligonucleotide
ggacgucaaugcuuacuau

SEQ ID NO: 650 - RNA, 19 nt, Artificial Sequence
  Description of Artificial Sequence Synthetic oligonucleotide
cagcagaucuucuuucgaa

SEQ ID NO: 651 - RNA, 19 nt, Artificial Sequence
  Description of Artificial Sequence Synthetic oligonucleotide
guaaauggacuuccuuaaa

SEQ ID NO: 652 - RNA, 19 nt, Artificial Sequence
  Description of Artificial Sequence Synthetic oligonucleotide
cgacaagaauacaacgaua

SEQ ID NO: 653 - RNA, 19 nt, Artificial Sequence
  Description of Artificial Sequence Synthetic oligonucleotide
gagggaaaguugggacguu

SEQ ID NO: 654 - RNA, 19 nt, Artificial Sequence
  Description of Artificial Sequence Synthetic oligonucleotide
ucaaguacgucuacaaacu

SEQ ID NO: 655 - RNA, 19 nt, Artificial Sequence
  Description of Artificial Sequence Synthetic oligonucleotide
ggaaggaaccguagcuugu

SEQ ID NO: 656 - RNA, 19 nt, Artificial Sequence
  Description of Artificial Sequence Synthetic oligonucleotide
cuacauacaacuagaacca

SEQ ID NO: 657 - RNA, 19 nt, Artificial Sequence
  Description of Artificial Sequence Synthetic oligonucleotide
ggacggaagccuaugagua

SEQ ID NO: 658 - RNA, 19 nt, Artificial Sequence
  Description of Artificial Sequence Synthetic oligonucleotide
cuaaucagccugcuaauuu

SEQ ID NO: 659 - RNA, 19 nt, Artificial Sequence
  Description of Artificial Sequence Synthetic oligonucleotide
gcggucagcuuaucaaagu

SEQ ID NO: 660 - RNA, 19 nt, Artificial Sequence
  Description of Artificial Sequence Synthetic oligonucleotide
ggagagcuuucgcgcugua

SEQ ID NO: 661 - RNA, 19 nt, Artificial Sequence
  Description of Artificial Sequence Synthetic oligonucleotide
gcccaaccgugaccaaagu

SEQ ID NO: 662 - RNA, 19 nt, Artificial Sequence
  Description of Artificial Sequence Synthetic oligonucleotide
ccgcagggaccuagaaaua

SEQ ID NO: 663 - RNA, 19 nt, Artificial Sequence
  Description of Artificial Sequence Synthetic oligonucleotide
gaggcgacaucuagacuaa

SEQ ID NO: 664 - RNA, 19 nt, Artificial Sequence
  Description of Artificial Sequence Synthetic oligonucleotide
gcagcuauauggauaaugu

SEQ ID NO: 665 - RNA, 19 nt, Artificial Sequence
  Description of Artificial Sequence Synthetic oligonucleotide
ucaaacaccaccucacuaa

SEQ ID NO: 666 - RNA, 19 nt, Artificial Sequence
  Description of Artificial Sequence Synthetic oligonucleotide
caacagauugcaccuagua

SEQ ID NO: 667 - RNA, 19 nt, Artificial Sequence
  Description of Artificial Sequence Synthetic oligonucleotide
caaaguuaccuacguguua

SEQ ID NO: 668 - RNA, 19 nt, Artificial Sequence
  Description of Artificial Sequence Synthetic oligonucleotide
ggacagaaagaugcucuaa

SEQ ID NO: 669 - RNA, 19 nt, Artificial Sequence
  Description of Artificial Sequence Synthetic oligonucleotide
uggaugggccaaugaguua

SEQ ID NO: 670 - RNA, 19 nt, Artificial Sequence
  Description of Artificial Sequence Synthetic oligonucleotide
gcuaagcuaaugucuaguu

SEQ ID NO: 671 - RNA, 19 nt, Artificial Sequence
  Description of Artificial Sequence Synthetic oligonucleotide
ucaaacagacguauuacug

SEQ ID NO: 672 - RNA, 19 nt, Artificial Sequence
  Description of Artificial Sequence Synthetic oligonucleotide
cuucggaucuauaguguaa

SEQ ID NO: 673 - RNA, 19 nt, Artificial Sequence
  Description of Artificial Sequence Synthetic oligonucleotide
gaagauggcccuuguacua

SEQ ID NO: 674 - RNA, 19 nt, Artificial Sequence
  Description of Artificial Sequence Synthetic oligonucleotide
gaaaggaaauacuggaaua

SEQ ID NO: 675 - RNA, 19 nt, Artificial Sequence
  Description of Artificial Sequence Synthetic oligonucleotide
aaagauggcuuaccaaaua

SEQ ID NO: 676 - RNA, 19 nt, Artificial Sequence
  Description of Artificial Sequence Synthetic oligonucleotide
gcacauugcuuuauaaaca

SEQ ID NO: 677 - RNA, 19 nt, Artificial Sequence
  Description of Artificial Sequence Synthetic oligonucleotide
gagcgaggaugcggucaaa

SEQ ID NO: 678 - RNA, 19 nt, Artificial Sequence
  Description of Artificial Sequence Synthetic oligonucleotide
gcugaucggaagcgcuuaa

SEQ ID NO: 679 - RNA, 19 nt, Artificial Sequence
  Description of Artificial Sequence Synthetic oligonucleotide
gcgagacggugcucaaugg

SEQ ID NO: 680 - RNA, 19 nt, Artificial Sequence
  Description of Artificial Sequence Synthetic oligonucleotide
gccaaacaguccuuagcua

SEQ ID NO: 681 - RNA, 19 nt, Artificial Sequence
  Description of Artificial Sequence Synthetic oligonucleotide
gaauuaggauuucaaagug

SEQ ID NO: 682 - RNA, 19 nt, Artificial Sequence
  Description of Artificial Sequence Synthetic oligonucleotide
ucaaguaccuaaucaucga

SEQ ID NO: 683 - RNA, 19 nt, Artificial Sequence
  Description of Artificial Sequence Synthetic oligonucleotide
gcagcaagauggccaguaa

SEQ ID NO: 684 - RNA, 19 nt, Artificial Sequence
  Description of Artificial Sequence Synthetic oligonucleotide
guacagggcugaaaacaua

SEQ ID NO: 685 - RNA, 19 nt, Artificial Sequence
  Description of Artificial Sequence Synthetic oligonucleotide
guagaucacuuccaaacac

SEQ ID NO: 686 - RNA, 19 nt, Artificial Sequence
  Description of Artificial Sequence Synthetic oligonucleotide
gaccuuagauagcguuaau

SEQ ID NO: 687 - RNA, 19 nt, Artificial Sequence
  Description of Artificial Sequence Synthetic oligonucleotide
cuccaaacguccacaacua

SEQ ID NO: 688 - RNA, 19 nt, Artificial Sequence
  Description of Artificial Sequence Synthetic oligonucleotide
ccaugaauuuacagucuaa

SEQ ID NO: 689 - RNA, 19 nt, Artificial Sequence
  Description of Artificial Sequence Synthetic oligonucleotide
aaucagagguuugguagua

SEQ ID NO: 690 - RNA, 19 nt, Artificial Sequence
  Description of Artificial Sequence Synthetic oligonucleotide
ucgagaugcuagaaugaau

SEQ ID NO: 691 - RNA, 19 nt, Artificial Sequence
  Description of Artificial Sequence Synthetic oligonucleotide
gaauucaagucauccaaga

SEQ ID NO: 692 - RNA, 19 nt, Artificial Sequence
  Description of Artificial Sequence Synthetic oligonucleotide
caacacuucuggaucuuaa

SEQ ID NO: 693 - RNA, 19 nt, Artificial Sequence
  Description of Artificial Sequence Synthetic oligonucleotide
cgaaagauaccugcacaua

SEQ ID NO: 694 - RNA, 19 nt, Artificial Sequence
  Description of Artificial Sequence Synthetic oligonucleotide
gaagcggugugagcacuuu

SEQ ID NO: 695 - RNA, 19 nt, Artificial Sequence
  Description of Artificial Sequence Synthetic oligonucleotide
agacauaccucaacacuau

SEQ ID NO: 696 - RNA, 19 nt, Artificial Sequence
  Description of Artificial Sequence Synthetic oligonucleotide
guucccggcuacuggauua

SEQ ID NO: 697 - RNA, 19 nt, Artificial Sequence
  Description of Artificial Sequence Synthetic oligonucleotide
ucacuguccuuaucuucaa

SEQ ID NO: 698 - RNA, 19 nt, Artificial Sequence
  Description of Artificial Sequence Synthetic oligonucleotide
uggcggugcuacagauuga

SEQ ID NO: 699 - RNA, 19 nt, Artificial Sequence
  Description of Artificial Sequence Synthetic oligonucleotide
gaagugcccaccuaucuca

SEQ ID NO: 700 - RNA, 19 nt, Artificial Sequence
  Description of Artificial Sequence Synthetic oligonucleotide
guucaucgagcacucagua

SEQ ID NO: 701 - RNA, 19 nt, Artificial Sequence
  Description of Artificial Sequence Synthetic oligonucleotide
gggaaagccugacucaauu

SEQ ID NO: 702 - RNA, 19 nt, Artificial Sequence
  Description of Artificial Sequence Synthetic oligonucleotide
gaguaaagcugcugcuaaa

SEQ ID NO: 703 - RNA, 19 nt, Artificial Sequence
  Description of Artificial Sequence Synthetic oligonucleotide
guacuggaaugccuauuaa

SEQ ID NO: 704 - RNA, 19 nt, Artificial Sequence
  Description of Artificial Sequence Synthetic oligonucleotide
gggaagcgguauacaauuu

SEQ ID NO: 705 - RNA, 19 nt, Artificial Sequence
  Description of Artificial Sequence Synthetic oligonucleotide
gaaggaaagucaaguuuac

SEQ ID NO: 706 - RNA, 19 nt, Artificial Sequence
  Description of Artificial Sequence Synthetic oligonucleotide
caacagaccaccuaucaga

SEQ ID NO: 707 - RNA, 19 nt, Artificial Sequence
  Description of Artificial Sequence Synthetic oligonucleotide
ggaggaagagaauucauua

SEQ ID NO: 708 - RNA, 19 nt, Artificial Sequence
  Description of Artificial Sequence Synthetic oligonucleotide
aaaccaggauggcacauuu

SEQ ID NO: 709 - RNA, 19 nt, Artificial Sequence
  Description of Artificial Sequence Synthetic oligonucleotide
gaagugggccgagaacugg

SEQ ID NO: 710 - RNA, 19 nt, Artificial Sequence
  Description of Artificial Sequence Synthetic oligonucleotide
gccaggagauguacauuga

SEQ ID NO: 711 - RNA, 19 nt, Artificial Sequence
  Description of Artificial Sequence Synthetic oligonucleotide
gacagugcaucgccucgga

SEQ ID NO: 712 - RNA, 19 nt, Artificial Sequence
  Description of Artificial Sequence Synthetic oligonucleotide
gcaccucccucaucgacuc

SEQ ID NO: 713 - RNA, 19 nt, Artificial Sequence
  Description of Artificial Sequence Synthetic oligonucleotide
cuucauagcugccacgaua

SEQ ID NO: 714 - RNA, 19 nt, Artificial Sequence
  Description of Artificial Sequence Synthetic oligonucleotide
gugaacagcuuggcauuaa

SEQ ID NO: 715 - RNA, 19 nt, Artificial Sequence
  Description of Artificial Sequence Synthetic oligonucleotide
ggacauacauucugcuaca

SEQ ID NO: 716 - RNA, 19 nt, Artificial Sequence
  Description of Artificial Sequence Synthetic oligonucleotide
ccaaguggcucuucauaaa

SEQ ID NO: 717 - RNA, 19 nt, Artificial Sequence
  Description of Artificial Sequence Synthetic oligonucleotide
ccaucgaugcuuugagcua

SEQ ID NO: 718 - RNA, 19 nt, Artificial Sequence
  Description of Artificial Sequence Synthetic oligonucleotide
gaaaagggcuacucacuua

SEQ ID NO: 719 - RNA, 19 nt, Artificial Sequence
  Description of Artificial Sequence Synthetic oligonucleotide
ccacuuggguagccuaugg

SEQ ID NO: 720 - RNA, 19 nt, Artificial Sequence
  Description of Artificial Sequence Synthetic oligonucleotide
gaguugagauuuggauuaa

SEQ ID NO: 721 - RNA, 19 nt, Artificial Sequence
  Description of Artificial Sequence Synthetic oligonucleotide
cagaggagcucccuacuug

SEQ ID NO: 722 - RNA, 19 nt, Artificial Sequence
  Description of Artificial Sequence Synthetic oligonucleotide
caucagagaaacaagcaga

SEQ ID NO: 723 - RNA, 19 nt, Artificial Sequence
  Description of Artificial Sequence Synthetic oligonucleotide
gaaauucccacccaccuac

SEQ ID NO: 724 - RNA, 19 nt, Artificial Sequence
  Description of Artificial Sequence Synthetic oligonucleotide
ggguaucucuggauggguu

SEQ ID NO: 725 - RNA, 19 nt, Artificial Sequence
  Description of Artificial Sequence Synthetic oligonucleotide
cucaguguguuauuuguaa

SEQ ID NO: 726 - skipped
  Description of Artificial Sequence Synthetic oligonucleotide

SEQ ID NO: 727 - RNA, 19 nt, Artificial Sequence
  Description of Artificial Sequence Synthetic oligonucleotide
ggaauagugaagugaaaua

SEQ ID NO: 728 - RNA, 19 nt, Artificial Sequence
  Description of Artificial Sequence Synthetic oligonucleotide
gugcaagacguuauaauga

SEQ ID NO: 729 - RNA, 19 nt, Artificial Sequence
  Description of Artificial Sequence Synthetic oligonucleotide
gcgcauaguuggccaauau

SEQ ID NO: 730 - RNA, 19 nt, Artificial Sequence
  Description of Artificial Sequence Synthetic oligonucleotide
ggucauagcugcaggugcc

SEQ ID NO: 731 - RNA, 19 nt, Artificial Sequence
  Description of Artificial Sequence Synthetic oligonucleotide
ggauuggugugcccuagug

SEQ ID NO: 732 - RNA, 19 nt, Artificial Sequence
  Description of Artificial Sequence Synthetic oligonucleotide
ggacacauccaugggcaca

SEQ ID NO: 733 - RNA, 19 nt, Artificial Sequence
  Description of Artificial Sequence Synthetic oligonucleotide
cagcacaagcuaugcacag

SEQ ID NO: 734 - RNA, 19 nt, Artificial Sequence
  Description of Artificial Sequence Synthetic oligonucleotide
gugaccagaucuccaguaa

SEQ ID NO: 735 - RNA, 19 nt, Artificial Sequence
  Description of Artificial Sequence Synthetic oligonucleotide
ggaaagggugugucgauga

SEQ ID NO: 736 - RNA, 19 nt, Artificial Sequence
  Description of Artificial Sequence Synthetic oligonucleotide
gcucaguguucgaaacgug

SEQ ID NO: 737 - RNA, 19 nt, Artificial Sequence
  Description of Artificial Sequence Synthetic oligonucleotide
ugaaaguggacgaauguaa

SEQ ID NO: 738 - RNA, 19 nt, Artificial Sequence
  Description of Artificial Sequence Synthetic oligonucleotide
gaagagugauucauacuuu

SEQ ID NO: 739 - RNA, 19 nt, Artificial Sequence
  Description of Artificial Sequence Synthetic oligonucleotide
gcaacaaucugggcuauga

SEQ ID NO: 740 - RNA, 19 nt, Artificial Sequence
  Description of Artificial Sequence Synthetic oligonucleotide
gaaccagacuccaauguca

SEQ ID NO: 741 - RNA, 19 nt, Artificial Sequence
  Description of Artificial Sequence Synthetic oligonucleotide
caggaagucugaccaauaa

SEQ ID NO: 742 - RNA, 19 nt, Artificial Sequence
  Description of Artificial Sequence Synthetic oligonucleotide
gagaguugcggcaccuuaa

SEQ ID NO: 743 - RNA, 19 nt, Artificial Sequence
  Description of Artificial Sequence Synthetic oligonucleotide
ggugggauguguuuaugga

SEQ ID NO: 744 - RNA, 19 nt, Artificial Sequence
  Description of Artificial Sequence Synthetic oligonucleotide
gaugauaggacuugaaucu

SEQ ID NO: 745 - RNA, 19 nt, Artificial Sequence
  Description of Artificial Sequence Synthetic oligonucleotide
uugagcaucugauugguua

SEQ ID NO: 746 - RNA, 19 nt, Artificial Sequence
  Description of Artificial Sequence Synthetic oligonucleotide
gcaacaaacuaccaacacu

SEQ ID NO: 747 - RNA, 19 nt, Artificial Sequence
  Description of Artificial Sequence Synthetic oligonucleotide
gaggcagauuacaugcuua

SEQ ID NO: 748 - RNA, 19 nt, Artificial Sequence
  Description of Artificial Sequence Synthetic oligonucleotide
gaugagcgaauuuauguga

SEQ ID NO: 749 - RNA, 19 nt, Artificial Sequence
  Description of Artificial Sequence Synthetic oligonucleotide
uaaauaugaucuugggaga

SEQ ID NO: 750 - RNA, 19 nt, Artificial Sequence
  Description of Artificial Sequence Synthetic oligonucleotide
gaagaaggacugugcggag

SEQ ID NO: 751 - RNA, 19 nt, Artificial Sequence
  Description of Artificial Sequence Synthetic oligonucleotide
gcaagaccacgaacacagu

SEQ ID NO: 752 - RNA, 19 nt, Artificial Sequence
  Description of Artificial Sequence Synthetic oligonucleotide
cugcaagcauucugugguc

SEQ ID NO: 753 - RNA, 19 nt, Artificial Sequence
  Description of Artificial Sequence Synthetic oligonucleotide
gcaaugagaagcugcacaa

SEQ ID NO: 754 - RNA, 19 nt, Artificial Sequence
  Description of Artificial Sequence Synthetic oligonucleotide
gcgcaaaucuccuaccuug

SEQ ID NO: 755 - RNA, 19 nt, Artificial Sequence
  Description of Artificial Sequence Synthetic oligonucleotide
ggucuuaucucugcaacaa

SEQ ID NO: 756 - RNA, 19 nt, Artificial Sequence
  Description of Artificial Sequence Synthetic oligonucleotide
ggugugcucgugaacauaa

SEQ ID NO: 757 - RNA, 19 nt, Artificial Sequence
  Description of Artificial Sequence Synthetic oligonucleotide
gcgagcacaugaaggauug

SEQ ID NO: 758 - RNA, 19 nt, Artificial Sequence
  Description of Artificial Sequence Synthetic oligonucleotide
ggcauaacccagauaguug

SEQ ID NO: 759 - RNA, 19 nt, Artificial Sequence
  Description of Artificial Sequence Synthetic oligonucleotide
ugaaggaccuguuuaauga

SEQ ID NO: 760 - RNA, 19 nt, Artificial Sequence
  Description of Artificial Sequence Synthetic oligonucleotide
ccacgcaucuucaugauac

SEQ ID NO: 761 - RNA, 19 nt, Artificial Sequence
  Description of Artificial Sequence Synthetic oligonucleotide
aaucagaacuccgcgaaua

SEQ ID NO: 762 - RNA, 19 nt, Artificial Sequence
  Description of Artificial Sequence Synthetic oligonucleotide
gacagagccugguaaugau

SEQ ID NO: 763 - RNA, 19 nt, Artificial Sequence
  Description of Artificial Sequence Synthetic oligonucleotide
cgaaaguaauggcuaacua

SEQ ID NO: 764 - RNA, 19 nt, Artificial Sequence
  Description of Artificial Sequence Synthetic oligonucleotide
guaaucaaccuaacacaua

SEQ ID NO: 765 - RNA, 19 nt, Artificial Sequence
  Description of Artificial Sequence Synthetic oligonucleotide
agaccaaauuuaacccaua

SEQ ID NO: 766 - RNA, 19 nt, Artificial Sequence
  Description of Artificial Sequence Synthetic oligonucleotide
ggacaugucuccacuauca

SEQ ID NO: 767 - RNA, 19 nt, Artificial Sequence
  Description of Artificial Sequence Synthetic oligonucleotide
ggaaucacccaccaaauua

SEQ ID NO: 768 - RNA, 19 nt, Artificial Sequence
  Description of Artificial Sequence Synthetic oligonucleotide
cgaggaggauucuguguua

SEQ ID NO: 769 - RNA, 19 nt, Artificial Sequence
  Description of Artificial Sequence Synthetic oligonucleotide
caacaccaguuccaauuaa

SEQ ID NO: 770 - RNA, 19 nt, Artificial Sequence
  Description of Artificial Sequence Synthetic oligonucleotide
gaaauuugauggacuuuga

SEQ ID NO: 771 - RNA, 19 nt, Artificial Sequence
  Description of Artificial Sequence Synthetic oligonucleotide
gcaauugugucucaacuuu

SEQ ID NO: 772 - RNA, 19 nt, Artificial Sequence
  Description of Artificial Sequence Synthetic oligonucleotide
gaaauacccuuguuagaau

SEQ ID NO: 773 - RNA, 19 nt, Artificial Sequence
  Description of Artificial Sequence Synthetic oligonucleotide
ggucauggcuguuaaauug

SEQ ID NO: 774 - RNA, 19 nt, Artificial Sequence
  Description of Artificial Sequence Synthetic oligonucleotide
ugagugggcugaugcguga

SEQ ID NO: 775 - RNA, 19 nt, Artificial Sequence
  Description of Artificial Sequence Synthetic oligonucleotide
cagaaaccagagcaaguua

SEQ ID NO: 776 - RNA, 19 nt, Artificial Sequence
  Description of Artificial Sequence Synthetic oligonucleotide
gcggaaagaugcacuaguc

SEQ ID NO: 777 - RNA, 19 nt, Artificial Sequence
  Description of Artificial Sequence Synthetic oligonucleotide
guacuuugguggacgaguu

SEQ ID NO: 778 - RNA, 19 nt, Artificial Sequence
  Description of Artificial Sequence Synthetic oligonucleotide
caagauaaagucagaccua

SEQ ID NO: 779 - RNA, 19 nt, Artificial Sequence
  Description of Artificial Sequence Synthetic oligonucleotide
gaaagaggcagaaccuaua

SEQ ID NO: 780 - RNA, 19 nt, Artificial Sequence
  Description of Artificial Sequence Synthetic oligonucleotide
cccacuagcugauuacuau

SEQ ID NO: 781 - RNA, 19 nt, Artificial Sequence
  Description of Artificial Sequence Synthetic oligonucleotide
caacagacagugagugcua

SEQ ID NO: 782 - RNA, 19 nt, Artificial Sequence
  Description of Artificial Sequence Synthetic oligonucleotide
ccaccuauccgcugcugaa

SEQ ID NO: 783 - RNA, 19 nt, Artificial Sequence
  Description of Artificial Sequence Synthetic oligonucleotide
gagaaagugaggaccaaug

SEQ ID NO: 784 - RNA, 19 nt, Artificial Sequence
  Description of Artificial Sequence Synthetic oligonucleotide
uggacaagcugcugcauua

SEQ ID NO: 785 - RNA, 19 nt, Artificial Sequence
  Description of Artificial Sequence Synthetic oligonucleotide
ggaggccuaugaccgagau

SEQ ID NO: 786 - RNA, 19 nt, Artificial Sequence
  Description of Artificial Sequence Synthetic oligonucleotide
gcacacaucucuuaccuag

SEQ ID NO: 787 - RNA, 19 nt, Artificial Sequence
  Description of Artificial Sequence Synthetic oligonucleotide
cugcgucacuuccucuaua

SEQ ID NO: 788 - RNA, 19 nt, Artificial Sequence
  Description of Artificial Sequence Synthetic oligonucleotide
cuacagcguugcccaagug

SEQ ID NO: 789 - RNA, 19 nt, Artificial Sequence
  Description of Artificial Sequence Synthetic oligonucleotide
gaccgccucuucauucuca

SEQ ID NO: 790 - RNA, 19 nt, Artificial Sequence
  Description of Artificial Sequence Synthetic oligonucleotide
guggagucauucaaguuua

SEQ ID NO: 791 - RNA, 19 nt, Artificial Sequence
  Description of Artificial Sequence Synthetic oligonucleotide
uauguggacugauugauga

SEQ ID NO: 792 - RNA, 19 nt, Artificial Sequence
  Description of Artificial Sequence Synthetic oligonucleotide
ggaggaugauucucugcga

SEQ ID NO: 793 - RNA, 19 nt, Artificial Sequence
  Description of Artificial Sequence Synthetic oligonucleotide
caggucagauugaguugua

SEQ ID NO: 794 - RNA, 19 nt, Artificial Sequence
  Description of Artificial Sequence Synthetic oligonucleotide
gauaagauaaggcuguaua

SEQ ID NO: 795 - RNA, 19 nt, Artificial Sequence
  Description of Artificial Sequence Synthetic oligonucleotide
ugagugagaucaagacuga

SEQ ID NO: 796 - RNA, 19 nt, Artificial Sequence
  Description of Artificial Sequence Synthetic oligonucleotide
cgauacaacuccuaugaua

SEQ ID NO: 797 - RNA, 19 nt, Artificial Sequence
  Description of Artificial Sequence Synthetic oligonucleotide
gcacaacgcugagcauuac

SEQ ID NO: 798 - RNA, 19 nt, Artificial Sequence
  Description of Artificial Sequence Synthetic oligonucleotide
gagaccaacuugcuccugg

SEQ ID NO: 799 - RNA, 19 nt, Artificial Sequence
  Description of Artificial Sequence Synthetic oligonucleotide
aguucaggcuguugaguga

SEQ ID NO: 800 - RNA, 19 nt, Artificial Sequence
  Description of Artificial Sequence Synthetic oligonucleotide
gacugaaccgggagcacaa

SEQ ID NO: 801 - RNA, 19 nt, Artificial Sequence
  Description of Artificial Sequence Synthetic oligonucleotide
cagcaagacuccacgcgca

SEQ ID NO: 802 - RNA, 19 nt, Artificial Sequence
  Description of Artificial Sequence Synthetic oligonucleotide
cccaagagauggucgucaa

SEQ ID NO: 803 - RNA, 19 nt, Artificial Sequence
  Description of Artificial Sequence Synthetic oligonucleotide
gaacccauaugcccacauc

SEQ ID NO: 804 - RNA, 19 nt, Artificial Sequence
  Description of Artificial Sequence Synthetic oligonucleotide
ugacaucgcccaccacugc

SEQ ID NO: 805 - RNA, 19 nt, Artificial Sequence
  Description of Artificial Sequence Synthetic oligonucleotide
gggcagcaguuagaggugg

SEQ ID NO: 806 - RNA, 19 nt, Artificial Sequence
  Description of Artificial Sequence Synthetic oligonucleotide
ucucgaaacucacgcgcaa

SEQ ID NO: 807 - RNA, 19 nt, Artificial Sequence
  Description of Artificial Sequence Synthetic oligonucleotide
gcaaaucugcgacaccuac

SEQ ID NO: 808 - RNA, 19 nt, Artificial Sequence
  Description of Artificial Sequence Synthetic oligonucleotide
cagaagcacucgcucuuua

SEQ ID NO: 809 - RNA, 19 nt, Artificial Sequence
  Description of Artificial Sequence Synthetic oligonucleotide
gagaucggcuucggcaauu

SEQ ID NO: 810 - RNA, 19 nt, Artificial Sequence
  Description of Artificial Sequence Synthetic oligonucleotide
gcaaucacacguacuguuu

SEQ ID NO: 811 - RNA, 19 nt, Artificial Sequence
  Description of Artificial Sequence Synthetic oligonucleotide
gagaggagauuugggauuc

SEQ ID NO: 812 - RNA, 19 nt, Artificial Sequence
  Description of Artificial Sequence Synthetic oligonucleotide
ggaacucggugcagauaug

SEQ ID NO: 813 - RNA, 19 nt, Artificial Sequence
  Description of Artificial Sequence Synthetic oligonucleotide
ggaaacagcucaguucuca

SEQ ID NO: 814 - RNA, 19 nt, Artificial Sequence
  Description of Artificial Sequence Synthetic oligonucleotide
gcaaggcugcgccacgaua

SEQ ID NO: 815 - RNA, 19 nt, Artificial Sequence
  Description of Artificial Sequence Synthetic oligonucleotide
guggaucucaccucuuuga

SEQ ID NO: 816 - RNA, 19 nt, Artificial Sequence
  Description of Artificial Sequence Synthetic oligonucleotide
gaaagugucucaaguacuc

SEQ ID NO: 817 - RNA, 19 nt, Artificial Sequence
  Description of Artificial Sequence Synthetic oligonucleotide
caaggcugcgccacgauaa

SEQ ID NO: 818 - RNA, 19 nt, Artificial Sequence
  Description of Artificial Sequence Synthetic oligonucleotide
uagaagauaugagaccuug

SEQ ID NO: 819 - RNA, 19 nt, Artificial Sequence
  Description of Artificial Sequence Synthetic oligonucleotide
ggacuggacucagaaacca

SEQ ID NO: 820 - RNA, 19 nt, Artificial Sequence
  Description of Artificial Sequence Synthetic oligonucleotide
agagagaccaugccaauua

SEQ ID NO: 821 - RNA, 19 nt, Artificial Sequence
  Description of Artificial Sequence Synthetic oligonucleotide
gcacaaagcagaagaaguu

SEQ ID NO: 822 - RNA, 19 nt, Artificial Sequence
  Description of Artificial Sequence Synthetic oligonucleotide
gaauguauugccuggcuaa

SEQ ID NO: 823 - RNA, 19 nt, Artificial Sequence
  Description of Artificial Sequence Synthetic oligonucleotide
cagagaaccucgcgccuga

SEQ ID NO: 824 - RNA, 19 nt, Artificial Sequence
  Description of Artificial Sequence Synthetic oligonucleotide
cagaguaaaucgcaaagaa

SEQ ID NO: 825 - RNA, 19 nt, Artificial Sequence
  Description of Artificial Sequence Synthetic oligonucleotide
cauaugacgggcaggauuu

SEQ ID NO: 826 - RNA, 19 nt, Artificial Sequence
  Description of Artificial Sequence Synthetic oligonucleotide
ggauggaccugaauaaaau

SEQ ID NO: 827 - RNA, 19 nt, Artificial Sequence
  Description of Artificial Sequence Synthetic oligonucleotide
cuacggacuuuguggcuga

SEQ ID NO: 828 - RNA, 19 nt, Artificial Sequence
  Description of Artificial Sequence Synthetic oligonucleotide
ggauuugggccugagguua

SEQ ID NO: 829 - RNA, 19 nt, Artificial Sequence
  Description of Artificial Sequence Synthetic oligonucleotide
ccaauaagaauuugacaag

SEQ ID NO: 830 - RNA, 19 nt, Artificial Sequence
  Description of Artificial Sequence Synthetic oligonucleotide
gaaccucgaugagaucuug

SEQ ID NO: 831 - RNA, 19 nt, Artificial Sequence
  Description of Artificial Sequence Synthetic oligonucleotide
ggaaaagacucaaaguaaa

SEQ ID NO: 832 - RNA, 19 nt, Artificial Sequence
  Description of Artificial Sequence Synthetic oligonucleotide
ggacggagaucuucaauau

SEQ ID NO: 833 - RNA, 19 nt, Artificial Sequence
  Description of Artificial Sequence Synthetic oligonucleotide
gcgcaccuaugacaugaug

SEQ ID NO: 834 - RNA, 19 nt, Artificial Sequence
  Description of Artificial Sequence Synthetic oligonucleotide
cggcagaguuggccaaagg

SEQ ID NO: 835 - RNA, 19 nt, Artificial Sequence
  Description of Artificial Sequence Synthetic oligonucleotide
gcaaggagcgcgagauggu

SEQ ID NO: 836 - RNA, 19 nt, Artificial Sequence
  Description of Artificial Sequence Synthetic oligonucleotide
gcaccgcgacuaugugaug

SEQ ID NO: 837 - RNA, 19 nt, Artificial Sequence
  Description of Artificial Sequence Synthetic oligonucleotide
caaccuucccgccagacua

SEQ ID NO: 838 - RNA, 19 nt, Artificial Sequence
  Description of Artificial Sequence Synthetic oligonucleotide
gacaaggaguuuacuauua

SEQ ID NO: 839 - RNA, 19 nt, Artificial Sequence
  Description of Artificial Sequence Synthetic oligonucleotide
ggagacagcucuggauauu

SEQ ID NO: 840 - RNA, 19 nt, Artificial Sequence
  Description of Artificial Sequence Synthetic oligonucleotide
ggaaggaccucuuugauuu

SEQ ID NO: 841 - RNA, 19 nt, Artificial Sequence
  Description of Artificial Sequence Synthetic oligonucleotide
gaagauggcugaggaguca

SEQ ID NO: 842 - RNA, 19 nt, Artificial Sequence
  Description of Artificial Sequence Synthetic oligonucleotide
gaggagcuguugaauguuu

SEQ ID NO: 843 - RNA, 19 nt, Artificial Sequence
  Description of Artificial Sequence Synthetic oligonucleotide
gaaauugcgaucugauaca

SEQ ID NO: 844 - RNA, 19 nt, Artificial Sequence
  Description of Artificial Sequence Synthetic oligonucleotide
agacaaagcuuaaaugacc

SEQ ID NO: 845 - RNA, 19 nt, Artificial Sequence
  Description of Artificial Sequence Synthetic oligonucleotide
gcgagugggccaauuguuu

SEQ ID NO: 846 - RNA, 19 nt, Artificial Sequence
  Description of Artificial Sequence Synthetic oligonucleotide
aaacaagagacggaaauua

SEQ ID NO: 847 - RNA, 19 nt, Artificial Sequence
  Description of Artificial Sequence Synthetic oligonucleotide
ccacacagaauauggagaa

SEQ ID NO: 848 - RNA, 19 nt, Artificial Sequence
  Description of Artificial Sequence Synthetic oligonucleotide
ccuuaaaccuucaaauaug

SEQ ID NO: 849 - RNA, 19 nt, Artificial Sequence
  Description of Artificial Sequence Synthetic oligonucleotide
gauaccaugguccaaauua

SEQ ID NO: 850 - RNA, 19 nt, Artificial Sequence
  Description of Artificial Sequence Synthetic oligonucleotide
gaaauugguuggguucauu

SEQ ID NO: 851 - RNA, 19 nt, Artificial Sequence
  Description of Artificial Sequence Synthetic oligonucleotide
acggcaaccugcaguauua

SEQ ID NO: 852 - RNA, 19 nt, Artificial Sequence
  Description of Artificial Sequence Synthetic oligonucleotide
gcagaaauaugaccaugca

SEQ ID NO: 853 - RNA, 19 nt, Artificial Sequence
  Description of Artificial Sequence Synthetic oligonucleotide
cagcaaacauccauaucua

SEQ ID NO: 854 - RNA, 19 nt, Artificial Sequence
  Description of Artificial Sequence Synthetic oligonucleotide
cguagccgcugccuaugua

SEQ ID NO: 855 - RNA, 19 nt, Artificial Sequence
  Description of Artificial Sequence Synthetic oligonucleotide
gaauaugccugccugaaag

SEQ ID NO: 856 - RNA, 19 nt, Artificial Sequence
  Description of Artificial Sequence Synthetic oligonucleotide
ggaauaugccugccugaaa

SEQ ID NO: 857 - RNA, 19 nt, Artificial Sequence
  Description of Artificial Sequence Synthetic oligonucleotide
gccauucucuacgcacuuc

SEQ ID NO: 858 - RNA, 19 nt, Artificial Sequence
  Description of Artificial Sequence Synthetic oligonucleotide
uaucagugcuguuauguua

SEQ ID NO: 859 - RNA, 19 nt, Artificial Sequence
  Description of Artificial Sequence Synthetic oligonucleotide
caucagagcuuauuuggaa

SEQ ID NO: 860 - RNA, 19 nt, Artificial Sequence
  Description of Artificial Sequence Synthetic oligonucleotide
gaaaguguauucgcaguua

SEQ ID NO: 861 - RNA, 19 nt, Artificial Sequence
  Description of Artificial Sequence Synthetic oligonucleotide
ggaaaucucuccaugguua

SEQ ID NO: 862 - RNA, 19 nt, Artificial Sequence
  Description of Artificial Sequence Synthetic oligonucleotide
ccaguaaucgggaaagaua

SEQ ID NO: 863 - RNA, 19 nt, Artificial Sequence
  Description of Artificial Sequence Synthetic oligonucleotide
ggagguaucccaaguagau

SEQ ID NO: 864 - RNA, 19 nt, Artificial Sequence
  Description of Artificial Sequence Synthetic oligonucleotide
uaacugagucugugaacua

SEQ ID NO: 865 - RNA, 19 nt, Artificial Sequence
  Description of Artificial Sequence Synthetic oligonucleotide
cauccgaacgguaauaaua

SEQ ID NO: 866 - RNA, 19 nt, Artificial Sequence
  Description of Artificial Sequence Synthetic oligonucleotide
gaggagagcucuaauauua

SEQ ID NO: 867 - RNA, 19 nt, Artificial Sequence
  Description of Artificial Sequence Synthetic oligonucleotide
ggaagaaagcugacaauga

SEQ ID NO: 868 - RNA, 19 nt, Artificial Sequence
  Description of Artificial Sequence Synthetic oligonucleotide
gaagcgagcccuuacauug

SEQ ID NO: 869 - RNA, 19 nt, Artificial Sequence
  Description of Artificial Sequence Synthetic oligonucleotide
caauucgucucaagcauua

SEQ ID NO: 870 - RNA, 19 nt, Artificial Sequence
  Description of Artificial Sequence Synthetic oligonucleotide
cauuugggaugcaagcuua

SEQ ID NO: 871 - RNA, 19 nt, Artificial Sequence
  Description of Artificial Sequence Synthetic oligonucleotide
ggacucagcuaugcuaauu

SEQ ID NO: 872 - RNA, 19 nt, Artificial Sequence
  Description of Artificial Sequence Synthetic oligonucleotide
acauagagcucuuuauagu

SEQ ID NO: 873 - RNA, 19 nt, Artificial Sequence
  Description of Artificial Sequence Synthetic oligonucleotide
caacucaggccacaaauau

SEQ ID NO: 874 - RNA, 19 nt, Artificial Sequence
  Description of Artificial Sequence Synthetic oligonucleotide
gaauaugcguggauugugc

SEQ ID NO: 875 - RNA, 19 nt, Artificial Sequence
  Description of Artificial Sequence Synthetic oligonucleotide
ggcaacacgggauuuauua

SEQ ID NO: 876 - RNA, 19 nt, Artificial Sequence
  Description of Artificial Sequence Synthetic oligonucleotide
gagccaaacuggauugaau

SEQ ID NO: 877 - RNA, 19 nt, Artificial Sequence
  Description of Artificial Sequence Synthetic oligonucleotide
ggacacaaauuacggcuug

SEQ ID NO: 878 - RNA, 19 nt, Artificial Sequence
  Description of Artificial Sequence Synthetic oligonucleotide
ggauguagauguuuacucu

SEQ ID NO: 879 - RNA, 19 nt, Artificial Sequence
  Description of Artificial Sequence Synthetic oligonucleotide
guacgccucgggacuguuu

SEQ ID NO: 880 - RNA, 19 nt, Artificial Sequence
  Description of Artificial Sequence Synthetic oligonucleotide
gaaagccguccgcaacaau

SEQ ID NO: 881 - RNA, 19 nt, Artificial Sequence
  Description of Artificial Sequence Synthetic oligonucleotide
ugacucggcucuuguacuc

SEQ ID NO: 882 - RNA, 19 nt, Artificial Sequence
  Description of Artificial Sequence Synthetic oligonucleotide
uaaugcaacuggauguuca

SEQ ID NO: 883 - RNA, 19 nt, Artificial Sequence
  Description of Artificial Sequence Synthetic oligonucleotide
gacaauaacagaagcgaag

SEQ ID NO: 884 - RNA, 19 nt, Artificial Sequence
  Description of Artificial Sequence Synthetic oligonucleotide
ucgcaaaggucacaaacaa

SEQ ID NO: 885 - RNA, 19 nt, Artificial Sequence
  Description of Artificial Sequence Synthetic oligonucleotide
gaaaucagggcuuaaauga

SEQ ID NO: 886 - RNA, 19 nt, Artificial Sequence
  Description of Artificial Sequence Synthetic oligonucleotide
gaacgcugguuaaauuggu

SEQ ID NO: 887 - RNA, 19 nt, Artificial Sequence
  Description of Artificial Sequence Synthetic oligonucleotide
uaauacugcuuaugggaaa

SEQ ID NO: 888 - RNA, 19 nt, Artificial Sequence
  Description of Artificial Sequence Synthetic oligonucleotide
uacccuauguugcugguua

SEQ ID NO: 889 - RNA, 19 nt, Artificial Sequence
  Description of Artificial Sequence Synthetic oligonucleotide
guggaacgcugguuaaauu

SEQ ID NO: 890 - RNA, 19 nt, Artificial Sequence
  Description of Artificial Sequence Synthetic oligonucleotide
gaccuuaacuugucgcuga

SEQ ID NO: 891 - RNA, 19 nt, Artificial Sequence
  Description of Artificial Sequence Synthetic oligonucleotide
gcaagugaguguuacugau

SEQ ID NO: 892 - RNA, 19 nt, Artificial Sequence
  Description of Artificial Sequence Synthetic oligonucleotide
ccagagaacucggcuauaa

SEQ ID NO: 893 - RNA, 19 nt, Artificial Sequence
  Description of Artificial Sequence Synthetic oligonucleotide
ggaauaaacggaguucaaa

SEQ ID NO: 894 - RNA, 19 nt, Artificial Sequence
  Description of Artificial Sequence Synthetic oligonucleotide
gaagugauaguucaaguaa

SEQ ID NO: 895 - RNA, 19 nt, Artificial Sequence
  Description of Artificial Sequence Synthetic oligonucleotide
gaugaaauuugcucugcua

SEQ ID NO: 896 - RNA, 19 nt, Artificial Sequence
  Description of Artificial Sequence Synthetic oligonucleotide
cgaauuaacuccaucgaau

SEQ ID NO: 897 - RNA, 19 nt, Artificial Sequence
  Description of Artificial Sequence Synthetic oligonucleotide
gacgacagcuuugauaaga

SEQ ID NO: 898 - RNA, 19 nt, Artificial Sequence
  Description of Artificial Sequence Synthetic oligonucleotide
ccaaugaacugacuuguac

SEQ ID NO: 899 - RNA, 19 nt, Artificial Sequence
  Description of Artificial Sequence Synthetic oligonucleotide
gaagguuccugaugcuaua

SEQ ID NO: 900 - RNA, 19 nt, Artificial Sequence
  Description of Artificial Sequence Synthetic oligonucleotide
gaaagacuguccaaucaca

SEQ ID NO: 901 - RNA, 19 nt, Artificial Sequence
  Description of Artificial Sequence Synthetic oligonucleotide
gcaaacagcugacaagaca

SEQ ID NO: 902 - RNA, 19 nt, Artificial Sequence
  Description of Artificial Sequence Synthetic oligonucleotide
ggacgaagucagccuguaa

SEQ ID NO: 903 - RNA, 19 nt, Artificial Sequence
  Description of Artificial Sequence Synthetic oligonucleotide
cgauaaggauccuaugcua

SEQ ID NO: 904 - RNA, 19 nt, Artificial Sequence
  Description of Artificial Sequence Synthetic oligonucleotide
ucaaguagguggcaggaua

SEQ ID NO: 905 - RNA, 19 nt, Artificial Sequence
  Description of Artificial Sequence Synthetic oligonucleotide
gagguugacucuaugucua

SEQ ID NO: 906 - RNA, 19 nt, Artificial Sequence
  Description of Artificial Sequence Synthetic oligonucleotide
gagcggagcuuuucggaua

SEQ ID NO: 907 - RNA, 19 nt, Artificial Sequence
  Description of Artificial Sequence Synthetic oligonucleotide
aaagaaagcacuaccuuca

SEQ ID NO: 908 - RNA, 19 nt, Artificial Sequence
  Description of Artificial Sequence Synthetic oligonucleotide
ucuuaggucugcuugguua

SEQ ID NO: 909 - RNA, 19 nt, Artificial Sequence
  Description of Artificial Sequence Synthetic oligonucleotide
ggucaaggauauugucauc

SEQ ID NO: 910 - RNA, 19 nt, Artificial Sequence
  Description of Artificial Sequence Synthetic oligonucleotide
ccagcuagccaaacuaaua

SEQ ID NO: 911 - RNA, 19 nt, Artificial Sequence
  Description of Artificial Sequence Synthetic oligonucleotide
ggacaucgcacugaucuuu

SEQ ID NO: 912 - RNA, 19 nt, Artificial Sequence
  Description of Artificial Sequence Synthetic oligonucleotide
gauauggccuguaauccua

SEQ ID NO: 913 - RNA, 19 nt, Artificial Sequence
  Description of Artificial Sequence Synthetic oligonucleotide
guacggaaaguggaaauua

SEQ ID NO: 914 - RNA, 19 nt, Artificial Sequence
  Description of Artificial Sequence Synthetic oligonucleotide
gggaauagcuuauccaauc

SEQ ID NO: 915 - RNA, 19 nt, Artificial Sequence
  Description of Artificial Sequence Synthetic oligonucleotide
gcucagauaugaagcauca

SEQ ID NO: 916 - RNA, 19 nt, Artificial Sequence
  Description of Artificial Sequence Synthetic oligonucleotide
guaagaagcaagaagaagu

SEQ ID NO: 917 - RNA, 19 nt, Artificial Sequence
  Description of Artificial Sequence Synthetic oligonucleotide
gaugggaucccuaauauga

SEQ ID NO: 918 - RNA, 19 nt, Artificial Sequence
  Description of Artificial Sequence Synthetic oligonucleotide
ccaaauuccuguacuguaa

SEQ ID NO: 919 - RNA, 19 nt, Artificial Sequence
  Description of Artificial Sequence Synthetic oligonucleotide
ggagauauacuugguguug

SEQ ID NO: 920 - RNA, 19 nt, Artificial Sequence
  Description of Artificial Sequence Synthetic oligonucleotide
ggcaagaccuauuuauaau

SEQ ID NO: 921 - RNA, 19 nt, Artificial Sequence
  Description of Artificial Sequence Synthetic oligonucleotide
gauagaagucuguaaauac

SEQ ID NO: 922 - RNA, 19 nt, Artificial Sequence
  Description of Artificial Sequence Synthetic oligonucleotide
gagaaggggucuacuauuu

SEQ ID NO: 923 - RNA, 19 nt, Artificial Sequence
  Description of Artificial Sequence Synthetic oligonucleotide
ggaccaacgucaaggguau

SEQ ID NO: 924 - RNA, 19 nt, Artificial Sequence
  Description of Artificial Sequence Synthetic oligonucleotide
gagaaauccuggcauacuu

SEQ ID NO: 925 - RNA, 19 nt, Artificial Sequence
  Description of Artificial Sequence Synthetic oligonucleotide
gagauugcguccucgauaa

SEQ ID NO: 926 - RNA, 19 nt, Artificial Sequence
  Description of Artificial Sequence Synthetic oligonucleotide
gaaccaguaucuacacuuc

SEQ ID NO: 927 - RNA, 19 nt, Artificial Sequence
  Description of Artificial Sequence Synthetic oligonucleotide
ucacaacgcuugaggaaug

SEQ ID NO: 928 - RNA, 19 nt, Artificial Sequence
  Description of Artificial Sequence Synthetic oligonucleotide
gcauaaagauggcaguuug

SEQ ID NO: 929 - RNA, 19 nt, Artificial Sequence
  Description of Artificial Sequence Synthetic oligonucleotide
cgaagaaccuacucagaua

SEQ ID NO: 930 - RNA, 19 nt, Artificial Sequence
  Description of Artificial Sequence Synthetic oligonucleotide
gagucaacuuuccaccaaa

SEQ ID NO: 931 - RNA, 19 nt, Artificial Sequence
  Description of Artificial Sequence Synthetic oligonucleotide
cagauacgcugcugaccaa

SEQ ID NO: 932 - RNA, 19 nt, Artificial Sequence
  Description of Artificial Sequence Synthetic oligonucleotide
uaaacaagauccacgccuu

SEQ ID NO: 933 - RNA, 19 nt, Artificial Sequence
  Description of Artificial Sequence Synthetic oligonucleotide
cgagagaggguuugggaca

SEQ ID NO: 934 - RNA, 19 nt, Artificial Sequence
  Description of Artificial Sequence Synthetic oligonucleotide
gggagaaacucagccuuaa

SEQ ID NO: 935 - RNA, 19 nt, Artificial Sequence
  Description of Artificial Sequence Synthetic oligonucleotide
acaagggccuggacuauga

SEQ ID NO: 936 - RNA, 19 nt, Artificial Sequence
  Description of Artificial Sequence Synthetic oligonucleotide
gccuuaaucuggaucauaa

SEQ ID NO: 937 - RNA, 19 nt, Artificial Sequence
  Description of Artificial Sequence Synthetic oligonucleotide
gacauggccuucuguaaga

SEQ ID NO: 938 - RNA, 19 nt, Artificial Sequence
  Description of Artificial Sequence Synthetic oligonucleotide
gcugauggcaguacacuua

SEQ ID NO: 939 - RNA, 19 nt, Artificial Sequence
  Description of Artificial Sequence Synthetic oligonucleotide
gauccaccuucuccuagug

SEQ ID NO: 940 - RNA, 19 nt, Artificial Sequence
  Description of Artificial Sequence Synthetic oligonucleotide
uaugagcaaccaaagauaa

SEQ ID NO: 941 - RNA, 19 nt, Artificial Sequence
  Description of Artificial Sequence Synthetic oligonucleotide
ggagauggcccgugucuga

SEQ ID NO: 942 - RNA, 19 nt, Artificial Sequence
  Description of Artificial Sequence Synthetic oligonucleotide
ggacaugguuauaacucau

SEQ ID NO: 943 - RNA, 19 nt, Artificial Sequence
  Description of Artificial Sequence Synthetic oligonucleotide
cuuaucaggucccagguua

SEQ ID NO: 944 - RNA, 19 nt, Artificial Sequence
  Description of Artificial Sequence Synthetic oligonucleotide
ccucaaucuagaaauguua

SEQ ID NO: 945 - RNA, 19 nt, Artificial Sequence
  Description of Artificial Sequence Synthetic oligonucleotide
cucaagagcucaaguaugg

SEQ ID NO: 946 - RNA, 19 nt, Artificial Sequence
  Description of Artificial Sequence Synthetic oligonucleotide
gcacaaauugagcauuaug

SEQ ID NO: 947 - RNA, 19 nt, Artificial Sequence
  Description of Artificial Sequence Synthetic oligonucleotide
gccaaugauuccuauguua

SEQ ID NO: 948 - RNA, 19 nt, Artificial Sequence
  Description of Artificial Sequence Synthetic oligonucleotide
gaacggauuaggcuucuga

SEQ ID NO: 949 - RNA, 19 nt, Artificial Sequence
  Description of Artificial Sequence Synthetic oligonucleotide
gcagagauauccgcuauaa

SEQ ID NO: 950 - RNA, 19 nt, Artificial Sequence
  Description of Artificial Sequence Synthetic oligonucleotide
gcgaauugggaaucaguaa

SEQ ID NO: 951 - RNA, 19 nt, Artificial Sequence
  Description of Artificial Sequence Synthetic oligonucleotide
ucauuugccucaucacaug

SEQ ID NO: 952 - RNA, 19 nt, Artificial Sequence
  Description of Artificial Sequence Synthetic oligonucleotide
caguagccaucaauagguu

SEQ ID NO: 953 - RNA, 19 nt, Artificial Sequence
  Description of Artificial Sequence Synthetic oligonucleotide
caacaggacuucauuauuc

SEQ ID NO: 954 - RNA, 19 nt, Artificial Sequence
  Description of Artificial Sequence Synthetic oligonucleotide
gcaaguaggagcuuuguac

SEQ ID NO: 955 - RNA, 19 nt, Artificial Sequence
  Description of Artificial Sequence Synthetic oligonucleotide
uaggauacacccagacaaa

SEQ ID NO: 956 - RNA, 19 nt, Artificial Sequence
  Description of Artificial Sequence Synthetic oligonucleotide
gaacucaggcggaaaagua

SEQ ID NO: 957 - RNA, 19 nt, Artificial Sequence
  Description of Artificial Sequence Synthetic oligonucleotide
gaaagagcaucuacgguga

SEQ ID NO: 958 - RNA, 19 nt, Artificial Sequence
  Description of Artificial Sequence Synthetic oligonucleotide
gaaaggauuuggcuacaaa

SEQ ID NO: 959 - RNA, 19 nt, Artificial Sequence
  Description of Artificial Sequence Synthetic oligonucleotide
ggaaagacuguuccaaaaa

SEQ ID NO: 960 - RNA, 19 nt, Artificial Sequence
  Description of Artificial Sequence Synthetic oligonucleotide
acagcaaauuccaucgugu

SEQ ID NO: 961 - RNA, 19 nt, Artificial Sequence
  Description of Artificial Sequence Synthetic oligonucleotide
gaaauuacagacaggaguu

SEQ ID NO: 962 - RNA, 19 nt, Artificial Sequence
  Description of Artificial Sequence Synthetic oligonucleotide
gcagaugauuugcggauua

SEQ ID NO: 963 - RNA, 19 nt, Artificial Sequence
  Description of Artificial Sequence Synthetic oligonucleotide
uaucaagccugccaauaug

SEQ ID NO: 964 - RNA, 19 nt, Artificial Sequence
  Description of Artificial Sequence Synthetic oligonucleotide
uaugaugacuagagacuau

SEQ ID NO: 965 - RNA, 19 nt, Artificial Sequence
  Description of Artificial Sequence Synthetic oligonucleotide
gagauaagcaccucaagua

SEQ ID NO: 966 - RNA, 19 nt, Artificial Sequence
  Description of Artificial Sequence Synthetic oligonucleotide
gaagaccuacggagacaau

SEQ ID NO: 967 - RNA, 19 nt, Artificial Sequence
  Description of Artificial Sequence Synthetic oligonucleotide
caguguguguauuguacua

SEQ ID NO: 968 - RNA, 19 nt, Artificial Sequence
  Description of Artificial Sequence Synthetic oligonucleotide
guucacagccuccagcaua

SEQ ID NO: 969 - RNA, 19 nt, Artificial Sequence
  Description of Artificial Sequence Synthetic oligonucleotide
aguggauauuccuggcuaa

SEQ ID NO: 970 - RNA, 19 nt, Artificial Sequence
  Description of Artificial Sequence Synthetic oligonucleotide
gggagaaacugcuaugagu

SEQ ID NO: 971 - RNA, 19 nt, Artificial Sequence
  Description of Artificial Sequence Synthetic oligonucleotide
gaacgaggcaucugaucug

SEQ ID NO: 972 - RNA, 19 nt, Artificial Sequence
  Description of Artificial Sequence Synthetic oligonucleotide
gucaacauguggaacgcaa

SEQ ID NO: 973 - RNA, 19 nt, Artificial Sequence
  Description of Artificial Sequence Synthetic oligonucleotide
gcaggagugucuaguuaua

SEQ ID NO: 974 - RNA, 19 nt, Artificial Sequence
  Description of Artificial Sequence Synthetic oligonucleotide
ggaaggcgguuuauaguga

SEQ ID NO: 975 - RNA, 19 nt, Artificial Sequence
  Description of Artificial Sequence Synthetic oligonucleotide
gcaguugucucaauaucua

SEQ ID NO: 976 - RNA, 19 nt, Artificial Sequence
  Description of Artificial Sequence Synthetic oligonucleotide
uaucgaagaugaagacuua

SEQ ID NO: 977 - RNA, 19 nt, Artificial Sequence
  Description of Artificial Sequence Synthetic oligonucleotide
gccuaaagcaggagcaaca

SEQ ID NO: 978 - RNA, 19 nt, Artificial Sequence
  Description of Artificial Sequence Synthetic oligonucleotide
gauagggauggccacauua

SEQ ID NO: 979 - RNA, 19 nt, Artificial Sequence
  Description of Artificial Sequence Synthetic oligonucleotide
gaacaaggcgauuaggaaa

SEQ ID NO: 980 - RNA, 19 nt, Artificial Sequence
  Description of Artificial Sequence Synthetic oligonucleotide
ggagcaacacguacacaau

SEQ ID NO: 981 - RNA, 19 nt, Artificial Sequence
  Description of Artificial Sequence Synthetic oligonucleotide
ccaaggagacucauguaau

SEQ ID NO: 982 - RNA, 19 nt, Artificial Sequence
  Description of Artificial Sequence Synthetic oligonucleotide
ugaaugccgucaagaccaa

SEQ ID NO: 983 - RNA, 19 nt, Artificial Sequence
  Description of Artificial Sequence Synthetic oligonucleotide
augcugagcuuuaucauau

SEQ ID NO: 984 - RNA, 19 nt, Artificial Sequence
  Description of Artificial Sequence Synthetic oligonucleotide
gaaaugcauucuggugauu

SEQ ID NO: 985 - RNA, 19 nt, Artificial Sequence
  Description of Artificial Sequence Synthetic oligonucleotide
acuacaggaugcuuucauu

SEQ ID NO: 986 - RNA, 19 nt, Artificial Sequence
  Description of Artificial Sequence Synthetic oligonucleotide
gcacugucaaguaaacuac

SEQ ID NO: 987 - RNA, 19 nt, Artificial Sequence
  Description of Artificial Sequence Synthetic oligonucleotide
gaaaauucuuccaaggaug

SEQ ID NO: 988 - RNA, 19 nt, Artificial Sequence
  Description of Artificial Sequence Synthetic oligonucleotide
gaaggaaucggacggaguc

SEQ ID NO: 989 - RNA, 19 nt, Artificial Sequence
  Description of Artificial Sequence Synthetic oligonucleotide
ggagaggauucaaauauua

SEQ ID NO: 990 - RNA, 19 nt, Artificial Sequence
  Description of Artificial Sequence Synthetic oligonucleotide
ucgaagagauuaacaagug

SEQ ID NO: 991 - RNA, 19 nt, Artificial Sequence
  Description of Artificial Sequence Synthetic oligonucleotide
ucagacagauuacaucaau

SEQ ID NO: 992 - RNA, 19 nt, Artificial Sequence
  Description of Artificial Sequence Synthetic oligonucleotide
gagaauaccuaucgugauu

SEQ ID NO: 993 - RNA, 19 nt, Artificial Sequence
  Description of Artificial Sequence Synthetic oligonucleotide
gcuucuaccuggacgugaa

SEQ ID NO: 994 - RNA, 19 nt, Artificial Sequence
  Description of Artificial Sequence Synthetic oligonucleotide
caacaagcgcuucuucuuc

SEQ ID NO: 995 - RNA, 19 nt, Artificial Sequence
  Description of Artificial Sequence Synthetic oligonucleotide
gaugugggcuccaacaagu

SEQ ID NO: 996 - RNA, 19 nt, Artificial Sequence
  Description of Artificial Sequence Synthetic oligonucleotide
ggacacaccuucugcaagu

SEQ ID NO: 997 - RNA, 19 nt, Artificial Sequence
  Description of Artificial Sequence Synthetic oligonucleotide
ccagcgagaacgucaauaa

SEQ ID NO: 998 - RNA, 19 nt, Artificial Sequence
  Description of Artificial Sequence Synthetic oligonucleotide
gcgccaagaaugccaccaa

SEQ ID NO: 999 - RNA, 19 nt, Artificial Sequence
  Description of Artificial Sequence Synthetic oligonucleotide
cagccaaggaguuugcaga

SEQ ID NO: 1000 - RNA, 19 nt, Artificial Sequence
  Description of Artificial Sequence Synthetic oligonucleotide
gaccauggcugcugaaauc

SEQ ID NO: 1001 - RNA, 19 nt, Artificial Sequence
  Description of Artificial Sequence Synthetic oligonucleotide
gaaggagucuuugacauua

SEQ ID NO: 1002 - RNA, 19 nt, Artificial Sequence
  Description of Artificial Sequence Synthetic oligonucleotide
gauauuacacggagagaua

SEQ ID NO: 1003 - RNA, 19 nt, Artificial Sequence
  Description of Artificial Sequence Synthetic oligonucleotide
ugaccuuacuauuggugua

SEQ ID NO: 1004 - RNA, 19 nt, Artificial Sequence
  Description of Artificial Sequence Synthetic oligonucleotide
cgaaugauaacuauugaug

SEQ ID NO: 1005 - RNA, 19 nt, Artificial Sequence
  Description of Artificial Sequence Synthetic oligonucleotide
gagcauaugcgaacaauaa

SEQ ID NO: 1006 - RNA, 19 nt, Artificial Sequence
  Description of Artificial Sequence Synthetic oligonucleotide
ugaaguacccggaagaaga

SEQ ID NO: 1007 - RNA, 19 nt, Artificial Sequence
  Description of Artificial Sequence Synthetic oligonucleotide
gguauacuguggugaagaa

SEQ ID NO: 1008 - RNA, 19 nt, Artificial Sequence
  Description of Artificial Sequence Synthetic oligonucleotide
gaauguuacccuucaaauu

SEQ ID NO: 1009 - RNA, 19 nt, Artificial Sequence
  Description of Artificial Sequence Synthetic oligonucleotide
aagcagagaagauaugauu

SEQ ID NO: 1010 - RNA, 19 nt, Artificial Sequence
  Description of Artificial Sequence Synthetic oligonucleotide
gagcaaccagucagugaag

SEQ ID NO: 1011 - RNA, 19 nt, Artificial Sequence
  Description of Artificial Sequence Synthetic oligonucleotide
ccaaagagcugaauguuau

SEQ ID NO: 1012 - RNA, 19 nt, Artificial Sequence
  Description of Artificial Sequence Synthetic oligonucleotide
gagaagauaugauugacau

SEQ ID NO: 1013 - RNA, 19 nt, Artificial Sequence
  Description of Artificial Sequence Synthetic oligonucleotide
agacggaccuggcugauaa

SEQ ID NO: 1014 - RNA, 19 nt, Artificial Sequence
  Description of Artificial Sequence Synthetic oligonucleotide
ggaauccacugagaaaauu

SEQ ID NO: 1015 - RNA, 19 nt, Artificial Sequence
  Description of Artificial Sequence Synthetic oligonucleotide
ccaaagaacugagcgucau

SEQ ID NO: 1016 - RNA, 19 nt, Artificial Sequence
  Description of Artificial Sequence Synthetic oligonucleotide
ggaagacgucucugauuac

SEQ ID NO: 1017 - RNA, 19 nt, Artificial Sequence
  Description of Artificial Sequence Synthetic oligonucleotide
aauauuggcuugaaccuuu

SEQ ID NO: 1018 - RNA, 19 nt, Artificial Sequence
  Description of Artificial Sequence Synthetic oligonucleotide
gggcugaauguuacguuua

SEQ ID NO: 1019 - RNA, 19 nt, Artificial Sequence
  Description of Artificial Sequence Synthetic oligonucleotide
cugcagcuguaguaguuua

SEQ ID NO: 1020 - RNA, 19 nt, Artificial Sequence
  Description of Artificial Sequence Synthetic oligonucleotide
cuacaaaguggauugauga

SEQ ID NO: 1021 - RNA, 19 nt, Artificial Sequence
  Description of Artificial Sequence Synthetic oligonucleotide
gaaaccagcuauaugucuu

SEQ ID NO: 1022 - RNA, 19 nt, Artificial Sequence
  Description of Artificial Sequence Synthetic oligonucleotide
ggauucagcugacaaagua

SEQ ID NO: 1023 - RNA, 19 nt, Artificial Sequence
  Description of Artificial Sequence Synthetic oligonucleotide
ggaaaucgaaauugucuac

SEQ ID NO: 1024 - RNA, 19 nt, Artificial Sequence
  Description of Artificial Sequence Synthetic oligonucleotide
ccacagcuguucauauuua

SEQ ID NO: 1025 - RNA, 19 nt, Artificial Sequence
  Description of Artificial Sequence Synthetic oligonucleotide
gaaaggacauguaucacug

SEQ ID NO: 1026 - RNA, 19 nt, Artificial Sequence
  Description of Artificial Sequence Synthetic oligonucleotide
gaaagaaggucacugggau

SEQ ID NO: 1027 - RNA, 19 nt, Artificial Sequence
  Description of Artificial Sequence Synthetic oligonucleotide
cgaaacagcugucaagaaa

SEQ ID NO: 1028 - RNA, 19 nt, Artificial Sequence
  Description of Artificial Sequence Synthetic oligonucleotide
gaauaacuaucacagaaug

SEQ ID NO: 1029 - RNA, 19 nt, Artificial Sequence
  Description of Artificial Sequence Synthetic oligonucleotide
gcaaagcuguauuaugaag

SEQ ID NO: 1030 - RNA, 19 nt, Artificial Sequence
  Description of Artificial Sequence Synthetic oligonucleotide
uuacaggcguucaguggaa

SEQ ID NO: 1031 - RNA, 19 nt, Artificial Sequence
  Description of Artificial Sequence Synthetic oligonucleotide
gaaguccugcaauuuauaa

SEQ ID NO: 1032 - RNA, 19 nt, Artificial Sequence
  Description of Artificial Sequence Synthetic oligonucleotide
guuccaagaccaaagauua

SEQ ID NO: 1033 - RNA, 19 nt, Artificial Sequence
  Description of Artificial Sequence Synthetic oligonucleotide
aguauaagcuagucguucu

SEQ ID NO: 1034 - RNA, 19 nt, Artificial Sequence
  Description of Artificial Sequence Synthetic oligonucleotide
gaccuagugcggcaaauua

SEQ ID NO: 1035 - RNA, 19 nt, Artificial Sequence
  Description of Artificial Sequence Synthetic oligonucleotide
caaugauucuuguugguaa

SEQ ID NO: 1036 - RNA, 19 nt, Artificial Sequence
  Description of Artificial Sequence Synthetic oligonucleotide
gaacaacugugcauucuua

SEQ ID NO: 1037 - RNA, 19 nt, Artificial Sequence
  Description of Artificial Sequence Synthetic oligonucleotide
ggacacaucuaggcacugu

SEQ ID NO: 1038 - RNA, 19 nt, Artificial Sequence
  Description of Artificial Sequence Synthetic oligonucleotide
gcacaagacuucagaugca

SEQ ID NO: 1039 - RNA, 19 nt, Artificial Sequence
  Description of Artificial Sequence Synthetic oligonucleotide
gaaaauggguugcagaaaa

SEQ ID NO: 1040 - RNA, 19 nt, Artificial Sequence
  Description of Artificial Sequence Synthetic oligonucleotide
agaggcagcuccuuaauaa

SEQ ID NO: 1041 - RNA, 19 nt, Artificial Sequence
  Description of Artificial Sequence Synthetic oligonucleotide
gaggaacgacgacauuaua

SEQ ID NO: 1042 - RNA, 19 nt, Artificial Sequence
  Description of Artificial Sequence Synthetic oligonucleotide
gcagaacgauccucgaauu

SEQ ID NO: 1043 - RNA, 19 nt, Artificial Sequence
  Description of Artificial Sequence Synthetic oligonucleotide
augcugagcucuucuauaa

SEQ ID NO: 1044 - RNA, 19 nt, Artificial Sequence
  Description of Artificial Sequence Synthetic oligonucleotide
guacagauaugagaaauuc

SEQ ID NO: 1045 - RNA, 19 nt, Artificial Sequence
  Description of Artificial Sequence Synthetic oligonucleotide
ggacaaagaauacaugaaa

SEQ ID NO: 1046 - RNA, 19 nt, Artificial Sequence
  Description of Artificial Sequence Synthetic oligonucleotide
ggaagagcauucaguagua

SEQ ID NO: 1047 - RNA, 19 nt, Artificial Sequence
  Description of Artificial Sequence Synthetic oligonucleotide
ggaagaaagugcccguaaa

SEQ ID NO: 1048 - RNA, 19 nt, Artificial Sequence
  Description of Artificial Sequence Synthetic oligonucleotide
ucaaguggcucccaugaua

SEQ ID NO: 1049 - RNA, 19 nt, Artificial Sequence
  Description of Artificial Sequence Synthetic oligonucleotide
ggcuauaacucgccuagug

SEQ ID NO: 1050 - RNA, 19 nt, Artificial Sequence
  Description of Artificial Sequence Synthetic oligonucleotide
gaugagaucuuccuacugu

SEQ ID NO: 1051 - RNA, 19 nt, Artificial Sequence
  Description of Artificial Sequence Synthetic oligonucleotide
ggauugaggagaaacguaa

SEQ ID NO: 1052 - RNA, 19 nt, Artificial Sequence
  Description of Artificial Sequence Synthetic oligonucleotide
cucaagaucugccgaguga

SEQ ID NO: 1053 - RNA, 19 nt, Artificial Sequence
  Description of Artificial Sequence Synthetic oligonucleotide
gaagugggcgcaucagaca

SEQ ID NO: 1054 - RNA, 19 nt, Artificial Sequence
  Description of Artificial Sequence Synthetic oligonucleotide
gaaggugguucccaugucu

SEQ ID NO: 1055 - RNA, 19 nt, Artificial Sequence
  Description of Artificial Sequence Synthetic oligonucleotide
cuucguagaccgcaaguua

SEQ ID NO: 1056 - RNA, 19 nt, Artificial Sequence
  Description of Artificial Sequence Synthetic oligonucleotide
uuaauucacugcagaagga

SEQ ID NO: 1057 - RNA, 19 nt, Artificial Sequence
  Description of Artificial Sequence Synthetic oligonucleotide
cuuaguagaccgcaaguua

SEQ ID NO: 1058 - RNA, 19 nt, Artificial Sequence
  Description of Artificial Sequence Synthetic oligonucleotide
gcucagacuaucuggaaaa

SEQ ID NO: 1059 - RNA, 19 nt, Artificial Sequence
  Description of Artificial Sequence Synthetic oligonucleotide
ccauggucucucagaggaa

SEQ ID NO: 1060 - RNA, 19 nt, Artificial Sequence
  Description of Artificial Sequence Synthetic oligonucleotide
gagaaucuguucacugcaa

SEQ ID NO: 1061 - RNA, 19 nt, Artificial Sequence
  Description of Artificial Sequence Synthetic oligonucleotide
gaagauaggacggaaauua

SEQ ID NO: 1062 - RNA, 19 nt, Artificial Sequence
  Description of Artificial Sequence Synthetic oligonucleotide
ggaaagagcuuguacaguu

SEQ ID NO: 1063 - RNA, 19 nt, Artificial Sequence
  Description of Artificial Sequence Synthetic oligonucleotide
ucacucagcuucagccuua

SEQ ID NO: 1064 - RNA, 19 nt, Artificial Sequence
  Description of Artificial Sequence Synthetic oligonucleotide
gaaacugaguccauucuuu

SEQ ID NO: 1065 - RNA, 19 nt, Artificial Sequence
  Description of Artificial Sequence Synthetic oligonucleotide
caaagucgcucgcaagucg

SEQ ID NO: 1066 - RNA, 19 nt, Artificial Sequence
  Description of Artificial Sequence Synthetic oligonucleotide
gcucagagggcaaccuuaa

SEQ ID NO: 1067 - RNA, 19 nt, Artificial Sequence
  Description of Artificial Sequence Synthetic oligonucleotide
agacacugccagcggccua

SEQ ID NO: 1068 - RNA, 19 nt, Artificial Sequence
  Description of Artificial Sequence Synthetic oligonucleotide
gaguuugccuggcgucgga

SEQ ID NO: 1069 - RNA, 19 nt, Artificial Sequence
  Description of Artificial Sequence Synthetic oligonucleotide
gaacugugcuuaaaaguaa

SEQ ID NO: 1070 - RNA, 19 nt, Artificial Sequence
  Description of Artificial Sequence Synthetic oligonucleotide
cagagagacuuuacuguuu

SEQ ID NO: 1071 - RNA, 19 nt, Artificial Sequence
  Description of Artificial Sequence Synthetic oligonucleotide
gggaaaagcuugacgauua

SEQ ID NO: 1072 - RNA, 19 nt, Artificial Sequence
  Description of Artificial Sequence Synthetic oligonucleotide
gcacauguaucgaauuuac

SEQ ID NO: 1073 - RNA, 19 nt, Artificial Sequence
  Description of Artificial Sequence Synthetic oligonucleotide
gaaacuggaugaugauuca

SEQ ID NO: 1074 - RNA, 19 nt, Artificial Sequence
  Description of Artificial Sequence Synthetic oligonucleotide
gagauugcaucaggauauu

SEQ ID NO: 1075 - RNA, 19 nt, Artificial Sequence
  Description of Artificial Sequence Synthetic oligonucleotide
ggccaaauaucaaggugaa

SEQ ID NO: 1076 - RNA, 19 nt, Artificial Sequence
  Description of Artificial Sequence Synthetic oligonucleotide
gggcaacccagaucuauua

SEQ ID NO: 1077 - RNA, 19 nt, Artificial Sequence
  Description of Artificial Sequence Synthetic oligonucleotide
ugacuagucuucugcacuu

SEQ ID NO: 1078 - RNA, 19 nt, Artificial Sequence
  Description of Artificial Sequence Synthetic oligonucleotide
gccgagagaggcucaauga

SEQ ID NO: 1079 - RNA, 19 nt, Artificial Sequence
  Description of Artificial Sequence Synthetic oligonucleotide
gcagaucagaggcagaaga

SEQ ID NO: 1080 - RNA, 19 nt, Artificial Sequence
  Description of Artificial Sequence Synthetic oligonucleotide
cguaguggcugccuuccua

SEQ ID NO: 1081 - RNA, 19 nt, Artificial Sequence
  Description of Artificial Sequence Synthetic oligonucleotide
gcaaauugagagugucuug

SEQ ID NO: 1082 - RNA, 19 nt, Artificial Sequence
  Description of Artificial Sequence Synthetic oligonucleotide
gaauaaugucuggggugaa

SEQ ID NO: 1083 - RNA, 19 nt, Artificial Sequence
  Description of Artificial Sequence Synthetic oligonucleotide
uaccacgggugcaggaauu

SEQ ID NO: 1084 - RNA, 19 nt, Artificial Sequence
  Description of Artificial Sequence Synthetic oligonucleotide
acacuggcagggugggauu

SEQ ID NO: 1085 - RNA, 19 nt, Artificial Sequence
  Description of Artificial Sequence Synthetic oligonucleotide
gaggcuaggucaggaauua

SEQ ID NO: 1086 - RNA, 19 nt, Artificial Sequence
  Description of Artificial Sequence Synthetic oligonucleotide
agaccaaaguucucacuau

SEQ ID NO: 1087 - RNA, 19 nt, Artificial Sequence
  Description of Artificial Sequence Synthetic oligonucleotide
ccagagcuaucauuauggu

SEQ ID NO: 1088 - RNA, 19 nt, Artificial Sequence
  Description of Artificial Sequence Synthetic oligonucleotide
ggacaagccucucacuuua

SEQ ID NO: 1089 - RNA, 19 nt, Artificial Sequence
  Description of Artificial Sequence Synthetic oligonucleotide
gggaugaaacccucuauaa

SEQ ID NO: 1090 - RNA, 19 nt, Artificial Sequence
  Description of Artificial Sequence Synthetic oligonucleotide
gagaauggauccaccacua

SEQ ID NO: 1091 - RNA, 19 nt, Artificial Sequence
  Description of Artificial Sequence Synthetic oligonucleotide
uaucgaagcugaugaagua

SEQ ID NO: 1092 - RNA, 19 nt, Artificial Sequence
  Description of Artificial Sequence Synthetic oligonucleotide
caaauuauauugccagaga

SEQ ID NO: 1093 - RNA, 19 nt, Artificial Sequence
  Description of Artificial Sequence Synthetic oligonucleotide
gggagacauuucuaucaaa

SEQ ID NO: 1094 - RNA, 19 nt, Artificial Sequence
  Description of Artificial Sequence Synthetic oligonucleotide
uccaguaucuguaaaccuu

SEQ ID NO: 1095 - RNA, 19 nt, Artificial Sequence
  Description of Artificial Sequence Synthetic oligonucleotide
agauugaccuugccucaua

SEQ ID NO: 1096 - RNA, 19 nt, Artificial Sequence
  Description of Artificial Sequence Synthetic oligonucleotide
gacgcucucuugacgcauu

SEQ ID NO: 1097 - RNA, 19 nt, Artificial Sequence
  Description of Artificial Sequence Synthetic oligonucleotide
gcucagaucgaccaauaug

SEQ ID NO: 1098 - RNA, 19 nt, Artificial Sequence
  Description of Artificial Sequence Synthetic oligonucleotide
gggagugauuggucuauuc

SEQ ID NO: 1099 - RNA, 19 nt, Artificial Sequence
  Description of Artificial Sequence Synthetic oligonucleotide
gggagcagacggaacguuu

SEQ ID NO: 1100 - RNA, 19 nt, Artificial Sequence
  Description of Artificial Sequence Synthetic oligonucleotide
uagaagaccucguggauuc

SEQ ID NO: 1101 - RNA, 19 nt, Artificial Sequence
  Description of Artificial Sequence Synthetic oligonucleotide
ggugggagcacaagaccua

SEQ ID NO: 1102 - RNA, 19 nt, Artificial Sequence
  Description of Artificial Sequence Synthetic oligonucleotide
gguuuaaucaccucuauaa

SEQ ID NO: 1103 - RNA, 19 nt, Artificial Sequence
  Description of Artificial Sequence Synthetic oligonucleotide
gagccuagaccgaagauca

SEQ ID NO: 1104 - RNA, 19 nt, Artificial Sequence
  Description of Artificial Sequence Synthetic oligonucleotide
ggacaaguauacacgaaua

SEQ ID NO: 1105 - RNA, 19 nt, Artificial Sequence
  Description of Artificial Sequence Synthetic oligonucleotide
gggccuuggcuuugaucua

SEQ ID NO: 1106 - RNA, 19 nt, Artificial Sequence
  Description of Artificial Sequence Synthetic oligonucleotide
gcucgaagcugcaggaugc

SEQ ID NO: 1107 - RNA, 19 nt, Artificial Sequence
  Description of Artificial Sequence Synthetic oligonucleotide
acaaggaccaggaggugaa

SEQ ID NO: 1108 - RNA, 19 nt, Artificial Sequence
  Description of Artificial Sequence Synthetic oligonucleotide
agaaggagcucaccauugg

SEQ ID NO: 1109 - RNA, 19 nt, Artificial Sequence
  Description of Artificial Sequence Synthetic oligonucleotide
gaacgaacauuggcucggc

SEQ ID NO: 1110 - RNA, 19 nt, Artificial Sequence
  Description of Artificial Sequence Synthetic oligonucleotide
gugaacaacuucccaccau

SEQ ID NO: 1111 - RNA, 19 nt, Artificial Sequence
  Description of Artificial Sequence Synthetic oligonucleotide
caacuuuggccucgccuuu

SEQ ID NO: 1112 - RNA, 19 nt, Artificial Sequence
  Description of Artificial Sequence Synthetic oligonucleotide
guacuccaaugagauguga

SEQ ID NO: 1113 - RNA, 19 nt, Artificial Sequence
  Description of Artificial Sequence Synthetic oligonucleotide
gcaagaaaauuggauguau

SEQ ID NO: 1114 - RNA, 19 nt, Artificial Sequence
  Description of Artificial Sequence Synthetic oligonucleotide
cgaaacugaugacuauaga

SEQ ID NO: 1115 - RNA, 19 nt, Artificial Sequence
  Description of Artificial Sequence Synthetic oligonucleotide
uaaggagcuuguuucauau

SEQ ID NO: 1116 - RNA, 19 nt, Artificial Sequence
  Description of Artificial Sequence Synthetic oligonucleotide
gacaauaccgcuaagcuaa

SEQ ID NO: 1117 - RNA, 19 nt, Artificial Sequence
  Description of Artificial Sequence Synthetic oligonucleotide
ccagugcaccuacauugaa

SEQ ID NO: 1118 - RNA, 19 nt, Artificial Sequence
  Description of Artificial Sequence Synthetic oligonucleotide
caucccggcugugaucuuu

SEQ ID NO: 1119 - RNA, 19 nt, Artificial Sequence
  Description of Artificial Sequence Synthetic oligonucleotide
gcgccauucuccuaccaua

SEQ ID NO: 1120 - RNA, 19 nt, Artificial Sequence
  Description of Artificial Sequence Synthetic oligonucleotide
ucagguagaucgucggcau

SEQ ID NO: 1121 - RNA, 19 nt, Artificial Sequence
  Description of Artificial Sequence Synthetic oligonucleotide
auacuaccuucgccaauua

SEQ ID NO: 1122 - RNA, 19 nt, Artificial Sequence
  Description of Artificial Sequence Synthetic oligonucleotide
caauauaccagcaacauua

SEQ ID NO: 1123 - RNA, 19 nt, Artificial Sequence
  Description of Artificial Sequence Synthetic oligonucleotide
gcacauugaggcuuauaau

SEQ ID NO: 1124 - RNA, 19 nt, Artificial Sequence
  Description of Artificial Sequence Synthetic oligonucleotide
ccgaccacaucaagagaua

SEQ ID NO: 1125 - RNA, 19 nt, Artificial Sequence
  Description of Artificial Sequence Synthetic oligonucleotide
ucaaagaacugguccguuu

SEQ ID NO: 1126 - RNA, 19 nt, Artificial Sequence
  Description of Artificial Sequence Synthetic oligonucleotide
gcgagggcaucaaguuauc

SEQ ID NO: 1127 - RNA, 19 nt, Artificial Sequence
  Description of Artificial Sequence Synthetic oligonucleotide
gacuaaacgucgacuugug

SEQ ID NO: 1128 - RNA, 19 nt, Artificial Sequence
  Description of Artificial Sequence Synthetic oligonucleotide
ggacaccaauggguuaugu

SEQ ID NO: 1129 - RNA, 19 nt, Artificial Sequence
  Description of Artificial Sequence Synthetic oligonucleotide
gaacagaucagauauagga

SEQ ID NO: 1130 - RNA, 19 nt, Artificial Sequence
  Description of Artificial Sequence Synthetic oligonucleotide
cuacaucgaugcgcaguuu

SEQ ID NO: 1131 - RNA, 19 nt, Artificial Sequence
  Description of Artificial Sequence Synthetic oligonucleotide
gaugagaggcccauaguug

SEQ ID NO: 1132 - RNA, 19 nt, Artificial Sequence
  Description of Artificial Sequence Synthetic oligonucleotide
uaagugagcugcagaggaa

SEQ ID NO: 1133 - RNA, 19 nt, Artificial Sequence
  Description of Artificial Sequence Synthetic oligonucleotide
uauagaaccaugccaauua

SEQ ID NO: 1134 - RNA, 19 nt, Artificial Sequence
  Description of Artificial Sequence Synthetic oligonucleotide
gaacuuaaucagcaaauug

SEQ ID NO: 1135 - RNA, 19 nt, Artificial Sequence
  Description of Artificial Sequence Synthetic oligonucleotide
gagaguacauagauuggaa

SEQ ID NO: 1136 - RNA, 19 nt, Artificial Sequence
  Description of Artificial Sequence Synthetic oligonucleotide
ggaugaaacuaguuaaucu

SEQ ID NO: 1137 - RNA, 19 nt, Artificial Sequence
  Description of Artificial Sequence Synthetic oligonucleotide
ggagugugcuggauauauu

SEQ ID NO: 1138 - RNA, 19 nt, Artificial Sequence
  Description of Artificial Sequence Synthetic oligonucleotide
caaacugcaucuacgauau

SEQ ID NO: 1139 - RNA, 19 nt, Artificial Sequence
  Description of Artificial Sequence Synthetic oligonucleotide
gagaggaccgcacuuuaug

SEQ ID NO: 1140 - RNA, 19 nt, Artificial Sequence
  Description of Artificial Sequence Synthetic oligonucleotide
ccuggacgaugacggauua

SEQ ID NO: 1141 - RNA, 19 nt, Artificial Sequence
  Description of Artificial Sequence Synthetic oligonucleotide
gggacuggcuguguuauuc

SEQ ID NO: 1142 - RNA, 19 nt, Artificial Sequence
  Description of Artificial Sequence Synthetic oligonucleotide
gaacuggauugcuguggcu

SEQ ID NO: 1143 - RNA, 19 nt, Artificial Sequence
  Description of Artificial Sequence Synthetic oligonucleotide
gggaugcaguuauuaaaug

SEQ ID NO: 1144 - RNA, 19 nt, Artificial Sequence
  Description of Artificial Sequence Synthetic oligonucleotide
uugcaguucuugucaauga

SEQ ID NO: 1145 - RNA, 19 nt, Artificial Sequence
  Description of Artificial Sequence Synthetic oligonucleotide
gaagcaaagugugaauauu

SEQ ID NO: 1146 - RNA, 19 nt, Artificial Sequence
  Description of Artificial Sequence Synthetic oligonucleotide
gaucgaagcagcucaaugu

SEQ ID NO: 1147 - RNA, 19 nt, Artificial Sequence
  Description of Artificial Sequence Synthetic oligonucleotide
uaacuagugucuuggaaua

SEQ ID NO: 1148 - RNA, 19 nt, Artificial Sequence
  Description of Artificial Sequence Synthetic oligonucleotide
gagcggaacuggcugguuu

SEQ ID NO: 1149 - RNA, 19 nt, Artificial Sequence
  Description of Artificial Sequence Synthetic oligonucleotide
gccaggagcucaaacuauu

SEQ ID NO: 1150 - RNA, 19 nt, Artificial Sequence
  Description of Artificial Sequence Synthetic oligonucleotide
ggagaacaccgaaaacaau

SEQ ID NO: 1151 - RNA, 19 nt, Artificial Sequence
  Description of Artificial Sequence Synthetic oligonucleotide
gaaggcgcauuacguggag

SEQ ID NO: 1152 - RNA, 19 nt, Artificial Sequence
  Description of Artificial Sequence Synthetic oligonucleotide
gcacaagaacgagagcgua

SEQ ID NO: 1153 - RNA, 19 nt, Artificial Sequence
  Description of Artificial Sequence Synthetic oligonucleotide
gauggagggucuguaguga

SEQ ID NO: 1154 - RNA, 19 nt, Artificial Sequence
  Description of Artificial Sequence Synthetic oligonucleotide
ugucuuagauggcaugguu

SEQ ID NO: 1155 - RNA, 19 nt, Artificial Sequence
  Description of Artificial Sequence Synthetic oligonucleotide
ccaguggaguuauccuuaa

SEQ ID NO: 1156 - RNA, 19 nt, Artificial Sequence
  Description of Artificial Sequence Synthetic oligonucleotide
guauacacgguuccuaaua

SEQ ID NO: 1157 - RNA, 19 nt, Artificial Sequence
  Description of Artificial Sequence Synthetic oligonucleotide
ggugaucacugugcacuuu

SEQ ID NO: 1158 - RNA, 19 nt, Artificial Sequence
  Description of Artificial Sequence Synthetic oligonucleotide
caucuuaacccuuuaugaa

SEQ ID NO: 1159 - RNA, 19 nt, Artificial Sequence
  Description of Artificial Sequence Synthetic oligonucleotide
cgguagagccgcuggucuu

SEQ ID NO: 1160 - RNA, 19 nt, Artificial Sequence
  Description of Artificial Sequence Synthetic oligonucleotide
ggaaacauuuagcccucua

SEQ ID NO: 1161 - RNA, 19 nt, Artificial Sequence
  Description of Artificial Sequence Synthetic oligonucleotide
guacauagaugauuuacca

SEQ ID NO: 1162 - RNA, 19 nt, Artificial Sequence
  Description of Artificial Sequence Synthetic oligonucleotide
gaaaucgaauuguugaugu

SEQ ID NO: 1163 - RNA, 19 nt, Artificial Sequence
  Description of Artificial Sequence Synthetic oligonucleotide
gaaguugucuuauggauga

SEQ ID NO: 1164 - RNA, 19 nt, Artificial Sequence
  Description of Artificial Sequence Synthetic oligonucleotide
caacugcaaucuauguuua

SEQ ID NO: 1165 - RNA, 19 nt, Artificial Sequence
  Description of Artificial Sequence Synthetic oligonucleotide
gaaucaagaccaucacuau

SEQ ID NO: 1166 - RNA, 19 nt, Artificial Sequence
  Description of Artificial Sequence Synthetic oligonucleotide
gagaucguauugauauugc

SEQ ID NO: 1167 - RNA, 19 nt, Artificial Sequence
  Description of Artificial Sequence Synthetic oligonucleotide
caacuaaaccgcauagaag

SEQ ID NO: 1168 - RNA, 19 nt, Artificial Sequence
  Description of Artificial Sequence Synthetic oligonucleotide
ggguuuagccauugagucu

SEQ ID NO: 1169 - RNA, 19 nt, Artificial Sequence
  Description of Artificial Sequence Synthetic oligonucleotide
ggaacaacgguuacaguca

SEQ ID NO: 1170 - RNA, 19 nt, Artificial Sequence
  Description of Artificial Sequence Synthetic oligonucleotide
ccaaguauaucaggcuauc

SEQ ID NO: 1171 - RNA, 19 nt, Artificial Sequence
  Description of Artificial Sequence Synthetic oligonucleotide
gugaauuacuuugccuuau

SEQ ID NO: 1172 - RNA, 19 nt, Artificial Sequence
  Description of Artificial Sequence Synthetic oligonucleotide
guacguaaauuggcaccua

SEQ ID NO: 1173 - RNA, 19 nt, Artificial Sequence
  Description of Artificial Sequence Synthetic oligonucleotide
gaauauacguugugaagga

SEQ ID NO: 1174 - RNA, 19 nt, Artificial Sequence
  Description of Artificial Sequence Synthetic oligonucleotide
caaauuaaccugcgugaau

SEQ ID NO: 1175 - RNA, 19 nt, Artificial Sequence
  Description of Artificial Sequence Synthetic oligonucleotide
cuggaagccuguagaaauu

SEQ ID NO: 1176 - RNA, 19 nt, Artificial Sequence
  Description of Artificial Sequence Synthetic oligonucleotide
aaagaugacucaacuugua

SEQ ID NO: 1177 - RNA, 19 nt, Artificial Sequence
  Description of Artificial Sequence Synthetic oligonucleotide
gaacuucaaccuucuauaa

SEQ ID NO: 1178 - RNA, 19 nt, Artificial Sequence
  Description of Artificial Sequence Synthetic oligonucleotide
ggaagacaaugcuggcuaa

SEQ ID NO: 1179 - RNA, 19 nt, Artificial Sequence
  Description of Artificial Sequence Synthetic oligonucleotide
aaacggacgucuauaauga

SEQ ID NO: 1180 - RNA, 19 nt, Artificial Sequence
  Description of Artificial Sequence Synthetic oligonucleotide
uauaagugcugcaaguuua

SEQ ID NO: 1181 - RNA, 19 nt, Artificial Sequence
  Description of Artificial Sequence Synthetic oligonucleotide
acacguaucugucccauuu

SEQ ID NO: 1182 - RNA, 19 nt, Artificial Sequence
  Description of Artificial Sequence Synthetic oligonucleotide
gaagugaucugggauuuau

SEQ ID NO: 1183 - RNA, 19 nt, Artificial Sequence
  Description of Artificial Sequence Synthetic oligonucleotide
gaaccaaguuguccuaugg

SEQ ID NO: 1184 - RNA, 19 nt, Artificial Sequence
  Description of Artificial Sequence Synthetic oligonucleotide
gaaauggucucaagggaaa

SEQ ID NO: 1185 - RNA, 19 nt, Artificial Sequence
  Description of Artificial Sequence Synthetic oligonucleotide
cagccuacaucuucaauag

SEQ ID NO: 1186 - RNA, 19 nt, Artificial Sequence
  Description of Artificial Sequence Synthetic oligonucleotide
ugacucaguucaaguaguu

SEQ ID NO: 1187 - RNA, 19 nt, Artificial Sequence
  Description of Artificial Sequence Synthetic oligonucleotide
gaacaagauuuccuuuggu

SEQ ID NO: 1188 - RNA, 19 nt, Artificial Sequence
  Description of Artificial Sequence Synthetic oligonucleotide
ggcaacagcaccaauaaua

SEQ ID NO: 1189 - RNA, 19 nt, Artificial Sequence
  Description of Artificial Sequence Synthetic oligonucleotide
gagaggaacugauuacaaa

SEQ ID NO: 1190 - RNA, 19 nt, Artificial Sequence
  Description of Artificial Sequence Synthetic oligonucleotide
gcaaagaucuuaccaaugu

SEQ ID NO: 1191 - RNA, 19 nt, Artificial Sequence
  Description of Artificial Sequence Synthetic oligonucleotide
caacagagguaaggauuua

SEQ ID NO: 1192 - RNA, 19 nt, Artificial Sequence
  Description of Artificial Sequence Synthetic oligonucleotide
gcaagaagcuguccgauga

SEQ ID NO: 1193 - RNA, 19 nt, Artificial Sequence
  Description of Artificial Sequence Synthetic oligonucleotide
gacgagaucuuguggguug

SEQ ID NO: 1194 - RNA, 19 nt, Artificial Sequence
  Description of Artificial Sequence Synthetic oligonucleotide
cgagugcaaugaaaccaaa

SEQ ID NO: 1195 - RNA, 19 nt, Artificial Sequence
  Description of Artificial Sequence Synthetic oligonucleotide
cggaagagaucgccaauua

SEQ ID NO: 1196 - RNA, 19 nt, Artificial Sequence
  Description of Artificial Sequence Synthetic oligonucleotide
cuuauguggugacuuauua

SEQ ID NO: 1197 - RNA, 19 nt, Artificial Sequence
  Description of Artificial Sequence Synthetic oligonucleotide
caaaucgagcucugcgcga

SEQ ID NO: 1198 - RNA, 19 nt, Artificial Sequence
  Description of Artificial Sequence Synthetic oligonucleotide
cacuacagcucaucugguu

SEQ ID NO: 1199 - RNA, 19 nt, Artificial Sequence
  Description of Artificial Sequence Synthetic oligonucleotide
gaaucucgcuacggcggau

SEQ ID NO: 1200 - RNA, 19 nt, Artificial Sequence
  Description of Artificial Sequence Synthetic oligonucleotide
cgaucucgcuauagggguu

SEQ ID NO: 1201 - RNA, 19 nt, Artificial Sequence
  Description of Artificial Sequence Synthetic oligonucleotide
gaacauugcauaaagcauu

SEQ ID NO: 1202 - RNA, 19 nt, Artificial Sequence
  Description of Artificial Sequence Synthetic oligonucleotide
gcuaagaaauuuguagaga

SEQ ID NO: 1203 - RNA, 19 nt, Artificial Sequence
  Description of Artificial Sequence Synthetic oligonucleotide
agauaaaggucaaguacua

SEQ ID NO: 1204 - RNA, 19 nt, Artificial Sequence
  Description of Artificial Sequence Synthetic oligonucleotide
gaaaugaaaucucuagaag

SEQ ID NO: 1205 - RNA, 19 nt, Artificial Sequence
  Description of Artificial Sequence Synthetic oligonucleotide
caauggcgcuguuauuuga

SEQ ID NO: 1206 - RNA, 19 nt, Artificial Sequence
  Description of Artificial Sequence Synthetic oligonucleotide
gugcaccaguugagggaua

SEQ ID NO: 1207 - RNA, 19 nt, Artificial Sequence
  Description of Artificial Sequence Synthetic oligonucleotide
gaccaugcauaaugugaca

SEQ ID NO: 1208 - RNA, 19 nt, Artificial Sequence
  Description of Artificial Sequence Synthetic oligonucleotide
cggaaguucuccaguaaag

SEQ ID NO: 1209 - RNA, 19 nt, Artificial Sequence
  Description of Artificial Sequence Synthetic oligonucleotide
ggacggauaccucggagug

SEQ ID NO: 1210 - RNA, 19 nt, Artificial Sequence
  Description of Artificial Sequence Synthetic oligonucleotide
ggcaaagacaggaagauau

SEQ ID NO: 1211 - RNA, 19 nt, Artificial Sequence
  Description of Artificial Sequence Synthetic oligonucleotide
gaucaauguuuauggcaug

SEQ ID NO: 1212 - RNA, 19 nt, Artificial Sequence
  Description of Artificial Sequence Synthetic oligonucleotide
gggcacggacauucaauau

SEQ ID NO: 1213 - RNA, 19 nt, Artificial Sequence
  Description of Artificial Sequence Synthetic oligonucleotide
caagaucggcgaccggguu

SEQ ID NO: 1214 - RNA, 19 nt, Artificial Sequence
  Description of Artificial Sequence Synthetic oligonucleotide
aaacagggcuugcgaugua

SEQ ID NO: 1215 - RNA, 19 nt, Artificial Sequence
  Description of Artificial Sequence Synthetic oligonucleotide
cgacaaggagccugaguga

SEQ ID NO: 1216 - RNA, 19 nt, Artificial Sequence
  Description of Artificial Sequence Synthetic oligonucleotide
gcaaggaugcugacggcuu

SEQ ID NO: 1217 - RNA, 19 nt, Artificial Sequence
  Description of Artificial Sequence Synthetic oligonucleotide
ccucuuauucguaacauua

SEQ ID NO: 1218 - RNA, 19 nt, Artificial Sequence
  Description of Artificial Sequence Synthetic oligonucleotide
agagagggcagcacuuaua

SEQ ID NO: 1219 - RNA, 19 nt, Artificial Sequence
  Description of Artificial Sequence Synthetic oligonucleotide
caggaggacuuucuguuug

SEQ ID NO: 1220 - RNA, 19 nt, Artificial Sequence
  Description of Artificial Sequence Synthetic oligonucleotide
gcauugagaacacauuaga

SEQ ID NO: 1221 - RNA, 19 nt, Artificial Sequence
  Description of Artificial Sequence Synthetic oligonucleotide
gacaauaacacauggaaua

SEQ ID NO: 1222 - RNA, 19 nt, Artificial Sequence
  Description of Artificial Sequence Synthetic oligonucleotide
ccacauacaugaagaaucu

SEQ ID NO: 1223 - RNA, 19 nt, Artificial Sequence
  Description of Artificial Sequence Synthetic oligonucleotide
uaaaggaccuggauaaucg

SEQ ID NO: 1224 - RNA, 19 nt, Artificial Sequence
  Description of Artificial Sequence Synthetic oligonucleotide
gcaguaggaucauauuuga

SEQ ID NO: 1225 - RNA, 19 nt, Artificial Sequence
  Description of Artificial Sequence Synthetic oligonucleotide
gagcuaacauauaucauca

SEQ ID NO: 1226 - RNA, 19 nt, Artificial Sequence
  Description of Artificial Sequence Synthetic oligonucleotide
agucgaaacuggcuucuau

SEQ ID NO: 1227 - RNA, 19 nt, Artificial Sequence
  Description of Artificial Sequence Synthetic oligonucleotide
gcaagucccucuuugauga

SEQ ID NO: 1228 - RNA, 19 nt, Artificial Sequence
  Description of Artificial Sequence Synthetic oligonucleotide
gagcccagcuggacguuga

SEQ ID NO: 1229 - RNA, 19 nt, Artificial Sequence
  Description of Artificial Sequence Synthetic oligonucleotide
ucgcugaugauuacgauaa

SEQ ID NO: 1230 - RNA, 19 nt, Artificial Sequence
  Description of Artificial Sequence Synthetic oligonucleotide
uaauugagcauccggaaua

SEQ ID NO: 1231 - RNA, 19 nt, Artificial Sequence
  Description of Artificial Sequence Synthetic oligonucleotide
gugaaggcguuuaagaaaa

SEQ ID NO: 1232 - RNA, 19 nt, Artificial Sequence
  Description of Artificial Sequence Synthetic oligonucleotide
uaauucagcuacaggguga

SEQ ID NO: 1233 - RNA, 19 nt, Artificial Sequence
  Description of Artificial Sequence Synthetic oligonucleotide
gaggcaaguugucuaauga

SEQ ID NO: 1234 - RNA, 19 nt, Artificial Sequence
  Description of Artificial Sequence Synthetic oligonucleotide
gagaggaggucgaacagau

SEQ ID NO: 1235 - RNA, 19 nt, Artificial Sequence
  Description of Artificial Sequence Synthetic oligonucleotide
gaggagaacaugcuacuua

SEQ ID NO: 1236 - RNA, 19 nt, Artificial Sequence
  Description of Artificial Sequence Synthetic oligonucleotide
uagcaaauauggacucaga

SEQ ID NO: 1237 - RNA, 19 nt, Artificial Sequence
  Description of Artificial Sequence Synthetic oligonucleotide
ccaaguaucucgucacaaa

SEQ ID NO: 1238 - RNA, 19 nt, Artificial Sequence
  Description of Artificial Sequence Synthetic oligonucleotide
gaacagacccggaaauuag

SEQ ID NO: 1239 - RNA, 19 nt, Artificial Sequence
  Description of Artificial Sequence Synthetic oligonucleotide
ggucacacaugucuuauac

SEQ ID NO: 1240 - RNA, 19 nt, Artificial Sequence
  Description of Artificial Sequence Synthetic oligonucleotide
gaacaaaugucuggcuaua

SEQ ID NO: 1241 - RNA, 19 nt, Artificial Sequence
  Description of Artificial Sequence Synthetic oligonucleotide
agaaagaggugucacagaa

SEQ ID NO: 1242 - RNA, 19 nt, Artificial Sequence
  Description of Artificial Sequence Synthetic oligonucleotide
gcagcacuguuuccuauga

SEQ ID NO: 1243 - RNA, 19 nt, Artificial Sequence
  Description of Artificial Sequence Synthetic oligonucleotide
guaaauagcuugcgaaaac

SEQ ID NO: 1244 - RNA, 19 nt, Artificial Sequence
  Description of Artificial Sequence Synthetic oligonucleotide
gaaaggugccugauguguu

SEQ ID NO: 1245 - RNA, 19 nt, Artificial Sequence
  Description of Artificial Sequence Synthetic oligonucleotide
cggaguggcagguauauaa

SEQ ID NO: 1246 - RNA, 19 nt, Artificial Sequence
  Description of Artificial Sequence Synthetic oligonucleotide
ucuucuacgucgcacaaua

SEQ ID NO: 1247 - RNA, 19 nt, Artificial Sequence
  Description of Artificial Sequence Synthetic oligonucleotide
gaagaauugcccagcguug

SEQ ID NO: 1248 - RNA, 19 nt, Artificial Sequence
  Description of Artificial Sequence Synthetic oligonucleotide
ccaagaagcuaagggugau

SEQ ID NO: 1249 - RNA, 19 nt, Artificial Sequence
  Description of Artificial Sequence Synthetic oligonucleotide
ggauuccagucccucaaga

SEQ ID NO: 1250 - RNA, 19 nt, Artificial Sequence
  Description of Artificial Sequence Synthetic oligonucleotide
gaaggugcccucuuugcaa

SEQ ID NO: 1251 - RNA, 19 nt, Artificial Sequence
  Description of Artificial Sequence Synthetic oligonucleotide
gcccacauguacccggaaa

SEQ ID NO: 1252 - RNA, 19 nt, Artificial Sequence
  Description of Artificial Sequence Synthetic oligonucleotide
gcagacagccgaguacauc

SEQ ID NO: 1253 - RNA, 19 nt, Artificial Sequence
  Description of Artificial Sequence Synthetic oligonucleotide
ggauagaacggauaaagca

SEQ ID NO: 1254 - RNA, 19 nt, Artificial Sequence
  Description of Artificial Sequence Synthetic oligonucleotide
cgaaaucccuggugccaaa

SEQ ID NO: 1255 - RNA, 19 nt, Artificial Sequence
  Description of Artificial Sequence Synthetic oligonucleotide
cacaggaccuucuugguua

SEQ ID NO: 1256 - RNA, 19 nt, Artificial Sequence
  Description of Artificial Sequence Synthetic oligonucleotide
ugagauccaugaugacaua

SEQ ID NO: 1257 - RNA, 19 nt, Artificial Sequence
  Description of Artificial Sequence Synthetic oligonucleotide
ggcagcaggugaaacagua

SEQ ID NO: 1258 - RNA, 19 nt, Artificial Sequence
  Description of Artificial Sequence Synthetic oligonucleotide
gcucaagccucccaauauc

SEQ ID NO: 1259 - RNA, 19 nt, Artificial Sequence
  Description of Artificial Sequence Synthetic oligonucleotide
cgcaggcgauucaacauua

SEQ ID NO: 1260 - RNA, 19 nt, Artificial Sequence
  Description of Artificial Sequence Synthetic oligonucleotide
ggaaucugcuugaugugua

SEQ ID NO: 1261 - RNA, 19 nt, Artificial Sequence
  Description of Artificial Sequence Synthetic oligonucleotide
aaacauggacacugcacuu

SEQ ID NO: 1262 - RNA, 19 nt, Artificial Sequence
  Description of Artificial Sequence Synthetic oligonucleotide
gcaagaaccuaaagcacaa

SEQ ID NO: 1263 - RNA, 19 nt, Artificial Sequence
  Description of Artificial Sequence Synthetic oligonucleotide
ccacgguguucgccuuuca

SEQ ID NO: 1264 - RNA, 19 nt, Artificial Sequence
  Description of Artificial Sequence Synthetic oligonucleotide
aggaaugggugcugaacau

SEQ ID NO: 1265 - RNA, 19 nt, Artificial Sequence
  Description of Artificial Sequence Synthetic oligonucleotide
ggagagacguguucaauau

SEQ ID NO: 1266 - RNA, 19 nt, Artificial Sequence
  Description of Artificial Sequence Synthetic oligonucleotide
cagcauagauaucgucugu

SEQ ID NO: 1267 - RNA, 19 nt, Artificial Sequence
  Description of Artificial Sequence Synthetic oligonucleotide
gaaauaaggcugaucaauu

SEQ ID NO: 1268 - RNA, 19 nt, Artificial Sequence
  Description of Artificial Sequence Synthetic oligonucleotide
aaagaacgccgaagucuga

SEQ ID NO: 1269 - RNA, 19 nt, Artificial Sequence
  Description of Artificial Sequence Synthetic oligonucleotide
gaacuucaggcgucacaua

SEQ ID NO: 1270 - RNA, 19 nt, Artificial Sequence
  Description of Artificial Sequence Synthetic oligonucleotide
uaagagagccgucauacug

SEQ ID NO: 1271 - RNA, 19 nt, Artificial Sequence
  Description of Artificial Sequence Synthetic oligonucleotide
guguaaggaucgccuggua

SEQ ID NO: 1272 - RNA, 19 nt, Artificial Sequence
  Description of Artificial Sequence Synthetic oligonucleotide
cgaccuaaauucuguucua

SEQ ID NO: 1273 - RNA, 19 nt, Artificial Sequence
  Description of Artificial Sequence Synthetic oligonucleotide
gaacagacccagaaaucca

SEQ ID NO: 1274 - RNA, 19 nt, Artificial Sequence
  Description of Artificial Sequence Synthetic oligonucleotide
uggacaagccugggccaaa

SEQ ID NO: 1275 - RNA, 19 nt, Artificial Sequence
  Description of Artificial Sequence Synthetic oligonucleotide
guugagcgcuucauugaua

SEQ ID NO: 1276 - RNA, 19 nt, Artificial Sequence
  Description of Artificial Sequence Synthetic oligonucleotide
acugaacuuuguugggaga

SEQ ID NO: 1277 - RNA, 19 nt, Artificial Sequence
  Description of Artificial Sequence Synthetic oligonucleotide
ggaaagcgcccaucugaaa

SEQ ID NO: 1278 - RNA, 19 nt, Artificial Sequence
  Description of Artificial Sequence Synthetic oligonucleotide
gaauuuggcuggaaacuug

SEQ ID NO: 1279 - RNA, 19 nt, Artificial Sequence
  Description of Artificial Sequence Synthetic oligonucleotide
gcacaaagauauugcuaga

SEQ ID NO: 1280 - RNA, 19 nt, Artificial Sequence
  Description of Artificial Sequence Synthetic oligonucleotide
ccuauuggcuguuacauua

SEQ ID NO: 1281 - RNA, 19 nt, Artificial Sequence
  Description of Artificial Sequence Synthetic oligonucleotide
gaacaagcuagaagaaaug

SEQ ID NO: 1282 - RNA, 19 nt, Artificial Sequence
  Description of Artificial Sequence Synthetic oligonucleotide
gacaagauauggaaacaga

SEQ ID NO: 1283 - RNA, 19 nt, Artificial Sequence
  Description of Artificial Sequence Synthetic oligonucleotide
ucuacuaccugaagagauu

SEQ ID NO: 1284 - RNA, 19 nt, Artificial Sequence
  Description of Artificial Sequence Synthetic oligonucleotide
ggauggaacauacaaauuu

SEQ ID NO: 1285 - RNA, 19 nt, Artificial Sequence
  Description of Artificial Sequence Synthetic oligonucleotide
caauaaccgugcuagauga

SEQ ID NO: 1286 - RNA, 19 nt, Artificial Sequence
  Description of Artificial Sequence Synthetic oligonucleotide
gcgcugugacuacaucuuu

SEQ ID NO: 1287 - RNA, 19 nt, Artificial Sequence
  Description of Artificial Sequence Synthetic oligonucleotide
gacaggagcagcaguuuau

SEQ ID NO: 1288 - RNA, 19 nt, Artificial Sequence
  Description of Artificial Sequence Synthetic oligonucleotide
gcagaugaacuuugaaaua

SEQ ID NO: 1289 - RNA, 19 nt, Artificial Sequence
  Description of Artificial Sequence Synthetic oligonucleotide
ggaggaccgaggcuuacua

SEQ ID NO: 1290 - RNA, 19 nt, Artificial Sequence
  Description of Artificial Sequence Synthetic oligonucleotide
cuacgagauguuugaguga

SEQ ID NO: 1291 - RNA, 19 nt, Artificial Sequence
  Description of Artificial Sequence Synthetic oligonucleotide
gcggaaguguucaagcaug

SEQ ID NO: 1292 - RNA, 19 nt, Artificial Sequence
  Description of Artificial Sequence Synthetic oligonucleotide
cgauuguccuguggcguua

SEQ ID NO: 1293 - RNA, 19 nt, Artificial Sequence
  Description of Artificial Sequence Synthetic oligonucleotide
gaaccacgauguacauuca

SEQ ID NO: 1294 - RNA, 19 nt, Artificial Sequence
  Description of Artificial Sequence Synthetic oligonucleotide
cggaugaugaugugauuua

SEQ ID NO: 1295 - RNA, 19 nt, Artificial Sequence
  Description of Artificial Sequence Synthetic oligonucleotide
gaguugauaccggaaauuu

SEQ ID NO: 1296 - RNA, 19 nt, Artificial Sequence
  Description of Artificial Sequence Synthetic oligonucleotide
gcccagaguugccacuaaa

SEQ ID NO: 1297 - RNA, 19 nt, Artificial Sequence
  Description of Artificial Sequence Synthetic oligonucleotide
gaacauggguggcauccaa

SEQ ID NO: 1298 - RNA, 19 nt, Artificial Sequence
  Description of Artificial Sequence Synthetic oligonucleotide
gaacagcucucaaguugua

SEQ ID NO: 1299 - RNA, 19 nt, Artificial Sequence
  Description of Artificial Sequence Synthetic oligonucleotide
gcaaagauguacuaucaga

SEQ ID NO: 1300 - RNA, 19 nt, Artificial Sequence
  Description of Artificial Sequence Synthetic oligonucleotide
ggaagaagcuaucaccuua

SEQ ID NO: 1301 - RNA, 19 nt, Artificial Sequence
  Description of Artificial Sequence Synthetic oligonucleotide
gaggguaucagaccuggua

SEQ ID NO: 1302 - RNA, 19 nt, Artificial Sequence
  Description of Artificial Sequence Synthetic oligonucleotide
gcagugauauuuaggcaua

SEQ ID NO: 1303 - RNA, 19 nt, Artificial Sequence
  Description of Artificial Sequence Synthetic oligonucleotide
ggagauccuuacaguguua

SEQ ID NO: 1304 - RNA, 19 nt, Artificial Sequence
  Description of Artificial Sequence Synthetic oligonucleotide
gaagggaugugugcaaaca

SEQ ID NO: 1305 - RNA, 19 nt, Artificial Sequence
  Description of Artificial Sequence Synthetic oligonucleotide
caacaaagcuauuaaccug

SEQ ID NO: 1306 - RNA, 19 nt, Artificial Sequence
  Description of Artificial Sequence Synthetic oligonucleotide
gcccaucuguauucacuuu

SEQ ID NO: 1307 - RNA, 19 nt, Artificial Sequence
  Description of Artificial Sequence Synthetic oligonucleotide
cgaaggcuaugcacuauac

SEQ ID NO: 1308 - RNA, 19 nt, Artificial Sequence
  Description of Artificial Sequence Synthetic oligonucleotide
gcaaagaaauacggauuaa

SEQ ID NO: 1309 - RNA, 19 nt, Artificial Sequence
  Description of Artificial Sequence Synthetic oligonucleotide
guucagcucuacagccuua

SEQ ID NO: 1310 - RNA, 19 nt, Artificial Sequence
  Description of Artificial Sequence Synthetic oligonucleotide
gcuacaagaaggaucugaa

SEQ ID NO: 1311 - RNA, 19 nt, Artificial Sequence
  Description of Artificial Sequence Synthetic oligonucleotide
cgggaccaauuaccgcaaa

SEQ ID NO: 1312 - RNA, 19 nt, Artificial Sequence
  Description of Artificial Sequence Synthetic oligonucleotide
ugucauuccgccugguuga

SEQ ID NO: 1313 - RNA, 19 nt, Artificial Sequence
  Description of Artificial Sequence Synthetic oligonucleotide
cgacuaccaucugcacuua

SEQ ID NO: 1314 - RNA, 19 nt, Artificial Sequence
  Description of Artificial Sequence Synthetic oligonucleotide
cuaagaagcuauccucauc

SEQ ID NO: 1315 - RNA, 19 nt, Artificial Sequence
  Description of Artificial Sequence Synthetic oligonucleotide
acaaggagcucucauuaug

SEQ ID NO: 1316 - RNA, 19 nt, Artificial Sequence
  Description of Artificial Sequence Synthetic oligonucleotide
ugacagcucucccuauagu

SEQ ID NO: 1317 - RNA, 19 nt, Artificial Sequence
  Description of Artificial Sequence Synthetic oligonucleotide
ggacggacucaacauguua

SEQ ID NO: 1318 - RNA, 19 nt, Artificial Sequence
  Description of Artificial Sequence Synthetic oligonucleotide
gaagccaacuuuccuuucu

SEQ ID NO: 1319 - RNA, 19 nt, Artificial Sequence
  Description of Artificial Sequence Synthetic oligonucleotide
caacgcaucugcucuucac

SEQ ID NO: 1320 - RNA, 19 nt, Artificial Sequence
  Description of Artificial Sequence Synthetic oligonucleotide
ggaauagugugaacaacau

SEQ ID NO: 1321 - RNA, 19 nt, Artificial Sequence
  Description of Artificial Sequence Synthetic oligonucleotide
guuacaaacuccauuacua

SEQ ID NO: 1322 - RNA, 19 nt, Artificial Sequence
  Description of Artificial Sequence Synthetic oligonucleotide
ccacaaccuguaccuguuu

SEQ ID NO: 1323 - RNA, 19 nt, Artificial Sequence
  Description of Artificial Sequence Synthetic oligonucleotide
ggaucaaaguugucaugaa

SEQ ID NO: 1324 - RNA, 19 nt, Artificial Sequence
  Description of Artificial Sequence Synthetic oligonucleotide
cgacuggacuccuauguuc

SEQ ID NO: 1325 - RNA, 19 nt, Artificial Sequence
  Description of Artificial Sequence Synthetic oligonucleotide
ggaugggagaccuacauua

SEQ ID NO: 1326 - RNA, 19 nt, Artificial Sequence
  Description of Artificial Sequence Synthetic oligonucleotide
gcagauguuuacuggugug

SEQ ID NO: 1327 - RNA, 19 nt, Artificial Sequence
  Description of Artificial Sequence Synthetic oligonucleotide
cuaaagacauggccacuua

SEQ ID NO: 1328 - RNA, 19 nt, Artificial Sequence
  Description of Artificial Sequence Synthetic oligonucleotide
gggaacagccuaucuacau

SEQ ID NO: 1329 - RNA, 19 nt, Artificial Sequence
  Description of Artificial Sequence Synthetic oligonucleotide
gaauucaguuuauggauga

SEQ ID NO: 1330 - RNA, 19 nt, Artificial Sequence
  Description of Artificial Sequence Synthetic oligonucleotide
gaaguuugauuaccuguau

SEQ ID NO: 1331 - RNA, 19 nt, Artificial Sequence
  Description of Artificial Sequence Synthetic oligonucleotide
gcgcaucucugaauuacaa

SEQ ID NO: 1332 - RNA, 19 nt, Artificial Sequence
  Description of Artificial Sequence Synthetic oligonucleotide
gaaaugugccagugauauu

SEQ ID NO: 1333 - RNA, 19 nt, Artificial Sequence
  Description of Artificial Sequence Synthetic oligonucleotide
gauuggaguuugagaagca

SEQ ID NO: 1334 - RNA, 19 nt, Artificial Sequence
  Description of Artificial Sequence Synthetic oligonucleotide
gaaaguccucgcugcauug

SEQ ID NO: 1335 - RNA, 19 nt, Artificial Sequence
  Description of Artificial Sequence Synthetic oligonucleotide
ggaaagaguuggaggacgc

SEQ ID NO: 1336 - RNA, 19 nt, Artificial Sequence
  Description of Artificial Sequence Synthetic oligonucleotide
cuaugaagccggugaaauu

SEQ ID NO: 1337 - RNA, 19 nt, Artificial Sequence
  Description of Artificial Sequence Synthetic oligonucleotide
ccacagaucucuaaauaca

SEQ ID NO: 1338 - RNA, 19 nt, Artificial Sequence
  Description of Artificial Sequence Synthetic oligonucleotide
gugcaucacguuucagauu

SEQ ID NO: 1339 - RNA, 19 nt, Artificial Sequence
  Description of Artificial Sequence Synthetic oligonucleotide
gacaauauguaccgaaauu

SEQ ID NO: 1340 - RNA, 19 nt, Artificial Sequence
  Description of Artificial Sequence Synthetic oligonucleotide
ggaaagaaauagucaguaa

SEQ ID NO: 1341 - RNA, 19 nt, Artificial Sequence
  Description of Artificial Sequence Synthetic oligonucleotide
gagcauuucugcacuggua

SEQ ID NO: 1342 - RNA, 19 nt, Artificial Sequence
  Description of Artificial Sequence Synthetic oligonucleotide
gcacacaaaugcaucuuuc

SEQ ID NO: 1343 - RNA, 19 nt, Artificial Sequence
  Description of Artificial Sequence Synthetic oligonucleotide
uaugauauaugccaucgua

SEQ ID NO: 1344 - RNA, 19 nt, Artificial Sequence
  Description of Artificial Sequence Synthetic oligonucleotide
gaauguagaccuaauugaa

SEQ ID NO: 1345 - RNA, 19 nt, Artificial Sequence
  Description of Artificial Sequence Synthetic oligonucleotide
cgacuuugcugcucagaug

SEQ ID NO: 1346 - RNA, 19 nt, Artificial Sequence
  Description of Artificial Sequence Synthetic oligonucleotide
caauaacccugaacucaug

SEQ ID NO: 1347 - RNA, 19 nt, Artificial Sequence
  Description of Artificial Sequence Synthetic oligonucleotide
ggucagggauguucaauag

SEQ ID NO: 1348 - RNA, 19 nt, Artificial Sequence
  Description of Artificial Sequence Synthetic oligonucleotide
gagccucggucaaggaguu

SEQ ID NO: 1349 - RNA, 19 nt, Artificial Sequence
  Description of Artificial Sequence Synthetic oligonucleotide
ccacaaagcuggagguaaa

SEQ ID NO: 1350 - RNA, 19 nt, Artificial Sequence
  Description of Artificial Sequence Synthetic oligonucleotide
ccacacauuggaucagaua

SEQ ID NO: 1351 - RNA, 19 nt, Artificial Sequence
  Description of Artificial Sequence Synthetic oligonucleotide
gcacaagacuuccguugga

SEQ ID NO: 1352 - RNA, 19 nt, Artificial Sequence
  Description of Artificial Sequence Synthetic oligonucleotide
gaagauaccucacuuuguc

SEQ ID NO: 1353 - RNA, 19 nt, Artificial Sequence
  Description of Artificial Sequence Synthetic oligonucleotide
gaaccugauugacgggauc

SEQ ID NO: 1354 - RNA, 19 nt, Artificial Sequence
  Description of Artificial Sequence Synthetic oligonucleotide
caacggaccuggauauagg

SEQ ID NO: 1355 - RNA, 19 nt, Artificial Sequence
  Description of Artificial Sequence Synthetic oligonucleotide
gagcggaaggacauacuua

SEQ ID NO: 1356 - RNA, 19 nt, Artificial Sequence
  Description of Artificial Sequence Synthetic oligonucleotide
gcggagagguucacaacaa

SEQ ID NO: 1357 - RNA, 19 nt, Artificial Sequence
  Description of Artificial Sequence Synthetic oligonucleotide
ugaaauuggcacuuaauca

SEQ ID NO: 1358 - RNA, 19 nt, Artificial Sequence
  Description of Artificial Sequence Synthetic oligonucleotide
gagaagagagacagugaua

SEQ ID NO: 1359 - RNA, 19 nt, Artificial Sequence
  Description of Artificial Sequence Synthetic oligonucleotide
ugaacaauccuuccacuaa

SEQ ID NO: 1360 - RNA, 19 nt, Artificial Sequence
  Description of Artificial Sequence Synthetic oligonucleotide
cuagggaacuuauagaaau

SEQ ID NO: 1361 - RNA, 19 nt, Artificial Sequence
  Description of Artificial Sequence Synthetic oligonucleotide
ucacagaguaucuuagaga

SEQ ID NO: 1362 - RNA, 19 nt, Artificial Sequence
  Description of Artificial Sequence Synthetic oligonucleotide
ggacgacaauaaugagaac

SEQ ID NO: 1363 - RNA, 19 nt, Artificial Sequence
  Description of Artificial Sequence Synthetic oligonucleotide
ggagggaauugucgagaau

SEQ ID NO: 1364 - RNA, 19 nt, Artificial Sequence
  Description of Artificial Sequence Synthetic oligonucleotide
gauggcggaugcuuugaua

SEQ ID NO: 1365 - RNA, 19 nt, Artificial Sequence
  Description of Artificial Sequence Synthetic oligonucleotide
ucacagauaugacuaagga

SEQ ID NO: 1366 - RNA, 19 nt, Artificial Sequence
  Description of Artificial Sequence Synthetic oligonucleotide
ggagaggcauggacauuaa

SEQ ID NO: 1367 - RNA, 19 nt, Artificial Sequence
  Description of Artificial Sequence Synthetic oligonucleotide
caagauggcauaugaauug

SEQ ID NO: 1368 - RNA, 19 nt, Artificial Sequence
  Description of Artificial Sequence Synthetic oligonucleotide
aaacagcgcucgccuuaug

SEQ ID NO: 1369 - RNA, 19 nt, Artificial Sequence
  Description of Artificial Sequence Synthetic oligonucleotide
gcgccgaccucuuugucua

SEQ ID NO: 1370 - RNA, 19 nt, Artificial Sequence
  Description of Artificial Sequence Synthetic oligonucleotide
gcaauuagaucacgccaaa

SEQ ID NO: 1371 - RNA, 19 nt, Artificial Sequence
  Description of Artificial Sequence Synthetic oligonucleotide
agaaguaccuccgagaaua

SEQ ID NO: 1372 - RNA, 19 nt, Artificial Sequence
  Description of Artificial Sequence Synthetic oligonucleotide
gaaaggagauuuagaucaa

SEQ ID NO: 1373 - RNA, 19 nt, Artificial Sequence
  Description of Artificial Sequence Synthetic oligonucleotide
gauccgcgcucgcauugag

SEQ ID NO: 1374 - RNA, 19 nt, Artificial Sequence
  Description of Artificial Sequence Synthetic oligonucleotide
acaauaauaccguugcuua

SEQ ID NO: 1375 - RNA, 19 nt, Artificial Sequence
  Description of Artificial Sequence Synthetic oligonucleotide
uaagaacuccaucaaguag

SEQ ID NO: 1376 - RNA, 19 nt, Artificial Sequence
  Description of Artificial Sequence Synthetic oligonucleotide
gauuacaagucucaugugu

SEQ ID NO: 1377 - RNA, 19 nt, Artificial Sequence
  Description of Artificial Sequence Synthetic oligonucleotide
gugcacgaccugacaguaa

SEQ ID NO: 1378 - RNA, 19 nt, Artificial Sequence
  Description of Artificial Sequence Synthetic oligonucleotide
gacauccgcaugauccaua

SEQ ID NO: 1379 - RNA, 19 nt, Artificial Sequence
  Description of Artificial Sequence Synthetic oligonucleotide
caccauaccuggagcgugu

SEQ ID NO: 1380 - RNA, 19 nt, Artificial Sequence
  Description of Artificial Sequence Synthetic oligonucleotide
cuagagaccucauccguag

SEQ ID NO: 1381 - RNA, 19 nt, Artificial Sequence
  Description of Artificial Sequence Synthetic oligonucleotide
ccacgaaccugcuuacaga

SEQ ID NO: 1382 - RNA, 19 nt, Artificial Sequence
  Description of Artificial Sequence Synthetic oligonucleotide
gcguuuaucuucgcuauca

SEQ ID NO: 1383 - RNA, 19 nt, Artificial Sequence
  Description of Artificial Sequence Synthetic oligonucleotide
caaacagcggcgucugaua

SEQ ID NO: 1384 - RNA, 19 nt, Artificial Sequence
  Description of Artificial Sequence Synthetic oligonucleotide
ucagaagguuccaucgaau

SEQ ID NO: 1385 - RNA, 19 nt, Artificial Sequence
  Description of Artificial Sequence Synthetic oligonucleotide
guacgaaaccacuuuauaa

SEQ ID NO: 1386 - RNA, 19 nt, Artificial Sequence
  Description of Artificial Sequence Synthetic oligonucleotide
cgaccgcgaucagaaauuc

SEQ ID NO: 1387 - RNA, 19 nt, Artificial Sequence
  Description of Artificial Sequence Synthetic oligonucleotide
gcaggcuauucauuuacua

SEQ ID NO: 1388 - RNA, 19 nt, Artificial Sequence
  Description of Artificial Sequence Synthetic oligonucleotide
ccgcaaaggcaagcaucua

SEQ ID NO: 1389 - RNA, 19 nt, Artificial Sequence
  Description of Artificial Sequence Synthetic oligonucleotide
ggacuaggaggaguauuua

SEQ ID NO: 1390 - RNA, 19 nt, Artificial Sequence
  Description of Artificial Sequence Synthetic oligonucleotide
cagcauaguuuacuuggua

SEQ ID NO: 1391 - RNA, 19 nt, Artificial Sequence
  Description of Artificial Sequence Synthetic oligonucleotide
gcaaauagauaccuaacug

SEQ ID NO: 1392 - RNA, 19 nt, Artificial Sequence
  Description of Artificial Sequence Synthetic oligonucleotide
gcaggcauguauccuaauu

SEQ ID NO: 1393 - RNA, 19 nt, Artificial Sequence
  Description of Artificial Sequence Synthetic oligonucleotide
cgacccgguucgauuagaa

SEQ ID NO: 1394 - RNA, 19 nt, Artificial Sequence
  Description of Artificial Sequence Synthetic oligonucleotide
caggugaaucggacaaaua

SEQ ID NO: 1395 - RNA, 19 nt, Artificial Sequence
  Description of Artificial Sequence Synthetic oligonucleotide
gaucaucaguugagacaag

SEQ ID NO: 1396 - RNA, 19 nt, Artificial Sequence
  Description of Artificial Sequence Synthetic oligonucleotide
agacgaagggcgauccauu

SEQ ID NO: 1397 - RNA, 19 nt, Artificial Sequence
  Description of Artificial Sequence Synthetic oligonucleotide
gcaaauggacaaaggaguu

SEQ ID NO: 1398 - RNA, 19 nt, Artificial Sequence
  Description of Artificial Sequence Synthetic oligonucleotide
ggaaauacauugccuacgu

SEQ ID NO: 1399 - RNA, 19 nt, Artificial Sequence
  Description of Artificial Sequence Synthetic oligonucleotide
gaagaugucaagaauauga

SEQ ID NO: 1400 - RNA, 19 nt, Artificial Sequence
  Description of Artificial Sequence Synthetic oligonucleotide
ccaagaagaugaccaccuu

SEQ ID NO: 1401 - RNA, 19 nt, Artificial Sequence
  Description of Artificial Sequence Synthetic oligonucleotide
ggacuucaaacacuuagga

SEQ ID NO: 1402 - RNA, 19 nt, Artificial Sequence
  Description of Artificial Sequence Synthetic oligonucleotide
ugacuacguuggagagaga

SEQ ID NO: 1403 - RNA, 19 nt, Artificial Sequence
  Description of Artificial Sequence Synthetic oligonucleotide
gaggaggaaucauaugcaa

SEQ ID NO: 1404 - RNA, 19 nt, Artificial Sequence
  Description of Artificial Sequence Synthetic oligonucleotide
gcaaguagcccuuaggaga

SEQ ID NO: 1405 - RNA, 19 nt, Artificial Sequence
  Description of Artificial Sequence Synthetic oligonucleotide
acagaagcuugaucuaauu

SEQ ID NO: 1406 - RNA, 19 nt, Artificial Sequence
  Description of Artificial Sequence Synthetic oligonucleotide
cgauaaacacgaaucauuu

SEQ ID NO: 1407 - RNA, 19 nt, Artificial Sequence
  Description of Artificial Sequence Synthetic oligonucleotide
caaaguucauggcacauua

SEQ ID NO: 1408 - RNA, 19 nt, Artificial Sequence
  Description of Artificial Sequence Synthetic oligonucleotide
caagagugcucgugacugu

SEQ ID NO: 1409 - RNA, 19 nt, Artificial Sequence
  Description of Artificial Sequence Synthetic oligonucleotide
gcacauguacugaucuuau

SEQ ID NO: 1410 - RNA, 19 nt, Artificial Sequence
  Description of Artificial Sequence Synthetic oligonucleotide
gcaggaaggcuuucaguca

SEQ ID NO: 1411 - RNA, 19 nt, Artificial Sequence
  Description of Artificial Sequence Synthetic oligonucleotide
gagaaaaccuuuagugugu

SEQ ID NO: 1412 - RNA, 19 nt, Artificial Sequence
  Description of Artificial Sequence Synthetic oligonucleotide
guucugaucucauuaacca

SEQ ID NO: 1413 - RNA, 19 nt, Artificial Sequence
  Description of Artificial Sequence Synthetic oligonucleotide
ggccuuuacucgagucaau

SEQ ID NO: 1414 - RNA, 19 nt, Artificial Sequence
  Description of Artificial Sequence Synthetic oligonucleotide
guaaaucccuuggccguaa

SEQ ID NO: 1415 - RNA, 19 nt, Artificial Sequence
  Description of Artificial Sequence Synthetic oligonucleotide
ggagguaugucaaucagau

SEQ ID NO: 1416 - RNA, 19 nt, Artificial Sequence
  Description of Artificial Sequence Synthetic oligonucleotide
cgacguagccauagacuuu

SEQ ID NO: 1417 - RNA, 19 nt, Artificial Sequence
  Description of Artificial Sequence Synthetic oligonucleotide
ggauguggcugugcuguuu

SEQ ID NO: 1418 - RNA, 19 nt, Artificial Sequence
  Description of Artificial Sequence Synthetic oligonucleotide
ggaauguaccuugggauuu

SEQ ID NO: 1419 - RNA, 19 nt, Artificial Sequence
  Description of Artificial Sequence Synthetic oligonucleotide
gaauguaccuugggauuug

SEQ ID NO: 1420 - RNA, 19 nt, Artificial Sequence
  Description of Artificial Sequence Synthetic oligonucleotide
aaagggaaguuuucagaua

SEQ ID NO: 1421 - RNA, 19 nt, Artificial Sequence
  Description of Artificial Sequence Synthetic oligonucleotide
ggucagaucuaauuaaaca

SEQ ID NO: 1422 - RNA, 19 nt, Artificial Sequence
  Description of Artificial Sequence Synthetic oligonucleotide
cauccgcuaucauauaugu

SEQ ID NO: 1423 - RNA, 19 nt, Artificial Sequence
  Description of Artificial Sequence Synthetic oligonucleotide
ggaaauguugucucacuag

SEQ ID NO: 1424 - RNA, 19 nt, Artificial Sequence
  Description of Artificial Sequence Synthetic oligonucleotide
ggagugagaacgagguaaa

SEQ ID NO: 1425 - RNA, 19 nt, Artificial Sequence
  Description of Artificial Sequence Synthetic oligonucleotide
caccaaacccauuacggua

SEQ ID NO: 1426 - RNA, 19 nt, Artificial Sequence
  Description of Artificial Sequence Synthetic oligonucleotide
ggugaaguguccaaacuca

SEQ ID NO: 1427 - RNA, 19 nt, Artificial Sequence
  Description of Artificial Sequence Synthetic oligonucleotide
gcaucuacgggcucaagua

SEQ ID NO: 1428 - RNA, 19 nt, Artificial Sequence
  Description of Artificial Sequence Synthetic oligonucleotide
ggacaaggcccgaguucac

SEQ ID NO: 1429 - RNA, 19 nt, Artificial Sequence
  Description of Artificial Sequence Synthetic oligonucleotide
caaguaagcucgacccuuu

SEQ ID NO: 1430 - RNA, 19 nt, Artificial Sequence
  Description of Artificial Sequence Synthetic oligonucleotide
ccacguggacccugcauuu

SEQ ID NO: 1431 - RNA, 19 nt, Artificial Sequence
  Description of Artificial Sequence Synthetic oligonucleotide
cuacaguucugauggcuua

SEQ ID NO: 1432 - RNA, 19 nt, Artificial Sequence
  Description of Artificial Sequence Synthetic oligonucleotide
ggacaucccaucaccuuaa

SEQ ID NO: 1433 - RNA, 19 nt, Artificial Sequence
  Description of Artificial Sequence Synthetic oligonucleotide
gaacauagcguuaaagaca

SEQ ID NO: 1434 - RNA, 19 nt, Artificial Sequence
  Description of Artificial Sequence Synthetic oligonucleotide
cgcaagaauuguuguacua

SEQ ID NO: 1435 - RNA, 19 nt, Artificial Sequence
  Description of Artificial Sequence Synthetic oligonucleotide
ccacagaugucaaaucaca

SEQ ID NO: 1436 - RNA, 19 nt, Artificial Sequence
  Description of Artificial Sequence Synthetic oligonucleotide
gcgcacaugugaugaugca

SEQ ID NO: 1437 - RNA, 19 nt, Artificial Sequence
  Description of Artificial Sequence Synthetic oligonucleotide
caagagaagucugccaaau

SEQ ID NO: 1438 - RNA, 19 nt, Artificial Sequence
  Description of Artificial Sequence Synthetic oligonucleotide
aauuguaguucacgccuua

SEQ ID NO: 1439 - RNA, 19 nt, Artificial Sequence
  Description of Artificial Sequence Synthetic oligonucleotide
acucaaggccucuuaagaa

SEQ ID NO: 1440 - RNA, 19 nt, Artificial Sequence
  Description of Artificial Sequence Synthetic oligonucleotide
ggucguaccuaacuaaaca

SEQ ID NO: 1441 - RNA, 19 nt, Artificial Sequence
  Description of Artificial Sequence Synthetic oligonucleotide
caaggacacucugacccga

SEQ ID NO: 1442 - RNA, 19 nt, Artificial Sequence
  Description of Artificial Sequence Synthetic oligonucleotide
gcgcugggauuuucaguuc

SEQ ID NO: 1443 - RNA, 19 nt, Artificial Sequence
  Description of Artificial Sequence Synthetic oligonucleotide
gcaagagucgcuucacuua

SEQ ID NO: 1444 - RNA, 19 nt, Artificial Sequence
  Description of Artificial Sequence Synthetic oligonucleotide
gcacuuuccagauauauuu

SEQ ID NO: 1445 - RNA, 19 nt, Artificial Sequence
  Description of Artificial Sequence Synthetic oligonucleotide
gauacgagcuauuugagaa

SEQ ID NO: 1446 - RNA, 19 nt, Artificial Sequence
  Description of Artificial Sequence Synthetic oligonucleotide
ugagaaugcacaaucgaua

SEQ ID NO: 1447 - RNA, 19 nt, Artificial Sequence
  Description of Artificial Sequence Synthetic oligonucleotide
gaagaagacugccggagua

SEQ ID NO: 1448 - RNA, 19 nt, Artificial Sequence
  Description of Artificial Sequence Synthetic oligonucleotide
gggaagacccgaauguuga

SEQ ID NO: 1449 - RNA, 19 nt, Artificial Sequence
  Description of Artificial Sequence Synthetic oligonucleotide
caucaacguucgggacuuu

SEQ ID NO: 1450 - RNA, 19 nt, Artificial Sequence
  Description of Artificial Sequence Synthetic oligonucleotide
gucaugaaggaauggcaua

SEQ ID NO: 1451 - RNA, 19 nt, Artificial Sequence
  Description of Artificial Sequence Synthetic oligonucleotide
ggaccuggauuucuguuca

SEQ ID NO: 1452 - RNA, 19 nt, Artificial Sequence
  Description of Artificial Sequence Synthetic oligonucleotide
gggaagguugugaagacuu

SEQ ID NO: 1453 - RNA, 19 nt, Artificial Sequence
  Description of Artificial Sequence Synthetic oligonucleotide
cgaaacacccguauaacaa

SEQ ID NO: 1454 - RNA, 19 nt, Artificial Sequence
  Description of Artificial Sequence Synthetic oligonucleotide
auaacuagcugcuguucua

SEQ ID NO: 1455 - RNA, 19 nt, Artificial Sequence
  Description of Artificial Sequence Synthetic oligonucleotide
guaucgcacuccagauuua

SEQ ID NO: 1456 - RNA, 19 nt, Artificial Sequence
  Description of Artificial Sequence Synthetic oligonucleotide
gaagaggauuuugggauua

SEQ ID NO: 1457 - DNA, 20 bp, Artificial Sequence
  Description of Artificial Sequence Synthetic oligonucleotide
cgcctgaagtctctgattaa

SEQ ID NO: 1458 - DNA, 19 bp, Artificial Sequence
  Description of Artificial Sequence Synthetic oligonucleotide
ctctttcacatgtgcttta

SEQ ID NO: 1459 - DNA, 19 bp, Artificial Sequence
  Description of Artificial Sequence Synthetic oligonucleotide
cctgctgaatttatgttgt

SEQ ID NO: 1460 - DNA, 19 bp, Artificial Sequence
  Description of Artificial Sequence Synthetic oligonucleotide
ccattggaattatccttta

SEQ ID NO: 1461 - DNA, 19 bp, Artificial Sequence
  Description of Artificial Sequence Synthetic oligonucleotide
cgtacgcggaatacttcga

SEQ ID NO: 1462 - RNA, 21 nt, Artificial Sequence
  Description of Artificial Sequence Synthetic oligonucleotide
cugcuuguaccaauugcuauu

SEQ ID NO: 1463 - RNA, 21 nt, Artificial Sequence
  Description of Artificial Sequence Synthetic oligonucleotide
ccaaagagcugaauguuauuu

SEQ ID NO: 1464 - RNA, 21 nt, Artificial Sequence
  Description of Artificial Sequence Synthetic oligonucleotide
cuacaaaguggauugaugauu

SEQ ID NO: 1465 - RNA, 21 nt, Artificial Sequence
  Description of Artificial Sequence Synthetic oligonucleotide
gagcaaccagucagugaaguu

SEQ ID NO: 1466 - RNA, 21 nt, Artificial Sequence
  Description of Artificial Sequence Synthetic oligonucleotide
gagaagauaugauugacauuu

SEQ ID NO: 1467 - RNA, 21 nt, Artificial Sequence
  Description of Artificial Sequence Synthetic oligonucleotide
gagcaaccagucagugaaguu

SEQ ID NO: 1468 - RNA, 21 nt, Artificial Sequence
  Description of Artificial Sequence Synthetic oligonucleotide
aagcagagaagauaugauuuu

SEQ ID NO: 1469 - RNA, 21 nt, Artificial Sequence
  Description of Artificial Sequence Synthetic oligonucleotide
ccaaagagcugaauguuauuu

SEQ ID NO: 1470 - RNA, 21 nt, Artificial Sequence
  Description of Artificial Sequence Synthetic oligonucleotide
cuacaaaguggauugaugauu

SEQ ID NO: 1471 - RNA, 21 nt, Artificial Sequence
  Description of Artificial Sequence Synthetic oligonucleotide
gcagugauauuuaggcauauu

SEQ ID NO: 1472 - RNA, 21 nt, Artificial Sequence
  Description of Artificial Sequence Synthetic oligonucleotide
ggagauccuuacaguguuauu

SEQ ID NO: 1473 - RNA, 21 nt, Artificial Sequence
  Description of Artificial Sequence Synthetic oligonucleotide
gaagggaugugugcaaacauu

SEQ ID NO: 1474 - RNA, 21 nt, Artificial Sequence
  Description of Artificial Sequence Synthetic oligonucleotide
gaggguaucagaccugguauu

SEQ ID NO: 1475 - RNA, 21 nt, Artificial Sequence
  Description of Artificial Sequence Synthetic oligonucleotide
cgacauugcagcucguguauu

SEQ ID NO: 1476 - RNA, 21 nt, Artificial Sequence
  Description of Artificial Sequence Synthetic oligonucleotide
gagugaagucguugaucgauu

SEQ ID NO: 1477 - RNA, 21 nt, Artificial Sequence
  Description of Artificial Sequence Synthetic oligonucleotide
ucaccagguuguuucgauauu

SEQ ID NO: 1478 - RNA, 21 nt, Artificial Sequence
  Description of Artificial Sequence Synthetic oligonucleotide
guacaaaacuaacgaugaauu

SEQ ID NO: 1479 - RNA, 21 nt, Artificial Sequence
  Description of Artificial Sequence Synthetic oligonucleotide
gcggaaagaugcacuagucuu

SEQ ID NO: 1480 - RNA, 21 nt, Artificial Sequence
  Description of Artificial Sequence Synthetic oligonucleotide
guacuuugguggacgaguuuu

SEQ ID NO: 1481 - RNA, 21 nt, Artificial Sequence
  Description of Artificial Sequence Synthetic oligonucleotide
ugagugggcugaugcgugauu

SEQ ID NO: 1482 - RNA, 21 nt, Artificial Sequence
  Description of Artificial Sequence Synthetic oligonucleotide
cagaaaccagagcaaguuauu

SEQ ID NO: 1483 - RNA, 21 nt, Artificial Sequence
  Description of Artificial Sequence Synthetic oligonucleotide
gaaaggagauuuagaucaauu

SEQ ID NO: 1484 - RNA, 21 nt, Artificial Sequence
  Description of Artificial Sequence Synthetic oligonucleotide
gcaauuagaucacgccaaauu

SEQ ID NO: 1485 - RNA, 21 nt, Artificial Sequence
  Description of Artificial Sequence Synthetic oligonucleotide
agaaguaccuccgagaauauu

SEQ ID NO: 1486 - RNA, 21 nt, Artificial Sequence
  Description of Artificial Sequence Synthetic oligonucleotide
gcgccgaccucuuugucuauu

SEQ ID NO: 1487 - RNA, 21 nt, Artificial Sequence
  Description of Artificial Sequence Synthetic oligonucleotide
gaaguccugcaauuuauaauu

SEQ ID NO: 1488 - DNA, 23 bp, Artificial Sequence
  Description of Artificial Sequence Synthetic primer
ggagccaaacgggtcatcatctc

SEQ ID NO: 1489 - DNA, 22 bp, Artificial Sequence
  Description of Artificial Sequence Synthetic primer
gaggggccatccacagtcttct

SEQ ID NO: 1490 - DNA, 18 bp, Artificial Sequence
  Description of Artificial Sequence Synthetic primer
cctggaagggatgtgtgc

SEQ ID NO: 1491 - DNA, 23 bp, Artificial Sequence
  Description of Artificial Sequence Synthetic primer
aaaaaggcaaagaagtcacatca

SEQ ID NO: 1492 - protein, 4 aa, Artificial Sequence
  Description of Artificial Sequence Synthetic peptide
DEAD

(19) United States
(12) Patent Application Publication
(10) Pub. No.: US 2010/0273859 A1
(43) Pub. Date: Oct. 28, 2010

(54) TREATMENT AND PREVENTION OF HIV INFECTION

(75) Inventors: Stephen Elledge, Brookline, MA (US); Judy Lieberman, Brookline, MA (US); Abraham L. Brass, Newton, MA (US)

Correspondence Address: DAVID S. RESNICK, NIXON PEABODY LLP, 100 SUMMER STREET, BOSTON, MA, 02110-2131, US

(73) Assignees: THE BRIGHAM AND WOMEN'S HOSPITAL, INC., Boston, MA (US); IMMUNE DISEASE INSTITUTE, INC., Boston, MA (US); THE GENERAL HOSPITAL CORPORATION, Boston, MA (US)

(21) Appl. No.: 12/747,041

(22) PCT Filed: Dec. 15, 2008
(86) PCT No.: PCT/US08/86821

§ 371 Date: Jun. 9, 2010

Related U.S. Application Data
(60) Provisional application No. 61/011,157, filed on Jan. 15, 2008.
(60) Provisional application No. 61/195,006, filed on Oct. 2, 2008.

(51) Int. Cl. A61K 31/713; C07H 21/02; A61P 31/18

(52) U.S. Cl. 514/44 A; 536/24.5

(57) ABSTRACT

Disclosed herein are methods for treating and/or preventing HIV infection in a cell. The methods involve downmodulating one or more of the HIV-dependency factors (HDFs) disclosed herein to thereby treat and/or prevent HIV infection in the cell. Downmodulating the HDFs can be by contacting the cell with an agent that downmodulates the HDF. Also disclosed herein are methods for treating and/or preventing HIV infection in a subject comprising downmodulating one or more of the HIV-dependency factors (HDFs), disclosed herein, to thereby treat and/or prevent HIV infection in the subject. The method may further comprise selecting a subject diagnosed with or at risk for HIV infection, prior to downmodulating. Downmodulating the HDFs may comprise administering an agent that downmodulates the HDF to the subject such that the agent contacts HIV host cells of the subject. The agent may inhibit HDF gene expression, protein synthesis, HDF function or HDF activity, or combinations thereof.

### CROSS REFERENCE TO RELATED APPLICATIONS

This Application claims priority to U.S. Provisional Application 61/195,006, filed Oct. 2, 2008, and U.S. Provisional Application 61/007,766, filed Dec. 14, 2007, and U.S. Provisional Application 61/011,157, filed Jan. 15, 2008, the contents of each of which are incorporated by reference herein in their entirety.

### BACKGROUND OF THE INVENTION

The HIV-1 genomic RNA encodes only fifteen proteins [1, 2]. To complete its lifecycle, HIV-1 exploits multiple host cell biologic processes in each step of infection [2-6]. Viral entry depends on binding of the HIV envelope proteins to the cellular receptor CD4 and either of two co-receptors, CXCR4 or CCR5. The viral core, containing the viral capsid and nucleocapsid along with the viral genome, reverse transcriptase (RT), integrase (IN), protease (PR) and the viral accessory proteins Vif, Nef and Vpr, is released into the cytoplasm after fusion of the viral and cellular membranes. Collectively called the reverse transcription complex (RTC), this assembly binds to actin, triggering the synthesis of a double stranded viral DNA complement [7]. Once reverse transcription is complete, the RTC becomes the preintegration complex (PIC). In association with dynein, the PIC moves along microtubules to the nucleus, and enters via a nuclear pore [8]. The cellular and viral requirements for PIC nuclear import remain undefined.

In the nucleus HIV preferentially integrates into areas actively transcribed by Polymerase I (Pol II, [9]). Integration is facilitated by tethering of IN by the host cell protein, LEDGF [10-12]. The integrated proviral long terminal repeat (LTR) binds host transcription factors which recruit RNA Pol II and the transcriptional machinery [13]. Transcription of the provirus depends on the viral factor, Tat, which binds to the transactivation response element (TAR) in the proviral RNA. Tat promotes elongation by recruiting Cyclin T1, HTATSF1 and Cdk9, stimulating phosphorylation of the RNA Pol II carboxy terminal tail. Unspliced and partially spliced transcripts require the viral Rev protein for nuclear export. Rev first binds the rev response element (RRE) in the proviral RNA, and then adheres to the cellular export mediator CRM1 [14]. HIV assembly is directed to the plasma membrane by the myristoylation of the viral Gag protein. In T cells and HeLa cells, viruses bud through both multi vesicular bodies (MVBs) and late-endosome-to-trans-Golgi trafficking to the plasma membrane; the latter pathway requires Rab9p40 [15]. Because of the complexity of the retroviral life cycle and the small number of virally encoded proteins, important viral-host relationships likely remain to be discovered.

### SUMMARY

Aspects of the present invention relate to a method for treating and/or preventing HIV infection in a cell comprising downmodulating one or more of the HIV-dependency factors (HDFs) listed in Table 2 and/or Table 3 and/or Table 4 to thereby treat and/or prevent HIV infection in the cell. In the various embodiments of the invention, downmodulating the HDFs may comprise contacting the cell with an agent that downmodulates the HDF. Another aspect of the invention relates to a method for treating and/or preventing HIV infection in a subject comprising downmodulating one or more of the HIV-dependency factors (HDFs) listed in Table 2 and/or Table 3 and/or Table 4, to thereby treat and/or prevent HIV infection in the subject. In the various embodiments of the invention, the method may further comprise selecting a subject diagnosed with or at risk for HIV infection, prior to downmodulating. In the various embodiments of the invention, downmodulating the HDFs may comprise administering an agent that downmodulates the HDF to the subject such that the agent contacts HIV host cells of the subject. In the various embodiments of the invention, the agent may inhibit HDF gene expression, protein synthesis, HDF function or HDF activity, or combinations thereof.

### BRIEF DESCRIPTION OF THE DRAWINGS

FIG. 1A-FIG. 1C. siRNA screen for host factors required for HIV infection. (A) Schematic representation of screen. Arrayed pools of siRNAs were transfected into TZM-bl cells in a 384-well format. 72 h after transfection, HIV-IIIB virus was added and 48 h thereafter, cultured supernatant was removed and added to a fresh plate of TZM-bl cells. In part one of the screen, the siRNA transfected cells 48 h after infection were then fixed, permeabilized, stained and imaged for HIV p24 protein and DNA (part one of screen). In part two, cells were cultured for 24 h after the addition of supernatant, then lysed, exposed to fluorescent beta-galactosidase substrate, and relative light units (RLU) recorded on a plate reader. (B) Screen part one with the indicated siRNAs, as described above measuring relative p24 staining. (C) Screen part two measuring functional virus production with the indicated siRNAs, as described above. Relative Light Units (RLU). Error bars represent standard deviation of the mean (SD).

FIG. 2A-FIG. 2D. Enrichment analysis of HIV dependencies. (A) Subcellular localization of HDFs. Proteins. were manually curated based on subcellular localization annotated in UniProt and Gene ontology. If no annotation was available prediction programs were employed to identify transmembrane or mitochondrial proteins. The localization for each protein is provided in supplementary Table 3. (B-C) Gene ontology biological process (B) and molecular function (C) analysis. Of the 275 identified genes, 103 were assigned with 136 statistically significant (p<0.05) biological processes and 86 with 44 molecular functions. Gene ontology terms were processed to reduce redundancy (see methods) and curated manually (Table 3). The biological processes are ordered clockwise with ascending p-values and the molecular function significance threshold is indicated by a red line at 1.3=−log(P=0.05). (D) Pathway enrichment analysis obtained from the Ingenuity program using the right-tailed Fisher's exact test. Threshold is at 1.3=−log(P=0.05).

FIG. 3A-FIG. 3G. Rab6 is required for HIV infection. (A, B) TZM-bl HeLa cells stably expressing the indicated shRNAs, and either the control green fluorescence protein (GFP) or a Rab6-GFP fusion (Rab6-GFP), were infected with HIV and analyzed for (A) p24 at 48 h post infection or (B) Tat-dependent beta-galactosidase reporter expression 20 h post infection. Empty vector (mir30), firefly luciferase (FF), shRNAs against Rab6 (shRab6-1, 2 and 3) (C) Rab6 depletion specifically inhibits native-enveloped HIV. The indicated cell lines were infected with either HIV-IIIB, VSV-G pseudotyped MLV-EGFP (Moloney leukemia virus) or VSV-G pseudotyped HIV-YFP (an HIV virus engineered to express YFP). Infection was monitored with immunoflourescence (IF) of p24 (HIV-IIIB) or the respective reporter genes (EGFP, YFP), at 48 h post infection. (D) HIV infection involving either the CXCR4 or CCR5 co-receptor is attenuated by Rab6 depletion. Cell lines from (A) were infected with either HIV-IIIB or Bal viral strains and monitored by p24 staining after 48 h. (E) Rab6 depletion blocks HIV prior to late reverse transcription. Cells from (A) were infected with HIV and the late reverse transcription products (late RT) were assessed by quantitative PCR. (F) Rab6 is required for cell fusion. The shRab6 cell lines containing a Tat-dependent β-galactosidase reporter were layered for 6 h. with HL2/3 cells expressing HIV-1 Gag, Env, Tat, Rev, and Nef proteins from a stably expressed molecular clone HXB2/3gpt provirus. The relative amount of cell fusion was quantitated by assaying β-gal activity. (G) Rab6 depletion protects T cells from HIV infection. Jurkat T cells were transiently transfected with the indicated siRNAs for 72 h, then infected with HIV-IIIB and analyzed by FACS by staining with either anti-p24 antibody, or an isotype matched control antibody (IgG1), 48 h after infection. Error bars represent mean standard deviation (SD) throughout.

FIG. 4A-FIG. 4G. TNPO3-depleted cells resist HIV infection. (A) TZM-bl HeLa cells were transfected with indicated siRNAs for 72 h, then infected with HIV-IIIB. After 20 h, beta-galactosidase activity was measured. (B) TZM-bl HeLa cells were transiently transfected with the indicated siRNAs, and then either infected with HIV-IIIB or HIV-YFP VSV-G virus, or transiently transfected with the HIV-YFP plasmid 72 h after siRNA transfection. HIV infection was monitored for p24 (HIV-IIIB), or YFP expression 48 h post infection or transfection. (C) TNPO3 depletion preferentially affects lentiviruses. TZM-bl cells were transfected with the indicated siRNAs for 72 h then infected with the indicated viruses or transfected with the Tat-independent pHAGE-CMV-ZSG plasmid. After 48 h, levels of p24, ZSG, or EGFP were determined by IF. (D) TNPO3 depletion protects T cells from HIV infection. Jurkat T cells were transfected with the indicated siRNAs for 72 h then infected with HIV. After 48 h cells were analyzed for p24 expression. (E) TNPO3 mRNA reduction by siRNAs. TZM-bl HeLa cells were transfected with the indicated siRNAs for 72 h, then cDNA was prepared and TNPO3 expression levels were measured by quantitative real time PCR. (F and G) TNPO3 depletion blocks HIV after reverse transcription, but prior to integration. TZM-bl cells were transfected with the indicated siRNA (TNPO3, siRNAs 5-8 pooled), and infected with HIV. 72 h later, reverse transcription products (late RT) were assessed by quantitative PCR, and integrated viral DNA was quantitated by nested Alu-PCR. Error bars throughout represent standard deviation of the mean (SD).

FIG. 5A-FIG. 5F. Med28 is required for HIV replication. (A) SiRNAs were transfected into TZM-bl cells for 72 h, then infected with HIV. After 20 h, cells were analyzed for level of Tat activity by beta-galactosidase activity. (B) Loss of Med28 inhibits both native-enveloped HIV and a VSV-G pseudotyped HIV-YFP. TZM-bl cells were transfected with the indicated siRNAs, and then infected with HIV-IIIB, MLV-EGFP, or HIV-YFP 72 h post transfection. HIV infection was monitored with IF staining for p24 or reporter expression at 48 h. (C) Med28 depletion protects T cells from HIV infection. Jurkat T cells were transiently transfected with the indicated siRNAs, then infected with HIV 72 h later. After an additional 48 h, the T cells were analyzed by FACS, with staining for either p24 or an isotyped matched control antibody (IgG1). (D, E) Med28 is required for HIV transcription. The noted siRNA pools were transfected into TZM-bl cells for 72 h then infected with HIV-IIIB, with late reverse transcription products (late RT) assessed by quantitative PCR (D), and integrated proviral DNA quantitated by nested Alu-PCR (E). (F) TZM-bl cells were treated with the noted siRNA pools, after 72 h they were infected with HIV-YFP virus or transfected with a HIV-YFP plasmid. Levels of YFP reporter protein were monitored by IF 48 h later. Error bars represent standard deviation of the mean (SD).

FIG. 6A-FIG. 6D. Targeting of Vps53 inhibits HIV. (A) TZM-bl cells received the noted siRNA treatment. 72 h later these cells were infected with HIV-IIIB; After 20 h of infection, the cells were analyzed for level of Tat activity by determining beta-gal expression in cell lysate. (B) Vps53 depletion inhibits only native-enveloped HIV and not the VSV-G pseudotyped HIV-YFP or MLV-EGFP viruses. TZM-bl HeLa cells were transiently transfected with the indicated siRNAs, and then infected with HIV-IIIB, MLV-EGFP, or HIV-YFP 72 h post transfection. HIV infection was monitored with IF staining for p24 (HIV), or the respective reporter genes at 48 h post infection. (C) Decreased Vps53 levels prevent cell fusion. TZM-bl cells were transfected with the noted siRNAs, at a high cell density. 72 h later these transfected cells were layered with HL2/3 cells. The co-culture was then incubated for 6 h. at 37 C. This permits fusion between the two cells lines to occur. The relative amount of cell fusion is then quantitated by lysing the cells and determining Tat-dependent beta-gal activity (red bars). To illustrate the similarities in the fusion defect and resistance to HIV infection conferred after siRNA transfection, we have shown the percentage of cells infected vs. controls at 48 h after HIV exposure (blue bars). (D) Vps53 depletion does not significantly change CXCR4 levels. TZM-bl HeLa cells treated with the listed sRNAs against Vps53 (1-4) or Luciferase, were stained with anti-CXCR4-PE conjugated antibody, or an isotype matched-PE control antibody, and analyzed by FACS.

FIG. 7. Mapping of gene candidates to HIV life cycle. Using annotation databases (UniProt, OMIM, RefSeq, NCBI GeneRIF and KEGG-see methods) the function and subcellular location of each candidate gene was evaluated. Considering current knowledge of the HIV life cycle and known interacting host factors, each gene was placed at the most likely position to elicit HIV dependency. Note, some genes may be placed in multiple locations to represent our interpretation that they may have more than one significant role in the HIV lifecycle.

### DETAILED DESCRIPTION OF THE INVENTION

Aspects of the present invention stem from the identification of host factors involved in HIV infection. 387 such host factors, herein referred to as HIV-dependency factors (HDFs), were identified in a primary genome wide screen. These HDF's are listed in Table 2. 275 of these HDF's were further verified in a validation screen. Validation further indicates that the 275 factors are involved and necessary for optimal HIV infection. It should be noted that lack of validation of an HDF does not necessarily invalidate the HDF, as validation may be possible with other means, or simply repeated performance of the validation screen and optimization of conditions and/or reagents used. Of the 275 validated HDFs, 237 HDFs had not previously been identified as involved in HIV infection. Inhibition of these HDFs inhibited HIV infection. This inhibition takes place at the first phase of the viral life cycle (entry to transcription of the integrated provirus) and/or at the late stage of viral replication (viral replication), as is reflected in the part of the screen in which the specific HDF was identified.

In a follow-up screen, using the same methods as the earlier screen, an additional 82 host factors involved in HIV infection were identified and verified in a validation screen. These HDFs are listed in Table 3. 14 strong candidates for HIV therapeutics are listed in Table 4.

The identified HDFs described herein serve as effective targets for treatment and/or prevention of HIV infection in a cell. As such, aspects of the present invention relate to methods of treating and/or preventing HIV infection in a cell. The method involves downmodulating one or more of the HDFs identified herein in the cell to thereby treat and/or prevent HIV infection in the cell. In one embodiment, the HDF corresponds to an HDF listed in Table 2 and/or Table 3 and/or Table 4.

Downmodulation occurs in the HIV host cells of the individual to thereby inhibit or prevent successful HIV infection in the host cells of the subject.

Downmodulation can be achieved by contacting the cell with an agent that downmodulates the HDF. The agent can be formulated to enhance specific uptake or delivery to the interior of the cell as required.

The identified HDFs described herein also serve as effective targets for treatment and/or prevention of HIV infection in an individual. As such, aspects of the present invention relate to methods of treating and/or preventing HIV infection in a subject. The method involves downmodulating one or more of the HDFs identified herein to thereby inhibit successful HIV infection. In one embodiment, the HDF corresponds to an HDF listed in Table 2 and/or Table 3 and/or Table 4.

In one embodiment, the method involves first selecting a subject which is diagnosed with, or at risk for, HIV infection. Such a selection is performed, for instance, by routine examination and diagnosis by the skilled medical practitioner. In another embodiment, the methods involves first selecting a subject who has symptoms of HIV infection, in lieu of a conclusive diagnosis. Such symptoms include, without limitation, conditions, syndromes and infections routinely associated with autoimmune deficiency syndrome (AIDS) in a subject. This could also be performed, for instance through routine examination by the skilled medical practitioner who would then make the appropriate determination of the presence of symptoms.

In a subject, downmodulation can be achieved by administration to the subject, of an agent that downmodulates the HDF in cells of the subject. Administration is performed such that the agent contacts cells of the subject which HIV has infected or could potentially infect. Such cells are referred to herein as HIV host cells. Typically HIV host cells will express CD4 and either of two co-receptors, CXCR4 or CCR5 on their cell surface. The agent can be formulated to enhance specific uptake or delivery to the interior of the cell as required.

Administration of the agent is by means which it will contact the host cell. Examples of such routes include parenteral, enteral, and topical administration. Parenteral administration is usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. Administration can be systemic administration, or localized, as determined necessary by the skilled practitioner. Topical administration is preferably by a route of entry of HIV in initial infection (e.g., vaginal, skin, anal, etc.).

Downmodulation refers to reducing the function of the HDF. This can be accomplished by directly affecting the HDF itself, (e.g., by reducing HDF gene expression or protein synthesis), or alternatively by reducing HDF function/activity. HDF function/activity can be reduced by directly inhibiting the HDF protein itself. As such, an agent useful in the present invention is one that inhibits HDF gene expression or protein synthesis, or inhibits HDF function or activity.

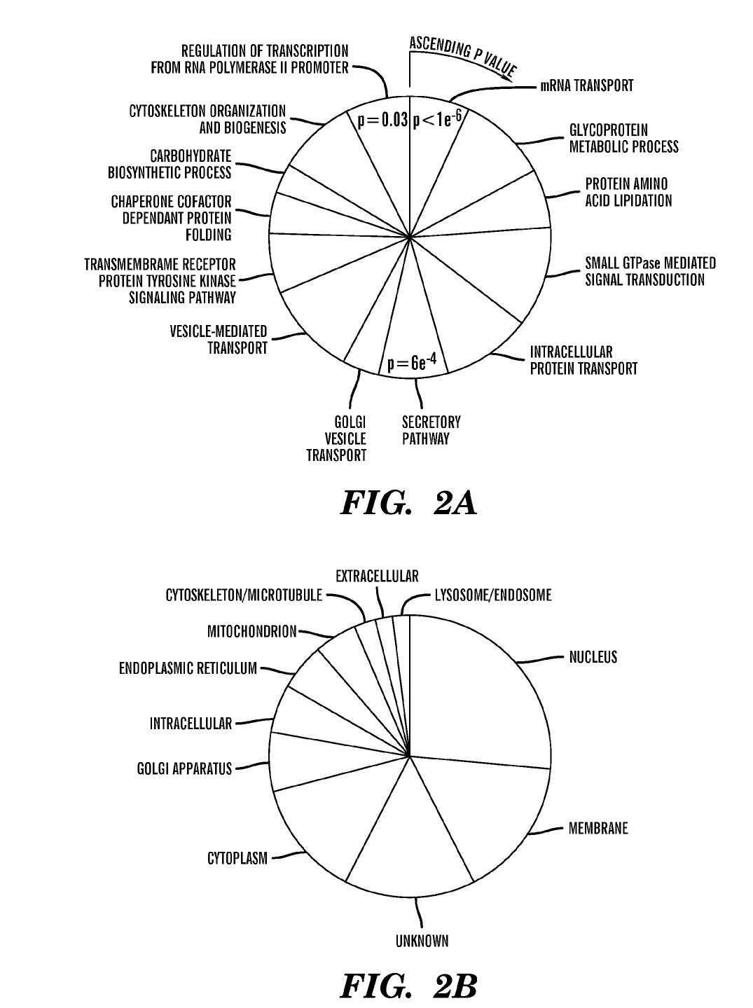

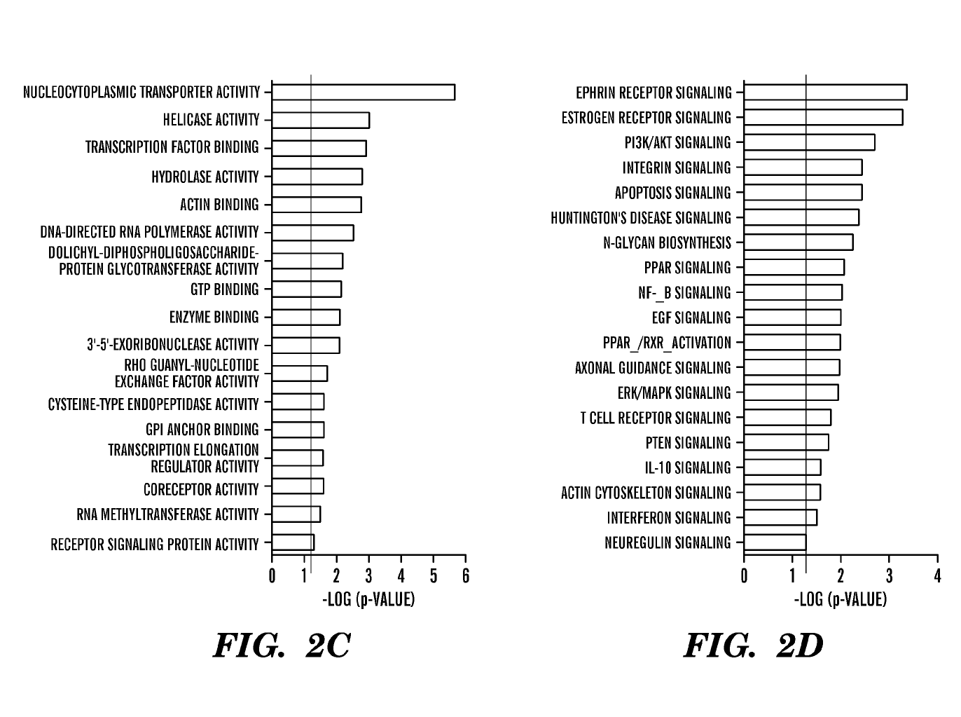

Analysis of the HDFs identified in the genomic screen identified various cellular functions (cellular processes, also referred to herein as biological processes) that were not previously known to be involved in the HIV infection/replication cycle (listed in FIG. 2). Analysis also identified many HDFs as components of macromolecular complexes. In addition, analysis of the HDFs identified specific signal transduction pathways involved in HIV infection. Interference (e.g., inhibition) of such cellular machinery is also expected to reduce HIV infection. As such, inhibition of one or more of the macromolecular complexes and/or cellular functions and/or signal transduction pathways identified herein is expected to downmodulate the HDF to produce an inhibitory effect on HIV infection. Examples of such macromolecular complexes include, without limitation, nup 160 subcomplex of the nuclear pore, mediator, Conserved oligomeric golgi (COG) complex, Transport protein particle (TRAPP) I complex, and Golgi-associated retrograde protein (GARP) complex. Cellular functions include, without limitation, protein conjugation pathways involved in autophage (HDF: Atg7, Atg8, Atg12, and Atg16L2), lysosomal functions involved in autophagy (HDF: CLN3, LapTM5), functions involved in vesicular transport and GTPase activity (HDFs: Rab1b, Rab2, Rab6a and Rab28), functions involved in retrograde golgi-to-ER transport such as recycling of Golgi glycosyltransferases, and endosomal trafficking. Interference with one or more of the cellular processes identified herein, to produce inhibition of HIV infection, may involve partial to complete inhibition of the process, and may be temporary or permanent interference.

Inhibition of HIV infection by the methods disclosed herein is applicable at the cellular level and also at the whole organism level. Inhibition at the cellular level of HIV infection refers to a specific cell or group of cells (e.g., a cell type). Inhibition at the whole organism level refers to inhibition of HIV infection of an individual (e.g., to prevent an individual from being afflicted with HIV, or to reduce that individual's viral load, or infectivity of others). The term “inhibition” is used to reflect complete inhibition and also partial inhibition of infection. Complete inhibition indicates that the HIV virus is completely unable to successfully infect and/or replicate and/or further infect other cells. This can be determined in a number of ways, at the cellular and/or whole organism level, by the skilled practitioner. One such determination is by an inability to obtain infectious HIV from a host cell. Another such determination is by an inability to determine that HIV has entered the host cell. At the whole organism level, standard methods for assaying for HIV infection can be used (e.g., assaying for antibodies to HIV in the individual). Partial inhibition refers to a measurable, statistically significant reduction in the ability of HIV to infect and/or replicate and/or further infect other cells, as compared to an appropriate control which has not been subjected to the therapeutics described herein. One example would be a requirement for higher levels of exposure or longer period of exposure to HIV for successful infection.

As used herein, the term “treating” and “treatment” and/or “palliating” refers to administering to a subject an effective amount of a the composition so that the subject has an improvement in the disease, for example, beneficial or desired clinical results. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. This includes symptoms of any of the AIDS-related conditions such as AIDS-related complex (ARC), progressive generalized lymphadenopathy (PGL), anti-HIV antibody positive conditions, and HIV-positive conditions, AIDS-related neurological conditions (such as dementia or tropical paraparesis), Kaposi's sarcoma, thrombocytopenia purpurea and associated opportunistic infections such as Pneumocystis carinii pneumonia, Mycobacterial tuberculosis, esophageal candidiasis, toxoplasmosis of the brain, CMV retinitis, HIV-related encephalopathy, HIV-related wasting syndrome. Treating can refer to prolonging survival as compared to expected survival if not receiving treatment. Thus, one of skill in the art realizes that a treatment may improve the disease condition, but may not be a complete cure for the disease.

Standard methods for measuring in vivo HIV infection and progression to AIDS can be used to determine effective treatment with the agents described herein. For example, after treatment with an HIV-inhibiting compound of the invention, a subject's CD4+ T cell count can be monitored. A rise in CD4+ T cells indicates successful treatment of the subject. This, as well as other methods known to the art, may be used to determine the extent to which the agents and therapeutic compositions and formulations of the present invention are effective at treating HIV infection and AIDS in a subject.

The agents of the present invention (alone or within compositions or formulations described herein) can also be combined with or used in association with other therapeutic agents. In some applications, a first agent is used in combination with a second HIV-inhibiting compound in order to inhibit HIV infection to a more extensive degree than can be achieved when one agent or HIV-inhibiting compound is used individually. An HIV-inhibiting compound can be an agent identified herein or a known anti-HIV drug such as AZT (generic name zidovudine). Any number of combinations of agents described herein and/or known-anti-HIV drugs are envisioned as providing therapeutic benefit.

HDF downmodulation can be achieved by inhibition of HDF protein expression (e.g., transcription, translation, post-translational processing) or protein function. Any composition known to inhibit or downmodulate one or more of the HDF disclosed herein can be used for HDF downmodulation. Inhibition of one or more of these molecular functions is expected to inhibit HIV via a downmodulatory effect on the HDF.

Another mechanism of a downmodulatory agent of the present invention is gene silencing of the target HDF gene, such as with an RNAi molecule (e.g., siRNA or miRNA). This entails a decrease in the mRNA level in a cell for a target HDF by at least about 5%, about 10%, about 20%, about 30%, about 40%, about 50%, about 60%, about 70%, about 80%, about 90%, about 95%, about 99%, about 100% of the mRNA level found in the cell without the presence of the RNAi. In one preferred embodiment, the mRNA levels are decreased by at least about 70%, about 80%, about 90%, about 95%, about 99%, about 100%.

Another aspect of the invention relates to the agent that downmodulates the HDF, and formulations and compositions in which it is contained. Any known inhibitor or downmodulator of the HDFs identified herein can be used as a downmodulating agent in the present methods. In addition, new agents are identified herein as useful as a downmodulatory agent in the treatment of HIV in a subject.

Agents useful in the methods as disclosed herein may inhibit gene expression (i.e. suppress and/or repress the expression of a gene of interest (e.g., the HDF gene)). Such agents are referred to in the art as “gene silencers” and are commonly known to those of ordinary skill in the art. Examples include, but are not limited to a nucleic acid sequence, (e.g., for an RNA, DNA, or nucleic acid analogue). These can be single or double stranded. They can encode a protein of interest, can be an oligonucleotide, a nucleic acid analogue. Included in the term “nucleic acid sequences” are general and/or specific inhibitors. Some known nucleic acid analogs are peptide nucleic acid (PNA), pseudo-complementary PNA (pc-PNA), locked nucleic acids (LNA) and derivatives thereof. Nucleic acid sequence agents can also be nucleic acid sequences encoding proteins that act as transcriptional repressors, antisense molecules, ribozymes, small inhibitory nucleic acid sequences, such as RNAi, shRNAi, siRNA, micro RNAi (miRNA), antisense oligonucleotides. Many of these molecular functions are known in the art. As such these inhibiting can function as an agent in the present invention. In one embodiment, the RNAi comprises the nucleic acid sequences listed in Table 3 for use in downmodulating the corresponding HDF listed in Table 3. Additional sequences may also be present. In another embodiment, the RNAi comprises a fragment of at least 5 consecutive nucleic acids of the sequences listed in Table 3 for use in downmodulating the corresponding HDF listed in Table 3. Longer fragments of the nucleic acid sequences listed in Table 3, for downmodulating of the corresponding HDF listed in Table 3, may also be used, (e.g., at least 6, 7, 8, 9, 10, 11, 12, 13, 14, 15, 16, 17, or 18 nucleic acids). In one embodiment, the RNAi sequence directly corresponds to the siRNA listed in Table 6 or Table 9, for use in downmodulating the corresponding HDF listed in Table 6 or Table 9, respectively. In addition to the sequences specified herein, the agent may further comprise other moieties, or non-nucleic acid components.

Such an agent can take the form of any entity which is normally not present or not present at the levels being administered to the cell or oganism. Agents such as chemicals; small molecules; nucleic acid sequences; nucleic acid analogues; proteins; peptides; aptamers; antibodies; or fragments thereof, can be identified or generated for use to downmodulate a HDF.

Agents in the form of a protein and/or peptide or fragment thereof can also be designed to downmodulate a HDF. Such agents encompass proteins which are normally absent or proteins that are normally edogenously expressed in the host cell. Examples of useful proteins are mutated proteins, genetically engineered proteins, peptides, synthetic peptides, recombinant proteins, chimeric proteins, antibodies, midibodies, minibodies, triabodies, humanized proteins, humanized antibodies, chimeric antibodies, modified proteins and fragments thereof. Agents also include antibodies (polyclonal or monoclonal), neutralizing antibodies, antibody fragments, peptides, proteins, peptide-mimetics, aptamers, small molecules, carbohydrates or variants thereof that function to inactivate the nucleic acid and/or protein of the gene products identified herein, and those as yet unidentified. Inhibitory agents can also be a chemical, small molecule, chemical entity, nucleic acid sequences, nucleic acid analogues or protein or polypeptide or analogue or fragment thereof.

The agent may function directly in the form in which it is administered. Alternatively, the agent can be modified or utilized intracellularly to produce something which downmodulates an HDF, such as introduction of a nucleic acid sequence into the cell and its transcription resulting in the production of the nucleic acid and/or protein inhibitor of HDF within the cell. In some embodiments, the agent is any chemical, entity or moiety, including without limitation synthetic and naturally-occurring non-proteinaceous entities. In certain embodiments the agent is a small molecule having a chemical moiety. For example, chemical moieties included unsubstituted or substituted alkyl, aromatic, or heterocyclyl moieties including macrolides, leptomycins and related natural products or analogues thereof. Agents can be known to have a desired activity and/or property, or can be selected from a library of diverse compounds.

The agent may comprise a vector. Many such vectors useful for transferring exogenous genes into target mammalian cells are available. The vectors may be episomal, e.g., plasmids, virus derived vectors such cytomegalovirus, adenovirus, etc., or may be integrated into the target cell genome, through homologous recombination or random integration, e.g., retrovirus derived vectors such MMLV, HIV-1, ALV, etc. For modification of stem cells, lentiviral vectors are preferred. Lentiviral vectors such as those based on HIV or FIV gag sequences can be used to transfect non-dividing cells, such as the resting phase of human stem cells (see Uchida et al. (1998) P.N.A.S. 95(20): 11939-44). In some embodiments, combinations of retroviruses and an appropriate packaging cell line may also find use, where the capsid proteins will be functional for infecting the target cells. Usually, the cells and virus will be incubated for at least about 24 hours in the culture medium. The cells are then allowed to grow in the culture medium for short intervals in some applications, e.g. 24-73 hours, or for at least two weeks, and may be allowed to grow for five weeks or more, before analysis. Commonly used retroviral vectors are “defective”, i.e. unable to produce viral proteins required for productive infection. Replication of the vector requires growth in the packaging cell line.

As used herein, the term “vector” refers to a nucleic acid molecule capable of transporting another nucleic acid to which it has been linked. Preferred vectors are those capable of autonomous replication and/or expression of nucleic acids to which they are linked. Vectors capable of directing the expression of genes to which they are operatively linked are referred to herein as “expression vectors”.

Many viral vectors or virus-associated vectors are known in the art. Such vectors can be used as carriers of a nucleic acid construct into the cell. Constructs may be integrated and packaged into non-replicating, defective viral genomes like Adenovirus, Adeno-associated virus (AAV), or Herpes simplex virus (HSV) or others, including reteroviral and lentiviral vectors, for infection or transduction into cells. The vector may or may not be incorporated into the cells genome. The constructs may include viral sequences for transfection, if desired. Alternatively, the construct may be incorporated into vectors capable of episomal replication, e.g. EPV and EBV vectors. An HIV based vector would be particularly useful in targeting HIV host cells.

The inserted material of the vectors described herein may be operatively linked to an expression control sequence when the expression control sequence controls and regulates the transcription and translation of that polynucleotide sequence. The term “operatively linked” includes having an appropriate start signal (e.g., ATG) in front of the polynucleotide sequence to be expressed, and maintaining the correct reading frame to permit expression of the polynucleotide sequence under the control of the expression control sequence, and production of the desired polypeptide encoded by the polynucleotide sequence. In some examples, transcription of an inserted material is under the control of a promoter sequence (or other transcriptional regulatory sequence) which controls the expression of the recombinant gene in a cell-type in which expression is intended. It will also be understood that the inserted material can be under the control of transcriptional regulatory sequences which are the same or which are different from those sequences which control transcription of the naturally-occurring form of a protein. In some instances the promoter sequence is recognized by the synthetic machinery of the cell, or introduced synthetic machinery, required for initiating transcription of a specific gene.

The promoter sequence may be a “tissue-specific promoter,” which means a nucleic acid sequence that serves as a promoter, i.e., regulates expression of a selected nucleic acid sequence operably linked to the promoter, and which affects expression of the selected nucleic acid sequence in specific cells, preferably in HIV host cells. The term also covers so-called “leaky” promoters, which regulate expression of a selected nucleic acid primarily in one tissue, but cause expression in other tissues as well.

The term “RNAi” as used herein refers to interfering RNA or RNA interference. RNAi refers to a means of selective post-transcriptional gene silencing by destruction of specific mRNA by molecules that bind and inhibit the processing of mRNA, for example inhibit mRNA translation or result in mRNA degradation. As used herein, the term “RNAi” refers to any type of interfering RNA, including but are not limited to, siRNAi, shRNAi, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein).

In one embodiment, the agent is an RNA interference molecule. The term “RNAi” and “RNA interfering” with respect to an agent of the invention, are used interchangeably herein.

RNAi molecules are typically comprised of a sequence of nucleic acids or nucleic acid analogs, specific for a target gene. A nucleic acid sequence can be RNA or DNA, and can be single or double stranded, and can be selected from a group comprising; nucleic acid encoding a protein of interest, oligonucleotides, nucleic acid analogues, for example peptide-nucleic acid (PNA), pseudo-complementary PNA (pc-PNA), locked nucleic acid (LNA).

As used herein an “siRNA” refers to a nucleic acid that forms a double stranded RNA, which double stranded RNA has the ability to reduce or inhibit expression of a gene or target gene when the siRNA is present or expressed in the same cell as the target gene, for example an HDF gene. The double stranded RNA siRNA can be formed by the complementary strands. In one embodiment, a siRNA refers to a nucleic acid that can form a double stranded siRNA. The sequence of the siRNA can correspond to the full length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length). An siRNA can be chemically synthesized, it can be produced by in vitro transcription, or it can be produced within a cell specifically utilized for such production.

As used herein “shRNA” or “small hairpin RNA” (also called stem loop) is a type of siRNA. In one embodiment, these shRNAs are composed of a short, e.g. about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow. shRNAs functions as RNAi and/or siRNA species but differs in that shRNA species are double stranded hairpin-like structure for increased stability. These shRNAs, as well as other such agents described herein, can be contained in plasmids, retroviruses, and lentiviruses and expressed from, for example, the pol III U6 promoter, or another promoter (see, e.g., Stewart, et al. (2003) RNA April; 9(4):493-501, incorporated by reference herein in its entirety).

The terms “microRNA” or “miRNA” are used interchangeably herein are endogenous RNAs, some of which are known to regulate the expression of protein-coding genes at the posttranscriptional level. Endogenous microRNA are small RNAs naturally present in the genome which are capable of modulating the productive utilization of mRNA. The term artificial microRNA includes any type of RNA sequence, other than endogenous microRNA, which is capable of modulating the productive utilization of mRNA. MicroRNA sequences have been described in publications such as Lim, et al., Genes & Development, 17, p. 991-1008 (2003), Lim et al Science 299, 1540 (2003), Lee and Ambros Science, 294, 862 (2001), Lau et al., Science 294, 858-861 (2001), Lagos-Quintana et al, Current Biology, 12, 735-739 (2002), Lagos Quintana et al, Science 294, 853-857 (2001), and Lagos-Quintana et al, RNA, 9, 175-179 (2003), which are incorporated by reference. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al. 2004. Cell 116:281-297), comprises a dsRNA molecule.

In one embodiment, siRNA is a double stranded RNA (dsRNA) molecule of about 15, 16, 17, 18, 19, 20, 21, 22, 23, 24, 25, 26, 27, 28, or 30 nucleotides in length, preferably about 15 to about 28 nucleotides, more preferably about 19, 20, 21, 22, 23, 24, or nucleotides in length, and can contain a 3′ and/or 5′ overhang on each strand having a length of about 1, 2, 3, 4, or 5 nucleotides. The length of the overhang is independent between the two strands, i.e., the length of the over hang on one strand is not dependent on the length of the overhang on the second strand. Preferably the siRNA is capable of promoting RNA interference through degradation or specific post-transcriptional gene silencing (PTGS) of the target messenger RNA (mRNA).

In the course of the screen, RNA interference (RNAi) target sites on the nucleic acid encoding each HDF were identified. These target sites, correspond to the regions of the HDF gene which are contacted by (e.g. hybridized) the siRNA. These sites, or portions of these target sites, can be used to reduce the expression of the HDF, to thereby decrease/prevent HIV infection of a cell. As such, aspects of the present invention relate to methods and compositions for modulating the expression of HDFs and more particularly to the down regulation of HDF mRNA and HDF protein levels by agents which are RNA interference (RNAi) molecules which utilize these target sites, or a portion thereof. Such downmodulation of expression of HDFs is applied in the present invention to cells which HIV is capable of infecting, for prevention or reduction of HIV infection of a cell. Application of such downmodulation to an entire organism (e.g. human or primate) can constitute an effective therapeutic treatment of the organism for HIV infection.

In one embodiment, the RNAi agent targets at least 5 contiguous nucleotides in the identified target sequence. In one embodiment, those continguous nucleotides correspond to at least 5 contiguous nucleotides of an siRNA sequence listed in Table 3, for inhibition of the corresponding HDF listed in Table 3. In one embodiment, the RNAi agent targets at least 6, 7, 8, 9 or 10 contiguous nucleotides in the identified target sequence (e.g., wherein those contiguous nucleotides correspond to a like number of contiguous nucleotides of an siRNA sequence listed in Table 3, for inhibition of the corresponding HDF listed in Table 3). In one embodiment, the RNAi agent targets at least 11, 12, 13, 14, 15, 16, 17, 18 or 19 contiguous nucleotides in the identified target sequence (e.g., wherein those contiguous nucleotides correspond to a like number of contiguous nucleotides of an siRNA sequence listed in Table 3, for inhibition of the corresponding HDF listed in Table 3). In combination with any one of these number of contiguous nucleotides, the RNAi agent may also further comprise additional sequences not identified herein, which correspond to the target gene, but are not identified herein as target sites.

Methods of delivering RNAi interfering (RNAi) agents, e.g., an siRNA, or vectors containing an RNA interfering agent, to the target cells (e.g., HIV host cells) can include, for example (i) injection of a composition containing the RNA interfering agent, e.g., an siRNA, or (ii) directly contacting the cell, e.g., a hematopoietic cell, with a composition comprising an RNA interfering agent, e.g., an siRNA. In another embodiment, RNA interfering agents, e.g., an siRNA can be injected directly into any blood vessel, such as vein, artery, venule or arteriole, via, e.g., hydrodynamic injection or catheterization. In some embodiments RNAi agents such as siRNA can delivered to specific organs (e.g. bone marrow) or by systemic administration.

Colloidal dispersion systems may be used as delivery vehicles to enhance the in vivo stability of the agents (e.g. RNA9) to a particular organ, tissue or cell type. Colloidal dispersion systems include, but are not limited to, macromolecule complexes, nanocapsules, microspheres, beads and lipid-based systems including oil-in-water emulsions, micelles, mixed micelles, liposomes and lipid:oligonucleotide complexes of uncharacterized structure. A preferred colloidal dispersion system is a plurality of liposomes. Liposomes are microscopic spheres having an aqueous core surrounded by one or more outer layers made up of lipids arranged in a bilayer configuration (see, generally, Chonn et al., Current Op. Biotech. 1995, 6, 698-708). Other examples of cellular uptake or membrane-disruption moieties include polyamines, e.g. spermidine or spermine groups, or polylysines; lipids and lipophilic groups; polymyxin or polymyxin-derived peptides; octapeptin; membrane pore-forming peptides; ionophores; protamine; aminoglycosides; polyenes; and the like. Other potentially useful functional groups include intercalating agents; radical generators; alkylating agents; detectable labels; chelators; or the like.

Other colloidal dispersion systems lipid particle or vesicle, such as a liposome or microcrystal, which may be suitable for parenteral administration. The particles may be of any suitable structure, such as unilamellar or plurilamellar, so long as the antisense oligonucleotide is contained therein. Positively charged lipids such as N-[I-(2,3dioleoyloxi)propyl]-N,N,N-trimethyl-anunoniummethylsulfate, or “DOTAP,” are particularly preferred for such particles and vesicles. The preparation of such lipid particles is well known. See, e.g., U.S. Pat. Nos. 4,880,635; 4,906,477; 4,911,928; 4,917,951; 4,920,016; and 4,921,757 which are incorporated herein by reference. Other non-toxic lipid based vehicle components may likewise be utilized to facilitate uptake of the antisense compound by the cell.

In some embodiments, in order to increase nuclease resistance in an RNAi agent as disclosed herein, one can incorporate non-phosphodiester backbone linkages, as for example methylphosphonate, phosphorothioate or phosphorodithioate linkages or mixtures thereof, into one or more non-RNASE H-activating regions of the RNAi agents. Such non-activating regions may additionally include 2′-substituents and can also include chirally selected backbone linkages in order to increase binding affinity and duplex stability. Other functional groups may also be joined to the oligonucleoside sequence to instill a variety of desirable properties, such as to enhance uptake of the oligonucleoside sequence through cellular membranes, to enhance stability or to enhance the formation of hybrids with the target nucleic acid, or to promote cross-linking with the target (as with a psoralen photo-cross-linking substituent). See, for example, PCT Publication No. WO 92/02532 which is incorporated herein in by reference.

In one embodiment, the agent described herein is an active ingredient in a composition comprising a pharmaceutically acceptable carrier. A “pharmaceutically acceptable carrier” means any pharmaceutically acceptable means to mix and/or deliver the targeted delivery composition to a subject. The term “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the composition and is compatible with administration to a subject, for example a human. Such compositions can be specifically formulated for administration via one or more of a number of routes, such as the routes of administration described herein. Supplementary active ingredients also can be incorporated into the compositions. In one embodiment, the supplementary active ingredient is a known treatment for HIV (e.g. AZT).

When an agent, formulation or pharmaceutical composition described herein, is administered to a subject, preferably, a therapeutically effective amount is administered. As used herein, the term “therapeutically effective amount” refers to an amount that results in an improvement or remediation of the disease, disorder, or symptoms of the disease or condition. One example is a reduction in pathology of HIV. The term “pathology” as used herein, refers to symptoms, for example, structural and functional changes in a cell, tissue, or organs, which contribute to a disease or disorder.

The methods and compositions described herein are particularly applicable to treatment and/or prevention of HIV-1 infection in an individual. However, other strains of HIV which cause AIDS are known to exist, and are highly homologous to HIV-1. As such, the methods and compositions described herein are also expected to be readily adaptable by the skilled practitioner to treatment and/or prevention of these infections (e.g. HIV-2 and HIV-3) in an individual. Accordingly, aspects of the present invention relate to methods and compositions, and identification of compositions described herein, for the treatment and/or prevention of HIV-2 or HIV-3 infection in an individual.

The identification of the HDFs described herein allows for rapid screening for additional therapeutics for treatment or prevention of HIV by identification of new downmodulators of a given HDF. Such an agent will have therapeutic use in the prevention and/or treatment of HIV infection in a cell and in an individual. As such, aspects of the invention relate to methods for identifying therapeutic agents for the prevention/treatment of HIV infection, comprising identifying an agent which downmodulates an HDF specified herein, by administering a candidate agent and assaying for downmodulation of one or more target HDFs.

The newly identified HDFs disclosed herein provide novel targets to screen for compounds that inhibit HIV infections. A method for identifying inhibitors of HIV infection is by identifying agents that downmodulate (e.g. directly inhibit) an HDF.

Various biochemical and molecular biology techniques or assays well known in the art can be employed to practice the present invention. Such techniques are described in, e.g., Handbook of Drug Screening, Seethala et al. (eds.), Marcel Dekker (1st ed., 2001); High Throughput Screening Methods and Protocols (Methods in Molecular Biology, 190), Janzen (ed.), Humana Press (1st ed., 2002); Current Protocols in Immunology, Coligan et al. (Ed.), John Wiley & Sons Inc (2002); Sambrook et al., Molecular Cloning: A Laboratory Manual, Cold Spring Harbor Press (3rd ed., 2001); and Brent et al., Current Protocols in Molecular Biology, John Wiley & Sons, Inc. (ringbou ed., 2003). Screens involve a test agent, which is a candidate molecule which is to be used in a screen and/or applied in an assay for a desired activity (e.g., downmodulation of HDF, inhibition of HDF protein activity, etc.)

Typically, test agents are first screened for their ability to downmodulate a biological activity of an HDF (“the first assay step”). Modulating agents thus identified are then subject to further screening for ability to inhibit HIV infection, typically in the presence of the HIV-interacting host factor (“the second testing step”). Depending on the HDF employed in the method, modulation of different biological activities of the HIV-interacting host factor can be assayed in the first step. For example, a test agent can be assayed for binding to the HDF. The test agent can be assayed for activity to downmodulate expression of the HDF, e.g., transcription or translation. The test agent can also be assayed for activities in modulating expression or cellular level of the HDF, e.g., post-translational modification or proteolysis. Test agents can be screened for ability to either up-regulate or down-regulate a biological activity of the HDF in the first assay step.

Once test agents that inhibit HDF are identified, they are typically further tested for ability to inhibit HIV infection. This further testing step is often needed to confirm that their modulatory effect on the HDF would indeed lead to inhibition of HIV infection. For example, a test agent which inhibits a biological activity, molecular activity or biological process of an HDF needs to be further tested in order to confirm that such modulation can result in suppressed or reduced HIV infection.

In both the first assaying step and the second testing step, either an intact HDF, or a fragment thereof, may be employed. Molecules with sequences that are substantially identical to that of the HDF can also be employed. Analogs or functional derivatives of the HDF could similarly be used in the screening. The fragments or analogs that can be employed in these assays usually retain one or more of the biological activities of the HDF (e.g., kinase activity if the HDF employed in the first assaying step is a kinase). Fusion proteins containing such fragments or analogs can also be used for the screening of test agents. Functional derivatives of an HDF usually have amino acid deletions and/or insertions and/or substitutions while maintaining one or more of the bioactivities and therefore can also be used in practicing the screening methods of the present invention. A functional derivative can be prepared from an HIV-interacting host factor by proteolytic cleavage followed by conventional purification procedures known to those skilled in the art. Alternatively, the functional derivative can be produced by recombinant DNA technology by expressing only fragments of an HIV-interacting host factor that retain one or more of their bioactivities.

Test agents or compounds that can be screened with methods of the present invention include polypeptides, beta-turn mimetics, polysaccharides, phospholipids, hormones, prostaglandins, steroids, aromatic compounds, heterocyclic compounds, benzodiazepines, oligomeric N-substituted glycines, oligocarbamates, polypeptides, saccharides, fatty acids, steroids, purines, pyrimidines, derivatives, structural analogs or combinations thereof. Some test agents are synthetic molecules, and others natural molecules.

Test agents are obtained from a wide variety of sources including libraries of synthetic or natural compounds. Combinatorial libraries can be produced for many types of compound that can be synthesized in a step-by-step fashion. Large combinatorial libraries of compounds can be constructed by the encoded synthetic libraries (ESL) method described in WO 95/12608, WO 93/06121, WO 94/08051, WO 95/35503 and WO 95/30642. Peptide libraries can also be generated by phage display methods (see, e.g., Devlin, WO 91/18980). Libraries of natural compounds in the form of bacterial, fungal, plant and animal extracts can be obtained from commercial sources or collected in the field. Known pharmacological agents can be subject to directed or random chemical modifications, such as acylation, alkylation, esterification, amidification to produce structural analogs.

Combinatorial libraries of peptides or other compounds can be fully randomized, with no sequence preferences or constants at any position. Alternatively, the library can be biased, i.e., some positions within the sequence are either held constant, or are selected from a limited number of possibilities. For example, in some cases, the nucleotides or amino acid residues are randomized within a defined class, for example, of hydrophobic amino acids, hydrophilic residues, sterically biased (either small or large) residues, towards the creation of cysteines, for cross-linking, prolines for SH-3 domains, serines, threonines, tyrosines or histidines for phosphorylation sites, or to purines.

The test agents can be naturally occurring proteins or their fragments. Such test agents can be obtained from a natural source, e.g., a cell or tissue lysate. Libraries of polypeptide agents can also be prepared, e.g., from a cDNA library commercially available or generated with routine methods. The test agents can also be peptides, e.g., peptides of from about 5 to about 30 amino acids, with from about 5 to about 20 amino acids being preferred, and from about 7 to about 15 being particularly preferred. The peptides can be digests of naturally occurring proteins, random peptides, or “biased” random peptides. In some methods, the test agents are polypeptides or proteins. The test agents can also be nucleic acids. Nucleic acid test agents can be naturally occurring nucleic acids, random nucleic acids, or “biased” random nucleic acids. For example, digests of prokaryotic or eukaryotic genomes can be similarly used as described above for proteins.

In some preferred methods, the test agents are small molecule organic compounds, e.g., chemical compounds with a molecular weight of not more than about 1,000 or not more than about 500. Preferably, high throughput assays are adapted and used to screen for such small molecules. In some methods, combinatorial libraries of small molecule test agents as described above can be readily employed to screen for small molecule compound that inhibit HIV infection. A number of assays are available for such screening, e.g., as described in Schultz (1998) BioorgMed Chem Lett 8:2409-2414; Weller (1997) MoI Divers. 3:61-70; Femandes (1998) Curr Opin Chem Biol 2:597-603; and Sittampalam (1997) Curr Opin Chem Biol 1:384-91.

Libraries of test agents to be screened with the claimed methods can also be generated based on structural studies of the HDFs discussed above or their fragments. Such structural studies allow the identification of test agents that are more likely to bind to the HDFs. The three-dimensional structures of the HDFs can be studied in a number of ways, e.g., crystal structure and molecular modeling. Methods of studying protein structures using x-ray crystallography are well known in the literature. See Physical Bio-chemistry, Van Holde, K. E. (Prentice-Hall, New Jersey 1971), pp. 221-239, and Physical Chemistry with Applications to the Life Sciences, D. Eisenberg & D. C. Crothers (Benjamin Cummings, Menlo Park 1979). Computer modeling of HDFs' structures provides another means for designing test agents to screen for modulators of HIV infections. Methods of molecular modeling have been described in the literature, e.g., U.S. Pat. No. 5,612,894 entitled “System and method for molecular modeling utilizing a sensitivity factor,” and U.S. Pat. No. 5,583,973 entitled “Molecular modeling method and system.” In addition, protein structures can also be determined by neutron diffraction and nuclear magnetic resonance (NMR). See, e.g., Physical Chemistry, 4th Ed. Moose, W. J. (Prentice-Hall, New Jersey 1972), and NMR of Proteins and Nucleic Acids, K. Wuthrich (Wiley-Interscience, New York 1986).

Downmodulators of the present invention also include antibodies that specifically bind to an HDF identified herein. Such antibodies can be monoclonal or polyclonal. Such antibodies can be generated using methods well known in the art. For example, the production of non-human monoclonal antibodies, e.g., murine or rat, can be accomplished by, for example, immunizing the animal with an HDF identified herein, or its fragment (See Harlow and Lane, Antibodies, A Laboratory Manual, Cold Spring Harbor Press, 3rd ed., 2000). Such an immunogen can be obtained from a natural source, by peptides synthesis or by recombinant expression.

Humanized forms of mouse antibodies can be generated by linking the CDR regions of non-human antibodies to human constant regions by recombinant DNA techniques. See Queen et al., Proc. Natl. Acad. Sci. USA 86, 10029-10033 (1989) and WO 90/07861. Human antibodies can be obtained using phage-display methods. See, e.g., Dower et al., WO 91/17271; McCafferty et al., WO 92/01047. In these methods, libraries of phage are produced in which members display different antibodies on their outer surfaces. Antibodies are usually displayed as Fv or Fab fragments. Phage displaying antibodies with a desired specificity are selected by affinity enrichment to a HDF.

Human antibodies against an HDF can also be produced from non-human transgenic mammals having transgenes encoding at least a segment of the human immunoglobulin locus and an inactivated endogenous immunoglobulin locus. See, e.g., Lonberg et al., WO93/12227 (1993); Kucherlapati, WO 91/10741 (1991). Human antibodies can be selected by competitive binding experiments, or otherwise, to have the same epitope specificity as a particular mouse antibody. Such antibodies are particularly likely to share the useful functional properties of the mouse antibodies. Human polyclonal antibodies can also be provided in the form of serum from humans immunized with an immunogenic agent. Optionally, such polyclonal antibodies can be concentrated by affinity purification using an HDF or its fragment.

Typically, test agents are first screened for ability to downmodulate a biological activity of an HDF identified herein. A number of assay systems can be employed in this screening step. The screening can utilize an in vitro assay system or a cell-based assay system. In this screening step, test agents can be screened for binding to an HDF, altering expression level of the HDF, or modulating other biological or molecular activities (e.g., enzymatic activities) of the HDF.

In some methods, binding of a test agent to an HDF is determined in the first screening step. Binding of test agents to an HIV-interacting host factor can be assayed by a number of methods including e.g., labeled in vitro protein-protein binding assays, electrophoretic mobility shift assays, immunoassays for protein binding, functional assays (phosphorylation assays, etc.), and the like. See, e.g., U.S. Pat. Nos. 4,366,241; 4,376,110; 4,517,288; and 4,837,168; and also Bevan et al., Trends in Biotechnology 13:115-122, 1995; Ecker et al., Bio/Technology 13:351-360, 1995; and Hodgson, Bio/Technology 10:973-980, 1992. The test agent can be identified by detecting a direct binding to the HDF, e.g., co-immunoprecipitation with the HDF by an antibody directed to the HDF. The test agent can also be identified by detecting a signal that indicates that the agent binds to the HDF, e.g., fluorescence quenching or FRET.

Competition assays provide a suitable format for identifying test agents that specifically bind to an HDF. In such formats, test agents are screened in competition with a compound already known to bind to the HDF. The known binding compound can be a synthetic compound. It can also be an antibody, which specifically recognizes the HDF, e.g., a monoclonal antibody directed against the HDF. If the test agent inhibits binding of the compound known to bind the HDF, then the test agent also binds the HDF.

Numerous types of competitive binding assays are known, for example: solid phase direct or indirect radioimmunoassay (RIA), solid phase direct or indirect enzyme immunoassay (EIA), sandwich competition assay (see Stahli et al., Methods in Enzymology 9:242-253, 1983); solid phase direct biotin-avidin EIA (see Kirkland et al., J. Immunol. 137:3614-3619, 1986); solid phase direct labeled assay, solid phase direct labeled sandwich assay (see, Harlow and Lane, Antibodies, A Laboratory Manual, Cold Spring Harbor Press, 3rd ed., 2000); solid phase direct label RIA using 1251 label (see Morel et al., MoI. Immunol. 25(1):7-15, 1988); solid phase direct biotin-avidin EIA (Cheung et al., Virology 176:546-552, 1990); and direct labeled RIA (Moldenhauer et al., Scand. J. Immunol. 32:77-82, 1990). Typically, such an assay involves the use of purified polypeptide bound to a solid surface or cells bearing either of these, an unlabelled test agent and a labeled reference compound. Competitive inhibition is measured by determining the amount of label bound to the solid surface or cells in the presence of the test agent. Usually the test agent is present in excess. Modulating agents identified by competition assay include agents binding to the same epitope as the reference compound and agents binding to an adjacent epitope sufficiently proximal to the epitope bound by the reference compound for steric hindrance to occur. Usually, when a competing agent is present in excess, it will inhibit specific binding of a reference compound to a common target polypeptide by at least 50 or 75%.

The screening assays can be either in insoluble or soluble formats. One example of the insoluble assays is to immobilize an HTV-interacting host factor or its fragment onto a solid phase matrix. The solid phase matrix is then put in contact with test agents, for an interval sufficient to allow the test agents to bind. After washing away any unbound material from the solid phase matrix, the presence of the agent bound to the solid phase allows identification of the agent. The methods can further include the step of eluting the bound agent from the solid phase matrix, thereby isolating the agent. Alternatively, other than immobilizing the cellular host factor, the test agents are bound to the solid matrix and the HDF is then added.

Soluble assays include some of the combinatory libraries screening methods described above. Under the soluble assay formats, neither the test agents nor the HDF are bound to a solid support. Binding of an HDF or fragment thereof to a test agent can be determined by, e.g., changes in fluorescence of either the HDF or the test agents, or both. Fluorescence may be intrinsic or conferred by labeling either component with a fluorophor.

In some binding assays, either the HDF, the test agent, or a third molecule (e.g., an antibody against the HDF) can be provided as labeled entities, i.e., covalently attached or linked to a detectable label or group, or cross-linkable group, to facilitate identification, detection and quantification of the polypeptide in a given situation. These detectable groups can comprise a detectable polypeptide group, e.g., an assayable enzyme or antibody epitope. Alternatively, the detectable group can be selected from a variety of other detectable groups or labels, such as radiolabels (e.g., ¹²⁵I, ³²P, ³⁵S) or a chemiluminescent or fluorescent group. Similarly, the detectable group can be a substrate, cofactor, inhibitor or affinity ligand.

Binding of a test agent to an HDF provides an indication that the agent can be a modulator of the HDF. It also suggests that the agent may inhibit HIV infection by acting on the HDF. Thus, a test agent that binds to an HDF can be tested for ability to inhibit an HIV infection related activity (i.e., in the second testing step outlined above). Alternatively, a test agent that binds to an HDF can be further examined to determine whether it indeed inhibitis a biological activity (e.g., an enzymatic activity) of the HDF. The existence, nature, and extent of such modulation can be tested with an activity assay. More often, such activity assays can be used independently to identify test agents that downmodulate activities of an HIV-interacting host factor (i.e., without first assaying their ability to bind to the HIV-interacting host factor).

In general, the methods involve adding a test agent to a sample containing an HDF in the presence or absence of other molecules or reagents which are necessary to test a biological activity of the HDF (e.g., enzymatic activity if the HDF is an enzyme), and determining an alteration in the biological activity of the HDF. If the HDF has a known biological or enzymatic function (e.g., kinase activity or protease activity), the biological activity monitored in the first screening step can also be the specific biochemical or enzymatic activity of the HDF. Any of these molecules can be employed in the first screening step. Methods for assaying the enzymatic activities of these molecules are well known and routinely practiced in the art. The substrates to be used in the screening can be a molecule known to be enzymatically modified by the enzyme (e.g., a kinase), or a molecule that can be easily identified from candidate substrates for a given class of enzymes.

Many other assays for monitoring protein kinase activities are described in the art. These include assays reported in, e.g., Chedid et al., J. Immunol. 147: 867-73, 1991; Kontny et al., Eur J. Pharmacol. 227: 333-8, 1992; Wang et al., Oncogene 13: 2639-47, 1996; Murakami et al., Oncogene 14: 2435-44, 1997; Pyrzynska et al., J. Neurochem. 74: 42-51, 2000; Berry et al., Biochem Pharmacol. 62: 581-91, 2001; Cai et al., Chin Med J (Engl). 114: 248-52, 2001. Any of these methods may be employed and modified to assay modulatory effect of a test agent on an HDF that is a kinase. Further, many kinase substrates are available in the art. See, e.g., www.emdbiosciences.com; and www.proteinkinase.de. In addition, a suitable substrate of a kinase can be screened for in high throughput format. For example, substrates of a kinase can be identified using the Kinase-Glo® luminescent kinase assay (Promega) or other kinase substrate screening kits (e.g., developed by Cell Signaling Technology, Beverly, Mass.).

In addition to assays for screening agents that downmodulate enzymatic or other biological activities of an HDF, the activity assays also encompass in vitro screening and in vivo screening for alterations in expression level of the HDF. Modulation of expression of an HDF can be examined in a cell-based system by transient or stable transfection of an expression vector into cultured cell lines. For example, test compounds can be assayed for ability to inhibit expression of a reporter gene (e.g., luciferase gene) under the control of a transcription regulatory element (e.g., promoter sequence) of an HDF. Many of the genes encoding the HDFs disclosed herein have been characterized in the art. Transcription regulatory elements such as promoter sequences of many of these genes have all been delineated.

Assay vector bearing the transcription regulatory element that is operably linked to the reporter gene can be transfected into any mammalian cell line for assays of promoter activity. Reporter genes typically encode polypeptides with an easily assayed enzymatic activity that is naturally absent from the host cell. Typical reporter polypeptides for eukaryotic promoters include, e.g., chloramphenicol acetyltransferase (CAT), firefly or Renilla luciferase, beta-galactosidase, beta-glucuronidase, alkaline phosphatase, and green fluorescent protein (GFP). Vectors expressing a reporter gene under the control of a transcription regulatory element of an HDF can be prepared using only routinely practiced techniques and methods of molecular biology (see, e.g., e.g., Samrbook et al., supra; Brent et al., supra). In addition to a reporter gene, the vector can also comprise elements necessary for propagation or maintenance in the host cell, and elements such as polyadenylation sequences and transcriptional terminators. Exemplary assay vectors include pGL3 series of vectors (Promega, Madison, Wis.; U.S. Pat. No. 5,670,356), which include a polylinker sequence 5′ of a luciferase gene. General methods of cell culture, transfection, and reporter gene assay have been described in the art, e.g., Samrbook et al., supra; and Transfection Guide, Promega Corporation, Madison, Wis. (1998). Any readily transfectable mammalian cell line may be used to assay expression of the reporter gene from the vector, e.g., HCT1 16, HEK 293, MCF-7, and HepG2 cells.

To identify novel inhibitors of HIV infection, compounds that downmodulate an HDF as described above are typically further tested to confirm their inhibitory effect on HIV infection. Typically, the compounds are screened for ability to downmodulate an activity that is indicative of HIV infection or HIV replication. The screening is performed in the presence of the HDF on which the modulating compounds act. The HDF against which the modulating agents are identified in the first screening step can be either expressed endogenously by the cell or expressed from second expression vector. Preferably, this screening step is performed in vivo using cells that endogenously express the HDF. As a control, effect of the modulating compounds on a cell that does not express the HDF may also be examined. For example, if the HDF (e.g., encoded by a mouse gene) used in the first screening step is not endogenously expressed by the cell line (e.g., a human cell line), a second vector expressing the polypeptide can be introduced into the cell. By comparing an HIV infection related activity in the presence or absence of a modulating compound, activities of the compounds on HIV infection can be identified.

Many assays and methods are available to examine HIV-inhibiting activity of the compounds. This usually involves testing the compounds for ability to inhibit HIV viral replication in vitro or a biochemical activity that is indicative of HIV infection. In some methods, potential inhibitory activity of the modulating compounds on HIV infection can be tested by examining their effect on HIV infection of a cultured cell in vitro, using methods routinely practiced in the art. For example, the compounds can be tested on HIV infection of a primary macrophage culture as described in Seddiki et al., AIDS Res Hum Retroviruses. 15:381-90, 1999. They can also be examined on HTV infection of other T cell and monocyte cell lines as reported in Fujii et al. J Vet Med. Sci. 66: 115-21, 2004. Additional in vitro systems for monitoring HIV infection have been described in the art. See, e.g., Li et al., Pediatr Res. 54:282-8, 2003; Steinberg et al., Virol. 193:524-7, 1993; Hansen et al., Antiviral Res. 16:233-42, 1991; and Piedimonte et al., AIDS Res Hum Retroviruses. 6:251-60, 1990.

In these assays, HIV infection of the cells can be monitored morphologically, e.g., by a microscopic cytopathic effect assay (see, e.g., Fujii et al., J Vet Med. Sci. 66:115-21, 2004). It can also be assessed enzymatically, e.g., by assaying HIV reverse transcriptase (RT) activity in the supernatant of the cell culture. Such assays are described in the art, e.g., Reynolds et al., Proc Natl Acad Sci USA. 100:1615-20, 2003; and Li et al., Pediatr Res. 54:282-8, 2003. Other assays monitor HIV infection by quantifying accumulation of viral nucleic acids or viral antigens. For example, Winters et al. (PCR Methods Appl. 1:257-62, 1992) described a method which assays HTV gag RNA and DNA from HIV infected cell cultures. Vanitharani et al. described an HIV infection assay which measures production of viral p24 antigen (Virology 289:334-42, 2001). Viral replication can also be monitored in vitro by a p24 antigen ELISA assay, as described in, e.g., Chargelegue et al., J Virol Methods. 38(3):323-32, 1992; and Klein et al., J Virol Methods. 107(2): 169-75, 2003. All these assays can be employed and modified to assess anti-HTV activity of the modulating compounds of the present invention.

In some methods, potential inhibiting effect of modulating compounds on HIV infection can be examined in engineered reporter cells which are permissive for HIV replication. In these cells, HIV infection and replication is monitored by examining expression of a reporter gene under the control of an HIV transcription regulatory element, e.g., HIV-LTR.

One example of such cells is HeLa-T4-βGal HIV reporter cell. The HeLa-T4-βGal reporter cell can be infected with HIV-HIb after being treated with a modulating compound. Virus infectivity from the compound treated cells, as monitored by measuring β-galactosidase activity, can be compared with that from control cells that have not been treated with the compound. A reduced virus titer or reduction in infectivity from cells treated with the modulating compound would confirm that the compound can indeed inhibit HIV infection or viral replication.

In addition to the Hela-T4-βGal cells exemplified herein, many similar reporter assays have also been described in the art. For example, Gervaix et al. (Proc Natl Acad Sci USA. 94:4653-8, 1997) developed a stable T-cell line expressing a plasmid encoding a humanized green fluorescent protein (GFP) under the control of an HIV-I LTR promoter. Upon infection with HIV-I, a 100- to 1,000-fold increase of fluorescence of infected cells can be observed as compared with uninfected cells. Any of these assay systems can be employed in the present invention to monitor effects of the modulating compounds on HIV infection in real time. These in vitro systems also allow quantitation of infected cells overtime and determination of susceptibility to the compounds.

In some other methods, effect of the modulating compounds on HIV replication can be examined by examining production of HIV-I pseudo virus in a cell treated with the compounds. The cell can express the HDF endogenously or exogenously. For example, a construct encoding the HDF can be transfected into the host cell that do not endogenously express the HIV-interacting host factor. Production of HIV-I pseudovirus can be obtained by transfecting a producer cell (e.g., a 293T HEK cell) with a reporter plasmid expressing the psi-positive RNA encoding a reporter gene (e.g., luciferase gene), a delta psi packaging construct encoding all structural proteins and the regulatory or accessory proteins such as Tat, Rev, Vpr, and Vif, and a VSV-g envelop expression plasmid. The pseudovirus produced in the producer cell encodes only the reporter gene. After infecting a target cell with pseudovirus in the supernatant from the producer cell, the reporter gene is expressed following retrotranscription and integration into the target cell genome.

To screen for inhibitors of HIV replication, the producer host cell can be treated with a modulating compound prior to, concurrently with, or subsequent to transfection of the pseudovirus plasmids. Preferably, the compound is administered to the host cell prior to transfection of the pseudovirus plasmids, and is present throughout the assay process. Titer of the produced pseudovirus can be monitored by infecting target cells with the pseudovirus in the supernatant from the producer cell and assaying an activity of the reporter (e.g., luciferase activity) in the target cells. As a control, reporter activity in target cells infected with supernatant from producer cells that have not been treated with the compound is also measured. If the modulating compound has an inhibitory effect on virus budding, target cells contacted with the supernatant from the producer cells that have been treated with the compound will have a reduced reporter activity relative to the control cells.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used in connection with percentages may mean±1%.

In one respect, the present invention relates to the herein described compositions, methods, and respective component(s) thereof, as essential to the invention, yet open to the inclusion of unspecified elements, essential or not (“comprising). In some embodiments, other elements to be included in the description of the composition, method or respective component thereof are limited to those that do not materially affect the basic and novel characteristic(s) of the invention (“consisting essentially of”). This applies equally to steps within a described method as well as compositions and components therein. In other embodiments, the inventions, compositions, methods, and respective components thereof, described herein are intended to be exclusive of any element not deemed an essential element to the component, composition or method (“consisting of”).

In one aspect, the present invention relates to the embodiments described herein, with the exclusion of one or more of the specific agents (e.g., siRNAs) described herein (e.g., listed in Table 3) and/or with the exclusion of one or more of said specific agents that inhibit one or more of the specific HDFs described herein.

All patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### EXAMPLES

### Example 1

### The siRNA Screen Design

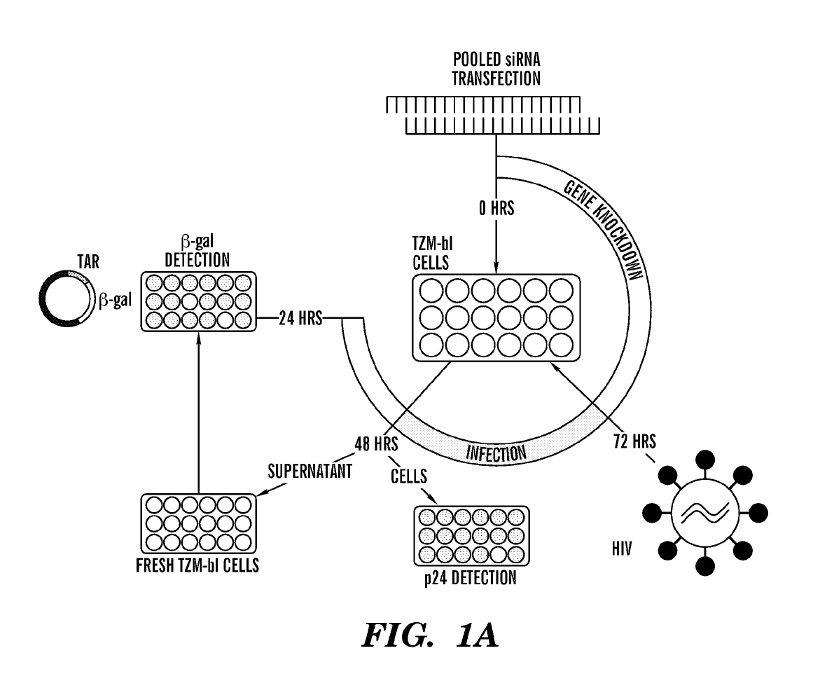

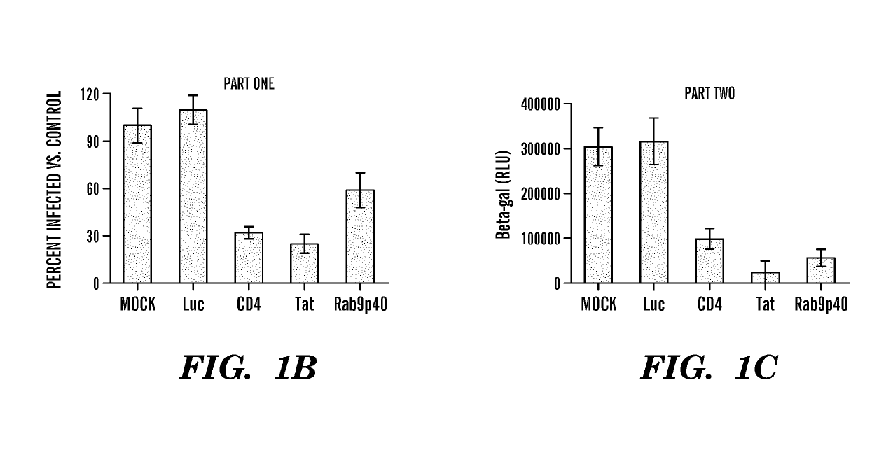

Using a genome-wide siRNA library, we developed a two part screening platform to detect host proteins needed for HIV infection (FIG. 1A). Part one of the image-based screen consisted of challenging siRNA transfected cells with the IIIB strain of HIV-1 (HIV-IIIB) and then 48 hours later staining for intracellular HIV capsid protein, p24, an indicator of expression of the late unspliced mRNA encoding the viral gag gene. This assay detects siRNAs that target host proteins needed from viral entry through translation of Gag, but would be less sensitive for factors that affect later stages of the viral lifecycle, i.e. viral assembly and budding. To identify late-acting factors, we carried out part two of the screen, by incubating culture supernatants from the HIV-infected siRNA transfected cells in part one with fresh reporter cells. These cells were then assayed for HIV infection after 24 hours by Tat-dependent reporter gene expression. The siRNA library is arrayed in 21,121 individual pools comprised of four 19mer siRNA duplexes, with the siRNAs within each pool targeting distinct sites within a single gene.

For the screen we chose TZM-bl cells, a HeLa-derived cell line, which expresses endogenous CXCR4, transgenic CD4 and CCR5, and an integrated Tat-dependent beta-galactosidase reporter gene (beta-gal, [25]). The transfection of siRNAs and detection of HIV infection were optimized in a 384 well format using robotics and positive control siRNAs that target viral Tat, needed for efficient transcription of the proviral genome, or the host factors, CD4 or Rab9p40, required for viral entry and budding, respectively. Cells transfected with siRNAs targeting CD4 or Tat, showed a 3 to 4-fold decrease in p24 expression (FIGS. 1B and C). However, less protection was seen upon depletion of Rab9p40 (FIG. 1C, part one). However, after incubation with transferred cultured supernatant for 24 hours, the depletion of Rab9p40, which minimally inhibited p24 intracellular staining, scored convincingly in the Tat-dependent beta-galactosidase luminescence assay, supporting the known post-transcriptional role for Rab9p40 in producing infectious virions.

This platform was then used for a genome wide screen. siRNA pools were classified as hits if they decreased the percentage of p24 positive cells or beta-galactosidase activity by two or greater standard deviations (SD) from the plate mean. Since a reduction in cell viability or proliferation would also lead to reduced HIV replication, we also required that the pooled siRNAs did not decrease the number of viable cells by greater than two SDs from the mean of the plate. These criteria were met by 387 of 21,121 total pools (1.8%) in the initial screen. We next performed a validation screen, in which the four individual siRNAs comprising each pool were placed into separate wells, and rescreened using the identical methods. In the validation screen, 275 of the pools (71%) reconfirmed with at least one of four possible siRNAs scoring in either part one or two of the screen. There was a strong correlation between parts one and two of the screen. The identified genes are listed in Table 2, which lists the gene symbol and the SMARTpool Catalog number. All hits in part one also scored in part two; only 26 genes appeared specifically in part two, reflecting a role for these factors in late stages of viral replication (Table 2).

### Bioinformatics Analysis of HDFs

Of the HDFs that confirmed, we identified 38 host factors (14%) previously implicated in HIV biology (Table 1).

TABLE I
Host Proteins Previously Implicated in HIV Infection
Recovered From siRNA Screen¹

| | | |
|---|---|---|
| A4GALT (2/4 siRNAs) | EGFR (1) | PolR3F (1) |
| AKT1 (2) | ERCC3 (3) | PPP2R2A (1) |
| AP2M1 (1) | FBXW11 (4) | PSME2 (1) |
| Arf1 (1) | GCN5L2 (1) | PURA (2) |
| CD4 (2/4) | H3F3A (1) | Rab9p40 (3) |
| CTDP1 (1) | HTATSF1 (1) | RANBP1 (SP) |
| CXCR-4 (2) | HRS (SP) | RelA (4) |
| CyclinT1 (SP) | IKBG (2) | SIP1 (1) |
| CYCLOB (PPIB, 2) | La Autoantigen (SSB, SP) | TCEB3 (2) |
| DDX3 (SP) | NMT1 (3) | TFAP4 (SP) |
| DNAJB1 (3) | Nup153(2) | VPRBP (1) |
| EGF (2) | Nup85 (2) | ZNRD1 (1) |
| NF2 (4) | PolR3A (1) | |

¹Numbers in parentheses indicate individual siRNAs out of a total of four possible, thatscored on retesting.
SP = SMARTpool scored, since the four oligos in the pool were not individually tested.

These known host factors spanned the HIV lifecycle from viral binding (CD4 and CXCR4), to Gag modification and budding (Rab9p40 and NMT1). 237 genes had not been previously implicated in HIV infection. Importantly, over 100 genes had two or more individual siRNAs score as positive, suggesting that the observed phenotype was due to depletion of the specific gene, and not off-target effects (Table 2). The subcellular localization of each protein was manually curated based on gene ontology (GO) cellular component terms, UniProt annotations and prediction software (FIG. 2A). The HDFs were further categorized using GO biological process and molecular function. Of the 275 genes that confirmed, 482 GO biological process terms could be assigned to 204 genes. An enrichment analysis identified 136 terms, assigned to 103 genes, to be relatively enriched in statistically significant manner (FIG. 2B). Analysis of GO molecular functions identified enrichment for 44 statistically significant terms assigned to 86 genes (FIG. 2C). We found enrichment for genes involved in many general cellular processes and functions, notably, mRNA transport, glycoprotein metabolic processing, GTPase signaling, intracellular transport, and secretion (FIG. 2A, B and C). Among HDFs, we observed enrichment for members of the nuclear factor-KB (NF-κB) pathway, Wnt pathway, as well as CREB and Sp1-associated coactivators. These were expected based on HIV's known exploitation of host transcription factors [14] (FIG. 2D). The validation of the screen through the identification of known factors, as well as functionally connected genes described below, lends support to the hypothesis that the novel candidates also play a role in HIV pathogenesis. In addition to known genes, several macromolecular complexes, not previously identified in promoting HIV infection scored with multiple hits. One of these was the Nup160 subcomplex of the nuclear pore, from which the screen identified 4 of the 6 subunits (Nup85, Nup107, Nup133, Nup160). Since the Nup160 complex serves as a scaffold for pore assembly, loss of this complex may impede HIV access to the nucleus by blocking entry via pores [26, 27]. Additionally, depletion of multiple components of Mediator, a multifactorial adaptor complex (Med4, Med6, Med7, Med14, Med27, and Med28), which directly couples transcription factors to the core RNA pol II holoenzyme [28], inhibited HIV infection (Table 2). Mediator transmits both positive and negative signals from a diverse group of regulatory factors, and is believed to either exchange or differentially present disparate subunits to promote specificity of gene activation [29, 30]. Therefore, the inhibition of HIV infection observed upon loss of these components may provide insights into the requirements of the multiple activators that bind the viral LTR and promote viral transcription. Two ER-Golgi-associated assemblages, the conserved oligomeric golgi (COG) complex [31] and the transport protein particle (TRAPP) I complex [32] also scored with multiple components, consistent with HIV's dependency on transmembrane glycoproteins and lipid rafts for cellular entry ([33]).

Several novel associations between HIV infection and cellular processes are evident in our data. Autophagy is an evolutionary conserved pathway essential for the degradation and recycling of cellular components. Targeted substrates are encapsulated in membrane bound autophagosome by the actions of two evolutionary conserved protein conjugation pathways [34]. Mature autophagosomes subsequently fuse with lysosomes, precipitating substrate destruction. We found that HIV infection depended on the presence of members of both these conjugation systems (Atg7, Atg8, Atg12, and Atg16L2, Table S2). In addition, HDFs involved in lysosomal functions (CLN3, and LapTM5) may also be required for effective autophagy.

The HeLa-derived cell line used for this study is not the natural host for HIV but must express the minimum number of HDFs to support HIV infection. We were interested in whether the HDF genes as a whole showed an expression bias in other cell types that might help explain its tropism. Tissue distribution in GNF was assessed. We assessed the expression patterns of a subset of 239 of the confirmed genes, that were expressed by at least one of the 79 tissues in the Genomic Institute of the Novartis Research Fund (GNF) expression profile dataset, and found that 79/239 (33%) were enriched for high expression in immune cells (p<0.001, top 7% expression), as compared to the 7% immune enrichment of ubiquitously expressed genes in the entire array. Of the 275 candidates, 239 had at least one probe in the Symatlas GNF expression panel. A single probe with maximum variation across tissues was selected for each gene and the 79 tissues were classified to immune, central nervous system and others. Expression values were converted to standard score (Z score) and genes were clustered using hierarchical clustering. Immune enrichment was calculated using the Wilcoxon rank sum test and p-values were corrected using the Bonferroni method. Of the 239 probes in GNF 79, immune enriched probes with corrected p-values <0.05 were indicated.

Expression profiles for the set of 79 immune-enriched genes were then determined for relevant HIV-target cells, T cells, macrophages and dendritic cells (T helper 1, T helper 2, Yδ T cells, neutrophils, dendritic cells, and macrophages). Gene expression profiles were obtained from Chtanova et al. (Chtanova, 2004; Chtanova, 2005). All tissues were stimulated and performed in duplicate. The expression values for each duplicate were averaged after data normalization. A single probe with maximum variation on a linear scale across tissues was selected for each gene and expression values were converted to standard score (Z score). Clustering was performed for tissues and genes using K-means clustering with 3 clusters for tissues and 4 clusters for genes.

HIV's requirement for interaction with host genes highly expressed in immune cells suggests that HIV may have evolved to use those cells because they optimally perform the functions required for the HIV life cycle, thereby explaining in part its tropism.

### HIV Entry Involves an Unanticipated Role for Retrograde Vesicular Transport

Further validation focused on a subset of novel HDFs that scored with multiple siRNAs. The screen significantly enriched for host factors involved in vesicular transport and GTPase activity (FIGS. 2B and C). Rab GTPases play important roles in vesicular trafficking [35], and four scored in the validation round (Rab1b, Rab2, Rab6A, and Rab28, Table 2).

Three of four siRNAs confirmed in the validation round for both Rab6A and Rab6A′, which are alternatively spliced proteins from the same gene differing by only 3 amino acids (Table 2) [36, 38]. Rab6 regulates retrograde Golgi-to-ER transport [35, 36], and is important for proper recycling of Golgi-resident enzymes. Rab6A′ is believed to play a critical role in endosomal trafficking, and is the human homolog of the yeast GTPase, Ypt6 [36]; from herein both isoforms will be referred to as Rab6. Ypt6 mutants are viable, but display defects in retrograde Golgi transport, particularly recycling of Golgi glycosyltransferases [37, 39]. We further discovered that the human homolog of Rgp1p, a yeast guanine nucleotide exchange factor (GEF) required for Ypt6 function, is required for HIV infection (Table 2 [40]).

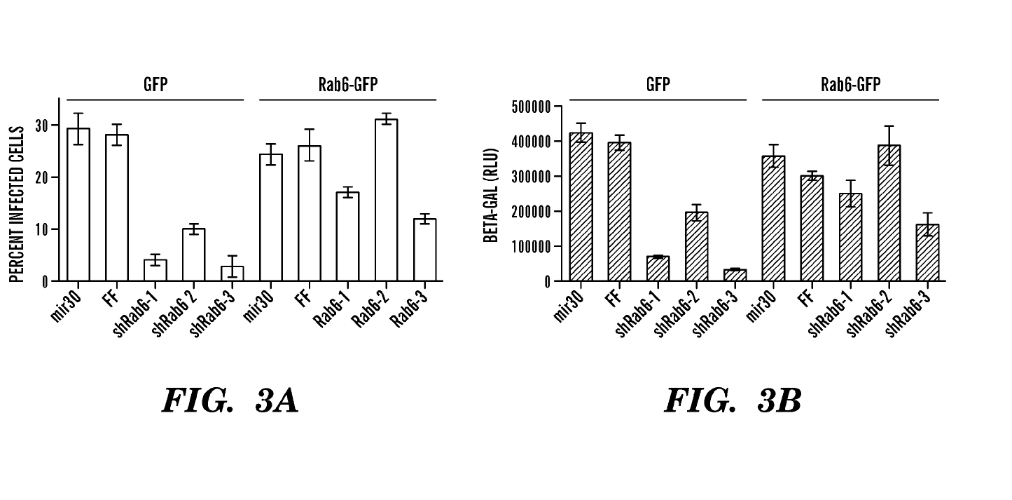

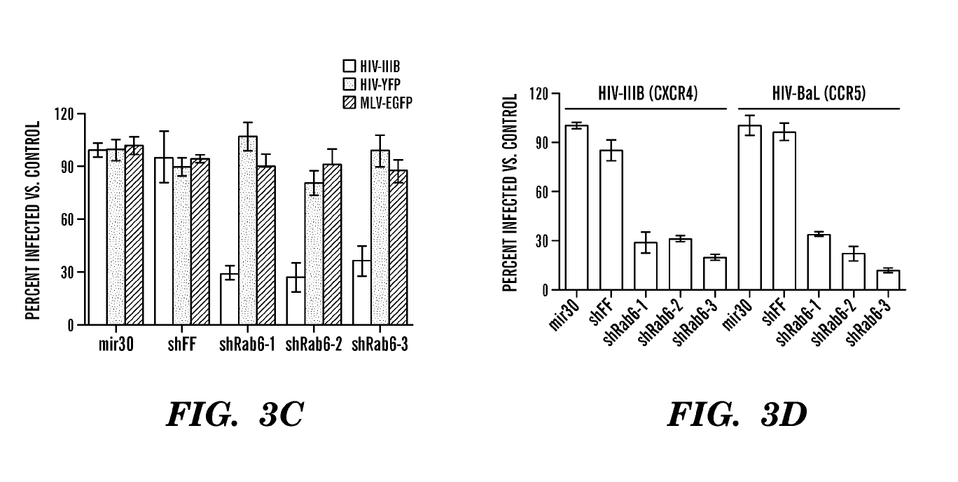

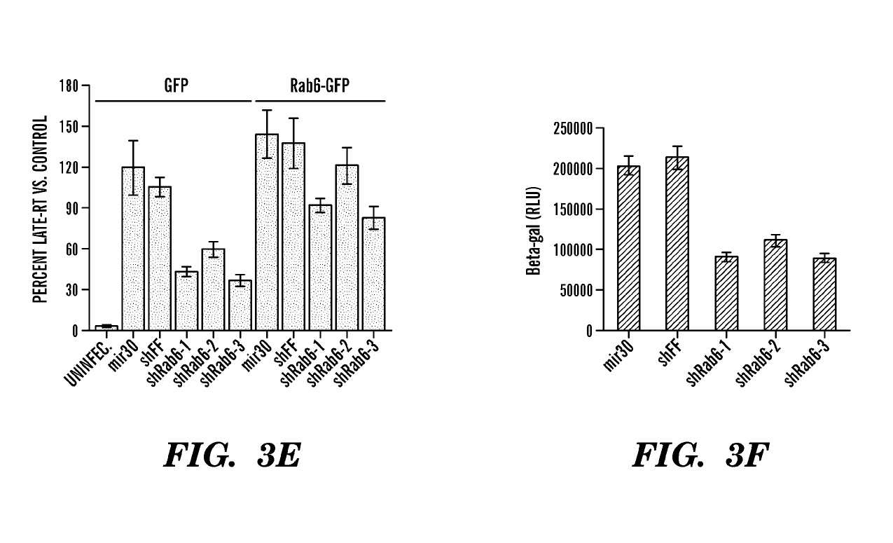

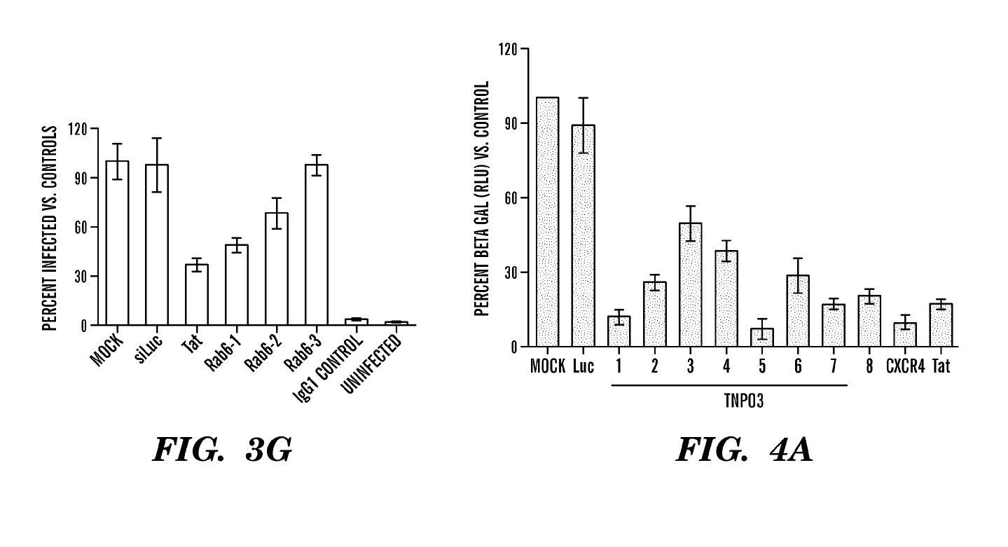

To assess the role of Rab6 in HIV infection, we generated TZM-bl cells stably expressing short hairpin RNA (shRNAs) directed against Rab6. All three shRNA plasmids directed against Rab6 decreased HIV infection, and the protection was proportional to the extent of Rab6 depletion (FIG. 3A, B). Western blot analysis for Rab6 and Rab6-GFP levels in the cells shown in FIGS. 3A and B was also performed, and confirmed the results. The block to infection was in the first phase of the viral lifecycle (from entry to transcription of the integrated provirus), because Rab6 depletion inhibited expression of the Tat-dependent beta-galactosidase reporter gene when tested 20 hours after viral challenge (FIG. 3B). A strong correlation was also seen between the level of p24 viral protein expression and the Tat-dependent reporter assay, confirming a specific effect on the virus (FIGS. 3A and B).

Stable expression of a Rab6-GFP fusion protein (Rab6A′ isoform), lacking the 3′UTR of the endogenous Rab6 mRNA targeted by the 3 Rab6 shRNAs, rescued susceptibility to HIV infection of cells expressing the shRNAs, further validating the role of Rab6 in HIV infection (FIG. 3, A, B). Given the role of Rab6 in vesicular transport, we examined surface expression of CD4 and CXCR4 by flow cytometry (FACS) in the Rab6 knockdown (Rab6-KD) cell lines. CD4 surface levels were unaltered for all Rab6-KD cells (data not shown). Subtle alterations of surface CXCR4 expression were noted in the Rab6-KD cells. However, these minor variations did not correlate with resistance to HIV infection or Rab6 depletion and were not restored with expression of Rab6-GFP (data not shown). Thus, something other than receptor expression is defective in Rab6-depleted cells.

To determine whether HIV envelope proteins are required for the block to HIV infection when Rab6 was depleted, we infected TZM-bl cells with either HIV-IIIB, or an HIV strain pseudotyped with the virus G envelope protein (VSV-G), that contains a yellow fluorescent (YFP) reporter in place of the nef gene (HIV-YFP). Only HIV-IIIB infection, and not the pseudotyped strain, was inhibited (FIG. 3C). In addition, the infectivity of a VSV-G pseudotyped γ-retrovirus, Moloney leukemia virus (MLV-EGFP), was unperturbed by diminished levels of Rab6 (FIG. 3C). HIV envelope proteins induce viral entry by promoting fusion of the viral envelope to the cell membrane. In contrast, VSV-G pseudotypes are taken up by endocytosis, with direct fusion triggered by endosomal acidification. Thus, the differential effect on infection suggested a role for Rab6 in a very early stage of infection, perhaps at the level of the interaction of the viral envelope with host receptors, or membrane fusion. HIV-IIIB has tropism for the chemokine receptor CXCR4 (X4). To determine whether inhibition was restricted to X4 virus, we also examined the effect of Rab6 silencing on infection with HIV-Bal, a CCR5 (R5) tropic virus. Targeting Rab6 did not alter surface CCR5 expression (data not shown), but did inhibit HIV-Bal infection (FIG. 3D). Therefore Rab6 plays a role in infection by both R5 and X4 viruses.

Viruses blocked for cell entry do not efficiently reverse transcribe their genome. Therefore, we measured the levels of late reverse-transcribed HIV cDNA (late-RT) using quantitative PCR after infection. Rab6-KD cell lines displayed less viral late-RT DNA than controls, and this inhibition was reversed by expression of Rab6-GFP (FIG. 3E). Thus, the block to HIV comes prior to the virus completing reverse transcription of its genome.

This early block in the viral life cycle prompted us to examine the ability of HIV to fuse to cells depleted for Rab6. We employed a commonly used cell fusion assay that mimics viral fusion to host cells. This assay involves co-culturing HL2/3 HeLa cells, which stably express HIV envelope proteins gp41 and gp120, as well as Tat [41], with TZM-bl cells. The viral receptors on the HL2/3 cell line interact with CD4 and CXCR4 on the TZM-bl cells, prompting fusion of the two cells via the same mechanism enveloped virus uses to fuse with the host plasma membrane. Upon cell fusion the Tat protein from the HL2/3 cells can activate beta-galactosidase expression in the TZM-bl cells. Decreased Rab6 levels in TZM-bl cells correlate with diminished beta-galactosidase activity, consistent with the block in HIV infection arising at the level of viral fusion to the host cell (FIG. 3F).

To establish that Rab6 has a role in HIV infection in a more relevant cell type, we transfected the human T cell line, Jurkat, with Rab6 siRNAs, then infected with HIV. A substantial reduction in infection was seen after transfection with two of three Rab6 siRNAs tested (FIG. 3G) and correlated with the level of Rab6 protein depletion as verified by examination of cells from FIG. 3G for Rab6 protein levels by Western blotting. Cell surface expression of CD4 and CXCR4 in these T cells was unaffected by transfection with any of the Rab6 siRNAs (data not shown).

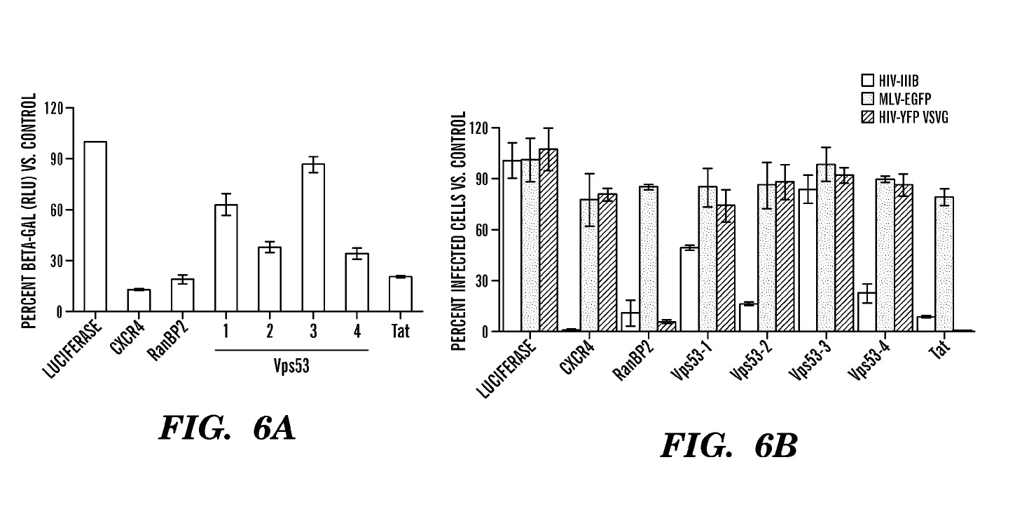

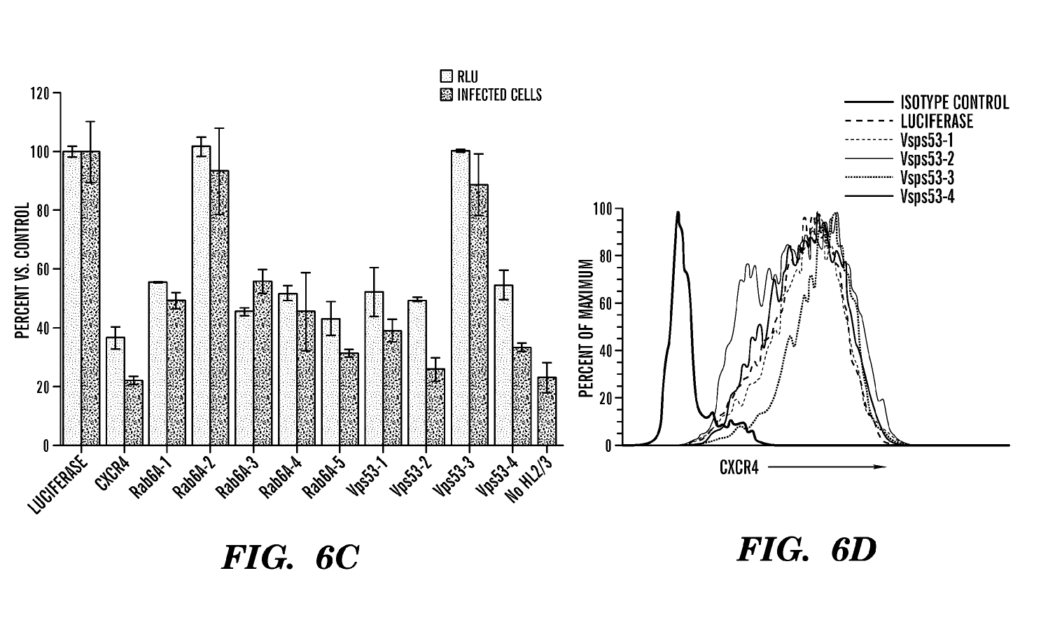

Another strong hit in the initial screen was Vps53, the human homologue of the yeast Vps53 protein, a component of the Golgi associated retrograde protein (GARP) complex [42, 43]. GARP comprises four subunits, Vps51-54, and is responsible for tethering transport vesicles emanating from endosomes that are destined for delivery to the trans-Golgi network (TGN, [44, 45]). Yeast GARP physically interacts with the GTP-bound form of Ypt6 (yeast Rab6), and deletion of Ypt6 blocks arrival of GARP at the TGN [44, 46, 47]. During the validation screen, 3 of 4 Vps53 siRNAs scored, and blocked HIV infection in a single round infection assay (FIG. 6A). Similar to Rab6, Vps53 depletion inhibited WT-enveloped HIV, but not VSV-G pseudotyped HIV or MLV infection (FIG. 6B). CXCR4 surface expression was only slightly decreased in cells transfected with one of the three active Vps53 siRNAs (FIG. 6D), and there was no difference in CD4 levels. Vps53 depletion inhibited cell fusion, which correlated closely with the ability of the individual siRNAs to curtail HIV infection (FIG. 6C). Together, these data suggest that interfering with retrograde trafficking of vesicles from early and/or late endosomes to the Golgi, either through the loss of the small GTPase, Rab6, or its functional partner, the GARP component, Vps53, inhibits infection prior to reverse transcription of the viral genome and perhaps at the level of viral entry.

### A Post Entry Role for a Karyopherin in HIV Replication

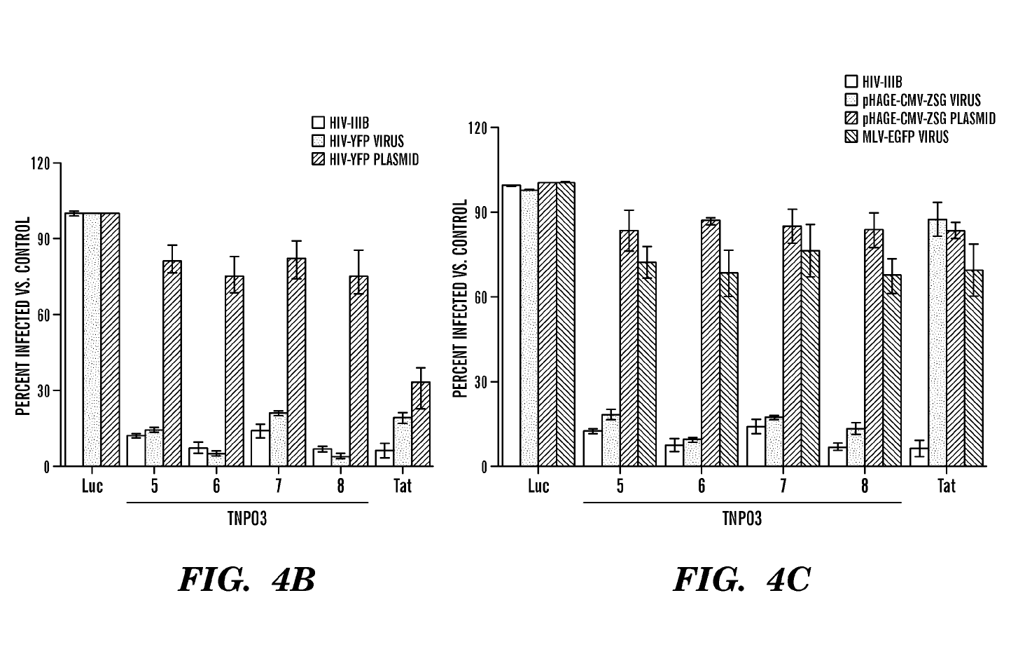

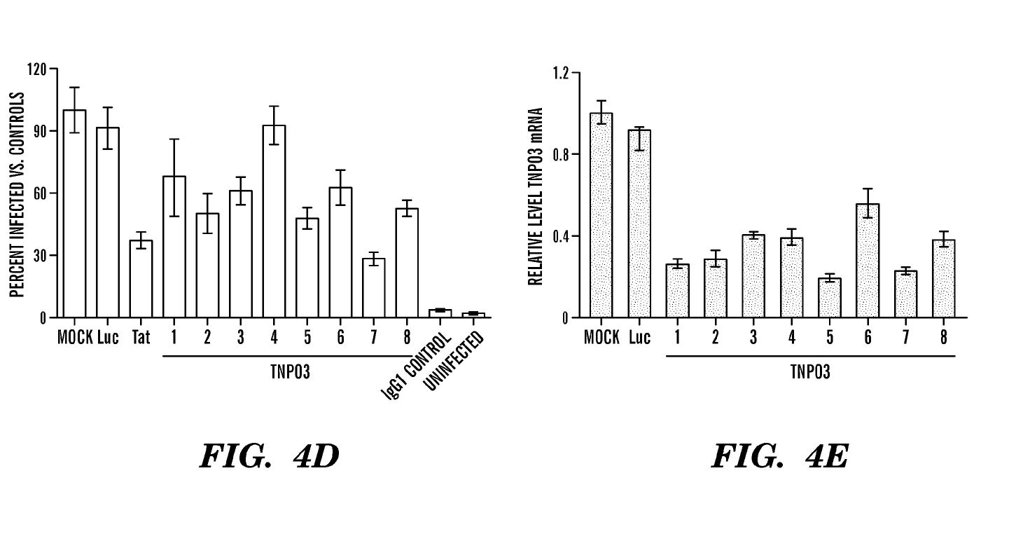

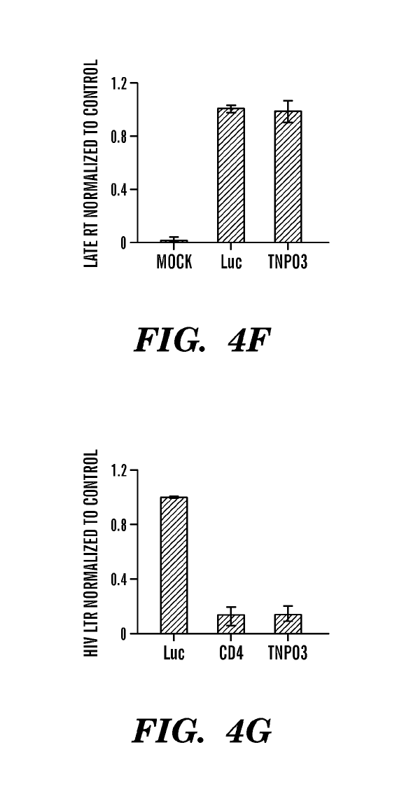

Having identified a block to viral fusion, we next sought to identify HDFs that function post viral entry. Multiple components of the nuclear pore scored in our screen, consistent with the known lentiviral nuclear entry through the NPC. A strong candidate that emerged for a nuclear import specificity factor is Transportin 3 (TNPO3). TNPO3, a member of the karyopherin family of nuclear import receptors, shuttles multiple proteins into the nucleus, including histone mRNA stem-loop binding protein (SLBP, [48], serine/arginine-rich proteins (SR proteins) that regulate splicing of mRNA [49, 50] and repressor of splicing factor (RSF1, [51]). All four siRNAs against TNPO3 blocked HIV infection with no appreciable effect on cell viability. We extended the initial four anti-TNPO3 siRNAs used to a total of eight, all of which silenced TNPO3 and effectively blocked infection in HeLa cells (FIG. 4A). Immunofluorescence images showed the block to HIV infection with loss of TNPO3. Cells were treated as described in B. Luciferase (Luc) negative control siRNA, TNPO3, siRNA #8 targeting TNPO3. A combined image for nuclei staining (blue) with Hoechst 33342 and anti-p24 HIV Gag protein (green) was generated. TNPO3 mRNA reduction, as determined by quantitative real time (RT)-PCR, correlated with the extent of inhibition of infection (FIG. 4E). The block imposed by TNPO3 silencing was independent of HIV envelope, since the VSV-G pseudotyped HIV-YFP virus was similarly impaired, indicating that TNPO3 functions, as expected, after viral entry (FIG. 4B). TNPO3 depletion by seven of eight siRNAs also inhibited viral infection of Jurkat cells (FIG. 4D), indicating TNPO3-dependency in a natural host cell for HIV.

Interestingly, TNPO3 depletion did not affect MLV-EGFP infection (FIG. 4C). These results could be explained if TNPO3 depletion impaired SR protein-dependent splicing of Tat, which is required for efficient HIV, but not γ-retroviral, transcription. However, this hypothesis was not supported by two observations; first, Tat-dependent reporter gene expression from a transiently transfected HIV-YFP plasmid was only weakly affected by TNPO3 depletion (FIG. 4B); second, an HIV derivative, pHAGE-CMV-ZSG, that contains HIV Gag and Pol, but expresses a fluorescent reporter protein cDNA (zoanthus species green, ZSG) from an internal CMV promoter, also showed a dependency on TNPO3 when infected, but not when its plasmid DNA was transfected (FIG. 4C).

Taken together, these observations suggested that TNPO3 might act before transcription, rather than by blocking viral mRNA splicing. Therefore, we examined the steps of reverse transcription and integration of proviral DNA. Assays for late RT-cDNA product and integrated viral DNA in TNPO3-depleted cells showed that the block in the viral lifecycle happened after reverse transcription but prior to integration (FIGS. 4F and G). Thus, diminished TNPO3 levels produce a lentiviral specific pre-integration block, most likely at the stage of nuclear import of the PIC. Our data are consistent with results that indicate HIV PICs utilize an active nuclear import mechanism in both dividing [52] and cycling cells [53]. In contrast, MLV and other 7-retroviruses predominantly enter the nucleus only after nuclear envelope breakdown during mitosis [54]. However, whether TNPO3 directly interacts with the virus or indirectly via altered import of an HDF or splicing of mRNA encoding an HDF required for integration, remains to be determined.

### A Role for the Mediator Complex in HIV Infection

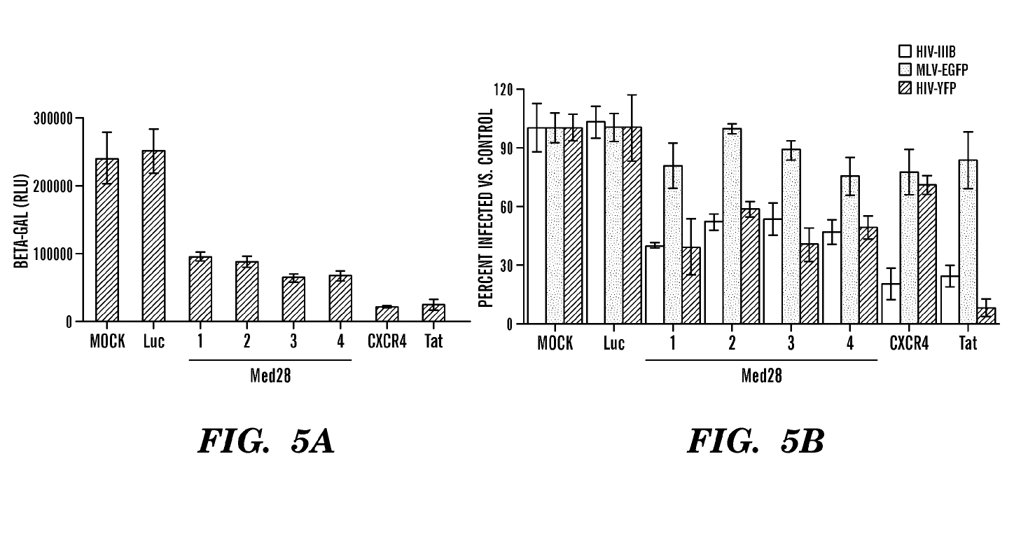

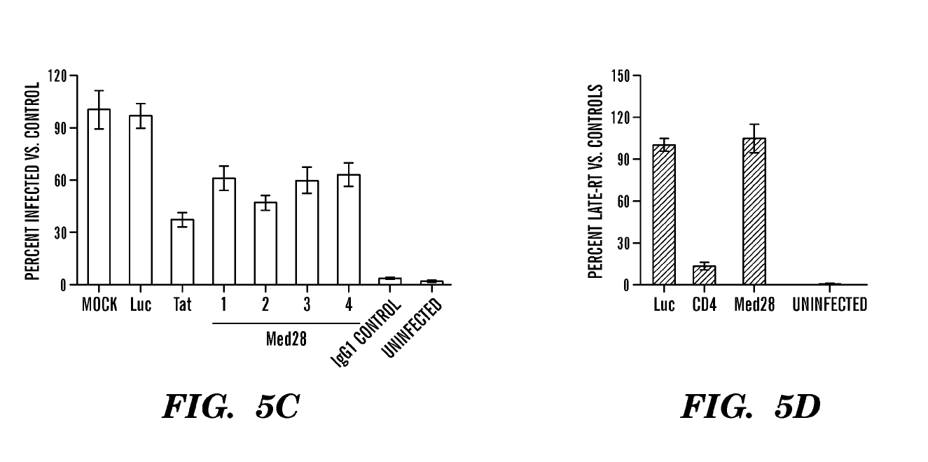

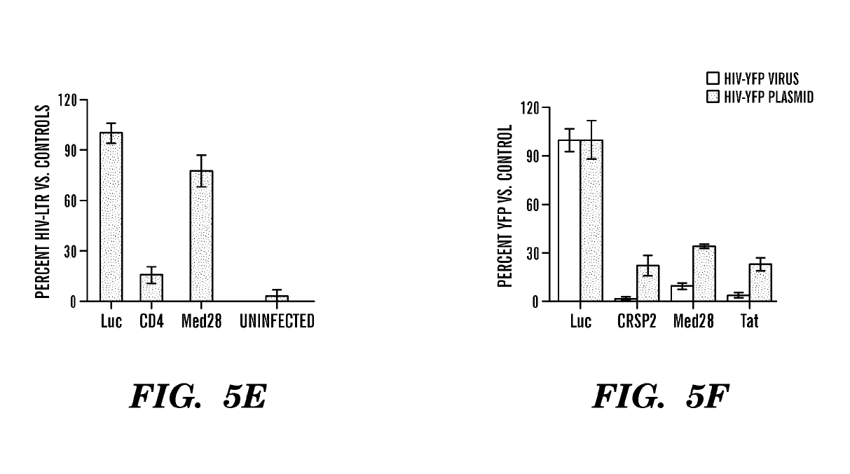

To search for host factors that function post integration we chose to investigate components of the Mediator complex. Depletion of several components of Mediator, which is essential for directly coupling transcription factors to the core RNA PolII, inhibited HIV infection. To examine this functional group, we focused on Med28, a higher-eukaryote restricted component of Mediator, because all four Med28 siRNAs strongly repressed viral infection in the validation screen, and cell viability was near wild type levels. The Med28 siRNAs efficiently inhibited first round HIV infection (FIG. 5A). Depletion of Med28 also protected Jurkat cells from HIV, and efficiently decreased target gene protein levels (FIG. 5C). Jurkat cells from these cultures were also assessed for Med28 protein by Western blotting. Med28 appeared to be specific for HIV infection as it significantly inhibited both HIV-IIIB and HIV-YFP, but not MLV-EGFP. (FIG. 5B). To determine where HIV was halted, we examined the levels of virally produced reverse transcribed cDNA, as well as the amount of integrated proviral DNA (FIGS. 5D and E). Both reverse transcription and integration were unaffected by Med28 depletion, indicating a block post-integration. Med28 loss also affected YFP expression from a transiently transfected HIV-YFP plasmid to a similar extent as seen with the integrated HIV-YFP virus (FIG. 5F). Therefore, we conclude that Med28 is required for transcription of viral genes, consistent with its connection to RNA polII.

### Discussion

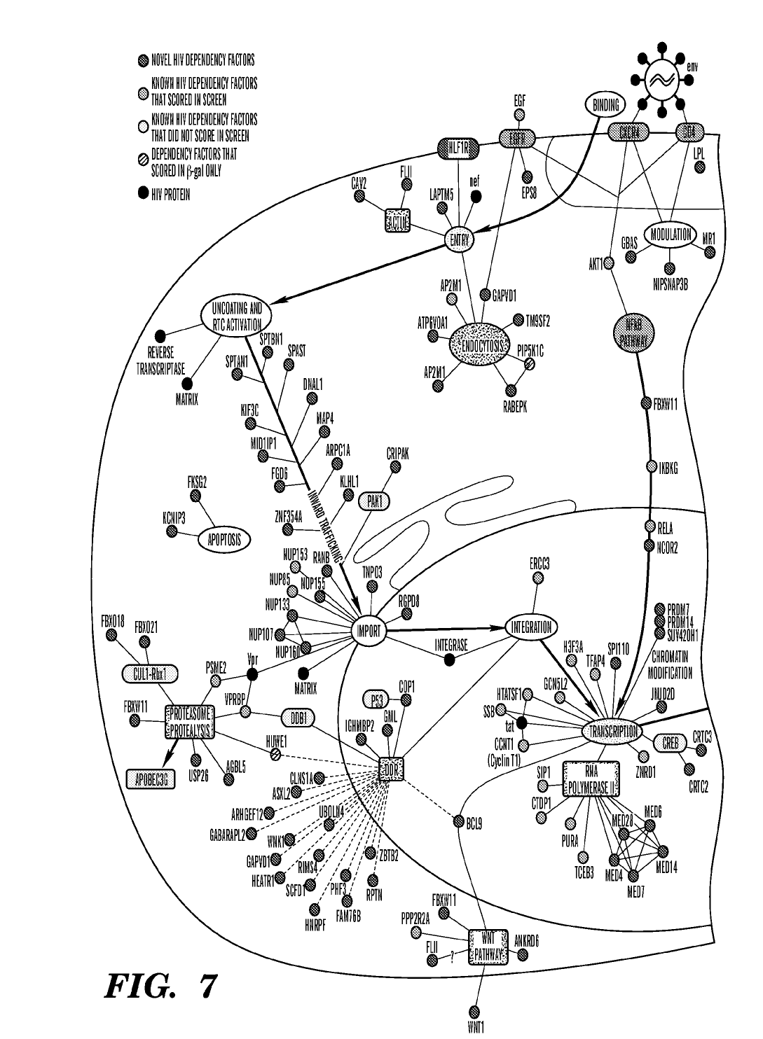

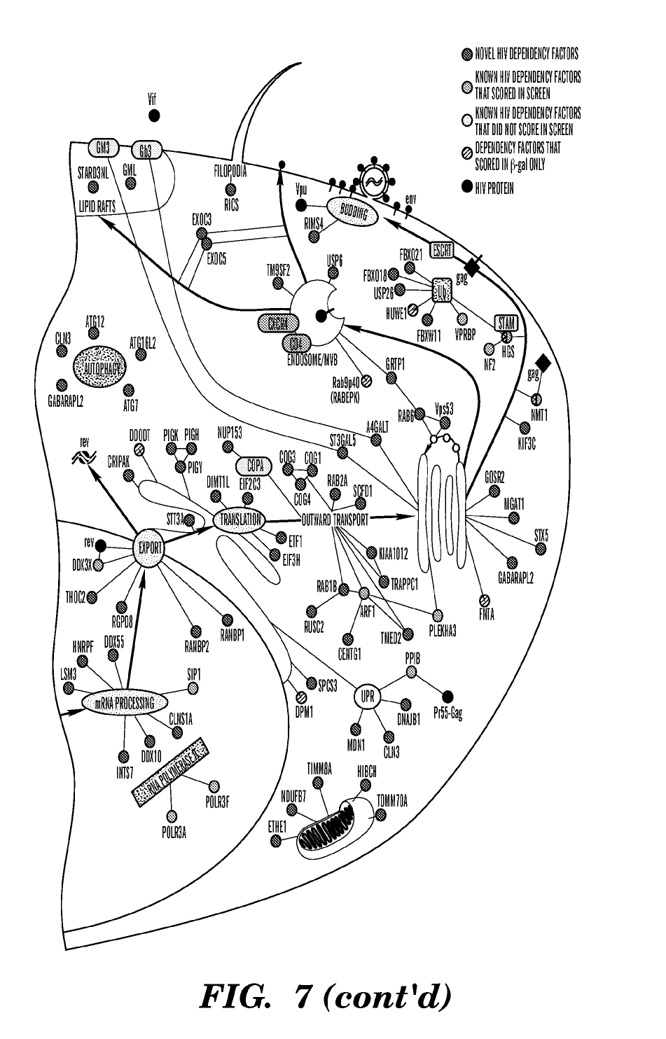

The functions of HIV encoded proteins have received extensive exploration and much progress has been made in understanding the HIV lifecycle. In this study we have used RNAi to investigate the host cell requirements for HIV. The exploitation of host cell functions by HIV is extensive as inferred from the diverse cellular processes detected in our screen. We undertook a comprehensive two part screening strategy using a fully infectious HIV strain, in an effort to uncover host-viral interactions occurring from the initial viral entry all the way to the production of infectious particles. The validity of this screen is supported by the large number of functional modules enriched among the screen hits. Modules involved in membrane synthesis, nuclear import, transcription, golgi function, vesicular trafficking, RNA transport, and exocytosis, were identified. Many of these hits make sense in terms of what was previously known about HIV function. In fact we identified 38 factors previously linked to HIV, although only a handful of these had been shown to be required for HIV function genetically. The functional clustering and previously known HIV factors suggest that the majority of the more than 200 proteins identified with no previous links to virus are likely to play relevant roles in HIV pathogenesis. We have portrayed the HIV viral lifecycle along with the presumed subcellular locations and functions of the novel and known HDFs found in the screen in FIG. 7.

Part two of the screen was designed to select for factors that affect later stages of the viral lifecycle and uncovered 26 HDFs that scored with two or more siRNAs (Table 2). These include two enzymes involved in post-translational addition of sugar, OST48 and DPM1 [55, 56]. HIV ENV must undergo glycosylation to be infectious [57]. Early studies described the efficacy of anti-HIV glycosylation inhibitors, demonstrating these drugs prevented ENV modification and blocked virion fusion with the host cell [58]; Similar efforts continue today [59]. Our screen now provides genetic evidence for this HDF-mediated modification and suggests specific protein targets for therapeutic efforts.

As noted, the host ESCRT machinery has been shown to be vital for HIV budding. Of the 28 host proteins published to be involved in this pathway we recovered only one, HRS. Review of our primary screen data revealed that only siRNAs against two more of these factors, Vps4A and 4B, resulted in extensive cell death. However, given the many factors involved in producing false negative results (incomplete knockdown, functional redundancy), we await the results of future genetic screens for insights into this portion of the viral lifecycle.

Independent verification of the validity of the screen comes from the analysis of the enrichment of genes that are directly or indirectly connected to known proteins implicated in HIV function. We find a strong enrichment for connectivity to this dataset. Furthermore, although the screen was performed in HeLa cells, we found that the genes identified were significantly enriched for high expression in immune cells, the natural host cells for HIV. This observation may be indicative of the virus evolving to better exploit the host environment, or that immune cells may be especially proficient for the functions HIV needs for optimal replication. It will be interesting to determine if the virus is especially reliant on this immune-enriched set of proteins and whether the tropism of other viruses towards their hosts will share a similar enrichment for their host's expression profile.

This collection of HDFs allows the generation of a plethora of testable hypotheses about the HIV life cycle. In this vein we extensively validated the role of four novel factors Rab6, Vps53, TNPO3 and Med28 in HIV infection. We discuss the potential roles in infection of three of these validated hits below.

### The Role of Rab6 and Vps53 in HIV Infection

The concentric barriers formed by the plasma and nuclear membranes serves in large measure to prevent pathogens from invading our cells. Our results suggest that Rab6 and Vps53 play a role in allowing HIV to penetrate the first of these cellular defenses. Loss of either the small GTPase, Rab6, or the GARP component, Vps53, inhibits HIV infection at the level of viral fusion to the membrane. How might Rab6 and Vps53 affect HIV entry? While we have ruled out alteration of host coreceptor cell surface expression, several alternative possibilities exist. There could exist a previously undetected novel co-receptor, dependent in some manner on Rab6 and Vps53. The screen identified 39 transmembrane proteins with no known association with HIV infection (Table 2). Perhaps modification of CD4 or the chemokine receptors may be aberrant. However, despite extensive efforts, no host receptor gylcosylation has been shown to be required for HIV infection [60-62]. Alternatively, the membrane environment, or the lipid composition of the cell's surface, may be affected, possibly due to alterations in the major supplier of membrane, the Golgi. Among the possible candidates for this proposed perturbation are the glycosphingolipids (GSLs). GSLs, composed of ceramide with an attached sugar, are sequentially synthesized by 11 ER and 3 Golgi enzymes [63]. Golgi-resident enzymes depend on retrograde vesicular transport mediated by Rab6 and Vps53 for recycling [39, 64]. Disruption of recycling results in vesicular scattering and inappropriate lysosomal degradation of many Golgi resident enzymes, such as glycotransferases [39, 42, 64]. GSLs are required for HIV fusion [65], possibly through direct interaction with HIV gp120 [66]. Importantly, reducing levels of the GSLs, Gb3 or GM3, inhibits HIV fusion with primary T cells [67]. Supporting this notion, we find that HIV infection is also decreased by siRNA-mediated depletion of the enzymes which synthesize Gb3 and GM3, A4GALT and SIAT9, respectively. Other components of the GSL synthesis pathway found by the screen include a recently identified GSL-transfer protein, FAPP1 (PLEKHA3, [68, 69] and the small GTPase, ARF1, which targets FAPPs to the Golgi (FIG. 7, Table 2).

Loss of Rab6 and Vps53 may also inhibit HIV infection by altering lipid raft assembly. Lipid rafts are microdomains within the plasma membrane, richly populated by GSLs, cholesterol, and transmembrane receptors, among them CD4 [70, 71], as well as multiple glycosyl-phosphatidylinositol (GPI)-linked proteins. Disruption of lipid rafts inhibits HIV infection [33, 72]. Several additional factors found in the screen, including 4 GPI-linked proteins (Table 2), enzymes which synthesize GPI-linked proteins (PIG-H, K, Y), and STARD3NL, may all contribute to lipid raft function [73].

### Requirement of the Nuclear Pore and TNPO3 in HIV Infection

The HIV PIC preferentially gains access to the nucleus through the nuclear pore. We identified six of the 30 proteins that form the NPC. One, Nup153, contains 40 phenylalanine-glycine enriched repeat motifs (FG-domains, [74, 75]). NPC proteins at the nuclear and cytosolic faces, and the central pore, possess FG-domains [76]. This lining of FG-domains permits macromolecules, such as the HIV PIC, to access the nucleus only if they are accompanied by a karyopherin [77]. Loss of Nup153 prevents the nuclear import, but not NPC binding, of a yeast retrotransposon Gag protein [78]. This suggests that Nup153 may be needed to send the HIV PIC through the mouth of the NPC, but not for the initial association of the PIC and NPC. A strong candidate from our screen for this docking function is RanBP2, a large tendrilous protein located on the cytosolic face of the NPC, which also contains numerous FG-domains [79]. An siRNA screen in Drosophila found that Nup153 and RanBP2 depletion altered selective import of different cargoes without altering CRM1-mediated nuclear export [80]. A candidate for the karyopherin is TNPO3, whose depletion profoundly blocked the infection of HIV post reverse-transcription but prior to integration. This phenotype could be indirect, as TPNO3 could be required for the activity of another HDF. However, a simple direct model consistent with the NPC data is that HIV nuclear entry involves binding of the HIV PIC to TNPO3 to form a karyopherin associated integration complex (KIC) that docks on RanBP2 via the latter's FG-domains. The KIC then transitions onto the contiguous FG-domain surface provided by Nup153, resulting in its passage through the pore. While speculative, these are examples of the kinds of detailed hypotheses that can be generated from a highly validated functionally-derived dataset such as the one resulting from this screen.

### Implications for Future HIV Therapies

A key pharmacologic strategy for treating individuals infected by HIV has been to target multiple virus-encoded enzymes required for replication. From this strategy have emerged a number of inhibitors that show good initial efficacy against HIV function. Unfortunately, due to the high mutability of the virus, drug resistant variants arise at a high frequency. To combat this, combinatorial regimens have been deployed to decrease the frequency of resistance. We have taken a parallel strategy to combat HIV function by identifying novel host factors involved in HIV infection, with the goal of finding all possible dependencies that this pathogen possesses. Here we have identified drug targets in the human proteome with which to disrupt the HIV life cycle. We anticipate that HIV would encounter a much greater problem evolving resistance to drugs targeting cellular proteins because it would have to evolve a new capability, not simply alter amino acids in a drug binding site. This is conceptually analogous to blocking angiogenesis in non-tumor cells to deprive cancer of it blood supply [82, 83].

### Addendum

The host ESCRT machinery has been shown to be vital for HIV budding. Of the 28 host proteins published to be involved in this pathway we recovered only one, HRS. Review of our primary screen data revealed that only siRNAs against two more of these factors, Vps4A and 4B, resulted in extensive cell death. LEDGF, a well confirmed HDF important for integration, was not detected in this screen, likely because its intracellular levels greatly exceed those required by the virus (M. C. Shun et al., Genes Dev 21, 1767 (Jul. 15, 2007)). However, given the many additional factors, other than insufficient knockdown of the target, involved in producing false negative results (functional redundancy, poor siRNA design, essential gene, off-target toxicities, HIV strain deficient in accessory proteins (please see below), and operator error), we await the results of future improved genetic screens for insights into these and other portions of the viral lifecycle. Furthermore, host factors that might affect the immune response to HIV would likely be missed in this cell-based screen.

As noted above, the HIV-IIIB lab strain used in this study is deficient in Nef, Vpu and contains a frame shift mutation which codes for a truncated Vpr protein. The predicted HIV-IIIB Vpr open reading frame would produce a 78 aa protein (wild-type 96 aa full length), with the first 72 residues identical to the NL4-3 wild-type Vpr protein and 6 additional amino acids, from 73-78, encoded by the shifted reading frame (L. Zhao, S. Mukherjee, O. Narayan, J Biol Chem 269, 15577 (1994)). This truncated Vpr is missing the six most C-terminal amino acids contained in a previously described deletion mutant, Vpr 78-87, which was demonstrated to maintain its interaction with the host factor, VPRBP (L. Zhao, S. Mukherjee, O. Narayan, J Biol Chem 269, 15577 (1994)). A conserved interaction domain Vpr aa 60-78 was defined (underlined below, based on homology to the viral sequence stated in the reference as being amplified from HIV-1/89.6 (L. Zhao, S. Mukherjee, O. Narayan, J Biol Chem 269, 15577 (1994); R. Collman et al., J Virol 66, 7517 (1992)). A truncated Vpr protein containing aa 1-84 was expressed in 293T cells, but unlike the wild-type Vpr, this mutant was unable to induce a G2 cell cycle arrest (P. Marzio, S. Choe, M. Ebright, R. Knoblaugh, N. R. Landau., J Virol 69, 7909 (1995)). Therefore, while the HIV-IIIB Vpr protein may exist at low levels during infection, it is unlikely to mediate its effect by inducing a G2 cell cycle arrest via interactions with VPRBP.

### Example 2

In a follow-up screen, using the same methods as detailed in Example 2, an additional 82 host factors involved in HIV infection were identified independently and verified in a validation screen, or were identified in Example 1, and verified in a validation screen in this follow-up. These HDFs are listed in Table 3, along with the earlier identified HDFs. The genes were verified by inhibition with one or more siRNAs. The sequences of the siRNA nucleic acids used to inhibit expression of the respective genes is shown in Table 3 as well.

### Tables

Table 1. Host Proteins Previously Implicated in HIV Infection Recovered From siRNA Screen. 38 genes were classified as known HIV dependency factors based on previous published evidence and/or inclusion in the HIV interaction data base (NCBI). Numbers in parentheses indicate individual siRNAs out of a total of four possible, that scored on retesting. SP=SMARTpool scored, since the four oligos in the pool were not individually tested.

Table 2. HIV dependency genes. A list of genes that scored in the screen and their annotation across various databases. The number of individual siRNAs that scored in either part one or just in part two of the screen are given, based on decreasing HIV infection by 2 SD from the mean of the negative controls. Genes which only scored with two or more hits in part two of the screen are listed as positive in beta gal only. Gene names, synonyms, description and genomic location were obtained from NCBI Reference Sequence (Revision October 2007). UniProt accession numbers were mapped to NCBI Gene IDs by accession numbers provided in UniProt cross-reference file. Gene ontology annotations (Revision October 2007) were obtained from the Gene Ontology Consortium (www.geneontology.org) and mapped to NCBI GeneIDs. Ortholog proteins were identified using NCBI HomoloGene. HIV interactions and their references were obtained from NCBI HIV interaction database.

Table 3. HDFs identified in the screen and follow-up screen and corresponding Gene ID, Dharmacon Catalogue Number, Accession Number, and nucleic acid sequences (siRNA sequences) which inhibit gene expression.

Table 4. 14 likely candidates of HIV therapeutics, their gene ID and T cell expression, their presumed activity and whether or not they are thought to be transmembrane proteins.

### Materials and Methods

siRNA screen: To identify host factors required for HIV infection, a high-throughput RNAi-based screen was undertaken on an arrayed library containing 21,121 siRNA pools targeting the vast majority of the human genome (Dharmacon Inc. Lafayette, Colo.).

Part one of the screen: siRNAs were transiently transfected into the TZM-bl cells at a 50 nM final concentration, using a reverse transfection protocol employing 0.45% Oligofectamine (Invitrogen, Carlsbad, Calif.) in a 384-well format. The Oligofectamine was diluted in Opti-MEM (Invitrogen) and allowed to incubate ten minutes. The lipid solution was then aliqouted into the wells (9 ul/well) using a liquid handing robot. The plates were spun down at 1000 RPM and the arrayed siRNAs were added robotically, 1.5 ul of a 1 uM stock per well. After a twenty minute incubation, approximately 440 TZM-bl cells were added per well, in 20 ul of Dulbecco's modified minimal essential media (DMEM, Invitrogen), supplemented with 15% fetal bovine serum (FBS, Invitrogen). The plates were next spun at 1000 RPM and then placed in a tissue culture incubator at 37 C and 5% CO2. After 72 h of siRNA-mediated gene knockdown, the medium was removed and the cells are treated with HIV-IIIB (NIH AIDS Research and Reference Reagent Program (NARRRP)) at an MOI of 0.5 in 100 ul DMEM with 10% FBS. After an additional 48 h incubation (when silencing is still operative), 20 ul of media was removed and replica plated onto a new 384 well plate containing 1800 TZM-bl cells per well (beginning of part two of screen). The “part one” cells were then fixed with 4% Formalin, permeabilized with 0.2% Triton-X100 and stained for p24, using purified anti-HIV-1 p24 (mab-183-H12-5C, generously provided by the NARRRP, Reagent 3537, kindly contributed by Dr. Bruce Chesebro and Kathy Wehrly) and an Alexa 488 goat anti-mouse secondary (A11001) and rabbit anti-goat tertiary (A11078) antibodies (Invitrogen), and for DNA (Hoechst 33342, Invitrogen). Each step was followed by two washes with buffer containing 10 mM Tris pH 7.5, 150 mM NaCl, 2 mM EDTA pH 8, and 1% FBS. The cells were then imaged on an automated Image Express Micro (IXM) microscope (Molecular Dynamics) at 4× magnification, using two wavelengths, 488 nm to detect HIV infected cells expressing p24, and 350 nm for nuclear DNA bound by Hoecsht 33342. Images were then analyzed using the Metamorph Cell Scoring software program (Molecular Dynamics Inc.) to determine the total cells per well, and the percentage of p24 positive cells in each well (percent infected). A negative control luciferase siRNA (Luc) and positive control siRNA SMARTpools against CD4 and Rab9p40 (Dharmacon) were present on each plate. In addition wells containing either buffer alone, a non-targeting control siRNA (siCONTROL Non-Targeting siRNA #2, Dharmacon), and an siRNA pool directed against Polo like kinase one (PLK1, Dharmacon) were present on all plates transfected. The screen was performed in duplicates.

Part two: To search for host factors whose depletion leads to defects in producing infectious particles, 20 ul of conditioned media containing HIV from each well in the first round screen was removed prior to fixation and transferred to a new well containing uninfected TZM-bl cells. 20 h later these cells were treated with Gal-Screen chemiluminescence reagent (Applied Biosystems, Foster City, Calif.), and assessed with an Envision 2 plate reader (Perkin Elmer, Waltham, Mass.) for Tat-dependent transcription of the stably integrated beta-galactosidase reporter gene. These results were normalized to cell number present in the first round donor well, as recorded by the IXM microscope. Control experiments using HeLa-CD4 cells (which do not contain a Tat-dependent reporter gene) in the recipient wells showed that no significant beta-gal activity was transferred along with the supernatant. siRNA pools were classified hits if they decreased the percentage of p24 positive cells or beta-gal light units by two or greater standard deviations (SD) from the plate mean on both of the duplicate plates, and viable cells were not decreased by greater than two SDs from the mean of the plate. We next performed a validation screen, in which the four individual oligos comprising each pool were placed into separate wells, and screened again using identical methods as above. Visual spot inspections of control images were done throughout the screen to confirm the accuracy of the automated imaging and cell scoring systems.

Cell Culture. TZM-bl and HL2/3 HeLa cells were generously provided by the NARRRP, and kindly contributed by Dr. John C. Kappes, Dr. Xiaoyun Wu and Tranzyme Inc. (TZM-bl), and Dr. Barbara K. Felber and Dr. George N. Pavlakis (HL 2/3, [41]). HeLa cells were grown in DMEM supplemented with 10% FBS. Jurkat cells were grown in RPMI-1640, with 10% FBS and 0.1% beta-mercaptoethanol (Invitrogen). TZM-bl cells were chosen due to limitations in experimental methods using more relevant T and macrophage cell lines. They proved useful for screening because they are easily transfected with siRNA, are hardy enough to survive high throughput manipulations and support a full HIV lifecycle to produce infectious virions.

Viral propagation. HIV-1-IIIB was propagated in the T cell line H9, grown in DMEM supplemented with 10% heat-inactivated fetal calf serum, 2 mM L-glutamine, 50 U/ml of penicillin and 50 μg/ml streptomycin by treating the cells with a 0.2 MOI of virus. The viral infection was monitored until >80% of the cells stained positively for p24, after which the supernatant containing the progeny virus was harvested in 24 h intervals. The CCR5-tropic HIV-Bal was propagated on human monocyte-derived macrophage cells. Briefly, peripheral blood mononuclear cells were isolated from whole blood obtained from healthy donors by Ficoll-Hypaque (Pharmacia) density centrifugation. The isolated cells were washed extensively in PBS and plated in RPMI containing 10% heat inactivated human AB serum, 2 mM L-glutamine, 50 U/ml of penicillin and 50 μg/ml streptomycin and plated a 2×10⁶ cells/ml in 24 well plates. The non-adherent cells were removed after 5 days of culture by washing with warm media. The macrophage cells were infected with a 0.2 MOI of HIV-1-Bal and the infection was monitored until >90% of the cells were infected. The virus containing supernatant was harvested by centrifugation (1,500×g for 10 min), aliquoted and stored at −80° C. The viral titers for both HIV-1 strains were determined by treating Magi (IIIb) or Magi-CCR5 (Bal) cells (NIH AIDS research and reference reagent program) with increasing amounts of viral supernatant. 48 h post infection the cells were stained for HIV-1 p24 expression.

Plasmids, shRNA and siRNA Reagents. The coding sequence for Rab6′ was PCR-amplified, fully sequence confirmed as correct, and then recombined into a Gateway-compatible entry vector using BP-clonase (Invitrogen); This insert was then recombined in frame into a N-terminal GFP fusion expression vector with a Blasticidin selectable marker (gift from Jianping Jin, Harvard Medical School) using LR recombinase (Invitrogen), to produce p203-GFP-Rab6. A GFP only version of the expression vector was used as control plasmid, p203-GFP. The HIV-YFP plasmid was previously described and created by replacing the alkaline phosphatase gene (AP) with the YFP gene (Clontech, Mountain View, Calif.) in pHIV-AP□env□vif□vpr, which was in turn derived from the HIV-1 strain NL4-3 clone (Accession number AF033819) by deleting vif and vpr (0.62 kb section removed) and 1.45-kb of env [84-86]. HIV-YFP contains an intact TAR and is Tat-dependent for transcription (Personal communication, Dr. Richard Sutton, Baylor College of Medicine, Houston). The pHAGE-CMV-ZSG plasmid is a derivative of HRST-CMV, and contains self inactivating LTRs, an internal CMV promoter driving expression of a the ZSG reporter gene, a rev response element (RRE), and a woodchuck hepatitis post-transcriptional regulatory element (WPRE, gift of A. Balazs and R. C. Mulligan, Harvard Medical School). The MLV-EGFP plasmid contains and MLV-LTR and the humanized form of Renilla green fluorescence protein (Invitrogen) and was kindly provided by F. Diaz-Griffero and J. Sodroski, Harvard Medical School.

The EcoRI site of pMSCV-puro vector, containing the puromycin resistance gene (Invitrogen) was modified to an MluI site to generate pMSCV-PM (pMSCV-Puro-MluI). shRNAs against Rab6A from the second generation Hannon-Elledge shRNA library [87] were subcloned from the SalI/MluI sites of pSM2c into the XhoI/MluI sites of pMSCV-PM to generate pMSCV-PM-shRNA plasmids, amenable to packaging into retroviruses. The following shRNAs were used:

(SEQ ID NO: 1457)

Luciferase control (FF) CGCCTGAAGTCTCTGATTAA

(SEQ ID NO: 1458)

shRab6-1 CTCTTTCACATGTGCTTTA
Rab6A 3′UTR 1887-1905

(SEQ ID NO: 1459)

shRab6-2 CCTGCTGAATTTATGTTGT
Rab6A 3′UTR 2776-2794

(SEQ ID NO: 1460)

shRab6-3 CCATTGGAATTATCCTTTA
Rab6A 3′UTR 1642-1660

The following custom siRNA oligonucleotides (Dharmacon) were used in this study:

(SEQ ID NO: 1461)

Luciferase control (Luc) CGTACGCGGAATACTTCGA

(SEQ ID NO: 1462)

HIV-1 Tat CUGCUUGUACCAAUUGCUAUU

All of the following are Dharmacon siRNAs, catalogue numbers are provided, however in the case of the individual duplex oligos these have been subject to change and we suggest following the sequence information given,

all are human-sequence reagents:

CD4 (SMARTpool M-005234-01), Rab9p40 (SMARTpool
M-019457-00), PLK1 (M-003290-01)

(SEQ ID NO: 1463)

Rab6-1 D-008975-06 CCAAAGAGCUGAAUGUUAUUU

(SEQ ID NO: 1464)

Rab6-2 D-009031-03 CUACAAAGUGGAUUGAUGAUU

(SEQ ID NO: 1465)

Rab6-3 D-008975-04 GAGCAACCAGUCAGUGAAGUU

(SEQ ID NO: 1466)

Rab6-1 D-008975-01 GAGAAGAUAUGAUUGACAUUU

(SEQ ID NO: 1467)

Rab6-2 D-008975-04 GAGCAACCAGUCAGUGAAGUU

(SEQ ID NO: 1468)

Rab6-3 D-008975-05 AAGCAGAGAAGAUAUGAUUUU

(SEQ ID NO: 1469)

Rab6-4 D-008975-06 CCAAAGAGCUGAAUGUUAUUU

(SEQ ID NO: 1470)

Rab6-5 D-009031-03 CUACAAAGUGGAUUGAUGAUU

(SEQ ID NO: 1471)

TNPO3-1 D-019949-01 GCAGUGAUAUUUAGGCAUAUU

(SEQ ID NO: 1472)

TNPO3-2 D-019949-02 GGAGAUCCUUACAGUGUUAUU

(SEQ ID NO: 1473)

TNPO3-3 D-019949-03 GAAGGGAUGUGUGCAAACAUU

(SEQ ID NO: 1474)

TNPO3-4 D-019949-04 GAGGGUAUCAGACCUGGUAUU

(SEQ ID NO: 1475)

TNPO3-5 J-019949-09 CGACAUUGCAGCUCGUGUAUU

(SEQ ID NO: 1476)

TNPO3-6 J-019949-10 GAGUGAAGUCGUUGAUCGAUU

(SEQ ID NO: 1477)

TNPO3-7 J-019949-11 UCACCAGGUUGUUUCGAUAUU

(SEQ ID NO: 1478)

TNPO3-8 J-019949-12 GUACAAAACUAACGAUGAAUU

(SEQ ID NO: 1479)

Med28-1 D-014606-01 GCGGAAAGAUGCACUAGUCUU

(SEQ ID NO: 1480)

Med28-2 D-014606-02 GUACUUUGGUGGACGAGUUUU

(SEQ ID NO: 1481)

Med28-3 D-014606-03 UGAGUGGGCUGAUGCGUGAUU

(SEQ ID NO: 1482)

Med28-4 D-014606-04 CAGAAACCAGAGCAAGUUAUU

(SEQ ID NO: 1483)

Vps53-1 D-017048-01 GAAAGGAGAUUUAGAUCAAUU

(SEQ ID NO: 1484)

Vps53-2 D-017048-02 GCAAUUAGAUCACGCCAAAUU

(SEQ ID NO: 1485)

Vps53-3 D-017048-03 AGAAGUACCUCCGAGAAUAUU

(SEQ ID NO: 1486)

Vps53-4 D-017048-04 GCGCCGACCUCUUUGUCUAUU

(SEQ ID NO: 1487)

RanBP2 (RanBP2L) D-012007-03 GAAGUCCUGCAAUUUAUAAUU

HeLa cells were transfected with siRNAs (50 nM) using Oligofectamine (Invitrogen) according to the manufacturer's protocol. Transfection of plasmids was performed using Exgene-500 per the manufacturer's instructions. Efficiency was determined by cotransfection of MSCV-DSred. Jurkat cells (2e6 per reaction) were transfected with 1.2 uM final concentration of siRNA using a Cell line nucleofactor kit V, with program setting T-14, as per the manufacturer's instructions (Amaxa Biosystems, Cologne, Germany). 72 h after transfection the Jurkat cells were infected with HIV-IIIB at an MOI of 0.2, see Flow cytometry section below for analysis.

Retrovirus production and infection. Retroviruses containing MSCV-PM empty vector (mir30), control (FF) or Rab6 shRNAs (shRab6-1, 2, and 3) were produced by transfecting 293T cells with the specific retroviral plasmid, pCG-Gag-Pol, and pCG-VSV-G using TransIT-293 (Minis) in OptiMEM per manufacturer's instructions. HIV-YFP virus was created by transfecting the HIV-YFP plasmid (kindly given by R. E. Sutton, Baylor School of Medicine) with pCG-VSV-G. p203-GFP-Rab6, p203-GFP, and pHAGE-CMV-ZSG virus was produced by transfecting the pHAGE plasmid, along with pHDM.Hgpm2 (a codon optimized HIV-1_{NL4-3} Gag-Pol), pHDM-VSV-G, pRC1 CMV-Rev1b, and pMD2btat1b (all kind gifts of J. W. Walsh and R. C. Mulligan, Harvard Medical School). MLV-EGFP virus was prepared by cotransfecting pVPack-GP (Stratagene, La Jolla, Calif.) and pcG-VSV-G. Retroviruses were harvested 48 h after transfection, filtered with a 0.45 μm filter, titered, and stored at −80° C. For generation of the stable shRab6-KD cell lines, TZM-bl cells were infected at an MOI ˜3 using 8 μg/ml polybrene (Sigma). The media was replaced 24 h after infection, and the cells were selected with Puromycin (Invitrogen) at 2 ug/ml. To rescue the shRab6-KD cell lines, cells were infected with either p203-GFP-Rab6 or p203-GFP, and 48 h later populations of cells were put under Blasticidin selection at 2 ug/ml.

HIV-IIIB and HIV_{Ba1} were obtained from the NARRRP. HIV-IIIB titer was determined by FACs analysis of H9 T cells stained with HIV-1 p24 after infection.

Western Analysis. Whole-cell extracts were prepared by cell lysis in SDS sample buffer, resolved by SDS/PAGE, transferred to Immobilon-P membrane (Millipore), and probed with the indicated antibodies. Rabbit anti-Rab6 (C-19, sc-310 Santa Cruz Biotechnology), mouse monoclonal anti-Med28 7E1 (very kind gift from Dr. Vijaya Ramesh, Massachusetts General Hospital).

Quantitative PCR. Total RNA was extracted using an RNeasy Plus RNA isolation kit (Qiagen, Valencia Calif.). cDNA was generated using a Quantitect Reverse Transcription kit (Qiagen). Specific cDNAs were quantitated by quantitative PCR with the primer combinations listed below, using a QuantiTect SYBR Green PCR Kit (Qiagen) on an ABI 7500 Real Time PCR system following the manufacturer's instruction (Applied Biosystems). Primers were designed using the Roche Applied Science Universal Probe Library web site (Roche, Indianapolis, Ind.). PCR parameters consisted of 1 cycle of 50° C.×30 s, then 94° C.×15 s, followed by 40 cycles of PCR at 95° C.×15 s, 56° C.×30 s, and 72° C.×30 s. The relative amount of target gene mRNA was normalized to GAPDH mRNA. Specificity was verified by melt curve analysis and agarose gel electrophoresis.

| | |
|---|---|
| Primer sequences | |
| GAPDH 5′ GGAGCCAAACGGGTCATCATCTC | (SEQ ID NO: 1488) |
| GAPDH 3′ GAGGGGCCATCCACAGTCTTCT | (SEQ ID NO: 1489) |
| TNPO3 5′ CCTGGAAGGGATGTGTGC | (SEQ ID NO: 1490) |
| TNPO3 3′ AAAAAGGCAAAGAAGTCACATCA | (SEQ ID NO: 1491) |

HIV Integration analyses. HeLa-T4 cells were transfected with siRNAs on day 1 and repeated on day 2. Cells were infected with HIV IIIB on day 3 and DNA was extracted using the Hirt method at both 7 h post-infection (hpi) and 24 hpi. Late RT products, 2-LTR formation and integrated HIV DNA were analyzed as described [13, 88]. Briefly, Late RT products in extrachromosomal DNA fractions at 7 hpi were analyzed by real-time PCR using MH531/MH532 primers [88]. Integrated HIV DNA at 24 hpi was measured by Alu-PCR followed by nested real-time PCR using AE989/AE990 primers [13].

Cell Fusion Assay. The target cells, TZM-bl shRab6 stable cells, were plated in 96-well plates, 20,000 cells per well. The cells were then cultured overnight. The following morning, the media was removed and 15,000 HL2/3 cells were added to each well in fresh media. The co-culture was then incubated at 37° C. for 6 hours to allow fusion to occur. Fusion was monitored by assaying for Tat-dependent beta-gal reporter gene activation stimulated by HIV-1 Tat from the HL2/3 cells. TZM-bl cells alone were used to determine background luminescence. For cell fusion experiments using siRNA transfected cells, TZM-bl cells were transfected as noted above, and after a 72 h knockdown, the HL2/3 cells were added in fresh media.

Flow Cytometry. To assess levels of the coreceptors on TZM-bl cells, the cells were harvested with cell dissociation buffer enzyme-free PBS-based (Invitrogen), washed and then stained with the following antibodies: Mouse monoclonal anti-Human-CD4, clone 13B8.2, conjugated with PE (Beckman Coulter, Fullerton Calif.), or mouse monoclonal anti-Human CXCR4 (CD184), conjugated with PE (BD Biosciences, Franklin Lakes, N.J.), or mouse isotype matched PE-conjugated control antibodies. To determine levels of HIV infection in Jurkat cells, the cells were fixed and permeabilized (Fix and Perm Kit, Invitrogen), then incubated with mouse anti-HIV-1 p24-PE antibody (KC57-PE, Beckman Coulter) or a mouse isotype matched PE control antibody. Fluorescence intensity was analyzed by using flow cytometry of 10,000 events (BD LSR II; Beckman Coulter).

### Bioinformatics Analysis:

Gene Ontology. Gene ontology terms and gene annotations were obtained from the gene ontology web site (www.geneontology.org; ontologies revision: 5.508; gene associations revision: Oct. 8, 2007). Uniprot and VEGA gene identifiers were mapped to NCBI gene identifiers. In cases where multiple ids matched the same NCBI gene, all gene ontology terms from these ids were combined and assigned to the NCBI gene. All gene ontology terms assigned to genes that scored positive in the screen were obtained and tested for over-representation using a hypergeometric distribution as described in the GOHyperGAll module of bioconductor [89]. Briefly, the hypergeometric distribution is a discrete probability distribution that describes the number of successes in a sequence of N draws from a finite population without replacement. In this context each gene ontology term can be viewed as a basket containing two types of balls: black balls, representing all human genes annotated with that term and white balls, representing genes from a list tested for enrichment. The hypergeometric distribution can be used to calculate the probability of sampling X white balls from that basket. Biological process terms which were assigned to more than 500 human genes were ignored since these term tend to be too generic and contribute little information.

Biological process. The Gene Ontology vocabulary is arranged in a tree structure with a single root node. To simplify the representation of terms, terms which were significantly enriched with a p-value <0.05 and connected in the tree hierarchy were combined to form an over-represented cluster of connected terms. All the genes annotated within that cluster of terms were represented by the most significant term in the cluster. To further reduce the redundancy within the Gene Ontology tree, the clusters were ordered based on p-values and if the genes in one cluster were fully contained within another more significant cluster that cluster was ignored. Finally, we excluded significant terms for which only one gene was assigned.

Molecular function. Gene ontology terms for the molecular function category were processed as described above for biological process. However, no clustering of terms was performed for this category.

Subcellular localization. The subcellular location of each gene was manually curated based on annotations from Swissprot [90] and Gene Ontology [91]. Prediction tools were applied for genes with no annotations. Namely, the program Phobius was used to predict trans-membrane domains [92]; Maestro to predict mitochondria proteins [93] and TargetP to predict secreted and mitochondria proteins [94].

Microarrays. Gene expression profiles across 79 tissues were obtained from the GNF consortium [51]. Expression profiles from Affymetrix U133A platform and GNF custom probes were used. Gene expression profiles performed on Affymetrix U133A platform of T cells, macrophages and dendritic cells were obtained from Chtanova et al. [95]. Expression profiles were normalized using the GCRMA method as implemented in bioconductor [89]. Affymetrix MASS module of bioconductor was used to identify present or absent transcripts [89] and probes with no single present call across all tissue or highest expression value below log2(100) were removed. Using this approach, the GNF dataset was reduced from 44,760 to 36,549 probes expressed in at least one tissue. The immune dataset from Chtanova et al. was reduced from 22,283 to 10,723 probes expressed in at least one tissue. All calculation and heatmaps were generated based on the set of expressed probes only. Expression profiles were clustered using Cluster 3 and visualized using JavaTreeView [96].

For the purpose of visualization and clustering, a single probe with the largest expression range across all tissues was selected for genes with multiple probes and replicates were collapsed to the average expression value for each probe.

Immune enrichment was calculated with the program R (version 2.5) using the Wilcoxon rank sum test for each probe and p-values were corrected using the Bonferroni method. The following tissues in the GNF dataset were classified as immune and tested versus all other tissues: bone marrow, CD19 B cells, tonsils, lymph nodes, thymus, CD4 T cells, CD8 T cells, CD56 T cells, whole blood, CD33 myeloid cells, CD14 monocytes, dendritic cells, fetal liver, CD105 endothelial cells, leukemia cell lines, lymphoma cell lines and erythroid cells.

Statistical significance for immune and brain enrichment in GNF was performed by randomly sampling the same number of probes as in the group being tested and calculating their enrichment. This process was iterated 1000 times and the number of times for which the same or higher enrichment was observed randomly was divided by 1000 to obtain a p-value.

HIV life cycle map. Genes were placed in the HIV life cycle based on annotations from UniProt [60], NCBI GeneRIF, NCBI OMIM database and Gene Ontology[60]. For each gene a PubMed search with the gene name and synonym was performed with keywords such as HIV, retrovirus and viral. We manually placed the genes on the map in places that make most sense in the context of inhibiting HIV infection. The level of confidence for placing each gene varies depending on the available information for that gene.

### LITERATURE CITED

1. Frankel, A. D. and J. A. Young, HIV-1: fifteen proteins and an RNA. Annu Rev Biochem, 1998. 67: p. 1-25.
2. Gomez, C. and T. J. Hope, The ins and outs of HIV replication. Cell Microbiol, 2005. 7(5): p. 621-6.
3. Nisole, S, and A. Saib, Early steps of retrovirus replicative cycle. Retrovirology, 2004. 1: p. 9.
4. Zheng, Y. H., N. Lovsin, and B. M. Peterlin, Newly identified host factors modulate HIV replication. Immunol Lett, 2005. 97(2): p. 225-34.
5. Zheng, Y. H. and B. M. Peterlin, Intracellular immunity to HIV-1: newly defined retroviral battles inside infected cells. Retrovirology, 2005. 2: p. 25.
6. Goff, S. P., Host factors exploited by retroviruses. Nat Rev Microbiol, 2007. 5(4): p. 253-63.
7. Komano, J., et al., Inhibiting the Arp2/3 complex limits infection of both intracellular mature vaccinia virus and primate lentiviruses. Mol Biol Cell, 2004. 15(12): p. 5197-207.
8. McDonald, D., et al., Visualization of the intracellular behavior of HIV in living cells. J Cell Biol, 2002. 159(3): p. 441-52.
9. Bukrinsky, M., A hard way to the nucleus. Mol Med, 2004. 10(1-6): p. 1-5.
10. Schroder, A. R., et al., HIV-1 integration in the human genome favors active genes and local hotspots. Cell, 2002. 110(4): p. 521-9.
11. Ciuffi, A., et al., A role for LEDGF/p75 in targeting HIV DNA integration. Nat Med, 2005. 11(12): p. 1287-9.
12. Llano, M., et al., An essential role for LEDGF/p75 in HIV integration. Science, 2006. 314(5798): p. 461-4.
13. Shun, M. C., et al., LEDGF/p75 functions downstream from preintegration complex formation to effect gene-specific HIV-1 integration. Genes Dev, 2007. 21(14): p. 1767-78.
14. Pereira, L. A., et al., A compilation of cellular transcription factor interactions with the HIV-1 LTR promoter. Nucleic Acids Res, 2000. 28(3): p. 663-8.
15. Cullen, B. R., Nuclear RNA export. J Cell Sci, 2003. 116(Pt 4): p. 587-97.
16. Modem, S., et al., Sam68 is absolutely required for Rev function and HIV-1 production. Nucleic Acids Res, 2005. 33(3): p. 873-9.
17. Yedavalli, V. S., et al., Requirement of DDX3 DEAD box RNA helicase for HIV-1 Rev-RRE export function. Cell, 2004. 119(3): p. 381-92.
18. Strack, B., et al., AIP1/ALIX is a binding partner for HIV-1 p6 and EIAV p9 functioning in virus budding. Cell, 2003. 114(6): p. 689-99.
19. Garrus, J. E., et al., Tsg101 and the vacuolar protein sorting pathway are essential for HIV-1 budding. Cell, 2001. 107(1): p. 55-65.
20. von Schwedler, U. K., et al., The protein network of HIV budding. Cell, 2003. 114(6): p. 701-13.
21. Bouamr, F., et al., The C-terminal portion of the Hrs protein interacts with Tsg101 and interferes with human immunodeficiency virus type 1 Gag particle production. J Virol, 2007. 81(6): p. 2909-22.
22. Deneka, M., et al., In macrophages, HIV-1 assembles into an intracellular plasma membrane domain containing the tetraspanins CD81, CD9, and CD53. J Cell Biol, 2007. 177(2): p. 329-41.
23. Welsch, S., et al., HIV-1 buds predominantly at the plasma membrane of primary human macrophages. PLoS Pathog, 2007. 3(3): p. e36.
24. Murray, J. L., et al., Rab9 GTPase is required for replication of human immunodeficiency virus type 1, filoviruses, and measles virus. J Virol, 2005. 79(18): p. 11742-51.
25. Wei, X., et al., Emergence of resistant human immunodeficiency virus type 1 in patients receiving fusion inhibitor (T-20) monotherapy. Antimicrob Agents Chemother, 2002. 46(6): p. 1896-905.
26. Harel, A., et al., Removal of a single pore subcomplex results in vertebrate nuclei devoid of nuclear pores. Mol Cell, 2003. 11(4): p. 853-64.
27. Krull, S., et al., Nucleoporins as components of the nuclear pore complex core structure and Tpr as the architectural element of the nuclear basket. Mol Biol Cell, 2004. 15(9): p. 4261-77.
28. Kornberg, R. D., Mediator and the mechanism of transcriptional activation. Trends Biochem Sci, 2005. 30(5): p. 235-9.
29. Hampsey, M. and D. Reinberg, RNA polymerase II as a control panel for multiple coactivator complexes. Curr Opin Genet Dev, 1999. 9(2): p. 132-9.
30. Taatjes, D. J., T. Schneider-Poetsch, and R. Tjian, Distinct conformational states of nuclear receptor-bound CRSP-Med complexes. Nat Struct Mol Biol, 2004. 11(7): p. 664-71.
31. Oka, T., et al., Genetic analysis of the subunit organization and function of the conserved oligomeric golgi (COG) complex: studies of COG5- and COG7-deficient mammalian cells. J Biol Chem, 2005. 280(38): p. 32736-45.
32. Kim, Y. G., et al., The architecture of the multisubunit TRAPP I complex suggests a model for vesicle tethering. Cell, 2006. 127(4): p. 817-30.
33. Alving, C. R., et al., HIV-1, lipid rafts, and antibodies to liposomes: implications for anti-viral-neutralizing antibodies. Mol Member Biol, 2006. 23(6): p. 453-65.
34. Levine, B. and D. J. Klionsky, Development by self-digestion: molecular mechanisms and biological functions of autophagy. Dev Cell, 2004. 6(4): p. 463-77.
35. Grosshans, B. L., D. Ortiz, and P. Novick, Rabs and their effectors: achieving specificity in membrane traffic. Proc Natl Acad Sci USA, 2006. 103(32): p. 11821-7.
36. Del Nery, E., et al., Rab6A and Rab6A′ GTPases play non-overlapping roles in membrane trafficking. Traffic, 2006. 7(4): p. 394-407.
37. Girod, A., et al., Evidence for a COP-I-independent transport route from the Golgi complex to the endoplasmic reticulum. Nat Cell Biol, 1999. 1(7): p. 423-30.
38. Utskarpen, A., et al., Transport of ricin from endosomes to the Golgi apparatus is regulated by Rab6A and Rab6A′. Traffic, 2006. 7(6): p. 663-72.
39. Luo, Z. and D. Gallwitz, Biochemical and genetic evidence for the involvement of yeast Ypt6-GTPase in protein retrieval to different Golgi compartments. J Biol Chem, 2003. 278(2): p. 791-9.
40. Siniossoglou, S., S. Y. Peak-Chew, and H. R. Pelham, Ric1p and Rgp1p form a complex that catalyses nucleotide exchange on Ypt6p. EMBO J, 2000. 19(18): p. 4885-94.
41. Ciminale, V., et al., A bioassay for HIV-1 based on Env-CD4 interaction. AIDS Res Hum Retroviruses, 1990. 6(11): p. 1281-7.
42. Conibear, E. and T. H. Stevens, Vps52p, Vps53p, and Vps54p form a novel multisubunit complex required for protein sorting at the yeast late Golgi. Mol Biol Cell, 2000. 11(1): p. 305-23.
43. Quenneville, N. R., et al., Domains within the GARP subunit Vps54 confer separate functions in complex assembly and early endosome recognition. Mol Biol Cell, 2006. 17(4): p. 1859-70.
44. Conibear, E., J. N. Cleck, and T. H. Stevens, Vps51p mediates the association of the GARP (Vps52/53/54) complex with the late Golgi t-SNARE Tlg1p. Mol Biol Cell, 2003. 14(4): p. 1610-23.
45. Reggiori, F., et al., Vps51 is part of the yeast Vps fifty-three tethering complex essential for retrograde traffic from the early endosome and Cvt vesicle completion. J Biol Chem, 2003. 278(7): p. 5009-20.
46. Siniossoglou, S, and H. R. Pelham, An effector of Ypt6p binds the SNARE Tlg1p and mediates selective fusion of vesicles with late Golgi membranes. EMBO J, 2001. 20(21): p. 5991-8.
47. Siniossoglou, S, and H. R. Pelham, Vps51p links the VFT complex to the SNARE Tlg1p. J Biol Chem, 2002. 277(50): p. 48318-24.
48. Erkmann, J. A., et al., Nuclear import of the stem-loop binding protein and localization during the cell cycle. Mol Biol Cell, 2005. 16(6): p. 2960-71.
49. Kataoka, N., J. L. Bachorik, and G. Dreyfuss, Transportin-SR, a nuclear import receptor for SR proteins. J Cell Biol, 1999. 145(6): p. 1145-52.
50. Lai, M. C., et al., A human importin-beta family protein, transportin-5R2, interacts with the phosphorylated RS domain of SR proteins. J Biol Chem, 2000. 275(11): p. 7950-7.
51. Allemand, E., et al., A conserved Drosophila transportin-serine/arginine-rich (SR) protein permits nuclear import of Drosophila SR protein splicing factors and their antagonist repressor splicing factor 1. Mol Biol Cell, 2002. 13(7): p. 2436-47.
52. Bukrinsky, M. I., et al., Active nuclear import of human immunodeficiency virus type 1 preintegration complexes. Proc Natl Acad Sci USA, 1992. 89(14): p. 6580-4.
53. Katz, R. A., et al., Human immunodeficiency virus type 1 DNA nuclear import and integration are mitosis independent in cycling cells. J Virol, 2003. 77(24): p. 13412-7.
54. Roe, T., et al., Integration of murine leukemia virus DNA depends on mitosis. EMBO J, 1993. 12(5): p. 2099-108.
55. Ashida, H., Y. Maeda, and T. Kinoshita, DPM1, the catalytic subunit of dolichol-phosphate mannose synthase, is tethered to and stabilized on the endoplasmic reticulum membrane by DPM3. J Biol Chem, 2006. 281(2): p. 896-904.
56. Kelleher, D. J. and R. Gilmore, An evolving view of the eukaryotic oligosaccharyltransferase. Glycobiology, 2006. 16(4): p. 47R-62R.
57. Wolk, T. and M. Schreiber, N-Glycans in the gp120 V1/V2 domain of the HIV-1 strain NL4-3 are indispensable for viral infectivity and resistance against antibody neutralization. Med Microbiol Immunol, 2006. 195(3): p. 165-72.
58. Blough, H. A., et al., Glycosylation inhibitors block the expression of LAV/HTLV-III (HIV) glycoproteins. Biochem Biophys Res Commun, 1986. 141(1): p. 33-8.
59. Balzarini, J., Targeting the glycans of glycoproteins: a novel paradigm for antiviral therapy. Nat Rev Microbiol, 2007. 5(8): p. 583-97.
60. Chabot, D. J., et al., N-linked glycosylation of CXCR4 masks coreceptor function for CCR5-dependent human immunodeficiency virus type 1 isolates. J Virol, 2000. 74(9): p. 4404-13.
61. Wang, J., et al., N-linked glycosylation in the CXCR4 N-terminus inhibits binding to HIV-1 envelope glycoproteins. Virology, 2004. 324(1): p. 140-50.
62. Tifft, C. J., R. L. Proia, and R. D. Camerini-Otero, The folding and cell surface expression of CD4 requires glycosylation. J Biol Chem, 1992. 267(5): p. 3268-73.
63. Futerman, A. H. and H. Riezman, The ins and outs of sphingolipid synthesis. Trends Cell Biol, 2005. 15(6): p. 312-8.
64. Conde, R., et al., Screening for new yeast mutants affected in mannosylphosphorylation of cell wall mannoproteins. Yeast, 2003. 20(14): p. 1189-211.
65. Hug, P., et al., Glycosphingolipids promote entry of a broad range of human immunodeficiency virus type 1 isolates into cell lines expressing CD4, CXCR4, and/or CCR5. J Virol, 2000. 74(14): p. 6377-85.
66. Nehete, P. N., et al., A post-CD4-binding step involving interaction of the V3 region of viral gp120 with host cell surface glycosphingolipids is common to entry and infection by diverse HIV-1 strains. Antiviral Res, 2002. 56(3): p. 233-51.
67. Puri, A., et al., An inhibitor of glycosphingolipid metabolism blocks HIV-1 infection of primary T-cells. AIDS, 2004. 18(6): p. 849-58.
68. D'Angelo, G., et al., Glycosphingolipid synthesis requires FAPP2 transfer of glucosylceramide. Nature, 2007. 449(7158): p. 62-7.
69. Godi, A., et al., FAPPs control Golgi-to-cell-surface membrane traffic by binding to ARF and PtdIns (4)P. Nat Cell Biol, 2004. 6(5): p. 393-404.
70. Parolini, I., et al., Signal transduction and glycophosphatidylinositol-linked proteins (lyn, lck, CD4, CD45, G proteins, and CD55) selectively localize in Triton-insoluble plasma membrane domains of human leukemic cell lines and normal granulocytes. Blood, 1996. 87(9): p. 3783-94.
71. Parolini, I., et al., Phorbol ester-induced disruption of the CD4-Lck complex occurs within a detergent-resistant microdomain of the plasma membrane. Involvement of the translocation of activated protein kinase C isoforms. J Biol Chem, 1999. 274(20): p. 14176-87.
72. Ono, A., A. A. Waheed, and E. O. Freed, Depletion of cellular cholesterol inhibits membrane binding and higher-order multimerization of human immunodeficiency virus type 1 Gag. Virology, 2007. 360(1): p. 27-35.
73. Alpy, F. and C. Tomasetto, MLN64 and MENTHO, two mediators of endosomal cholesterol transport. Biochem Soc Trans, 2006. 34(Pt 3): p. 343-5.
74. Fahrenkrog, B. and U. Aebi, The nuclear pore complex: nucleocytoplasmic transport and beyond. Nat Rev Mol Cell Biol, 2003. 4(10): p. 757-66.
75. Fahrenkrog, B., J. Koser, and U. Aebi, The nuclear pore complex: a jack of all trades? Trends Biochem Sci, 2004. 29(4): p. 175-82.
76. Tran, E. J. and S. R. Wente, Dynamic nuclear pore complexes: life on the edge. Cell, 2006. 125(6): p. 1041-53.
77. Stewart, M., Structural basis for the nuclear protein import cycle. Biochem Soc Trans, 2006. 34(Pt 5): p. 701-4.
78. Varadarajan, P., et al., The functionally conserved nucleoporins Nup124p from fission yeast and the human Nup153 mediate nuclear import and activity of the Tf1 retrotransposon and HIV-1 Vpr. Mol Biol Cell, 2005. 16(4): p. 1823-38.
79. Wu, J., et al., Nup358, a cytoplasmically exposed nucleoporin with peptide repeats, Ran-GTP binding sites, zinc fingers, a cyclophilin A homologous domain, and a leucine-rich region. J Biol Chem, 1995. 270(23): p. 14209-13.
80. Sabri, N., et al., Distinct functions of the Drosophila Nup153 and Nup214 FG domains in nuclear protein transport. J Cell Biol, 2007. 178(4): p. 557-65.
81. Neville, M., et al., The importin-beta family member Crm1p bridges the interaction between Rev and the nuclear pore complex during nuclear export. Curr Biol, 1997. 7(10): p. 767-75.
82. Folkman, J., Angiogenesis. Annu Rev Med, 2006. 57: p. 1-18.
83. Folkman, J., Antiangiogenesis in cancer therapy—endostatin and its mechanisms of action. Exp Cell Res, 2006. 312(5): p. 594-607.
84. He, J. and N. R. Landau, Use of a novel human immunodeficiency virus type 1 reporter virus expressing human placental alkaline phosphatase to detect an alternative viral receptor. J Virol, 1995. 69(7): p. 4587-92.
85. Schroers, R., et al., Transduction of human PBMC-derived dendritic cells and macrophages by an HIV-1-based lentiviral vector system. Mol Ther, 2000. 1(2): p. 171-9.
86. Sutton, R. E., et al., Human immunodeficiency virus type 1 vectors efficiently transduce human hematopoietic stem cells. J Virol, 1998. 72(7): p. 5781-8.
87. Silva, J. M., et al., Second-generation shRNA libraries covering the mouse and human genomes. Nat. Genet., 2005. 37(11): p. 1281-8.
88. Butler, S. L., M. S. Hansen, and F. D. Bushman, A quantitative assay for HIV DNA integration in vivo. Nat Med, 2001. 7(5): p. 631-4.
89. Gentleman, R. C., et al., Bioconductor: open software development for computational biology and bioinformatics. Genome Biol, 2004. 5(10): p. R80.
90. Bairoch, A., et al., Swiss-Prot: juggling between evolution and stability. Brief Bioinform, 2004. 5(1): p. 39-55.
91. Ashburner, M., et al., Gene ontology: tool for the unification of biology. The Gene Ontology Consortium. Nat. Genet, 2000. 25(1): p. 25-9.
92. Kall, L., A. Krogh, and E. L. Sonnhammer, A combined transmembrane topology and signal peptide prediction method. J Mol Biol, 2004. 338(5): p. 1027-36.
93. Calvo, S., et al., Systematic identification of human mitochondrial disease genes through integrative genomics. Nat Genet, 2006. 38(5): p. 576-82.
94. Emanuelsson, O., et al., Locating proteins in the cell using TargetP, SignalP and related tools. Nat Protoc, 2007. 2(4): p. 953-71.
95. Chtanova, T., et al., T follicular helper cells express a distinctive transcriptional profile, reflecting their role as non-Th1/Th2 effector cells that provide help for B cells. J Immunol, 2004. 173(1): p. 68-78.
96. Saldanha, A. J., Java Treeview—extensible visualization of microarray data. Bioinformatics, 2004. 20(17): p. 3246-8.

TABLE 2

| | | Beta- | | | | | | | |
|---|---|---|---|---|---|---|---|---|---|
| | | gal | | | | | | | |
| | | siRNAs | | | | | Beta-gal | | |
| | p24 | (scored | | | | | siRNAs | | |
| | siRNAs | in part | SMARTpool | | | | (scored in | | |
| | (part | two | Cat | Locu | | p24 siRNAs | part two | SMARTpool Cat | |
| Symbol | one) | only) | Number | link | Symbol | (part one) | only) | Number | Loculink |
| SEPT8 | 1 | | M-010647-00 | 23176 | LPL | 1 | 1 | M-008970-00 | 4023 |
| A4GALT | 2 | | M-016315-00 | 53947 | LRRC8D | | 1 | M-015747-00 | 55144 |
| ADAM10 | 2 | 1 | M-004503-01 | 102 | LSM3 | 1 | | M-020240-00 | 27258 |
| AGBL5 | 2 | | M-009468-00 | 60509 | LY6D | 1 | | M-012615-00 | 8581 |
| AKT1 | 2 | | M-003000-01 | 207 | LYPD4 | 1 | 1 | M-018514-00 | 147719 |
| ALKBH8 | 2 | | M-016544-00 | 91801 | MAP4 | 3 | | M-011724-00 | 4134 |
| ANKRD30A | 1 | | M-008466-00 | 91074 | MDN1 | | 2 | M-009786-00 | 23195 |
| ANKRD43 | 1 | | M-017945-00 | 134548 | MED14 | 3 | | M-011928-00 | 9282 |
| ANKRD6 | 1 | 1 | M-020396-00 | 22881 | MED28 | 4 | | M-014606-00 | 80306 |
| ANKRD9 | 2 | | M-015551-00 | 122416 | MED4 | 2 | | M-020687-00 | 29079 |
| AP2M1 | 1 | | M-008170-00 | 1173 | MED6 | 4 | | M-019963-00 | 10001 |
| ARF1 | 2 | | M-011580-00 | 375 | MED7 | 2 | 1 | M-017313-00 | 9443 |
| ARHGEF12 | 1 | | M-008480-00 | 23365 | MGAT1 | | 1 | M-011332-00 | 4245 |
| ARHGEF19 | 1 | | M-008370-01 | 128272 | MGC59937 | 1 | | M-027279-00 | 375791 |
| ARPC1A | | 1 | M-012263-00 | 10552 | MID1IP1 | 1 | 1 | M-015884-00 | 58526 |
| ASXL2 | | 1 | M-022638-00 | 55252 | MKRN2 | 2 | | M-006960-00 | 23609 |
| ATG12 | 1 | 1 | M-010212-01 | 9140 | MND1 | 1 | 1 | M-014779-00 | 84057 |
| ATG16L2 | | 2 | M-026687-00 | 89849 | MOS | 1 | 1 | M-003859-02 | 4342 |
| ATG7 | SP | | M-020112-00 | 10533 | MPHOSPH6 | | 1 | M-020018-00 | 10200 |
| ATP6V0A1 | | 2 | M-017618-00 | 535 | MR1 | 1 | | M-019619-00 | 3140 |
| BAHD1 | 1 | 1 | M-020357-00 | 22893 | NCOR2 | 2 | 1 | M-020145-01 | 9612 |
| BCL9 | 2 | 2 | M-007268-00 | 607 | NDUFB7 | | 1 | M-017213-00 | 4713 |
| C1orf103 | 2 | | M-018103-00 | 55791 | NF2 | 2 | 2 | M-003917-00 | 4771 |
| C20orf174 | 1 | | M-024186-00 | 128611 | NGLY1 | 2 | | M-016457-00 | 55768 |
| C2orf25 | 1 | 1 | M-013862-00 | 27249 | NIPSNAP3B | 1 | | M-015435-00 | 55335 |
| C4orf33 | 1 | | M-018450-00 | 132321 | NMT1 | | 3 | M-004316-00 | 4836 |
| C6orf1 | 1 | 1 | M-017897-00 | 221491 | NR0B2 | 1 | | M-003410-00 | 8431 |
| C8orf14 | 3 | | M-015204-00 | 83655 | NUP107 | 2 | | M-020440-00 | 57122 |
| C9orf131 | | 1 | M-031891-00 | 138724 | NUP133 | 1 | 1 | M-013322-00 | 55746 |
| CACNG1 | | 1 | M-011162-00 | 786 | NUP153 | 2 | | M-005283-00 | 9972 |
| CAPN6 | 2 | | M-009423-00 | 827 | NUP155 | 2 | | M-011967-00 | 9631 |
| CAV2 | 2 | 1 | M-010958-00 | 858 | NUP160 | 3 | | M-029990-00 | 23279 |
| CCDC134 | 1 | | M-014466-00 | 79879 | NUP85 | 2 | | M-014478-00 | 79902 |
| CCNT1 | SP | | M-003220-02 | 904 | OST48 | | 3 | M-015786-00 | 1650 |
| | | | | | (DDOST) | | | | |
| CD4 | 2 | | M-005234-01 | 920 | OTUD3 | 2 | | M-027582-00 | 23252 |
| CENTG1 | 2 | 1 | M-021010-00 | 116986 | PANK1 | SP | | M-004057-02 | 53354 |
| CLDND1 | 2 | | M-020682-00 | 56650 | PCDH11X | 1 | | M-013619-00 | 27328 |
| CLN3 | 1 | 1 | M-019282-00 | 1201 | PDIA6 | | 2 | M-020026-00 | 10130 |
| CLNS1A | 1 | | M-012571-00 | 1207 | PHF12 | 2 | 2 | M-009736-00 | 57649 |
| COG2 | 3 | | M-019487-00 | 22796 | PHF3 | 1 | | M-014075-00 | 23469 |
| COG3 | 4 | | M-013499-00 | 83548 | PIGH | | 1 | M-011885-00 | 5283 |
| COG4 | 3 | | M-013993-00 | 25839 | PIGK | 1 | | M-005996-01 | 10026 |
| COP1 | 1 | | M-004411-00 | 114769 | PIGY | 1 | 1 | M-015043-00 | 84992 |
| CRIPAK | 1 | | M-018504-00 | 285464 | PIP5K1C | | 3 | M-004782-00 | 23396 |
| CRTC2 | 1 | | M-018947-00 | 200186 | PKD1L2 | 1 | | M-013421-00 | 114780 |
| CRTC3 | 3 | | M-014210-00 | 64784 | PLOD3 | 1 | | M-004286-00 | 8985 |
| CSPP1 | 2 | | M-016485-00 | 79848 | PNRC1 | 1 | | M-019926-00 | 10957 |
| CTDP1 | 1 | | M-009326-01 | 9150 | POLR3A | 1 | | M-019741-00 | 11128 |
| CXCR4 | 2 | | M-005139-01 | 7852 | POLR3F | 1 | | M-019240-00 | 10621 |
| CXorf50 | | 2 | M-018780-00 | 203429 | POU1F1 | 1 | | M-012546-00 | 5449 |
| DDX10 | 1 | | M-011842-00 | 1662 | PPIB | 1 | 1 | M-004606-00 | 5479 |
| DDX3X | SP | | M-006874-00 | 1654 | PPP2R2A | | 2 | M-004824-01 | 5520 |
| DDX53 | 1 | | M-019305-00 | 168400 | PRDM14 | | 3 | M-014346-00 | 63978 |
| DDX55 | | 3 | M-027082-00 | 57696 | PRDM7 | | 2 | M-015181-00 | 11105 |
| DEPDC5 | 1 | | M-020708-00 | 9681 | PRKX | 1 | | M-004660-01 | 5613 |
| DHX33 | 2 | | M-017205-00 | 56919 | PSME2 | 1 | | M-011370-00 | 5721 |
| DIMT1L | 4 | | M-009476-00 | 27292 | PURA | 1 | 1 | M-012136-00 | 5813 |
| DKFZp686O24166 | 1 | 2 | M-030031-00 | 374383 | RAB1B | | 3 | M-008958-00 | 81876 |
| DMXL1 | 2 | | M-012091-00 | 1657 | RAB28 | 1 | | M-008582-00 | 9364 |
| DNAJB1 | 2 | 1 | M-012735-00 | 3337 | RAB2A | 3 | 1 | M-010533-01 | 5862 |
| DNAL1 | 1 | 1 | M-014722-00 | 83544 | RAB6A | 3 | | M-008975-01 | 5870 |
| DOK6 | 2 | | M-015595-00 | 220164 | Rab9p40 | | 3 | M-019457-00 | 10244 |
| DPM1 | | 2 | M-011535-00 | 8813 | RANBP1 | SP | | M-006627-01 | 5902 |
| DYSF | 1 | | M-003652-01 | 8291 | RANBP2 | 2 | | M-004746-01 | 5903 |
| EDNRA | | 2 | M-005485-00 | 1909 | RAP1B | 1 | 1 | M-010364-01 | 5908 |
| EFHC2 | 1 | 1 | M-018562-00 | 80258 | RAPGEF1 | 1 | | M-006840-00 | 2889 |
| EGF | 1 | | M-011650-00 | 1950 | RELA | 3 | 1 | M-003533-01 | 5970 |
| EGFR | 1 | | M-003114-01 | 1956 | RGP1 | SP | | M-021128-00 | 9827 |
| EIF2C3 | 2 | 2 | M-004640-00 | 192669 | RGPD5 | 3 | | M-012007-00 | 84220 |
| EIF3H | 1 | | M-003883-00 | 8667 | RICS | 2 | | M-008213-00 | 9743 |
| EPS8 | 1 | | M-017905-00 | 2059 | RIMS4 | | 3 | M-021322-00 | 140730 |
| ERCC3 | 1 | 2 | M-011028-00 | 2071 | RNF170 | 2 | 1 | M-007078-00 | 81790 |
| ERP27 | 1 | | M-015698-00 | 121506 | RNF26 | 1 | | M-007060-00 | 79102 |
| ETF1 | 1 | 1 | M-019840-00 | 2107 | RPTN | 2 | 1 | M-027449-00 | 126638 |
| ETHE1 | 3 | | M-012508-00 | 23474 | RRAGB | 1 | | M-012189-00 | 10325 |
| EXOD1 | 2 | | M-015252-00 | 112479 | RSL1D1 | 1 | 1 | M-022489-00 | 26156 |
| EXOSC3 | 1 | 1 | M-031955-00 | 51010 | RTN2 | 1 | | M-012717-00 | 6253 |
| EXOSC5 | 1 | 1 | M-020482-00 | 56915 | RUSC2 | 1 | 1 | M-026133-00 | 9853 |
| FAM5B | | 1 | M-014022-00 | 57795 | SCFD1 | 2 | 1 | M-010943-00 | 23256 |
| FAM76B | | 1 | M-015721-00 | 143684 | SEC14L1 | 2 | | M-011386-00 | 6397 |
| FAPP1 | 1 | | M-004319-00 | 65977 | SESTD1 | 2 | | M-018379-00 | 91404 |
| FBXO18 | 1 | | M-017404-00 | 84893 | SFT2D1 | 2 | | M-016199-00 | 113402 |
| FBXO21 | 1 | | M-012917-00 | 23014 | SIP1 | 1 | | M-019545-00 | 8487 |
| FBXW11 | 2 | 2 | M-003490-00 | 23291 | SLC46A1 | 1 | | M-018653-00 | 113235 |
| FGD6 | 1 | 2 | M-026895-00 | 55785 | SP110 | | 2 | M-011875-01 | 3431 |
| FHL3 | 2 | 2 | M-019805-00 | 2275 | SPAST | 2 | | M-014070-00 | 6683 |
| FKSG2 | 2 | | M-004427-01 | 59347 | SPCS3 | | 2 | M-010124-00 | 60559 |
| FLII | 2 | | M-017506-00 | 2314 | SPTAN1 | 1 | 2 | M-009933-00 | 6709 |
| FLJ10154 | | 2 | M-021093-00 | 55082 | SPTBN1 | 3 | | M-018149-00 | 6711 |
| FLJ32569 | 1 | 1 | M-016737-00 | 148811 | SSB | SP | | M-006877-00 | 6741 |
| FLJ46026 | 1 | 1 | M-032143-00 | 400627 | ST3GAL5 | 1 | | M-011546-00 | 8869 |
| FLJ46066 | 2 | | | 401103 | STAC2 | | 2 | M-027277-00 | 342667 |
| FLJ90680 | | 2 | M-032160-00 | 400926 | STARD3NL | 2 | | M-018591-00 | 83930 |
| FNTA | | 4 | M-008807-01 | 2339 | STT3A | | 2 | M-017073-00 | 3703 |
| GABARAPL2 | | 1 | M-006853-01 | 11345 | STX5 | 2 | 1 | M-017768-00 | 6811 |
| (ATG8) | | | | | | | | | |
| GAPVD1 | 1 | 1 | M-026206-00 | 26130 | SUV420H1 | 2 | | M-013366-00 | 51111 |
| GBAS | 2 | | M-011282-00 | 2631 | TAOK1 | 1 | 1 | M-004846-01 | 57551 |
| GCK | 1 | | M-010819-01 | 2645 | TCEB3 | 1 | 1 | M-005143-01 | 6924 |
| GCN5L2 | 1 | | M-009722-00 | 2648 | TFAP4 | SP | | M-009504-00 | 7023 |
| GML | 2 | | M-019639-00 | 2765 | TFDP2 | 1 | | M-003328-02 | 7029 |
| GOLPH3 | | 2 | M-006414-00 | 64083 | TFE3 | 2 | | M-009363-01 | 7030 |
| GOSR2 | 2 | 1 | M-010980-00 | 9570 | THAP3 | 1 | | M-031883-00 | 90326 |
| GRTP1 | 1 | 1 | M-014422-00 | 79774 | THOC2 | 1 | | M-025006-00 | 57187 |
| H3F3A | 1 | | M-011684-00 | 3020 | TIAM2 | | 1 | M-008434-00 | 26230 |
| HEATR1 | 3 | | M-015939-00 | 55127 | TIMM8A | | 2 | M-010342-00 | 1678 |
| HGS | | SP | | 9146 | TM9SF2 | 3 | | M-010221-00 | 9375 |
| HIBCH | 1 | | M-009852-00 | 26275 | TMED2 | 3 | 1 | M-008074-00 | 10959 |
| HIP1R | 1 | | M-027079-00 | 9026 | TMEM132C | | 2 | M-027086-00 | 92293 |
| HNRPF | 1 | | M-013449-00 | 3185 | TMEM163 | 1 | | M-014673-00 | 81615 |
| HTATSF1 | 1 | | M-016645-00 | 27336 | TMEM181 | 3 | | M-024897-00 | 57583 |
| HUWE1 | | 2 | M-007185-01 | 10075 | TMTC1 | 2 | | M-016838-00 | 83857 |
| IDH1 | 1 | | M-008294-00 | 3417 | TNPO3 | 4 | | M-019949-00 | 23534 |
| IGHMBP2 | 1 | | M-019657-00 | 3508 | TOMM70A | 2 | 1 | M-021243-00 | 9868 |
| IKBKG | 2 | | M-003767-00 | 8517 | TOR2A | | 1 | M-015292-00 | 27433 |
| INTS7 | 1 | 2 | M-013972-00 | 25896 | TRAPPC1 | 3 | | M-013781-00 | 58485 |
| IQUB | 1 | | M-018861-00 | 154865 | TRIM55 | 2 | | M-007092-00 | 84675 |
| ITPKA | 1 | 1 | M-006742-01 | 3706 | TRIM58 | 1 | | M-013985-00 | 25893 |
| JAK1 | 1 | | M-003145-01 | 3716 | TRMT5 | 1 | | M-021968-00 | 57570 |
| JHDM1D | | 1 | M-025357-00 | 80853 | TUBAL3 | | 1 | M-009010-00 | 79861 |
| JMJD2D | 1 | | M-020709-00 | 55693 | UBQLN4 | | 1 | M-021178-00 | 56893 |
| KBTBD7 | 2 | | M-015708-00 | 84078 | USP26 | 1 | 1 | M-006075-01 | 83844 |
| KCNIP3 | 1 | | M-017332-00 | 30818 | USP6 | 2 | | M-006096-02 | 9098 |
| KCNK9 | 1 | 1 | M-004891-01 | 51305 | VPRBP | 1 | | M-021119-00 | 9730 |
| KEL | 1 | | M-005903-00 | 3792 | VPS53 | 3 | | M-017048-00 | 55275 |
| KIAA1012 | 4 | | M-010645-00 | 22878 | WDTC1 | 1 | | M-016542-00 | 23038 |
| KIF3C | | 1 | M-009469-01 | 3797 | WNK1 | 2 | 1 | M-005362-00 | 65125 |
| KLHDC2 | | 2 | M-012839-00 | 23588 | WNT1 | 3 | | M-003937-00 | 7471 |
| KLHL1 | 1 | 1 | M-010912-00 | 57626 | WTH3 | 1 | 1 | M-009031-01 | 84084 |
| LAPTM5 | 2 | | M-019880-00 | 7805 | XKR4 | 1 | 1 | M-025942-00 | 114786 |
| LARS | 1 | | M-010171-00 | 51520 | YTHDC2 | 1 | | M-014220-00 | 64848 |
| LCP2 | 2 | 2 | M-012120-00 | 3937 | ZBTB2 | 1 | | M-014129-00 | 57621 |
| LEFTY1 | 1 | 2 | M-013114-00 | 10637 | ZNF12 | 1 | | M-032513-00 | 7559 |
| LNX2 | 1 | 2 | M-007164-00 | 222484 | ZNF182 | 1 | | M-024670-00 | 7569 |
| LOC26010 | 2 | | M-020248-00 | 26010 | ZNF354A | 2 | | M-007685-01 | 6940 |
| LOC284214 | | 2 | M-031009-00 | 284214 | ZNF436 | 1 | | M-014640-00 | 80818 |
| LOC285550 | | 1 | | 285550 | ZNF512B | | 1 | M-013934-00 | 57473 |
| LOC375190 | 1 | | M-027266-00 | 375190 | ZNF536 | 1 | | M-020506-00 | 9745 |
| LOC390530 | 1 | 1 | M-024218-00 | 390530 | ZNF720 | 2 | | M-022814-00 | 124411 |
| LOC402117 | 1 | | | 402117 | ZNF785 | | 1 | M-018331-00 | 146540 |
| ZNRD1 | 1 | 1 | M-017359-00 | 30834 | ZNF791 | 1 | | M-015752-00 | 163049 |

TABLE 3

| | | | | |
|---|---|---|---|---|
| | | SEQ ID | | |
| Gene Symbol | Gene ID | NO: | siRNA sequence | Dharmacon Cat # |

| | | | | |
|---|---|---|---|---|
| A4GALT | 53947 | 1 | GGACACGGACUUCAUUGUU | D-016315-02 |
| A4GALT | 53947 | 2 | GCACUCAUGUGGAAGUUCG | D-016315-04 |
| A4GALT | 53947 | 3 | AGAAAGGGCAGCUCUAUAA | D-016315-01 |
| A4GALT | 53947 | 4 | UGAAAGGGCUUCCGGGUGG | D-016315-03 |
| ADAM10 | 102 | 5 | CCCAAAGUCUCUCACAUUA | D-004503-04 |
| ADAM10 | 102 | 6 | GCAAGGGAAGGAAUAUGUA | D-004503-05 |
| ADAM10 | 102 | 7 | GGACAAACUUAACAACAAU | D-004503-03 |
| ADAM10 | 102 | 8 | GCUAAUGGCUGGAUUUAUU | D-004503-01 |
| AGBL5 | 60509 | 9 | CUACAAAGCCUCAGGGAUA | D-009468-01 |
| AGBL5 | 60509 | 10 | GCACAGCAGCCUUACUAAU | D-009468-04 |
| AGBL5 | 60509 | 11 | GAAUGUGGGUGUCAACAAG | D-009468-03 |
| AGBL5 | 60509 | 12 | GCUGAAGCCUGGAAACAAA | D-009468-02 |
| AK7 | 122481 | 13 | GGGCGAGAUUCCUGCAUUA | D-007257-01 |
| AK7 | 122481 | 14 | GAAAUUCACCCGAUACAUA | D-007257-02 |
| AK7 | 122481 | 15 | GAAAGUCUCAUCCUAAUUU | D-007257-03 |
| AK7 | 122481 | 16 | UGAAGAAGAUUAUCGAAGA | D-007257-04 |
| AKT1 | 207 | 17 | GACCGCCUCUGCUUUGUCA | D-003000-08 |
| AKT1 | 207 | 18 | GGACAAGGACGGGCACAUU | D-003000-06 |
| AKT1 | 207 | 19 | GACAAGGACGGGCACAUUA | D-003000-05 |
| AKT1 | 207 | 20 | GCUACUUCCUCCUCAAGAA | D-003000-07 |
| ALKBH8 | 91801 | 21 | GAGCCUGGUUGUUGCCAAU | D-016544-04 |
| ALKBH8 | 91801 | 22 | GCAUUGAGACAGUAUCCUA | D-016544-01 |
| ALKBH8 | 91801 | 23 | CGUACUCAUUUGCAAGAUA | D-016544-03 |
| ALKBH8 | 91801 | 24 | CAGCAACCAUCAAAGUAAU | D-016544-02 |
| ANKRD28 | 23243 | 25 | UCAGAAUGCUUACGGCUAU | D-023451-02 |
| ANKRD28 | 23243 | 26 | GUUCGAGCACUAAUAUUUA | D-023451-03 |
| ANKRD28 | 23243 | 27 | GUAAUCGACUGUGAGGAUA | D-023451-01 |
| ANKRD28 | 23243 | 28 | CUAGAGGUGCCAAUAUUAA | D-023451-04 |
| ANKRD30A | 91074 | 29 | GCAAGAGUAACAUCUAAUA | D-008466-04 |
| ANKRD30A | 91074 | 30 | UGAAGGACAUGCAAACUUU | D-008466-03 |
| ANKRD30A | 91074 | 31 | GCAGAUAUAUGUGGAGUAA | D-008466-01 |
| ANKRD30A | 91074 | 32 | CGAAUGCAGUUAAUAAGUA | D-008466-02 |
| ANKRD43 | 134548 | 33 | GCGCACCAGUCGACGUGAA | D-017945-04 |
| ANKRD43 | 134548 | 34 | GCCCAUGGCUCCACGUAAA | D-017945-01 |
| ANKRD43 | 134548 | 35 | GCCUUUAGCUGGUCUAGUG | D-017945-02 |
| ANKRD43 | 134548 | 36 | GCCCGAGGCUUGAAGAAGU | D-017945-03 |
| ANKRD6 | 22881 | 37 | CAAGAUAAGGCUACAUUGA | D-020396-02 |
| ANKRD6 | 22881 | 38 | UAUCAGCUCUACACAUUGU | D-020396-03 |
| ANKRD6 | 22881 | 39 | CAGAGGCACUCAAACUAAG | D-020396-04 |
| ANKRD6 | 22881 | 40 | GCAGAUACGACCAUUGUUA | D-020396-01 |
| ANKRD9 | 122416 | 41 | ACCAAGCGUACGCGCAUUA | D-015551-03 |
| ANKRD9 | 122416 | 42 | GCAAGUCGUCGUUCGCCUU | D-015551-01 |
| ANKRD9 | 122416 | 43 | CGUUGGACCUCACUGGCAA | D-015551-02 |
| ANKRD9 | 122416 | 44 | GCUACAACCGCGUGGGCAU | D-015551-04 |
| ANXA3 | 306 | 45 | GAAGAUGCCUUGAUUGAAA | D-011804-01 |
| ANXA3 | 306 | 46 | GGCCAUAGUUAAUUGUGUG | D-011804-03 |
| ANXA3 | 306 | 47 | UCAAGCCUAUUAUACAGUA | D-011804-02 |
| ANXA3 | 306 | 48 | AGGAAUAUCAAGCAGCAUA | D-011804-04 |
| AP2M1 | 1173 | 49 | GAAGAGCAGUCACAGAUCA | D-008170-02 |
| AP2M1 | 1173 | 50 | UAUAUGAGCUGCUGGAUGA | D-008170-01 |
| AP2M1 | 1173 | 51 | GGAGGCUUAUUCAUCUAUA | D-008170-04 |
| AP2M1 | 1173 | 52 | CGUGAUGGCUGCCUACUUU | D-008170-03 |
| APG7L | 10533 | 53 | GAUCAAAGGUUUUCACUAA | D-020112-02 |
| APG7L | 10533 | 54 | GAAGAUAACAAUUGGUGUA | D-020112-03 |
| APG7L | 10533 | 55 | CAACAUCCCUGGUUACAAG | D-020112-04 |
| APG7L | 10533 | 56 | CCAAAGUUCUUGAUCAAUA | D-020112-01 |
| ARF1 | 375 | 57 | ACGUGGAAACCGUGGAGUA | D-011580-04 |
| ARF1 | 375 | 58 | GACCACCAUUCCCACCAUA | D-011580-01 |
| ARF1 | 375 | 59 | ACAGAGAGCGUGUGAACGA | D-011580-02 |
| ARF1 | 375 | 60 | CGGCCGAGAUCACAGACAA | D-011580-03 |
| ARHGEF12 | 23365 | 61 | GAGAAGACCUGAGCUCAUU | D-008480-03 |
| ARHGEF12 | 23365 | 62 | GGAGGAAUGUGAAGUAGAA | D-008480-02 |
| ARHGEF12 | 23365 | 63 | AGACAGAGAUUUGGGAUUA | D-008480-01 |
| ARHGEF12 | 23365 | 64 | GACAGGAAGUGAUUAAUGA | D-008480-04 |
| ARHGEF19 | 128272 | 65 | AAUGGAGGCUCGAAGUGUA | D-008370-06 |
| ARHGEF19 | 128272 | 66 | GGACAAGCAGUGGCUGUUU | D-008370-03 |
| ARHGEF19 | 128272 | 67 | GAGUCUACCUGCCCUAUGU | D-008370-01 |
| ARHGEF19 | 128272 | 68 | GGAGUGCAAUGCUAGUGUA | D-008370-05 |
| ARPC1A | 10552 | 69 | GCAAGAUUGUCGCAAAUUU | D-012263-01 |
| ARPC1A | 10552 | 70 | GAAAAUGACUGGUGGGUGA | D-012263-02 |
| ARPC1A | 10552 | 71 | ACGAAGUGCACAUCUAUAA | D-012263-03 |
| ARPC1A | 10552 | 72 | GAAUUAAUCGCGCAGCUAC | D-012263-04 |
| ASXL2 | 55252 | 73 | GCUCAGCCCUUAACAAUGA | D-022638-01 |
| ASXL2 | 55252 | 74 | GAGAAUUACCUGUUCACUA | D-022638-03 |
| ASXL2 | 55252 | 75 | GCACGCCUUCGAAAUGUUA | D-022638-04 |
| ASXL2 | 55252 | 76 | CCACAGCUUCUAAUUAUAA | D-022638-02 |
| ATG12 | 9140 | 77 | GGGAAGGACUUACGGAUGU | D-010212-01 |
| ATG12 | 9140 | 78 | GAACACCAAGUUUCACUGU | D-010212-02 |
| ATG12 | 9140 | 79 | GCAGCUUCCUACUUCAAUU | D-010212-05 |
| ATG12 | 9140 | 80 | GCAGUAGAGCGAACACGAA | D-010212-03 |
| ATG16L2 | 89849 | 81 | GUAAUGACGUGGUGUGUGG | D-026687-03 |
| ATG16L2 | 89849 | 82 | GAGGAGAGGUCACUCAAUU | D-026687-01 |
| ATG16L2 | 89849 | 83 | GGACACAAGGAUAAGGUGA | D-026687-04 |
| ATG16L2 | 89849 | 84 | GAGCAGCGAUACCAGAUCA | D-026687-02 |
| ATG7 | 10533 | 85 | CCAAAGUUCUUGAUCAAUAUU | D-020112-01 |
| ATG7 | 10533 | 86 | GAUCAAAGGUUUUCACUAAUU | D-020112-02 |
| ATG7 | 10533 | 87 | GAAGAUAACAAUUGGUGUAUU | D-020112-03 |
| ATG7 | 10533 | 88 | CAACAUCCCUGGUUACAAGUU | D-020112-04 |
| ATP6V0A1 | 535 | 89 | CCAAUAAACUGACGUUCUU | D-017618-03 |
| ATP6V0A1 | 535 | 90 | GAAGAUGUCUGUUAUCCUU | D-017618-01 |
| ATP6V0A1 | 535 | 91 | GAACUUACCGAGAGAUAAA | D-017618-04 |
| ATP6V0A1 | 535 | 92 | GGAAGAGGCACUCCUUUAA | D-017618-02 |
| ATP6V1E2 | 90423 | 93 | GAAAGGACGCCUCGUGCAA | D-019427-03 |
| ATP6V1E2 | 90423 | 94 | GAAUGCAGCUGGAGGUGUG | D-019427-01 |
| ATP6V1E2 | 90423 | 95 | CCGAGUACAUGACAAUUUC | D-019427-04 |
| ATP6V1E2 | 90423 | 96 | AGGAAGAGUUUAACAUUGA | D-019427-02 |
| BAHD1 | 22893 | 97 | GAAAGUCCCUUCCCAUGCU | D-020357-03 |
| BAHD1 | 22893 | 98 | UUACAGACCUGAGCACUUA | D-020357-02 |
| BAHD1 | 22893 | 99 | GAACGCAGCUGCUUUCCUA | D-020357-01 |
| BAHD1 | 22893 | 100 | CCGCACUAAUGGCUGGGUA | D-020357-04 |
| BCL9 | 607 | 101 | GCCAGACGCUGCAAUAUUU | D-007268-02 |
| BCL9 | 607 | 102 | CAACUCAACUCCCAACAAU | D-007268-04 |
| BCL9 | 607 | 103 | GAACAUUUCUAACAACAAG | D-007268-03 |
| BCL9 | 607 | 104 | CUACUGAGAUGGCCAAUAA | D-007268-01 |
| BIRC6 | 57448 | 105 | ACAAGCACCUCUCGCAUUA | D-013857-04 |
| BIRC6 | 57448 | 106 | GGUCAAAGAUCACUUAGUA | D-013857-01 |
| BIRC6 | 57448 | 107 | GCAACGAUGUGCCAUGUUA | D-013857-02 |
| BIRC6 | 57448 | 108 | CCAAAGAUACGGUUACAUA | D-013857-03 |
| BSG | 682 | 109 | GGUCAGAGCUACACAUUGA | D-010737-01 |
| BSG | 682 | 110 | GUACAAGAUCACUGACUCU | D-010737-04 |
| BSG | 682 | 111 | GGACAAGGCCCUCAUGAAC | D-010737-05 |
| BSG | 682 | 112 | GAAGUCGUCAGAACACAUC | D-010737-03 |
| C14ORF125 | 25938 | 113 | GAAAUGCUGUUAGUUGUUA | D-021927-02 |
| C14ORF125 | 25938 | 114 | GGAGACAGAUGAGAGAUUA | D-021927-04 |
| C14ORF125 | 25938 | 115 | CAGAAGCGCUGUAUUGAUA | D-021927-03 |
| C14ORF125 | 25938 | 116 | GAACCUACCCUUUCUCUUA | D-021927-01 |
| C1orf103 | 55791 | 117 | GGAAUACUAUACCCAUGAG | D-018103-04 |
| C1orf103 | 55791 | 118 | UAACAGCCGUCAUUUAUGC | D-018103-02 |
| C1orf103 | 55791 | 119 | GCCAACCGUUAUUUAUGUA | D-018103-03 |
| C1orf103 | 55791 | 120 | GAAGAAACCAUUCGAGAUG | D-018103-01 |
| C20ORF106 | 200232 | 121 | GCAAUACGGAGAGCACUUU | D-024178-03 |
| C20ORF106 | 200232 | 122 | CAACUUCACUCUCCAUUAA | D-024178-01 |
| C20ORF106 | 200232 | 123 | CAAAGUGCGGAAUCUUAAA | D-024178-04 |
| C20ORF106 | 200232 | 124 | AGGUGGAGCUUAUGAAAUU | D-024178-02 |
| C20ORF174 | 128611 | 125 | GAUGAAGACCGAUUAGUUA | D-024186-01 |
| C20ORF174 | 128611 | 126 | GUACUUGGCGGUGCACUUU | D-024186-02 |
| C20ORF174 | 128611 | 127 | CGUCAGGUAUCUGGAUUAA | D-024186-03 |
| C20ORF174 | 128611 | 128 | UGACUGAACCCACUAAGCA | D-024186-04 |
| C2ORF25 | 27249 | 129 | CAUUUAGGAUUCUCUGUUG | D-013862-02 |
| C2ORF25 | 27249 | 130 | CCAGAUAUAUGCUCUCGAA | D-013862-01 |
| C2ORF25 | 27249 | 131 | GUAGGGAGUAUCUUCACUA | D-013862-03 |
| C2ORF25 | 27249 | 132 | GUAGAGUGUGCAAUACAAA | D-013862-04 |
| C4orf33 | 132321 | 133 | GCAAAGCUUAUCUCCCUUG | D-018450-02 |
| C4orf33 | 132321 | 134 | UCAAUGAACUGUGGGAUUA | D-018450-01 |
| C4orf33 | 132321 | 135 | AAUCAGACCUGUGGCUAAU | D-018450-04 |
| C4orf33 | 132321 | 136 | GAGUGAUGAUGGACAUUAG | D-018450-03 |
| C6orf1 | 221491 | 137 | CCAAGGUGGUACAUGGCUG | D-017897-03 |
| C6orf1 | 221491 | 138 | GGUGCUGGGUCUCCAUUUC | D-017897-02 |
| C6orf1 | 221491 | 139 | CUUUGUGGGUGUUGAGCUC | D-017897-04 |
| C6orf1 | 221491 | 140 | GGAUGCCACUGCUGGAUGU | D-017897-01 |
| C6ORF162 | 57150 | 141 | ACAACAACCUUAUUUCGUG | D-018820-03 |
| C6ORF162 | 57150 | 142 | AUCCAGAGCUCUUCAUUAA | D-018820-02 |
| C6ORF162 | 57150 | 143 | GGCAUAUAUUGGUUAUCUA | D-018820-04 |
| C6ORF162 | 57150 | 144 | GGACCUCUAUGAAGCUAUU | D-018820-01 |
| C8ORF14 | 83655 | 145 | CCACAAAGCACUUAUAUAG | D-015204-01 |
| C8ORF14 | 83655 | 146 | CAUGUCACCUCUCUACUCC | D-015204-04 |
| C8ORF14 | 83655 | 147 | CGAGAUCGGUUCACUUUGU | D-015204-02 |
| C8ORF14 | 83655 | 148 | UCUCAGAUCUACUAGCACA | D-015204-03 |
| C9orf131 | 138724 | 149 | GGAAGGAUACUGAGCAUUC | D-031891-03 |
| C9orf131 | 138724 | 150 | GCACCCAGCUUGUAACUUA | D-031891-01 |
| C9orf131 | 138724 | 151 | GAACCACACAGAAUCAAUC | D-031891-04 |
| C9orf131 | 138724 | 152 | CCUCAAGGCUAUAGGGAUA | D-031891-02 |
| CACNG1 | 786 | 153 | CCGUCUGGAUCGAGUACUA | D-011162-04 |
| CACNG1 | 786 | 154 | UGACAGCCGUGGUAACCGA | D-011162-03 |
| CACNG1 | 786 | 155 | GGAAGAAGAGGGACUAUCU | D-011162-02 |
| CACNG1 | 786 | 156 | GCUCGGAGAUCUUCGAAUU | D-011162-01 |
| CAPN6 | 827 | 157 | GAAUACAGUUCAUGCCAUU | D-009423-02 |
| CAPN6 | 827 | 158 | GGACUGAAGUGGUGAUUGA | D-009423-01 |
| CAPN6 | 827 | 159 | GGCCAUACCUAUACCAUGA | D-009423-04 |
| CAPN6 | 827 | 160 | AGAAGGACCUGCGCACUUA | D-009423-03 |
| CAV2 | 858 | 161 | UAUCAUUGCUCCAUUGUGU | D-010958-02 |
| CAV2 | 858 | 162 | GUAAAGACCUGCCUAAUGG | D-010958-01 |
| CAV2 | 858 | 163 | CGGCUCAACUCGCAUCUCA | D-010958-04 |
| CAV2 | 858 | 164 | GGACGUACAGCUCUUCAUG | D-010958-03 |
| CCDC134 | 79879 | 165 | CGAGGAUUGUGCACUAUUA | D-014466-01 |
| CCDC134 | 79879 | 166 | AGAGAAACGCCGAAAGAAA | D-014466-04 |
| CCDC134 | 79879 | 167 | GCUCACCGCUGCUGAUGUG | D-014466-03 |
| CCDC134 | 79879 | 168 | GCACAGAGUUCAUUCCCAG | D-014466-02 |
| CCRN4L | 25819 | 169 | GGUUACCUUCCUUCAAUUA | D-012414-02 |
| CCRN4L | 25819 | 170 | GGACAGAUUGCCCUAGUAC | D-012414-04 |
| CCRN4L | 25819 | 171 | GCACUCAAAUGGGAAGAAA | D-012414-01 |
| CCRN4L | 25819 | 172 | GACCACCUGUCUCUAGUGU | D-012414-03 |
| CD164L1 | 57124 | 173 | GCGCAUCACUGACUGCUAU | D-010720-01 |
| CD164L1 | 57124 | 174 | CCACCAGCCUCCUGUGAUC | D-010720-04 |
| CD164L1 | 57124 | 175 | GGACCUCGGAGAUGAGUUG | D-010720-03 |
| CD164L1 | 57124 | 176 | GCAGCCAACUAUCCAGAUC | D-010720-02 |
| CD4 | 920 | 177 | AAUCAGGGCUCCUUCUUAA | D-005234-02 |
| CD4 | 920 | 178 | UCAAGAGACUCCUCAGUGA | D-005234-05 |
| CD4 | 920 | 179 | GAACUGACCUGUACAGCUU | D-005234-01 |
| CD4 | 920 | 180 | GAAGAAGAGCAUACAAUUC | D-005234-03 |
| CENTG1 | 116986 | 181 | GAGAAACGAAGCUUGGAUA | D-021010-02 |
| CENTG1 | 116986 | 182 | AAACAGAGCUUCCUACUAA | D-021010-01 |
| CENTG1 | 116986 | 183 | UUAACGGGCUCGUCAAGGA | D-021010-03 |
| CENTG1 | 116986 | 184 | GAGCGCGAGUCGUGGAUUC | D-021010-04 |
| CHERP | 10523 | 185 | AGACCCAGCUAGACAUGAA | D-016176-03 |
| CHERP | 10523 | 186 | GAUAAGUGGGACCAGUAUA | D-016176-04 |
| CHERP | 10523 | 187 | GAGGCGAAUUCUACAGUUA | D-016176-01 |
| CHERP | 10523 | 188 | GCUGGAAGAUCACGAGUAC | D-016176-02 |
| CLDND1 | 56650 | 189 | CAAUGUAUCCGGUGAAUUU | D-020682-01 |
| CLDND1 | 56650 | 190 | AAACCACAAUAGCGGGAUU | D-020682-04 |
| CLDND1 | 56650 | 191 | GGACCUAUCUUUGGCGUUG | D-020682-02 |
| CLDND1 | 56650 | 192 | CCGGAAAGAGUACACCUUA | D-020682-03 |
| CLN3 | 1201 | 193 | GCAACAACUUCUCUUAUGU | D-019282-01 |
| CLN3 | 1201 | 194 | GGUCUUCGCUAGCAUCUCA | D-019282-03 |
| CLN3 | 1201 | 195 | CGGGAAAGGUGGACAGUAU | D-019282-04 |
| CLN3 | 1201 | 196 | UCAAGGGUCUGCUGUGGUA | D-019282-02 |
| CLNS1A | 1207 | 197 | GUGAGCAGCCAGUAUAAUA | D-012571-02 |
| CLNS1A | 1207 | 198 | AAUCAGCGUUGGAGGCAAU | D-012571-04 |
| CLNS1A | 1207 | 199 | UUAGAUUUGUGCCUAGUGA | D-012571-03 |
| CLNS1A | 1207 | 200 | GGACCGAAGUGACUGUCUA | D-012571-01 |
| COG2 | 22796 | 201 | GGACUUACGCCACGAUUGA | D-019487-01 |
| COG2 | 22796 | 202 | AAAGUAAGACCGCGUAUAG | D-019487-02 |
| COG2 | 22796 | 203 | GAUACUCUGUGUUUGUCAA | D-019487-03 |
| COG2 | 22796 | 204 | GUUAUUCGGUCAGUUGAGA | D-019487-04 |
| COG3 | 83548 | 205 | CCGAGUUCAUCUUGUUUAA | D-013499-04 |
| COG3 | 83548 | 206 | GACAAAGGUUUCAGCGUUA | D-013499-01 |
| COG3 | 83548 | 207 | CAUUGUCGGUGAAUAGUGA | D-013499-03 |
| COG3 | 83548 | 208 | CCAUUGAUCAUUCAUGUUA | D-013499-02 |
| COG4 | 25839 | 209 | GCAAAGUUCGUCAGCUUGA | D-013993-04 |
| COG4 | 25839 | 210 | CAGCACAUAUUCAUCGCUA | D-013993-03 |
| COG4 | 25839 | 211 | UCUAUACCCUGAUCAAAUA | D-013993-01 |
| COG4 | 25839 | 212 | GCUGGAGGCUGUAUACGAA | D-013993-02 |
| COMMD3 | 23412 | 213 | CCAAUCAACUUCAUAGGAU | D-020053-03 |
| COMMD3 | 23412 | 214 | GCAUAUUUGGUGACCUUAA | D-020053-04 |
| COMMD3 | 23412 | 215 | GCACGGAAUAUCAGAAUAA | D-020053-02 |
| COMMD3 | 23412 | 216 | GCACUUAUCUAGAAGACUG | D-020053-01 |
| COP | 114769 | 217 | GAAAGCUGUUUAUCCAUUC | D-004411-03 |
| COP | 114769 | 218 | UCCGAUACCUGGAAAUUAG | D-004411-04 |
| COP | 114769 | 219 | GCACAGGCAUGCCAAAUUU | D-004411-01 |
| COP | 114769 | 220 | GAUAAGACCCGAGCUUUGA | D-004411-02 |
| CRIPAK | 285464 | 221 | GCAUGGUGGUUCUGUAGGU | D-018504-03 |
| CRIPAK | 285464 | 222 | GUCAGACGCUGUUACCGUA | D-018504-01 |
| CRIPAK | 285464 | 223 | GUGCCGAUGUGGAGUGCCA | D-018504-04 |
| CRIPAK | 285464 | 224 | UCACACAUGUCGAUGCGGA | D-018504-02 |
| CRSP2 | 9282 | 225 | CCAUGCAAUUCGCUUAUUA | D-011928-03 |
| CRSP2 | 9282 | 226 | GAAGUUUCGUGUUGAAGGA | D-011928-04 |
| CRSP2 | 9282 | 227 | GGACCACCAUUUAAAGCUA | D-011928-02 |
| CRSP2 | 9282 | 228 | GAAUAGCAUUGCACGAUUA | D-011928-01 |
| CRSP8 | 9442 | 229 | GAAGAUGGCAAGCUUGAUA | D-011949-02 |
| CRSP8 | 9442 | 230 | GGAAAGGUGUUGAAAGUGA | D-011949-01 |
| CRSP8 | 9442 | 231 | GUAAAGGGAUAUAACGAGA | D-011949-04 |
| CRSP8 | 9442 | 232 | GGACAAAACUCCUCUCUAU | D-011949-03 |
| CRSP9 | 9443 | 233 | GAGAUCAGAUUAUAGAGAA | D-017313-01 |
| CRSP9 | 9443 | 234 | UCGAACGGCUUCAUCCUAU | D-017313-03 |
| CRSP9 | 9443 | 235 | GGCAAUCAGUUCCAAUGUG | D-017313-04 |
| CRSP9 | 9443 | 236 | GCACCUGGAACGAGUAAUU | D-017313-02 |
| CSPP1 | 79848 | 237 | CAUCCCAAGUGCUAAAGUA | D-016485-04 |
| CSPP1 | 79848 | 238 | GGAAAGGACUAGACAUUGA | D-016485-01 |
| CSPP1 | 79848 | 239 | AGACAUAUCCUGCCAUUGA | D-016485-03 |
| CSPP1 | 79848 | 240 | GAACGAAUGCGAAGACUGA | D-016485-02 |
| CTDP1 | 9150 | 241 | CGGGAAACCUUAGAAAUCU | D-009326-06 |
| CTDP1 | 9150 | 242 | AAAGAUGUCUGGAAGUUUG | D-009326-03 |
| CTDP1 | 9150 | 243 | GGGCACGGGUGAUAUGAAU | D-009326-05 |
| CTDP1 | 9150 | 244 | GAACAGCCCUGCGGCCUUU | D-009326-02 |
| CX36 | 57369 | 245 | GUUCCUGGUUGGCCAAUAU | D-020726-04 |
| CX36 | 57369 | 246 | CCAGAUUGUUUAGAGGUUA | D-020726-01 |
| CX36 | 57369 | 247 | GCAAAAUGCUAUUGUGAAU | D-020726-03 |
| CX36 | 57369 | 248 | CCACAUACGUUACUGGGUC | D-020726-02 |
| CXCR4 | 7852 | 249 | UAACUACACCGAGGAAAUG | D-005139-03 |
| CXCR4 | 7852 | 250 | GAAGCAUGACGGACAAGUA | D-005139-01 |
| CXCR4 | 7852 | 251 | CAAGCAAGGGUGUGAGUUU | D-005139-05 |
| CXCR4 | 7852 | 252 | GAGUCUGAGUCUUCAAGUU | D-005139-04 |
| CXorf50 | 203429 | 253 | GGAGGCACAUUUCGUCUAA | D-018780-01 |
| CXorf50 | 203429 | 254 | CCGAGUUAGUCUUUGGGUU | D-018780-04 |
| CXorf50 | 203429 | 255 | GCACUCCUAUGUCUUGAUC | D-018780-02 |
| CXorf50 | 203429 | 256 | GACCAGGGCACACCAAAGA | D-018780-03 |
| DC13 | 56942 | 257 | GAUCGGGAGUUGAGAAAAU | D-020330-04 |
| DC13 | 56942 | 258 | GAAUGCAACGUCUUGAUUA | D-020330-01 |
| DC13 | 56942 | 259 | GAAGAAUGAGUACGUAGAA | D-020330-02 |
| DC13 | 56942 | 260 | GCUUAAGGAAUGUCACAAA | D-020330-03 |
| DDOST | 1650 | 261 | CGACGUGUAUGGUGUAUUC | D-015786-02 |
| DDOST | 1650 | 262 | GGAAUUCCUCUAUGACAAU | D-015786-03 |
| DDOST | 1650 | 263 | GACCAUCAGUGCCUUUAUU | D-015786-01 |
| DDOST | 1650 | 264 | GACAGGCAACUAUGAACUA | D-015786-04 |
| DDX10 | 1662 | 265 | GAGGAUGCCAACACAUAUA | D-011842-03 |
| DDX10 | 1662 | 266 | GAGCCAAGCCGAUAAAGUA | D-011842-02 |
| DDX10 | 1662 | 267 | GAAUGGAAGUCUAUAAUGA | D-011842-01 |
| DDX10 | 1662 | 268 | CACAGGUGGUAUCAACUUA | D-011842-04 |
| DDX49 | 54555 | 269 | UCACACAGGUCAACGUGGU | D-017975-04 |
| DDX49 | 54555 | 270 | GUGCAAGACCUGCCAGAUU | D-017975-02 |
| DDX49 | 54555 | 271 | CAUCGUGGCUCGUGGAACA | D-017975-01 |
| DDX49 | 54555 | 272 | GCGAAGAGAGUGUGAGAUC | D-017975-03 |
| DDX53 | 168400 | 273 | CAUAAUAAGGGUAGGGAUU | D-019305-03 |
| DDX53 | 168400 | 274 | GGUCAUUGGUUACAGUGGA | D-019305-02 |
| DDX53 | 168400 | 275 | GGAAGAUCUUGUAGUAAUG | D-019305-01 |
| DDX53 | 168400 | 276 | GUACGUCAACUAGCACUUU | D-019305-04 |
| DDX55 | 57696 | 277 | GUGAAGGGCGUGAAGAUUA | D-027082-03 |
| DDX55 | 57696 | 278 | CAGCGGACCUUCUGCCAAA | D-027082-04 |
| DDX55 | 57696 | 279 | GGAAGAGGGUUCUGAUAUU | D-027082-01 |
| DDX55 | 57696 | 280 | UGUCAUAUGUCCAAGCUUA | D-027082-02 |
| DEPDC5 | 9681 | 281 | CUACGUCGGCUAUGGUUUA | D-020708-04 |
| DEPDC5 | 9681 | 282 | GGAGUUAGCAUAUCAUGAA | D-020708-01 |
| DEPDC5 | 9681 | 283 | GCGAGCACCUGUUUGAUAG | D-020708-03 |
| DEPDC5 | 9681 | 284 | GCACACAGGUUUGGGUUUG | D-020708-02 |
| DHX33 | 56919 | 285 | ACAGUGGUCUUGAGGUGUU | D-017205-04 |
| DHX33 | 56919 | 286 | CUACUAGAGUCUCAGAUGA | D-017205-02 |
| DHX33 | 56919 | 287 | CCAAAGGGCUAUCGCAAAG | D-017205-01 |
| DHX33 | 56919 | 288 | GUUAGGUGCUCUUGAACAU | D-017205-03 |
| DIABLO | 56616 | 289 | GCAGAUCAGGCCUCUAUAA | D-004447-02 |
| DIABLO | 56616 | 290 | UAGAAGAGCUCCGUCAGAA | D-004447-01 |
| DIABLO | 56616 | 291 | CCGACAAUAUACAAGUUUA | D-004447-03 |
| DIABLO | 56616 | 292 | GGAAACCACUUGGAUGACU | D-004447-04 |
| DKFZP686O24166 | 374383 | 293 | CGAGACAACCCAGAUCUUU | D-030031-03 |
| DKFZP686O24166 | 374383 | 294 | GCAAGUCCAUAGAUGAUAA | D-030031-02 |
| DKFZP686O24166 | 374383 | 295 | GGGCAAAUGGUCCGAGGUU | D-030031-04 |
| DKFZP686O24166 | 374383 | 296 | GGAAGAGUCUGACGUUUGA | D-030031-01 |
| DMXL1 | 1657 | 297 | GAGCUUGCCCGGAUUAAUU | D-012091-03 |
| DMXL1 | 1657 | 298 | UGAUUUAGCUUGCCACUCA | D-012091-04 |
| DMXL1 | 1657 | 299 | AGACAACCGUUCACUGUUA | D-012091-02 |
| DMXL1 | 1657 | 300 | GAAGUUAGCUGUGCACAUA | D-012091-01 |
| DNAJB1 | 3337 | 301 | CAACAACAUUCCAGCUGAU | D-012735-03 |
| DNAJB1 | 3337 | 302 | GCUCUGAUGUCAUUUAUCC | D-012735-04 |
| DNAJB1 | 3337 | 303 | GAAAGAGCAUUCGAAACGA | D-012735-01 |
| DNAJB1 | 3337 | 304 | GAGCAGGUUCUUCCAAUAU | D-012735-02 |
| DNAL1 | 83544 | 305 | GAACUGCCAUGCCUCGAAG | D-014722-02 |
| DNAL1 | 83544 | 306 | GCUAAUUGCGAGAAGCUUU | D-014722-04 |
| DNAL1 | 83544 | 307 | GAACUUAAAUGGACUGGAG | D-014722-01 |
| DNAL1 | 83544 | 308 | GUUGAAAGGGAUCCACAUA | D-014722-03 |
| DNAPTP6 | 26010 | 309 | UCAGCGAGCGUAAAUAUGA | D-020248-02 |
| DNAPTP6 | 26010 | 310 | AAUCAUCCACUCACAAUAA | D-020248-03 |
| DNAPTP6 | 26010 | 311 | AAAGAGGCCCAAAUAUUGA | D-020248-04 |
| DNAPTP6 | 26010 | 312 | GAUAUCGCGUCAUGAUUAA | D-020248-01 |
| DOK5L | 220164 | 313 | CAAUGCAGAUCACUCAUGA | D-015595-01 |
| DOK5L | 220164 | 314 | UCACAUCACUCGUCAGAAC | D-015595-03 |
| DOK5L | 220164 | 315 | GAGAUACGGUCGGGACUCA | D-015595-04 |
| DOK5L | 220164 | 316 | CAUGAAAGAUUAAUGCUAG | D-015595-02 |
| DOM3Z | 1797 | 317 | GUACAUGUGUGCAGACAAA | D-005004-02 |
| DOM3Z | 1797 | 318 | GACACAAGCUCCUGAAAUG | D-005004-03 |
| DOM3Z | 1797 | 319 | GUACAUGGGAUACAAAUUU | D-005004-04 |
| DOM3Z | 1797 | 320 | CCAUGAAGAUGUUUGAAUA | D-005004-01 |
| DPM1 | 8813 | 321 | GUAUAUGGCUGGGAUUUGA | D-011535-02 |
| DPM1 | 8813 | 322 | GAACAAAUAUUCGGUGCUU | D-011535-01 |
| DPM1 | 8813 | 323 | CUUCUAAGACCACGAGAGA | D-011535-04 |
| DPM1 | 8813 | 324 | GGAGAUGAUUGUUCGGGCA | D-011535-03 |
| DYSF | 8291 | 325 | GACAGACCGUGUAAUGUUU | D-003652-04 |
| DYSF | 8291 | 326 | CCUAUGAGAACGAGACUAA | D-003652-05 |
| DYSF | 8291 | 327 | GAAGUGCGCCUACAUCUAG | D-003652-06 |
| DYSF | 8291 | 328 | GGACAGACCGUGUAAUGUU | D-003652-02 |
| EDNRA | 1909 | 329 | CCAGACAGAUUGCUGAUAA | D-005485-02 |
| EDNRA | 1909 | 330 | GAAACCAGAAGGAUAUUUA | D-005485-03 |
| EDNRA | 1909 | 331 | GAACUGACCACCCUUAGAA | D-005485-04 |
| EDNRA | 1909 | 332 | GAACCGAUGUGAAUUACUU | D-005485-01 |
| EFHC2 | 80258 | 333 | GCACAGAGGUUGUCUUCUA | D-018562-01 |
| EFHC2 | 80258 | 334 | GGACUUCUAUCCGGCGUCA | D-018562-04 |
| EFHC2 | 80258 | 335 | GAAUGUGAAUGGUUACCUA | D-018562-02 |
| EFHC2 | 80258 | 336 | GCCCUUACGUCCCUACGAA | D-018562-03 |
| DVL2 | 1856 | 337 | GACAGAAACCGAGUCAGUA | D-004069-02 |
| DVL2 | 1856 | 338 | UGUGAGAGCUACCUAGUCA | D-004069-03 |
| DVL2 | 1856 | 339 | GAAACCGAGUCAGUAGUGU | D-004069-04 |
| DVL2 | 1856 | 340 | CGCUAAACAUGGAGAAGUA | D-004069-05 |
| EGF | 1950 | 341 | GAGAGAGUAUGUAAUAUAG | D-011650-01 |
| EGF | 1950 | 342 | GACCACCACUAUUCCGUAA | D-011650-03 |
| EGF | 1950 | 343 | GCUAUGCCAUCAGUAAUAA | D-011650-02 |
| EGF | 1950 | 344 | UCAAAACGCCGAAGACUUA | D-011650-04 |
| EGFR | 1956 | 345 | GUAACAAGCUCACGCAGUU | D-003114-08 |
| EGFR | 1956 | 346 | GGAAAUAUGUACUACGAAA | D-003114-06 |
| EGFR | 1956 | 347 | CCACAAAGCAGUGAAUUUA | D-003114-07 |
| EGFR | 1956 | 348 | GAAGGAAACUGAAUUCAAA | D-003114-05 |
| EIF2C3 | 192669 | 349 | GUAAGAAGUGCAAAUUAUG | D-004640-08 |
| EIF2C3 | 192669 | 350 | UCGGAGGGAUCAAUAAUAU | D-004640-06 |
| EIF2C3 | 192669 | 351 | GAAGUGACUCAUUGUGGAA | D-004640-07 |
| EIF2C3 | 192669 | 352 | GAACAGUAGCGCAGUAUUU | D-004640-05 |
| EIF3S3 | 8667 | 353 | UACUAUGGCUCAUUCGUUA | D-003883-03 |
| EIF3S3 | 8667 | 354 | AAGGAUCUCUCUCACUAAA | D-003883-04 |
| EIF3S3 | 8667 | 355 | GAAGAUCGGCUUGAAAUUA | D-003883-01 |
| EIF3S3 | 8667 | 356 | GAAGUGCCGAUUGUAAUUA | D-003883-02 |
| EIF4G2 | 1982 | 357 | GUGAACAUCUUAAUGACUA | D-011263-02 |
| EIF4G2 | 1982 | 358 | GCAGUUAGCUAAAUUACAA | D-011263-01 |
| EIF4G2 | 1982 | 359 | GAACGAGCCAAGUCCUUAA | D-011263-04 |
| EIF4G2 | 1982 | 360 | AGUCUAAACUCAUCCUUAA | D-011263-03 |
| EME1 | 146956 | 361 | GCUAAGCAGUGAAAGUGAA | D-016420-03 |
| EME1 | 146956 | 362 | GAAUUUGCUCGCAGACAUA | D-016420-04 |
| EME1 | 146956 | 363 | GCUCAAAGGCUUACAUGUA | D-016420-01 |
| EME1 | 146956 | 364 | GGAAAUGGCCAGUGCAGUU | D-016420-02 |
| EPS8 | 2059 | 365 | GCGAGAGUCUAUAGCCAAA | D-017905-02 |
| EPS8 | 2059 | 366 | UCGGAAAGAUGCUAUGAUC | D-017905-03 |
| EPS8 | 2059 | 367 | UGUCAAUAGUCUUGGAGUA | D-017905-04 |
| EPS8 | 2059 | 368 | AAACACGGAUUUAACCUUC | D-017905-01 |
| ERCC3 | 2071 | 369 | GGGAAUAUGUGGCAAUCAA | D-011028-03 |
| ERCC3 | 2071 | 370 | GAAUAUGACUUCCGGAAUG | D-011028-02 |
| ERCC3 | 2071 | 371 | GAGAAUGCCGCUUAAGAAA | D-011028-04 |
| ERCC3 | 2071 | 372 | GAACAAACCCUAUAUCUAC | D-011028-01 |
| ERP27 | 121506 | 373 | GGACAGUGGUAUGAAAGAA | D-015698-02 |
| ERP27 | 121506 | 374 | CAACAGCGUAAUUCAGAUU | D-015698-04 |
| ERP27 | 121506 | 375 | CCACGUGGCUCACAGAUGU | D-015698-01 |
| ERP27 | 121506 | 376 | CGAAGACAUUGAAAGCAUU | D-015698-03 |
| ETF1 | 2107 | 377 | GAUCAGAGGUUACAAUCAA | D-019840-01 |
| ETF1 | 2107 | 378 | GAAACACGGUAGAGGAGGU | D-019840-03 |
| ETF1 | 2107 | 379 | AAUGUUAGCGGAUGAGUUU | D-019840-04 |
| ETF1 | 2107 | 380 | UAACUAUGUUCGGAAAGUA | D-019840-02 |
| ETHE1 | 23474 | 381 | GCAGAUAGACUUUGCUGUU | D-012508-01 |
| ETHE1 | 23474 | 382 | GAUCUACCCUGCUCACGAU | D-012508-02 |
| ETHE1 | 23474 | 383 | CAGGCUGACUUACACAUUG | D-012508-04 |
| ETHE1 | 23474 | 384 | UCUGUCAUCUCCCGCCUUA | D-012508-03 |
| EXOD1 | 112479 | 385 | CGGCAGCUUGGAUUAAUUA | D-015252-04 |
| EXOD1 | 112479 | 386 | UACAAUGACUGCAUGUUAA | D-015252-02 |
| EXOD1 | 112479 | 387 | CCAAGCAGUUGUUUGACUA | D-015252-03 |
| EXOD1 | 112479 | 388 | GAUCAGAGAUGGUUGUGUA | D-015252-01 |
| EXOSC3 | 51010 | 389 | GGAGUGAGCCAGCUUCUUU | D-031955-01 |
| EXOSC3 | 51010 | 390 | ACUCUGGGCUUAAUUAGAA | D-031955-03 |
| EXOSC3 | 51010 | 391 | GGAGAUAGUAUUUGGAAUG | D-031955-02 |
| EXOSC3 | 51010 | 392 | GCCAGUUUGUGGUUGCUAA | D-031955-04 |
| EXOSC5 | 56915 | 393 | CAACAAGGCCACACUCGAA | D-020482-01 |
| EXOSC5 | 56915 | 394 | CAAAAUCCGUGCUGAAAAU | D-020482-03 |
| EXOSC5 | 56915 | 395 | CAACACGUCUUCCGUUUCU | D-020482-02 |
| EXOSC5 | 56915 | 396 | CAUGCGGGCUCUCUUCUGU | D-020482-04 |
| FAMSB | 57795 | 397 | GGAUAGCCGCAUUGAGGUA | D-014022-04 |
| FAM5B | 57795 | 398 | GUACAGGAUUUAUAGGGAG | D-014022-03 |
| FAM5B | 57795 | 399 | CCACCGCGCUCAGGAGUAU | D-014022-01 |
| FAM5B | 57795 | 400 | GAGACAAUCUACUAUGAGC | D-014022-02 |
| FAM76B | 143684 | 401 | GAACAGUGCAAACAGCAAU | D-015721-03 |
| FAM76B | 143684 | 402 | UAAAUCCUCUGCAACAAUU | D-015721-01 |
| FAM76B | 143684 | 403 | GAUGGAAAGUUAUUAUGCU | D-015721-04 |
| FAM76B | 143684 | 404 | GAUUGCACAUCCUAUUGUA | D-015721-02 |
| FBXO18 | 84893 | 405 | UCACGUGCCUAUUUGGUGU | D-017404-03 |
| FBXO18 | 84893 | 406 | GAGCCAAGCUUGUGUGUAA | D-017404-02 |
| FBXO18 | 84893 | 407 | GGAAAUAGCUUAUGUGGGA | D-017404-01 |
| FBXO18 | 84893 | 408 | GCACUUCAGAGUUGAGUCA | D-017404-04 |
| FBXO21 | 23014 | 409 | GAAACGGUGCAGAAUAUUU | D-012917-03 |
| FBXO21 | 23014 | 410 | GAACUGGUGUGUAUCCUAA | D-012917-01 |
| FBXO21 | 23014 | 411 | UGAAGGUGCUGUAUAUAUU | D-012917-02 |
| FBXO21 | 23014 | 412 | GAACAGGAAUCCCAAUCAG | D-012917-04 |
| FBXW11 | 23291 | 413 | GUAAAGGUGUCUACUGUUU | D-003490-01 |
| FBXW11 | 23291 | 414 | GAGCAAGGCUUAGAUCACA | D-003490-04 |
| FBXW11 | 23291 | 415 | GUUAGUGGAUCAUCAGAUA | D-003490-03 |
| FBXW11 | 23291 | 416 | GCACAUUGGUGGAACAUUC | D-003490-02 |
| FGD6 | 55785 | 417 | GAAGGGACCGGUUUUAUAA | D-026895-02 |
| FGD6 | 55785 | 418 | GCUCAAAGAUGCCUUAAUA | D-026895-01 |
| FGD6 | 55785 | 419 | GAAUUCCGAGUCUAAAGUA | D-026895-03 |
| FGD6 | 55785 | 420 | GCUCGUCUGUUACGCCAAA | D-026895-04 |
| FHL3 | 2275 | 421 | GAAGAUCCCUACUGUGUGG | D-019805-03 |
| FHL3 | 2275 | 422 | CGAGGGAGCUGUUCUAUGA | D-019805-02 |
| FHL3 | 2275 | 423 | GCAAGUAUGUGUCCUUUGA | D-019805-01 |
| FHL3 | 2275 | 424 | CGACAAGGGUGCUCACUAC | D-019805-04 |
| FKSG2 | 59347 | 425 | GUUACUGAAAGCACGAUAA | D-004427-05 |
| FKSG2 | 59347 | 426 | GAAAGCAUCUUCACAAAAG | D-004427-02 |
| FKSG2 | 59347 | 427 | GUAAGGAUGGUCAGUAUGA | D-004427-04 |
| FKSG2 | 59347 | 428 | CAACAGGGAACACUGAUGA | D-004427-03 |
| FLII | 2314 | 429 | CGUGAAGCCUCCAAUAUGG | D-017506-04 |
| FLII | 2314 | 430 | GAAUUGGGACGAUGUGUUG | D-017506-01 |
| FLII | 2314 | 431 | CAGGAUGUAUCGUGUGUAU | D-017506-02 |
| FLII | 2314 | 432 | UGCCACAGAUCAACUACAA | D-017506-03 |
| FLJ10154 | 55082 | 433 | AAGAGUAGAAGAAUUGGUA | D-021093-04 |
| FLJ10154 | 55082 | 434 | CCAAAUCUCGGGAAAGUAA | D-021093-02 |
| FLJ10154 | 55082 | 435 | CGUCUAAACUUAACAUUGC | D-021093-03 |
| FLJ10154 | 55082 | 436 | GGUGCUGACUGAUAACUUA | D-021093-01 |
| FLJ10774 | 55226 | 437 | CAACAUCACUCGGAUAGUC | D-014402-03 |
| FLJ10774 | 55226 | 438 | GGAAGGGUCGUUCGCAUUG | D-014402-04 |
| FLJ10774 | 55226 | 439 | GGAAUAUGGUGGACUAUCA | D-014402-01 |
| FLJ10774 | 55226 | 440 | UAAGAAGUGUCUCGUCAUU | D-014402-02 |
| FLJ20557 | 55659 | 441 | AGACAAAGCUAUACCCUCG | D-013825-04 |
| FLJ20557 | 55659 | 442 | CAAAGAACCACCCUCAAUA | D-013825-02 |
| FLJ20557 | 55659 | 443 | GGAAUAAGUCAUUACAAGU | D-013825-03 |
| FLJ20557 | 55659 | 444 | UAACUGCGCUGGUUUGUUG | D-013825-01 |
| FLJ21144 | 64789 | 445 | UGACAAACCCAUAAGCUUA | D-014212-02 |
| FLJ21144 | 64789 | 446 | CAGCAUACACCUAGCUAGA | D-014212-04 |
| FLJ21144 | 64789 | 447 | GAAGAUGCUUGGGCAAUUA | D-014212-01 |
| FLJ21144 | 64789 | 448 | GUACUGAGAUUGUAGCCUU | D-014212-03 |
| FLJ21908 | 79657 | 449 | GAACAGAGCGUCAGCAUAU | D-014385-01 |
| FLJ21908 | 79657 | 450 | GCAAUCGAAUUACAACUAC | D-014385-03 |
| FLJ21908 | 79657 | 451 | CAAAGACGAUAGUACCCAU | D-014385-04 |
| FLJ21908 | 79657 | 452 | GCAGUUGCCUUGAAUAGAA | D-014385-02 |
| FLJ30851 | 375190 | 453 | GGAAAUAAUCCAAGUGCCA | D-027266-04 |
| FLJ30851 | 375190 | 454 | GAGAACCGCUGUGCUAUCA | D-027266-01 |
| FLJ30851 | 375190 | 455 | GGCCAUCUCUGAAGGGUAU | D-027266-03 |
| FLJ30851 | 375190 | 456 | GAAACUGCCUACAAGAUAC | D-027266-02 |
| FLJ32569 | 148811 | 457 | GACAGCCGAUUCUUUACAA | D-016737-02 |
| FLJ32569 | 148811 | 458 | GCUAUUUGAACCACUUAUA | D-016737-03 |
| FLJ32569 | 148811 | 459 | CGAAGAACAUUGUGGCUGA | D-016737-01 |
| FLJ32569 | 148811 | 460 | GCUCUGAGAAGUCCAAUAC | D-016737-04 |
| FLJ46026 | 400627 | 461 | CUGCAAAGGGCGAGUGCUU | D-032143-04 |
| FLJ46026 | 400627 | 462 | GCGAUGCUGUUAAGAAAGG | D-032143-01 |
| FLJ46026 | 400627 | 463 | CUGCACAGCUACACAGCGA | D-032143-02 |
| FLJ46026 | 400627 | 464 | CGUCCUUGCCUGAAACAAA | D-032143-03 |
| FLJ46066 | 401103 | 465 | CAGGUAGGCUUAUUUAUCA | D-028203-01 |
| FLJ46066 | 401103 | 466 | GAAGCCAAGCAACUUAAUG | D-028203-03 |
| FLJ46066 | 401103 | 467 | GAAAGAACCCAAUCUCACA | D-028203-02 |
| FLJ46066 | 401103 | 468 | GUAUGAGAAGGCAACAUUU | D-028203-04 |
| FLJ90680 | 400926 | 469 | GGAAUGAAUUGCUGGACGU | D-032160-04 |
| FLJ90680 | 400926 | 470 | CCAAAGCUCCACACGGAUA | D-032160-03 |
| FLJ90680 | 400926 | 471 | GGACUUGAGUUUCAUGCUU | D-032160-02 |
| FLJ90680 | 400926 | 472 | CCAACAAGCUAACCCAUUG | D-032160-01 |
| FLNC | 2318 | 473 | GGGCAGAGCUCGAUGUGGA | D-011272-02 |
| FLNC | 2318 | 474 | GAACAAGCAUUCUCUGUGA | D-011272-01 |
| FLNC | 2318 | 475 | UGACAAGGAUCGCACCUAU | D-011272-03 |
| FLNC | 2318 | 476 | GAACCAUGACGGUACCUUU | D-011272-04 |
| FNTA | 2339 | 477 | CCAAAGAUACUUCGUUAUU | D-008807-04 |
| FNTA | 2339 | 478 | GAAAGUGCAUGGAACUAUU | D-008807-02 |
| FNTA | 2339 | 479 | GAGCAGAAUGGGCUGAUAU | D-008807-05 |
| FNTA | 2339 | 480 | GAAAAUGACUCACCAACAA | D-008807-03 |
| GABARAPL2 | 11345 | 481 | UCAUGUGGAUCAUCAGGAA | D-006853-04 |
| GABARAPL2 | 11345 | 482 | GUACUUGGUUCCAUCUGAU | D-006853-03 |
| GABARAPL2 | 11345 | 483 | UAACUAUGGGACAGCUUUA | D-006853-02 |
| GABARAPL2 | 11345 | 484 | GGUCUCAGGCUCUCAGAUU | D-006853-05 |
| GAJ | 84057 | 485 | GCUAACAGAUGGACUGAUA | D-014779-02 |
| GAJ | 84057 | 486 | AAAGAGAACUCGCAUGAUG | D-014779-03 |
| GAJ | 84057 | 487 | GAGAAAGGCAUUACUGCUA | D-014779-04 |
| GAJ | 84057 | 488 | GAUCGGAACUUCUAAUUAU | D-014779-01 |
| GAPVD1 | 26130 | 489 | GAAAGUUUAUCACCCUAUA | D-026206-04 |
| GAPVD1 | 26130 | 490 | GGAGUACAAUCAGCGCAUA | D-026206-03 |
| GAPVD1 | 26130 | 491 | GGACACAGCAAAUUCUUGG | D-026206-01 |
| GAPVD1 | 26130 | 492 | GCACCUCGGCCCAUUCCUA | D-026206-02 |
| GBAS | 2631 | 493 | GUCAAGAGGUGUUGCCAAA | D-011282-03 |
| GBAS | 2631 | 494 | GAUCCGGACCUAAUAUAUA | D-011282-01 |
| GBAS | 2631 | 495 | CCGGAAAGUUGAUCCAAGA | D-011282-04 |
| GBAS | 2631 | 496 | GAAACAAGCAAUCUAUACA | D-011282-02 |
| GCK | 2645 | 497 | GCAAGCAGAUCUACAACAU | D-010819-01 |
| GCK | 2645 | 498 | GCUCAUAGGUGGCAAGUAC | D-010819-02 |
| GCK | 2645 | 499 | GCACGAAGACAUCGAUAAG | D-010819-04 |
| GCK | 2645 | 500 | CCACGAUGAUCUCCUGCUA | D-010819-05 |
| GCN5L2 | 2648 | 501 | GCACAACAUUCUCUACUUC | D-009722-03 |
| GCN5L2 | 2648 | 502 | AGAAAGAGAUCAUCAAGAA | D-009722-01 |
| GCN5L2 | 2648 | 503 | CCAUGGAGCUGGUCAAUGA | D-009722-02 |
| GCN5L2 | 2648 | 504 | CCAAGCAGGUCUAUUUCUA | D-009722-04 |
| GLE1L | 2733 | 505 | GCACACAGAAUCUAUGGUA | D-011287-01 |
| GLE1L | 2733 | 506 | GAUUCACCCUCAUGGCUUA | D-011287-02 |
| GLE1L | 2733 | 507 | GAACAACUGAAGCGGUUUG | D-011287-04 |
| GLE1L | 2733 | 508 | AAUACAAACUGGCAGAGAA | D-011287-03 |
| GMEB2 | 26205 | 509 | GACAAGGUCUGCUCCAACA | D-012470-02 |
| GMEB2 | 26205 | 510 | GCUCAAGGAAGCCGUGUUA | D-012470-01 |
| GMEB2 | 26205 | 511 | GUACGACGAGCAUGUAAUC | D-012470-04 |
| GMEB2 | 26205 | 512 | GCAUCAAUGUGAAAUGUGU | D-012470-03 |
| GML | 2765 | 513 | GUAAUAGCAUGGUUUGCAA | D-019639-01 |
| GML | 2765 | 514 | CAUUAGAGUAUGUCCGUAU | D-019639-04 |
| GML | 2765 | 515 | GGACAUGUUACCCGAUGAA | D-019639-03 |
| GML | 2765 | 516 | UCAGUGGACUUACAGUUUG | D-019639-02 |
| GMPPA | 29926 | 517 | GGAGAAACCCAGCACAUUU | D-013667-01 |
| GMPPA | 29926 | 518 | CGGGAUGUCUUCCAGCGUA | D-013667-04 |
| GMPPA | 29926 | 519 | GAUCAAGUCCGCAGGUUCA | D-013667-03 |
| GMPPA | 29926 | 520 | GGACGCAAUCCCUCAACUA | D-013667-02 |
| GOLPH3 | 64083 | 521 | AAACAGAACUUCCUACUUU | D-006414-01 |
| GOLPH3 | 64083 | 522 | GAGAGGAAGGUUACAACUA | D-006414-03 |
| GOLPH3 | 64083 | 523 | UUACGUGGCUGUAUGUUAA | D-006414-02 |
| GOLPH3 | 64083 | 524 | UCAAGGACCGCGAGGGUUA | D-006414-04 |
| GOSR2 | 9570 | 525 | GAUCCAGUCUUGCAUGGGA | D-010980-03 |
| GOSR2 | 9570 | 526 | CGAAAUCCAAGCAAGCAUA | D-010980-04 |
| GOSR2 | 9570 | 527 | GAAGAAGAUCCUUGACAUU | D-010980-02 |
| GOSR2 | 9570 | 528 | ACGAAUCACUGCAGUUUAA | D-010980-01 |
| GPS2 | 2874 | 529 | GCGCUGCACCGGCACAUUA | D-004329-03 |
| GPS2 | 2874 | 530 | UGGAUAAGAUGAUGGAACA | D-004329-05 |
| GPS2 | 2874 | 531 | GCGAUUCUACCACAAGUGA | D-004329-04 |
| GPS2 | 2874 | 532 | UGACAGAGCCAAACAAAUG | D-004329-02 |
| GREM1 | 26585 | 533 | ACUCAACUGCCCUGAACUA | D-021492-03 |
| GREM1 | 26585 | 534 | GAAGCAGUGUCGUUGCAUA | D-021492-02 |
| GREM1 | 26585 | 535 | GCAAAUACCUGAAGCGAGA | D-021492-01 |
| GREM1 | 26585 | 536 | GCCGGCUGCUGAAGGGAAA | D-021492-04 |
| GRTP1 | 79774 | 537 | GCGAUAAGUUUAAGCAGAU | D-014422-03 |
| GRTP1 | 79774 | 538 | GAAGAAUACUACCAGAUUA | D-014422-01 |
| GRTP1 | 79774 | 539 | GCUUCGUGAUGGAGUGUCA | D-014422-04 |
| GRTP1 | 79774 | 540 | AACGAAGGCUCGAAGAUUA | D-014422-02 |
| GSDMDC1 | 79792 | 541 | GCACCUCAAUGAAUGUGUA | D-016207-01 |
| GSDMDC1 | 79792 | 542 | GUGUCAACCUGUCUAUCAA | D-016207-03 |
| GSDMDC1 | 79792 | 543 | CCUACUGCCUGGUGGUUAG | D-016207-04 |
| GSDMDC1 | 79792 | 544 | GAAGGAAGCUGCAGGGGUC | D-016207-02 |
| H3F3A | 3020 | 545 | CAAAUCGACCGGUGGUAAA | D-011684-02 |
| H3F3A | 3020 | 546 | GCGCAGCUAUCGGUGCUUU | D-011684-01 |
| H3F3A | 3020 | 547 | CUACAAAAGCCGCUCGCAA | D-011684-04 |
| H3F3A | 3020 | 548 | GCAAGUGAGGCCUAUCUGG | D-011684-03 |
| HDAC7A | 51564 | 549 | GAAGCUAGCGGAGGUGAUU | D-009330-04 |
| HDAC7A | 51564 | 550 | AGAAUCCACUGCUCCGAAA | D-009330-06 |
| HDAC7A | 51564 | 551 | GGAAGAACCUAUGAAUCUC | D-009330-02 |
| HDAC7A | 51564 | 552 | GACAAGAGCAAGCGAAGUG | D-009330-05 |
| HEATR1 | 55127 | 553 | UAAAGAAGCUUGAAAGUGU | D-015939-01 |
| HEATR1 | 55127 | 554 | UGGGUUAAGUUGCUUGAUA | D-015939-04 |
| HEATR1 | 55127 | 555 | GCUCAGAAGUCCUCAGAUA | D-015939-02 |
| HEATR1 | 55127 | 556 | CCGCUGACAUAUUAAUUAA | D-015939-03 |
| HERC3 | 8916 | 557 | GAGCUGAUCGCUUUAAAUA | D-007179-02 |
| HERC3 | 8916 | 558 | GAACUCAACUAGGGUGUUA | D-007179-01 |
| HERC3 | 8916 | 559 | GCAAAGUACUAGAUAACUG | D-007179-05 |
| HERC3 | 8916 | 560 | CGAGAAAGCUAUGGAGUGA | D-007179-03 |
| HERC6 | 55008 | 561 | UGAAAGAGAUCACCCAACA | D-005175-04 |
| HERC6 | 55008 | 562 | UCACCCAGAUUUAUACUUA | D-005175-02 |
| HERC6 | 55008 | 563 | GAAGUCGCCUGGUUAAAGA | D-005175-03 |
| HERC6 | 55008 | 564 | AGACAGCUCUUUCGGGAUA | D-005175-06 |
| HIBCH | 26275 | 565 | GCACUGACUCUUAAUAUGA | D-009852-03 |
| HIBCH | 26275 | 566 | GAAGAUAGCUCCAGUUUUC | D-009852-01 |
| HIBCH | 26275 | 567 | GAAACCAGCUGAUCUAAAA | D-009852-02 |
| HIBCH | 26275 | 568 | CAGAAGAGGUGCUAUUGGA | D-009852-04 |
| HIP1R | 9026 | 569 | CCGACAUGCUGUACUUCAA | D-027079-04 |
| HIP1R | 9026 | 570 | CAGCUCAACUCGUGAACUA | D-027079-01 |
| HIP1R | 9026 | 571 | CCUCUUCGAUCAGACGUUU | D-027079-03 |
| HIP1R | 9026 | 572 | CUGUGGAGAUGUUUGAUUA | D-027079-02 |
| HNRPF | 3185 | 573 | GAACAGCAUGGGUGGCUAU | D-013449-04 |
| HNRPF | 3185 | 574 | CGACCGAGAACGACAUUUA | D-013449-01 |
| HNRPF | 3185 | 575 | CCAAUAUGCAGCACAGAUA | D-013449-03 |
| HNRPF | 3185 | 576 | GGAUGUAUGACCACAGAUA | D-013449-02 |
| HRIHFB2122 | 11078 | 577 | CACCAAGGAUGCUGUCUAU | D-012342-02 |
| HRIHFB2122 | 11078 | 578 | GCACGGAUGUCACUGAGUA | D-012342-01 |
| HRIHFB2122 | 11078 | 579 | GGAUGUCGAUCUUGGACGA | D-012342-03 |
| HRIHFB2122 | 11078 | 580 | GGAUCGAGGCUCUGAGAAA | D-012342-04 |
| HSA9761 | 27292 | 581 | GACACUCUCUGCUGCAUUU | D-009476-03 |
| HSA9761 | 27292 | 582 | UGCAGACUCUCAAUUAAUA | D-009476-04 |
| HSA9761 | 27292 | 583 | GAUUUGCCAUUCUUUGAUA | D-009476-02 |
| HSA9761 | 27292 | 584 | GGAAUGGGAUGGUCUAGUA | D-009476-01 |
| HTATSF1 | 27336 | 585 | CAGGAUGGUUUCAUGUUGA | D-016645-04 |
| HTATSF1 | 27336 | 586 | GAUGAAGACUGCUCUGAAA | D-016645-02 |
| HTATSF1 | 27336 | 587 | GAAAUUAGAGGCUACAAAU | D-016645-03 |
| HTATSF1 | 27336 | 588 | CUACAUAUCAGGCCAAUUA | D-016645-01 |
| HUWE1 | 10075 | 589 | GGAAGAGGCUAAAUGUCUA | D-007185-04 |
| HUWE1 | 10075 | 590 | GCAAAGAAAUGGAUAUCAA | D-007185-01 |
| HUWE1 | 10075 | 591 | UAACAUCAAUUGUCCACUU | D-007185-05 |
| HUWE1 | 10075 | 592 | GAAAUGGAUAUCAAACGUA | D-007185-06 |
| IDH1 | 3417 | 593 | GAGCAAAGCUUGAUAACAA | D-008294-01 |
| IDH1 | 3417 | 594 | GUACAUAACUUUGAAGAAG | D-008294-03 |
| IDH1 | 3417 | 595 | CAAGAUAAGUCAAUUGAAG | D-008294-04 |
| IDH1 | 3417 | 596 | GGACUUGGCUGCUUGCAUU | D-008294-02 |
| IGHMBP2 | 3508 | 597 | GAAAUACACCCGCUGACAU | D-019657-01 |
| IGHMBP2 | 3508 | 598 | GAAGUCCGCCUCGUCAGUU | D-019657-03 |
| IGHMBP2 | 3508 | 599 | UGAUAACACCUGCGGCUUU | D-019657-04 |
| IGHMBP2 | 3508 | 600 | GUACGAUGCUGCUAAUGAG | D-019657-02 |
| IKBKG | 8517 | 601 | AAACAGGAGGUGAUCGAUA | D-003767-04 |
| IKBKG | 8517 | 602 | AACAGGAGGUGAUCGAUAA | D-003767-01 |
| IKBKG | 8517 | 603 | GGAAGAGCCAACUGUGUGA | D-003767-03 |
| IKBKG | 8517 | 604 | UGGAGAAGCUCGAUCUGAA | D-003767-02 |
| INTS7 | 25896 | 605 | GCUGUUUACUGAUAUCUCA | D-013972-04 |
| INTS7 | 25896 | 606 | UCAAGACGCUGCCCGGAUU | D-013972-03 |
| INTS7 | 25896 | 607 | CAACUUAUCUGUACUUGUA | D-013972-01 |
| INTS7 | 25896 | 608 | GAAGAAUGAUGUCUGUAUA | D-013972-02 |
| IQUB | 154865 | 609 | CCAAGACAAGUUUCAUAUA | D-018861-02 |
| IQUB | 154865 | 610 | GGAGUAGAGUAUCACAAUG | D-018861-01 |
| IQUB | 154865 | 611 | ACACAACACCUAAGAUUAU | D-018861-03 |
| IQUB | 154865 | 612 | GCAUAUACCGGUGUCGUAA | D-018861-04 |
| ITLN1 | 55600 | 613 | GGAAUUCACUGCGGGAUUU | D-009035-01 |
| ITLN1 | 55600 | 614 | GGAAAGUGUUGGACUGACA | D-009035-04 |
| ITLN1 | 55600 | 615 | CAGAUGAGGCUAAUACUUA | D-009035-02 |
| ITLN1 | 55600 | 616 | GGAAAUCAAAGACGAAUGU | D-009035-03 |
| ITPKA | 3706 | 617 | GGUCAUAAGCCCUUUCAAG | D-006742-07 |
| ITPKA | 3706 | 618 | CGACGGACCUUGUGUGCUC | D-006742-05 |
| ITPKA | 3706 | 619 | CGUCAGGACUUACCUAGAG | D-006742-06 |
| ITPKA | 3706 | 620 | GCACCGACUUCAAGACUAC | D-006742-04 |
| JAK1 | 3716 | 621 | UAAGGAACCUCUAUCAUGA | D-003145-07 |
| JAK1 | 3716 | 622 | UGAAAUCACUCACAUUGUA | D-003145-06 |
| JAK1 | 3716 | 623 | CCACAUAGCUGAUCUGAAA | D-003145-05 |
| JAK1 | 3716 | 624 | GCAGGUGGCUGUUAAAUCU | D-003145-08 |
| JHDM1D | 80853 | 625 | GCACAGACAUGACUACACA | D-025357-02 |
| JHDM1D | 80853 | 626 | GGAAACUUCGAGAUCAUAA | D-025357-04 |
| JHDM1D | 80853 | 627 | GGACAUACCUUAUUUGUUC | D-025357-01 |
| JHDM1D | 80853 | 628 | GAUACCAUGUCAAGACUGA | D-025357-03 |
| JMJD2D | 55693 | 629 | CCCAGAAUCCAAAUUGUAA | D-020709-03 |
| JMJD2D | 55693 | 630 | GGAAGAACCGCAUCUAUAA | D-020709-01 |
| JMJD2D | 55693 | 631 | UGUCAUAGAAGGCGUCAAU | D-020709-04 |
| JMJD2D | 55693 | 632 | AGAGAGACCUAUGAUAAUA | D-020709-02 |
| KBTBD7 | 84078 | 633 | GAGAGUGAGCGGACUGUAU | D-015708-04 |
| KBTBD7 | 84078 | 634 | GCACGGAGUGAGUCUAGUA | D-015708-03 |
| KBTBD7 | 84078 | 635 | GAAUGGAGGCGGAUUAGUA | D-015708-02 |
| KBTBD7 | 84078 | 636 | GAAGAUCAGUGGAUUAAUA | D-015708-01 |
| KBTBD7 | 84078 | 637 | GAAGAUCAGUGGAUUAAUA | D-015708-01 |
| KBTBD7 | 84078 | 638 | GAAUGGAGGCGGAUUAGUA | D-015708-02 |
| KBTBD7 | 84078 | 639 | GCACGGAGUGAGUCUAGUA | D-015708-03 |
| KBTBD7 | 84078 | 640 | GAGAGUGAGCGGACUGUAU | D-015708-04 |
| KCNIP3 | 30818 | 641 | CCACAGGGCUCAGAUAGCA | D-017332-01 |
| KCNIP3 | 30818 | 642 | GCACACACCACUUAGCAAG | D-017332-02 |
| KCNIP3 | 30818 | 643 | CGGAGCACGUGGAGAGGUU | D-017332-04 |
| KCNIP3 | 30818 | 644 | CCUUUAAUCUCUACGACAU | D-017332-03 |
| KCNK9 | 51305 | 645 | GAGGAGAUCUCACCAAGCA | D-004891-02 |
| KCNK9 | 51305 | 646 | GAUCUCACCAAGCACAUUA | D-004891-05 |
| KCNK9 | 51305 | 647 | GCGAGGAGGAGAAACUCAA | D-004891-06 |
| KCNK9 | 51305 | 648 | GCAACAGCAUGGUCAUUCA | D-004891-03 |
| KEL | 3792 | 649 | GGACGUCAAUGCUUACUAU | D-005903-02 |
| KEL | 3792 | 650 | CAGCAGAUCUUCUUUCGAA | D-005903-01 |
| KEL | 3792 | 651 | GUAAAUGGACUUCCUUAAA | D-005903-03 |
| KEL | 3792 | 652 | CGACAAGAAUACAACGAUA | D-005903-04 |
| KIAA0258 | 9827 | 653 | GAGGGAAAGUUGGGACGUU | D-021128-03 |
| KIAA0258 | 9827 | 654 | UCAAGUACGUCUACAAACU | D-021128-02 |
| KIAA0258 | 9827 | 655 | GGAAGGAACCGUAGCUUGU | D-021128-01 |
| KIAA0258 | 9827 | 656 | CUACAUACAACUAGAACCA | D-021128-04 |
| KIAA0310 | 9919 | 657 | GGACGGAAGCCUAUGAGUA | D-026032-02 |
| KIAA0310 | 9919 | 658 | CUAAUCAGCCUGCUAAUUU | D-026032-01 |
| KIAA0310 | 9919 | 659 | GCGGUCAGCUUAUCAAAGU | D-026032-03 |
| KIAA0310 | 9919 | 660 | GGAGAGCUUUCGCGCUGUA | D-026032-04 |
| KIAA0355 | 9710 | 661 | GCCCAACCGUGACCAAAGU | D-020920-04 |
| KIAA0355 | 9710 | 662 | CCGCAGGGACCUAGAAAUA | D-020920-03 |
| KIAA0355 | 9710 | 663 | GAGGCGACAUCUAGACUAA | D-020920-02 |
| KIAA0355 | 9710 | 664 | GCAGCUAUAUGGAUAAUGU | D-020920-01 |
| KIAA0586 | 9786 | 665 | UCAAACACCACCUCACUAA | D-020892-03 |
| KIAA0586 | 9786 | 666 | CAACAGAUUGCACCUAGUA | D-020892-02 |
| KIAA0586 | 9786 | 667 | CAAAGUUACCUACGUGUUA | D-020892-01 |
| KIAA0586 | 9786 | 668 | GGACAGAAAGAUGCUCUAA | D-020892-04 |
| KIAA0701 | 23074 | 669 | UGGAUGGGCCAAUGAGUUA | D-026913-06 |
| KIAA0701 | 23074 | 670 | GCUAAGCUAAUGUCUAGUU | D-026913-08 |
| KIAA0701 | 23074 | 671 | UCAAACAGACGUAUUACUG | D-026913-07 |
| KIAA0701 | 23074 | 672 | CUUCGGAUCUAUAGUGUAA | D-026913-05 |
| KIAA1012 | 22878 | 673 | GAAGAUGGCCCUUGUACUA | D-010645-04 |
| KIAA1012 | 22878 | 674 | GAAAGGAAAUACUGGAAUA | D-010645-01 |
| KIAA1012 | 22878 | 675 | AAAGAUGGCUUACCAAAUA | D-010645-02 |
| KIAA1012 | 22878 | 676 | GCACAUUGCUUUAUAAACA | D-010645-03 |
| KIAA1026 | 23254 | 677 | GAGCGAGGAUGCGGUCAAA | D-022166-01 |
| KIAA1026 | 23254 | 678 | GCUGAUCGGAAGCGCUUAA | D-022166-04 |
| KIAA1026 | 23254 | 679 | GCGAGACGGUGCUCAAUGG | D-022166-03 |
| KIAA1026 | 23254 | 680 | GCCAAACAGUCCUUAGCUA | D-022166-02 |
| KIF3C | 3797 | 681 | GAAUUAGGAUUUCAAAGUG | D-009469-03 |
| KIF3C | 3797 | 682 | UCAAGUACCUAAUCAUCGA | D-009469-05 |
| KIF3C | 3797 | 683 | GCAGCAAGAUGGCCAGUAA | D-009469-02 |
| KIF3C | 3797 | 684 | GUACAGGGCUGAAAACAUA | D-009469-04 |
| KLF12 | 11278 | 685 | GUAGAUCACUUCCAAACAC | D-013353-02 |
| KLF12 | 11278 | 686 | GACCUUAGAUAGCGUUAAU | D-013353-01 |
| KLF12 | 11278 | 687 | CUCCAAACGUCCACAACUA | D-013353-04 |
| KLF12 | 11278 | 688 | CCAUGAAUUUACAGUCUAA | D-013353-03 |
| KLHDC2 | 23588 | 689 | AAUCAGAGGUUUGGUAGUA | D-012839-03 |
| KLHDC2 | 23588 | 690 | UCGAGAUGCUAGAAUGAAU | D-012839-04 |
| KLHDC2 | 23588 | 691 | GAAUUCAAGUCAUCCAAGA | D-012839-02 |
| KLHDC2 | 23588 | 692 | CAACACUUCUGGAUCUUAA | D-012839-01 |
| KLHL1 | 57626 | 693 | CGAAAGAUACCUGCACAUA | D-010912-02 |
| KLHL1 | 57626 | 694 | GAAGCGGUGUGAGCACUUU | D-010912-03 |
| KLHL1 | 57626 | 695 | AGACAUACCUCAACACUAU | D-010912-01 |
| KLHL1 | 57626 | 696 | GUUCCCGGCUACUGGAUUA | D-010912-04 |
| LAPTM5 | 7805 | 697 | UCACUGUCCUUAUCUUCAA | D-019880-02 |
| LAPTM5 | 7805 | 698 | UGGCGGUGCUACAGAUUGA | D-019880-04 |
| LAPTM5 | 7805 | 699 | GAAGUGCCCACCUAUCUCA | D-019880-01 |
| LAPTM5 | 7805 | 700 | GUUCAUCGAGCACUCAGUA | D-019880-03 |
| LARS | 51520 | 701 | GGGAAAGCCUGACUCAAUU | D-010171-04 |
| LARS | 51520 | 702 | GAGUAAAGCUGCUGCUAAA | D-010171-01 |
| LARS | 51520 | 703 | GUACUGGAAUGCCUAUUAA | D-010171-03 |
| LARS | 51520 | 704 | GGGAAGCGGUAUACAAUUU | D-010171-02 |
| LCP2 | 3937 | 705 | GAAGGAAAGUCAAGUUUAC | D-012120-01 |
| LCP2 | 3937 | 706 | CAACAGACCACCUAUCAGA | D-012120-03 |
| LCP2 | 3937 | 707 | GGAGGAAGAGAAUUCAUUA | D-012120-02 |
| LCP2 | 3937 | 708 | AAACCAGGAUGGCACAUUU | D-012120-04 |
| LEFTB | 10637 | 709 | GAAGUGGGCCGAGAACUGG | D-013114-03 |
| LEFTB | 10637 | 710 | GCCAGGAGAUGUACAUUGA | D-013114-01 |
| LEFTB | 10637 | 711 | GACAGUGCAUCGCCUCGGA | D-013114-04 |
| LEFTB | 10637 | 712 | GCACCUCCCUCAUCGACUC | D-013114-02 |
| LNX2 | 222484 | 713 | CUUCAUAGCUGCCACGAUA | D-007164-03 |
| LNX2 | 222484 | 714 | GUGAACAGCUUGGCAUUAA | D-007164-04 |
| LNX2 | 222484 | 715 | GGACAUACAUUCUGCUACA | D-007164-02 |
| LNX2 | 222484 | 716 | CCAAGUGGCUCUUCAUAAA | D-007164-01 |
| LOC284214 | 284214 | 717 | CCAUCGAUGCUUUGAGCUA | D-031009-04 |
| LOC284214 | 284214 | 718 | GAAAAGGGCUACUCACUUA | D-031009-01 |
| LOC284214 | 284214 | 719 | CCACUUGGGUAGCCUAUGG | D-031009-03 |
| LOC284214 | 284214 | 720 | GAGUUGAGAUUUGGAUUAA | D-031009-02 |
| LOC285311 | 285311 | 721 | CAGAGGAGCUCCCUACUUG | D-023591-01 |
| LOC285311 | 285311 | 722 | CAUCAGAGAAACAAGCAGA | D-023591-04 |
| LOC285311 | 285311 | 723 | GAAAUUCCCACCCACCUAC | D-023591-03 |
| LOC285311 | 285311 | 724 | GGGUAUCUCUGGAUGGGUU | D-023591-02 |
| LOC285550 | 285550 | 725 | CUCAGUGUGUUAUUUGUAA | D-024218-04 |
| | | 726 | | |
| LOC285550 | 285550 | 727 | GGAAUAGUGAAGUGAAAUA | D-024218-02 |
| LOC285550 | 285550 | 728 | GUGCAAGACGUUAUAAUGA | D-024218-03 |
| LOC285550 | 285550 | 729 | GCGCAUAGUUGGCCAAUAU | D-024218-01 |
| LOC390530 | 390530 | 730 | GGUCAUAGCUGCAGGUGCC | D-030542-02 |
| LOC390530 | 390530 | 731 | GGAUUGGUGUGCCCUAGUG | D-030542-01 |
| LOC390530 | 390530 | 732 | GGACACAUCCAUGGGCACA | D-030542-03 |
| LOC390530 | 390530 | 733 | CAGCACAAGCUAUGCACAG | D-030542-04 |
| LOC402117 | 402117 | 734 | GUGACCAGAUCUCCAGUAA | D-028034-02 |
| LOC402117 | 402117 | 735 | GGAAAGGGUGUGUCGAUGA | D-028034-01 |
| LOC402117 | 402117 | 736 | GCUCAGUGUUCGAAACGUG | D-028034-04 |
| LOC402117 | 402117 | 737 | UGAAAGUGGACGAAUGUAA | D-028034-03 |
| LPL | 4023 | 738 | GAAGAGUGAUUCAUACUUU | D-008970-01 |
| LPL | 4023 | 739 | GCAACAAUCUGGGCUAUGA | D-008970-02 |
| LPL | 4023 | 740 | GAACCAGACUCCAAUGUCA | D-008970-04 |
| LPL | 4023 | 741 | CAGGAAGUCUGACCAAUAA | D-008970-03 |
| LRRC8D | 55144 | 742 | GAGAGUUGCGGCACCUUAA | D-015747-04 |
| LRRC8D | 55144 | 743 | GGUGGGAUGUGUUUAUGGA | D-015747-03 |
| LRRC8D | 55144 | 744 | GAUGAUAGGACUUGAAUCU | D-015747-02 |
| LRRC8D | 55144 | 745 | UUGAGCAUCUGAUUGGUUA | D-015747-01 |
| LSM3 | 27258 | 746 | GCAACAAACUACCAACACU | D-020240-02 |
| LSM3 | 27258 | 747 | GAGGCAGAUUACAUGCUUA | D-020240-04 |
| LSM3 | 27258 | 748 | GAUGAGCGAAUUUAUGUGA | D-020240-03 |
| LSM3 | 27258 | 749 | UAAAUAUGAUCUUGGGAGA | D-020240-01 |
| LY6D | 8581 | 750 | GAAGAAGGACUGUGCGGAG | D-012615-02 |
| LY6D | 8581 | 751 | GCAAGACCACGAACACAGU | D-012615-03 |
| LY6D | 8581 | 752 | CUGCAAGCAUUCUGUGGUC | D-012615-04 |
| LY6D | 8581 | 753 | GCAAUGAGAAGCUGCACAA | D-012615-01 |
| LYPD4 | 147719 | 754 | GCGCAAAUCUCCUACCUUG | D-018514-01 |
| LYPD4 | 147719 | 755 | GGUCUUAUCUCUGCAACAA | D-018514-02 |
| LYPD4 | 147719 | 756 | GGUGUGCUCGUGAACAUAA | D-018514-04 |
| LYPD4 | 147719 | 757 | GCGAGCACAUGAAGGAUUG | D-018514-03 |
| MAML1 | 9794 | 758 | GGCAUAACCCAGAUAGUUG | D-013417-05 |
| MAML1 | 9794 | 759 | UGAAGGACCUGUUUAAUGA | D-013417-01 |
| MAML1 | 9794 | 760 | CCACGCAUCUUCAUGAUAC | D-013417-04 |
| MAML1 | 9794 | 761 | AAUCAGAACUCCGCGAAUA | D-013417-02 |
| MAML2 | 84441 | 762 | GACAGAGCCUGGUAAUGAU | D-013568-04 |
| MAML2 | 84441 | 763 | CGAAAGUAAUGGCUAACUA | D-013568-02 |
| MAML2 | 84441 | 764 | GUAAUCAACCUAACACAUA | D-013568-01 |
| MAML2 | 84441 | 765 | AGACCAAAUUUAACCCAUA | D-013568-03 |
| MAP4 | 4134 | 766 | GGACAUGUCUCCACUAUCA | D-011724-01 |
| MAP4 | 4134 | 767 | GGAAUCACCCACCAAAUUA | D-011724-02 |
| MAP4 | 4134 | 768 | CGAGGAGGAUUCUGUGUUA | D-011724-04 |
| MAP4 | 4134 | 769 | CAACACCAGUUCCAAUUAA | D-011724-03 |
| MDN1 | 23195 | 770 | GAAAUUUGAUGGACUUUGA | D-009786-03 |
| MDN1 | 23195 | 771 | GCAAUUGUGUCUCAACUUU | D-009786-04 |
| MDN1 | 23195 | 772 | GAAAUACCCUUGUUAGAAU | D-009786-02 |
| MDN1 | 23195 | 773 | GGUCAUGGCUGUUAAAUUG | D-009786-01 |
| MED28 | 80306 | 774 | UGAGUGGGCUGAUGCGUGA | D-014606-03 |
| MED28 | 80306 | 775 | CAGAAACCAGAGCAAGUUA | D-014606-04 |
| MED28 | 80306 | 776 | GCGGAAAGAUGCACUAGUC | D-014606-01 |
| MED28 | 80306 | 777 | GUACUUUGGUGGACGAGUU | D-014606-02 |
| MED6 | 10001 | 778 | CAAGAUAAAGUCAGACCUA | D-019963-03 |
| MED6 | 10001 | 779 | GAAAGAGGCAGAACCUAUA | D-019963-01 |
| MED6 | 10001 | 780 | CCCACUAGCUGAUUACUAU | D-019963-04 |
| MED6 | 10001 | 781 | CAACAGACAGUGAGUGCUA | D-019963-02 |
| MGAT1 | 4245 | 782 | CCACCUAUCCGCUGCUGAA | D-011332-04 |
| MGAT1 | 4245 | 783 | GAGAAAGUGAGGACCAAUG | D-011332-02 |
| MGAT1 | 4245 | 784 | UGGACAAGCUGCUGCAUUA | D-011332-01 |
| MGAT1 | 4245 | 785 | GGAGGCCUAUGACCGAGAU | D-011332-03 |
| MGC13272 | 84315 | 786 | GCACACAUCUCUUACCUAG | D-014875-01 |
| MGC13272 | 84315 | 787 | CUGCGUCACUUCCUCUAUA | D-014875-02 |
| MGC13272 | 84315 | 788 | CUACAGCGUUGCCCAAGUG | D-014875-04 |
| MGC13272 | 84315 | 789 | GACCGCCUCUUCAUUCUCA | D-014875-03 |
| MGC14560 | 51184 | 790 | GUGGAGUCAUUCAAGUUUA | D-016860-03 |
| MGC14560 | 51184 | 791 | UAUGUGGACUGAUUGAUGA | D-016860-02 |
| MGC14560 | 51184 | 792 | GGAGGAUGAUUCUCUGCGA | D-016860-04 |
| MGC14560 | 51184 | 793 | CAGGUCAGAUUGAGUUGUA | D-016860-01 |
| MGC24039 | 160518 | 794 | GAUAAGAUAAGGCUGUAUA | D-017364-04 |
| MGC24039 | 160518 | 795 | UGAGUGAGAUCAAGACUGA | D-017364-03 |
| MGC24039 | 160518 | 796 | CGAUACAACUCCUAUGAUA | D-017364-01 |
| MGC24039 | 160518 | 797 | GCACAACGCUGAGCAUUAC | D-017364-02 |
| MGC27019 | 150483 | 798 | GAGACCAACUUGCUCCUGG | D-015660-01 |
| MGC27019 | 150483 | 799 | AGUUCAGGCUGUUGAGUGA | D-015660-03 |
| MGC27019 | 150483 | 800 | GACUGAACCGGGAGCACAA | D-015660-04 |
| MGC27019 | 150483 | 801 | CAGCAAGACUCCACGCGCA | D-015660-02 |
| MGC59937 | 375791 | 802 | CCCAAGAGAUGGUCGUCAA | D-027279-02 |
| MGC59937 | 375791 | 803 | GAACCCAUAUGCCCACAUC | D-027279-01 |
| MGC59937 | 375791 | 804 | UGACAUCGCCCACCACUGC | D-027279-03 |
| MGC59937 | 375791 | 805 | GGGCAGCAGUUAGAGGUGG | D-027279-04 |
| MID1IP1 | 58526 | 806 | UCUCGAAACUCACGCGCAA | D-015884-04 |
| MID1IP1 | 58526 | 807 | GCAAAUCUGCGACACCUAC | D-015884-03 |
| MID1IP1 | 58526 | 808 | CAGAAGCACUCGCUCUUUA | D-015884-01 |
| MID1IP1 | 58526 | 809 | GAGAUCGGCUUCGGCAAUU | D-015884-02 |
| MKRN2 | 23609 | 810 | GCAAUCACACGUACUGUUU | D-006960-02 |
| MKRN2 | 23609 | 811 | GAGAGGAGAUUUGGGAUUC | D-006960-04 |
| MKRN2 | 23609 | 812 | GGAACUCGGUGCAGAUAUG | D-006960-03 |
| MKRN2 | 23609 | 813 | GGAAACAGCUCAGUUCUCA | D-006960-01 |
| MOS | 4342 | 814 | GCAAGGCUGCGCCACGAUA | D-003859-01 |
| MOS | 4342 | 815 | GUGGAUCUCACCUCUUUGA | D-003859-03 |
| MOS | 4342 | 816 | GAAAGUGUCUCAAGUACUC | D-003859-05 |
| MOS | 4342 | 817 | CAAGGCUGCGCCACGAUAA | D-003859-04 |
| MPHOSPH6 | 10200 | 818 | UAGAAGAUAUGAGACCUUG | D-020018-04 |
| MPHOSPH6 | 10200 | 819 | GGACUGGACUCAGAAACCA | D-020018-03 |
| MPHOSPH6 | 10200 | 820 | AGAGAGACCAUGCCAAUUA | D-020018-02 |
| MPHOSPH6 | 10200 | 821 | GCACAAAGCAGAAGAAGUU | D-020018-01 |
| MR1 | 3140 | 822 | GAAUGUAUUGCCUGGCUAA | D-019619-03 |
| MR1 | 3140 | 823 | CAGAGAACCUCGCGCCUGA | D-019619-02 |
| MR1 | 3140 | 824 | CAGAGUAAAUCGCAAAGAA | D-019619-01 |
| MR1 | 3140 | 825 | CAUAUGACGGGCAGGAUUU | D-019619-04 |
| NALP12 | 91662 | 826 | GGAUGGACCUGAAUAAAAU | D-015092-01 |
| NALP12 | 91662 | 827 | CUACGGACUUUGUGGCUGA | D-015092-04 |
| NALP12 | 91662 | 828 | GGAUUUGGGCCUGAGGUUA | D-015092-03 |
| NALP12 | 91662 | 829 | CCAAUAAGAAUUUGACAAG | D-015092-02 |
| NCOR2 | 9612 | 830 | GAACCUCGAUGAGAUCUUG | D-020145-03 |
| NCOR2 | 9612 | 831 | GGAAAAGACUCAAAGUAAA | D-020145-04 |
| NCOR2 | 9612 | 832 | GGACGGAGAUCUUCAAUAU | D-020145-01 |
| NCOR2 | 9612 | 833 | GCGCACCUAUGACAUGAUG | D-020145-05 |
| NDUFB7 | 4713 | 834 | CGGCAGAGUUGGCCAAAGG | D-017213-03 |
| NDUFB7 | 4713 | 835 | GCAAGGAGCGCGAGAUGGU | D-017213-01 |
| NDUFB7 | 4713 | 836 | GCACCGCGACUAUGUGAUG | D-017213-02 |
| NDUFB7 | 4713 | 837 | CAACCUUCCCGCCAGACUA | D-017213-04 |
| NF2 | 4771 | 838 | GACAAGGAGUUUACUAUUA | D-003917-02 |
| NF2 | 4771 | 839 | GGAGACAGCUCUGGAUAUU | D-003917-03 |
| NF2 | 4771 | 840 | GGAAGGACCUCUUUGAUUU | D-003917-01 |
| NF2 | 4771 | 841 | GAAGAUGGCUGAGGAGUCA | D-003917-04 |
| NGLY1 | 55768 | 842 | GAGGAGCUGUUGAAUGUUU | D-016457-01 |
| NGLY1 | 55768 | 843 | GAAAUUGCGAUCUGAUACA | D-016457-04 |
| NGLY1 | 55768 | 844 | AGACAAAGCUUAAAUGACC | D-016457-02 |
| NGLY1 | 55768 | 845 | GCGAGUGGGCCAAUUGUUU | D-016457-03 |
| NIPSNAP3B | 55335 | 846 | AAACAAGAGACGGAAAUUA | D-015435-03 |
| NIPSNAP3B | 55335 | 847 | CCACACAGAAUAUGGAGAA | D-015435-04 |
| NIPSNAP3B | 55335 | 848 | CCUUAAACCUUCAAAUAUG | D-015435-02 |
| NIPSNAP3B | 55335 | 849 | GAUACCAUGGUCCAAAUUA | D-015435-01 |
| NMT1 | 4836 | 850 | GAAAUUGGUUGGGUUCAUU | D-004316-01 |
| NMT1 | 4836 | 851 | ACGGCAACCUGCAGUAUUA | D-004316-04 |
| NMT1 | 4836 | 852 | GCAGAAAUAUGACCAUGCA | D-004316-03 |
| NMT1 | 4836 | 853 | CAGCAAACAUCCAUAUCUA | D-004316-02 |
| NR0B2 | 8431 | 854 | CGUAGCCGCUGCCUAUGUA | D-003410-03 |
| NR0B2 | 8431 | 855 | GAAUAUGCCUGCCUGAAAG | D-003410-01 |
| NR0B2 | 8431 | 856 | GGAAUAUGCCUGCCUGAAA | D-003410-02 |
| NR0B2 | 8431 | 857 | GCCAUUCUCUACGCACUUC | D-003410-04 |
| NUP107 | 57122 | 858 | UAUCAGUGCUGUUAUGUUA | D-020440-04 |
| NUP107 | 57122 | 859 | CAUCAGAGCUUAUUUGGAA | D-020440-03 |
| NUP107 | 57122 | 860 | GAAAGUGUAUUCGCAGUUA | D-020440-02 |
| NUP107 | 57122 | 861 | GGAAAUCUCUCCAUGGUUA | D-020440-01 |
| NUP133 | 55746 | 862 | CCAGUAAUCGGGAAAGAUA | D-013322-02 |
| NUP133 | 55746 | 863 | GGAGGUAUCCCAAGUAGAU | D-013322-03 |
| NUP133 | 55746 | 864 | UAACUGAGUCUGUGAACUA | D-013322-01 |
| NUP133 | 55746 | 865 | CAUCCGAACGGUAAUAAUA | D-013322-04 |
| NUP153 | 9972 | 866 | GAGGAGAGCUCUAAUAUUA | D-005283-01 |
| NUP153 | 9972 | 867 | GGAAGAAAGCUGACAAUGA | D-005283-02 |
| NUP153 | 9972 | 868 | GAAGCGAGCCCUUACAUUG | D-005283-04 |
| NUP153 | 9972 | 869 | CAAUUCGUCUCAAGCAUUA | D-005283-03 |
| NUP155 | 9631 | 870 | CAUUUGGGAUGCAAGCUUA | D-011967-04 |
| NUP155 | 9631 | 871 | GGACUCAGCUAUGCUAAUU | D-011967-01 |
| NUP155 | 9631 | 872 | ACAUAGAGCUCUUUAUAGU | D-011967-03 |
| NUP155 | 9631 | 873 | CAACUCAGGCCACAAAUAU | D-011967-02 |
| NUP160 | 23279 | 874 | GAAUAUGCGUGGAUUGUGC | D-029990-04 |
| NUP160 | 23279 | 875 | GGCAACACGGGAUUUAUUA | D-029990-03 |
| NUP160 | 23279 | 876 | GAGCCAAACUGGAUUGAAU | D-029990-02 |
| NUP160 | 23279 | 877 | GGACACAAAUUACGGCUUG | D-029990-01 |
| NUP85 | 79902 | 878 | GGAUGUAGAUGUUUACUCU | D-014478-01 |
| NUP85 | 79902 | 879 | GUACGCCUCGGGACUGUUU | D-014478-03 |
| NUP85 | 79902 | 880 | GAAAGCCGUCCGCAACAAU | D-014478-04 |
| NUP85 | 79902 | 881 | UGACUCGGCUCUUGUACUC | D-014478-02 |
| OTUD3 | 23252 | 882 | UAAUGCAACUGGAUGUUCA | D-027582-03 |
| OTUD3 | 23252 | 883 | GACAAUAACAGAAGCGAAG | D-027582-04 |
| OTUD3 | 23252 | 884 | UCGCAAAGGUCACAAACAA | D-027582-02 |
| OTUD3 | 23252 | 885 | GAAAUCAGGGCUUAAAUGA | D-027582-01 |
| PANK1 | 53354 | 886 | GAACGCUGGUUAAAUUGGU | D-004057-02 |
| PANK1 | 53354 | 887 | UAAUACUGCUUAUGGGAAA | D-004057-06 |
| PANK1 | 53354 | 888 | UACCCUAUGUUGCUGGUUA | D-004057-07 |
| PANK1 | 53354 | 889 | GUGGAACGCUGGUUAAAUU | D-004057-08 |
| PCDH11X | 27328 | 890 | GACCUUAACUUGUCGCUGA | D-013619-02 |
| PCDH11X | 27328 | 891 | GCAAGUGAGUGUUACUGAU | D-013619-04 |
| PCDH11X | 27328 | 892 | CCAGAGAACUCGGCUAUAA | D-013619-01 |
| PCDH11X | 27328 | 893 | GGAAUAAACGGAGUUCAAA | D-013619-03 |
| PDIA6 | 10130 | 894 | GAAGUGAUAGUUCAAGUAA | D-020026-01 |
| PDIA6 | 10130 | 895 | GAUGAAAUUUGCUCUGCUA | D-020026-03 |
| PDIA6 | 10130 | 896 | CGAAUUAACUCCAUCGAAU | D-020026-02 |
| PDIA6 | 10130 | 897 | GACGACAGCUUUGAUAAGA | D-020026-04 |
| PHF12 | 57649 | 898 | CCAAUGAACUGACUUGUAC | D-009736-04 |
| PHF12 | 57649 | 899 | GAAGGUUCCUGAUGCUAUA | D-009736-02 |
| PHF12 | 57649 | 900 | GAAAGACUGUCCAAUCACA | D-009736-03 |
| PHF12 | 57649 | 901 | GCAAACAGCUGACAAGACA | D-009736-01 |
| PHF3 | 23469 | 902 | GGACGAAGUCAGCCUGUAA | D-014075-04 |
| PHF3 | 23469 | 903 | CGAUAAGGAUCCUAUGCUA | D-014075-03 |
| PHF3 | 23469 | 904 | UCAAGUAGGUGGCAGGAUA | D-014075-01 |
| PHF3 | 23469 | 905 | GAGGUUGACUCUAUGUCUA | D-014075-02 |
| PIGH | 5283 | 906 | GAGCGGAGCUUUUCGGAUA | D-011885-01 |
| PIGH | 5283 | 907 | AAAGAAAGCACUACCUUCA | D-011885-04 |
| PIGH | 5283 | 908 | UCUUAGGUCUGCUUGGUUA | D-011885-03 |
| PIGH | 5283 | 909 | GGUCAAGGAUAUUGUCAUC | D-011885-02 |
| PIGK | 10026 | 910 | CCAGCUAGCCAAACUAAUA | D-005996-06 |
| PIGK | 10026 | 911 | GGACAUCGCACUGAUCUUU | D-005996-04 |
| PIGK | 10026 | 912 | GAUAUGGCCUGUAAUCCUA | D-005996-02 |
| PIGK | 10026 | 913 | GUACGGAAAGUGGAAAUUA | D-005996-05 |
| PIGY | 84992 | 914 | GGGAAUAGCUUAUCCAAUC | D-015043-01 |
| PIGY | 84992 | 915 | GCUCAGAUAUGAAGCAUCA | D-015043-02 |
| PIGY | 84992 | 916 | GUAAGAAGCAAGAAGAAGU | D-015043-04 |
| PIGY | 84992 | 917 | GAUGGGAUCCCUAAUAUGA | D-015043-03 |
| PIP5K1C | 23396 | 918 | CCAAAUUCCUGUACUGUAA | D-004782-01 |
| PIP5K1C | 23396 | 919 | GGAGAUAUACUUGGUGUUG | D-004782-03 |
| PIP5K1C | 23396 | 920 | GGCAAGACCUAUUUAUAAU | D-004782-02 |
| PIP5K1C | 23396 | 921 | GAUAGAAGUCUGUAAAUAC | D-004782-04 |
| PKD1L2 | 114780 | 922 | GAGAAGGGGUCUACUAUUU | D-013421-02 |
| PKD1L2 | 114780 | 923 | GGACCAACGUCAAGGGUAU | D-013421-04 |
| PKD1L2 | 114780 | 924 | GAGAAAUCCUGGCAUACUU | D-013421-03 |
| PKD1L2 | 114780 | 925 | GAGAUUGCGUCCUCGAUAA | D-013421-01 |
| PLEKHA3 | 65977 | 926 | GAACCAGUAUCUACACUUC | D-004319-04 |
| PLEKHA3 | 65977 | 927 | UCACAACGCUUGAGGAAUG | D-004319-03 |
| PLEKHA3 | 65977 | 928 | GCAUAAAGAUGGCAGUUUG | D-004319-02 |
| PLEKHA3 | 65977 | 929 | CGAAGAACCUACUCAGAUA | D-004319-01 |
| PLEKHA4 | 57664 | 930 | GAGUCAACUUUCCACCAAA | D-020795-03 |
| PLEKHA4 | 57664 | 931 | CAGAUACGCUGCUGACCAA | D-020795-01 |
| PLEKHA4 | 57664 | 932 | UAAACAAGAUCCACGCCUU | D-020795-02 |
| PLEKHA4 | 57664 | 933 | CGAGAGAGGGUUUGGGACA | D-020795-04 |
| PLOD3 | 8985 | 934 | GGGAGAAACUCAGCCUUAA | D-004286-02 |
| PLOD3 | 8985 | 935 | ACAAGGGCCUGGACUAUGA | D-004286-04 |
| PLOD3 | 8985 | 936 | GCCUUAAUCUGGAUCAUAA | D-004286-03 |
| PLOD3 | 8985 | 937 | GACAUGGCCUUCUGUAAGA | D-004286-01 |
| PNRC1 | 10957 | 938 | GCUGAUGGCAGUACACUUA | D-019926-02 |
| PNRC1 | 10957 | 939 | GAUCCACCUUCUCCUAGUG | D-019926-04 |
| PNRC1 | 10957 | 940 | UAUGAGCAACCAAAGAUAA | D-019926-03 |
| PNRC1 | 10957 | 941 | GGAGAUGGCCCGUGUCUGA | D-019926-01 |
| PNRC2 | 55629 | 942 | GGACAUGGUUAUAACUCAU | D-015670-03 |
| PNRC2 | 55629 | 943 | CUUAUCAGGUCCCAGGUUA | D-015670-02 |
| PNRC2 | 55629 | 944 | CCUCAAUCUAGAAAUGUUA | D-015670-04 |
| POLR3A | 11128 | 945 | CUCAAGAGCUCAAGUAUGG | D-019741-03 |
| POLR3A | 11128 | 946 | GCACAAAUUGAGCAUUAUG | D-019741-04 |
| POLR3A | 11128 | 947 | GCCAAUGAUUCCUAUGUUA | D-019741-01 |
| POLR3A | 11128 | 948 | GAACGGAUUAGGCUUCUGA | D-019741-02 |
| POLR3F | 10621 | 949 | GCAGAGAUAUCCGCUAUAA | D-019240-01 |
| POLR3F | 10621 | 950 | GCGAAUUGGGAAUCAGUAA | D-019240-04 |
| POLR3F | 10621 | 951 | UCAUUUGCCUCAUCACAUG | D-019240-02 |
| POLR3F | 10621 | 952 | CAGUAGCCAUCAAUAGGUU | D-019240-03 |
| POU1F1 | 5449 | 953 | CAACAGGACUUCAUUAUUC | D-012546-01 |
| POU1F1 | 5449 | 954 | GCAAGUAGGAGCUUUGUAC | D-012546-03 |
| POU1F1 | 5449 | 955 | UAGGAUACACCCAGACAAA | D-012546-04 |
| POU1F1 | 5449 | 956 | GAACUCAGGCGGAAAAGUA | D-012546-02 |
| PPIB | 5479 | 957 | GAAAGAGCAUCUACGGUGA | D-004606-01 |
| PPIB | 5479 | 958 | GAAAGGAUUUGGCUACAAA | D-004606-02 |
| PPIB | 5479 | 959 | GGAAAGACUGUUCCAAAAA | D-004606-04 |
| PPIB | 5479 | 960 | ACAGCAAAUUCCAUCGUGU | D-004606-03 |
| PPP2R2A | 5520 | 961 | GAAAUUACAGACAGGAGUU | D-004824-02 |
| PPP2R2A | 5520 | 962 | GCAGAUGAUUUGCGGAUUA | D-004824-05 |
| PPP2R2A | 5520 | 963 | UAUCAAGCCUGCCAAUAUG | D-004824-03 |
| PPP2R2A | 5520 | 964 | UAUGAUGACUAGAGACUAU | D-004824-04 |
| PRDM14 | 63978 | 965 | GAGAUAAGCACCUCAAGUA | D-014346-01 |
| PRDM14 | 63978 | 966 | GAAGACCUACGGAGACAAU | D-014346-03 |
| PRDM14 | 63978 | 967 | CAGUGUGUGUAUUGUACUA | D-014346-04 |
| PRDM14 | 63978 | 968 | GUUCACAGCCUCCAGCAUA | D-014346-02 |
| PRDM7 | 11105 | 969 | AGUGGAUAUUCCUGGCUAA | D-015181-04 |
| PRDM7 | 11105 | 970 | GGGAGAAACUGCUAUGAGU | D-015181-01 |
| PRDM7 | 11105 | 971 | GAACGAGGCAUCUGAUCUG | D-015181-02 |
| PRDM7 | 11105 | 972 | GUCAACAUGUGGAACGCAA | D-015181-03 |
| PRKWNK1 | 65125 | 973 | GCAGGAGUGUCUAGUUAUA | D-005362-04 |
| PRKWNK1 | 65125 | 974 | GGAAGGCGGUUUAUAGUGA | D-005362-02 |
| PRKWNK1 | 65125 | 975 | GCAGUUGUCUCAAUAUCUA | D-005362-03 |
| PRKWNK1 | 65125 | 976 | UAUCGAAGAUGAAGACUUA | D-005362-01 |
| PRKX | 5613 | 977 | GCCUAAAGCAGGAGCAACA | D-004660-07 |
| PRKX | 5613 | 978 | GAUAGGGAUGGCCACAUUA | D-004660-04 |
| PRKX | 5613 | 979 | GAACAAGGCGAUUAGGAAA | D-004660-05 |
| PRKX | 5613 | 980 | GGAGCAACACGUACACAAU | D-004660-06 |
| PSME2 | 5721 | 981 | CCAAGGAGACUCAUGUAAU | D-011370-03 |
| PSME2 | 5721 | 982 | UGAAUGCCGUCAAGACCAA | D-011370-04 |
| PSME2 | 5721 | 983 | AUGCUGAGCUUUAUCAUAU | D-011370-02 |
| PSME2 | 5721 | 984 | GAAAUGCAUUCUGGUGAUU | D-011370-01 |
| PSPHL | 8781 | 985 | ACUACAGGAUGCUUUCAUU | D-032264-02 |
| PSPHL | 8781 | 986 | GCACUGUCAAGUAAACUAC | D-032264-03 |
| PSPHL | 8781 | 987 | GAAAAUUCUUCCAAGGAUG | D-032264-04 |
| PSPHL | 8781 | 988 | GAAGGAAUCGGACGGAGUC | D-032264-01 |
| PTPN9 | 5780 | 989 | GGAGAGGAUUCAAAUAUUA | D-008832-01 |
| PTPN9 | 5780 | 990 | UCGAAGAGAUUAACAAGUG | D-008832-03 |
| PTPN9 | 5780 | 991 | UCAGACAGAUUACAUCAAU | D-008832-04 |
| PTPN9 | 5780 | 992 | GAGAAUACCUAUCGUGAUU | D-008832-02 |
| PURA | 5813 | 993 | GCUUCUACCUGGACGUGAA | D-012136-04 |
| PURA | 5813 | 994 | CAACAAGCGCUUCUUCUUC | D-012136-03 |
| PURA | 5813 | 995 | GAUGUGGGCUCCAACAAGU | D-012136-02 |
| PURA | 5813 | 996 | GGACACACCUUCUGCAAGU | D-012136-01 |
| RAB1B | 81876 | 997 | CCAGCGAGAACGUCAAUAA | D-008958-01 |
| RAB1B | 81876 | 998 | GCGCCAAGAAUGCCACCAA | D-008958-02 |
| RAB1B | 81876 | 999 | CAGCCAAGGAGUUUGCAGA | D-008958-04 |
| RAB1B | 81876 | 1000 | GACCAUGGCUGCUGAAAUC | D-008958-03 |
| RAB2 | 5862 | 1001 | GAAGGAGUCUUUGACAUUA | D-010533-01 |
| RAB2 | 5862 | 1002 | GAUAUUACACGGAGAGAUA | D-010533-03 |
| RAB2 | 5862 | 1003 | UGACCUUACUAUUGGUGUA | D-010533-06 |
| RAB2 | 5862 | 1004 | CGAAUGAUAACUAUUGAUG | D-010533-05 |
| RAB28 | 9364 | 1005 | GAGCAUAUGCGAACAAUAA | D-008582-01 |
| RAB28 | 9364 | 1006 | UGAAGUACCCGGAAGAAGA | D-008582-04 |
| RAB28 | 9364 | 1007 | GGUAUACUGUGGUGAAGAA | D-008582-02 |
| RAB28 | 9364 | 1008 | GAAUGUUACCCUUCAAAUU | D-008582-03 |
| RAB6A | 5870 | 1009 | AAGCAGAGAAGAUAUGAUU | D-008975-05 |
| RAB6A | 5870 | 1010 | GAGCAACCAGUCAGUGAAG | D-008975-04 |
| RAB6A | 5870 | 1011 | CCAAAGAGCUGAAUGUUAU | D-008975-06 |
| RAB6A | 5870 | 1012 | GAGAAGAUAUGAUUGACAU | D-008975-01 |
| RAB6B | 51560 | 1013 | AGACGGACCUGGCUGAUAA | D-008548-01 |
| RAB6B | 51560 | 1014 | GGAAUCCACUGAGAAAAUU | D-008548-04 |
| RAB6B | 51560 | 1015 | CCAAAGAACUGAGCGUCAU | D-008548-03 |
| RAB6B | 51560 | 1016 | GGAAGACGUCUCUGAUUAC | D-008548-02 |
| RAB6C | 84084 | 1017 | AAUAUUGGCUUGAACCUUU | D-009031-02 |
| RAB6C | 84084 | 1018 | GGGCUGAAUGUUACGUUUA | D-009031-04 |
| RAB6C | 84084 | 1019 | CUGCAGCUGUAGUAGUUUA | D-009031-05 |
| RAB6C | 84084 | 1020 | CUACAAAGUGGAUUGAUGA | D-009031-03 |
| RAB9P40 | 10244 | 1021 | GAAACCAGCUAUAUGUCUU | D-019457-03 |
| RAB9P40 | 10244 | 1022 | GGAUUCAGCUGACAAAGUA | D-019457-02 |
| RAB9P40 | 10244 | 1023 | GGAAAUCGAAAUUGUCUAC | D-019457-04 |
| RAB9P40 | 10244 | 1024 | CCACAGCUGUUCAUAUUUA | D-019457-01 |
| RANBP2 | 5903 | 1025 | GAAAGGACAUGUAUCACUG | D-004746-07 |
| RANBP2 | 5903 | 1026 | GAAAGAAGGUCACUGGGAU | D-004746-06 |
| RANBP2 | 5903 | 1027 | CGAAACAGCUGUCAAGAAA | D-004746-05 |
| RANBP2 | 5903 | 1028 | GAAUAACUAUCACAGAAUG | D-004746-08 |
| RANBP2L1 | 84220 | 1029 | GCAAAGCUGUAUUAUGAAG | D-012007-01 |
| RANBP2L1 | 84220 | 1030 | UUACAGGCGUUCAGUGGAA | D-012007-04 |
| RANBP2L1 | 84220 | 1031 | GAAGUCCUGCAAUUUAUAA | D-012007-03 |
| RANBP2L1 | 84220 | 1032 | GUUCCAAGACCAAAGAUUA | D-012007-02 |
| RAP1B | 5908 | 1033 | AGUAUAAGCUAGUCGUUCU | D-010364-05 |
| RAP1B | 5908 | 1034 | GACCUAGUGCGGCAAAUUA | D-010364-04 |
| RAP1B | 5908 | 1035 | CAAUGAUUCUUGUUGGUAA | D-010364-03 |
| RAP1B | 5908 | 1036 | GAACAACUGUGCAUUCUUA | D-010364-01 |
| RAP80 | 51720 | 1037 | GGACACAUCUAGGCACUGU | D-006995-05 |
| RAP80 | 51720 | 1038 | GCACAAGACUUCAGAUGCA | D-006995-04 |
| RAP80 | 51720 | 1039 | GAAAAUGGGUUGCAGAAAA | D-006995-01 |
| RAP80 | 51720 | 1040 | AGAGGCAGCUCCUUAAUAA | D-006995-03 |
| RAPGEF1 | 2889 | 1041 | GAGGAACGACGACAUUAUA | D-006840-03 |
| RAPGEF1 | 2889 | 1042 | GCAGAACGAUCCUCGAAUU | D-006840-01 |
| RAPGEF1 | 2889 | 1043 | AUGCUGAGCUCUUCUAUAA | D-006840-04 |
| RAPGEF1 | 2889 | 1044 | GUACAGAUAUGAGAAAUUC | D-006840-02 |
| RAPGEF2 | 9693 | 1045 | GGACAAAGAAUACAUGAAA | D-009742-02 |
| RAPGEF2 | 9693 | 1046 | GGAAGAGCAUUCAGUAGUA | D-009742-01 |
| RAPGEF2 | 9693 | 1047 | GGAAGAAAGUGCCCGUAAA | D-009742-04 |
| RAPGEF2 | 9693 | 1048 | UCAAGUGGCUCCCAUGAUA | D-009742-03 |
| RELA | 5970 | 1049 | GGCUAUAACUCGCCUAGUG | D-003533-05 |
| RELA | 5970 | 1050 | GAUGAGAUCUUCCUACUGU | D-003533-01 |
| RELA | 5970 | 1051 | GGAUUGAGGAGAAACGUAA | D-003533-03 |
| RELA | 5970 | 1052 | CUCAAGAUCUGCCGAGUGA | D-003533-04 |
| RFPL2 | 10739 | 1053 | GAAGUGGGCGCAUCAGACA | D-006935-04 |
| RFPL2 | 10739 | 1054 | GAAGGUGGUUCCCAUGUCU | D-006935-03 |
| RFPL2 | 10739 | 1055 | CUUCGUAGACCGCAAGUUA | D-006935-01 |
| RFPL2 | 10739 | 1056 | UUAAUUCACUGCAGAAGGA | D-006935-02 |
| RFPL3 | 10738 | 1057 | CUUAGUAGACCGCAAGUUA | D-006934-02 |
| RFPL3 | 10738 | 1058 | GCUCAGACUAUCUGGAAAA | D-006934-01 |
| RFPL3 | 10738 | 1059 | CCAUGGUCUCUCAGAGGAA | D-006934-04 |
| RFPL3 | 10738 | 1060 | GAGAAUCUGUUCACUGCAA | D-006934-03 |
| RICS | 9743 | 1061 | GAAGAUAGGACGGAAAUUA | D-008213-02 |
| RICS | 9743 | 1062 | GGAAAGAGCUUGUACAGUU | D-008213-01 |
| RICS | 9743 | 1063 | UCACUCAGCUUCAGCCUUA | D-008213-04 |
| RICS | 9743 | 1064 | GAAACUGAGUCCAUUCUUU | D-008213-03 |
| RIMS4 | 140730 | 1065 | CAAAGUCGCUCGCAAGUCG | D-021322-01 |
| RIMS4 | 140730 | 1066 | GCUCAGAGGGCAACCUUAA | D-021322-02 |
| RIMS4 | 140730 | 1067 | AGACACUGCCAGCGGCCUA | D-021322-04 |
| RIMS4 | 140730 | 1068 | GAGUUUGCCUGGCGUCGGA | D-021322-03 |
| RNF139 | 11236 | 1069 | GAACUGUGCUUAAAAGUAA | D-006942-02 |
| RNF139 | 11236 | 1070 | CAGAGAGACUUUACUGUUU | D-006942-03 |
| RNF139 | 11236 | 1071 | GGGAAAAGCUUGACGAUUA | D-006942-01 |
| RNF139 | 11236 | 1072 | GCACAUGUAUCGAAUUUAC | D-006942-04 |
| RNF170 | 81790 | 1073 | GAAACUGGAUGAUGAUUCA | D-007078-01 |
| RNF170 | 81790 | 1074 | GAGAUUGCAUCAGGAUAUU | D-007078-04 |
| RNF170 | 81790 | 1075 | GGCCAAAUAUCAAGGUGAA | D-007078-03 |
| RNF170 | 81790 | 1076 | GGGCAACCCAGAUCUAUUA | D-007078-02 |
| RNF26 | 79102 | 1077 | UGACUAGUCUUCUGCACUU | D-007060-01 |
| RNF26 | 79102 | 1078 | GCCGAGAGAGGCUCAAUGA | D-007060-03 |
| RNF26 | 79102 | 1079 | GCAGAUCAGAGGCAGAAGA | D-007060-04 |
| RNF26 | 79102 | 1080 | CGUAGUGGCUGCCUUCCUA | D-007060-02 |
| RPL32P3 | 132241 | 1081 | GCAAAUUGAGAGUGUCUUG | D-028163-01 |
| RPL32P3 | 132241 | 1082 | GAAUAAUGUCUGGGGUGAA | D-028163-04 |
| RPL32P3 | 132241 | 1083 | UACCACGGGUGCAGGAAUU | D-028163-02 |
| RPL32P3 | 132241 | 1084 | ACACUGGCAGGGUGGGAUU | D-028163-03 |
| RPTN | 126638 | 1085 | GAGGCUAGGUCAGGAAUUA | D-027449-02 |
| RPTN | 126638 | 1086 | AGACCAAAGUUCUCACUAU | D-027449-03 |
| RPTN | 126638 | 1087 | CCAGAGCUAUCAUUAUGGU | D-027449-04 |
| RPTN | 126638 | 1088 | GGACAAGCCUCUCACUUUA | D-027449-01 |
| RRAGB | 10325 | 1089 | GGGAUGAAACCCUCUAUAA | D-012189-04 |
| RRAGB | 10325 | 1090 | GAGAAUGGAUCCACCACUA | D-012189-03 |
| RRAGB | 10325 | 1091 | UAUCGAAGCUGAUGAAGUA | D-012189-01 |
| RRAGB | 10325 | 1092 | CAAAUUAUAUUGCCAGAGA | D-012189-02 |
| RSL1D1 | 26156 | 1093 | GGGAGACAUUUCUAUCAAA | D-022489-02 |
| RSL1D1 | 26156 | 1094 | UCCAGUAUCUGUAAACCUU | D-022489-04 |
| RSL1D1 | 26156 | 1095 | AGAUUGACCUUGCCUCAUA | D-022489-01 |
| RSL1D1 | 26156 | 1096 | GACGCUCUCUUGACGCAUU | D-022489-03 |
| RTN2 | 6253 | 1097 | GCUCAGAUCGACCAAUAUG | D-012717-04 |
| RTN2 | 6253 | 1098 | GGGAGUGAUUGGUCUAUUC | D-012717-02 |
| RTN2 | 6253 | 1099 | GGGAGCAGACGGAACGUUU | D-012717-03 |
| RTN2 | 6253 | 1100 | UAGAAGACCUCGUGGAUUC | D-012717-01 |
| RUSC2 | 9853 | 1101 | GGUGGGAGCACAAGACCUA | D-026133-04 |
| RUSC2 | 9853 | 1102 | GGUUUAAUCACCUCUAUAA | D-026133-02 |
| RUSC2 | 9853 | 1103 | GAGCCUAGACCGAAGAUCA | D-026133-03 |
| RUSC2 | 9853 | 1104 | GGACAAGUAUACACGAAUA | D-026133-01 |
| S100A6 | 6277 | 1105 | GGGCCUUGGCUUUGAUCUA | D-013463-04 |
| S100A6 | 6277 | 1106 | GCUCGAAGCUGCAGGAUGC | D-013463-05 |
| S100A6 | 6277 | 1107 | ACAAGGACCAGGAGGUGAA | D-013463-03 |
| S100A6 | 6277 | 1108 | AGAAGGAGCUCACCAUUGG | D-013463-01 |
| SCAMPS | 192683 | 1109 | GAACGAACAUUGGCUCGGC | D-016650-04 |
| SCAMPS | 192683 | 1110 | GUGAACAACUUCCCACCAU | D-016650-03 |
| SCAMPS | 192683 | 1111 | CAACUUUGGCCUCGCCUUU | D-016650-01 |
| SCAMPS | 192683 | 1112 | GUACUCCAAUGAGAUGUGA | D-016650-02 |
| SCFD1 | 23256 | 1113 | GCAAGAAAAUUGGAUGUAU | D-010943-04 |
| SCFD1 | 23256 | 1114 | CGAAACUGAUGACUAUAGA | D-010943-02 |
| SCFD1 | 23256 | 1115 | UAAGGAGCUUGUUUCAUAU | D-010943-03 |
| SCFD1 | 23256 | 1116 | GACAAUACCGCUAAGCUAA | D-010943-01 |
| SCRN2 | 90507 | 1117 | CCAGUGCACCUACAUUGAA | D-015516-03 |
| SCRN2 | 90507 | 1118 | CAUCCCGGCUGUGAUCUUU | D-015516-02 |
| SCRN2 | 90507 | 1119 | GCGCCAUUCUCCUACCAUA | D-015516-01 |
| SCRN2 | 90507 | 1120 | UCAGGUAGAUCGUCGGCAU | D-015516-04 |
| SEC14L1 | 6397 | 1121 | AUACUACCUUCGCCAAUUA | D-011386-02 |
| SEC14L1 | 6397 | 1122 | CAAUAUACCAGCAACAUUA | D-011386-01 |
| SEC14L1 | 6397 | 1123 | GCACAUUGAGGCUUAUAAU | D-011386-03 |
| SEC14L1 | 6397 | 1124 | CCGACCACAUCAAGAGAUA | D-011386-04 |
| SECISBP2 | 79048 | 1125 | UCAAAGAACUGGUCCGUUU | D-015634-01 |
| SECISBP2 | 79048 | 1126 | GCGAGGGCAUCAAGUUAUC | D-015634-04 |
| SECISBP2 | 79048 | 1127 | GACUAAACGUCGACUUGUG | D-015634-03 |
| SECISBP2 | 79048 | 1128 | GGACACCAAUGGGUUAUGU | D-015634-02 |
| SEPT8 | 23176 | 1129 | GAACAGAUCAGAUAUAGGA | D-010647-01 |
| SEPT8 | 23176 | 1130 | CUACAUCGAUGCGCAGUUU | D-010647-04 |
| SEPT8 | 23176 | 1131 | GAUGAGAGGCCCAUAGUUG | D-010647-03 |
| SEPT8 | 23176 | 1132 | UAAGUGAGCUGCAGAGGAA | D-010647-02 |
| SESTD1 | 91404 | 1133 | UAUAGAACCAUGCCAAUUA | D-018379-02 |
| SESTD1 | 91404 | 1134 | GAACUUAAUCAGCAAAUUG | D-018379-01 |
| SESTD1 | 91404 | 1135 | GAGAGUACAUAGAUUGGAA | D-018379-03 |
| SESTD1 | 91404 | 1136 | GGAUGAAACUAGUUAAUCU | D-018379-04 |
| SET7 | 80854 | 1137 | GGAGUGUGCUGGAUAUAUU | D-014643-01 |
| SET7 | 80854 | 1138 | CAAACUGCAUCUACGAUAU | D-014643-02 |
| SET7 | 80854 | 1139 | GAGAGGACCGCACUUUAUG | D-014643-04 |
| SET7 | 80854 | 1140 | CCUGGACGAUGACGGAUUA | D-014643-03 |
| SFT2D1 | 113402 | 1141 | GGGACUGGCUGUGUUAUUC | D-016199-02 |
| SFT2D1 | 113402 | 1142 | GAACUGGAUUGCUGUGGCU | D-016199-04 |
| SFT2D1 | 113402 | 1143 | GGGAUGCAGUUAUUAAAUG | D-016199-01 |
| SFT2D1 | 113402 | 1144 | UUGCAGUUCUUGUCAAUGA | D-016199-03 |
| SIP1 | 8487 | 1145 | GAAGCAAAGUGUGAAUAUU | D-019545-03 |
| SIP1 | 8487 | 1146 | GAUCGAAGCAGCUCAAUGU | D-019545-04 |
| SIP1 | 8487 | 1147 | UAACUAGUGUCUUGGAAUA | D-019545-02 |
| SIP1 | 8487 | 1148 | GAGCGGAACUGGCUGGUUU | D-019545-01 |
| SIX1 | 6495 | 1149 | GCCAGGAGCUCAAACUAUU | D-020093-01 |
| SIX1 | 6495 | 1150 | GGAGAACACCGAAAACAAU | D-020093-04 |
| SIX1 | 6495 | 1151 | GAAGGCGCAUUACGUGGAG | D-020093-03 |
| SIX1 | 6495 | 1152 | GCACAAGAACGAGAGCGUA | D-020093-02 |
| SIX4 | 51804 | 1153 | GAUGGAGGGUCUGUAGUGA | D-020267-02 |
| SIX4 | 51804 | 1154 | UGUCUUAGAUGGCAUGGUU | D-020267-03 |
| SIX4 | 51804 | 1155 | CCAGUGGAGUUAUCCUUAA | D-020267-01 |
| SIX4 | 51804 | 1156 | GUAUACACGGUUCCUAAUA | D-020267-04 |
| SLC46A1 | 113235 | 1157 | GGUGAUCACUGUGCACUUU | D-018653-01 |
| SLC46A1 | 113235 | 1158 | CAUCUUAACCCUUUAUGAA | D-018653-03 |
| SLC46A1 | 113235 | 1159 | CGGUAGAGCCGCUGGUCUU | D-018653-04 |
| SLC46A1 | 113235 | 1160 | GGAAACAUUUAGCCCUCUA | D-018653-02 |
| SMBP | 56889 | 1161 | GUACAUAGAUGAUUUACCA | D-010220-03 |
| SMBP | 56889 | 1162 | GAAAUCGAAUUGUUGAUGU | D-010220-04 |
| SMBP | 56889 | 1163 | GAAGUUGUCUUAUGGAUGA | D-010220-01 |
| SMBP | 56889 | 1164 | CAACUGCAAUCUAUGUUUA | D-010220-02 |
| SNAP23 | 8773 | 1165 | GAAUCAAGACCAUCACUAU | D-017545-01 |
| SNAP23 | 8773 | 1166 | GAGAUCGUAUUGAUAUUGC | D-017545-03 |
| SNAP23 | 8773 | 1167 | CAACUAAACCGCAUAGAAG | D-017545-02 |
| SNAP23 | 8773 | 1168 | GGGUUUAGCCAUUGAGUCU | D-017545-04 |
| SNX27 | 81609 | 1169 | GGAACAACGGUUACAGUCA | D-017346-02 |
| SNX27 | 81609 | 1170 | CCAAGUAUAUCAGGCUAUC | D-017346-03 |
| SNX27 | 81609 | 1171 | GUGAAUUACUUUGCCUUAU | D-017346-04 |
| SNX27 | 81609 | 1172 | GUACGUAAAUUGGCACCUA | D-017346-01 |
| SP110 | 3431 | 1173 | GAAUAUACGUUGUGAAGGA | D-011875-05 |
| SP110 | 3431 | 1174 | CAAAUUAACCUGCGUGAAU | D-011875-03 |
| SP110 | 3431 | 1175 | CUGGAAGCCUGUAGAAAUU | D-011875-06 |
| SP110 | 3431 | 1176 | AAAGAUGACUCAACUUGUA | D-011875-01 |
| SPAST | 6683 | 1177 | GAACUUCAACCUUCUAUAA | D-014070-01 |
| SPAST | 6683 | 1178 | GGAAGACAAUGCUGGCUAA | D-014070-04 |
| SPAST | 6683 | 1179 | AAACGGACGUCUAUAAUGA | D-014070-02 |
| SPAST | 6683 | 1180 | UAUAAGUGCUGCAAGUUUA | D-014070-03 |
| SPCS3 | 60559 | 1181 | ACACGUAUCUGUCCCAUUU | D-010124-03 |
| SPCS3 | 60559 | 1182 | GAAGUGAUCUGGGAUUUAU | D-010124-02 |
| SPCS3 | 60559 | 1183 | GAACCAAGUUGUCCUAUGG | D-010124-04 |
| SPCS3 | 60559 | 1184 | GAAAUGGUCUCAAGGGAAA | D-010124-01 |
| SPPL3 | 121665 | 1185 | CAGCCUACAUCUUCAAUAG | D-006042-04 |
| SPPL3 | 121665 | 1186 | UGACUCAGUUCAAGUAGUU | D-006042-03 |
| SPPL3 | 121665 | 1187 | GAACAAGAUUUCCUUUGGU | D-006042-02 |
| SPPL3 | 121665 | 1188 | GGCAACAGCACCAAUAAUA | D-006042-01 |
| SPTAN1 | 6709 | 1189 | GAGAGGAACUGAUUACAAA | D-009933-04 |
| SPTAN1 | 6709 | 1190 | GCAAAGAUCUUACCAAUGU | D-009933-01 |
| SPTAN1 | 6709 | 1191 | CAACAGAGGUAAGGAUUUA | D-009933-03 |
| SPTAN1 | 6709 | 1192 | GCAAGAAGCUGUCCGAUGA | D-009933-02 |
| SPTBN1 | 6711 | 1193 | GACGAGAUCUUGUGGGUUG | D-018149-02 |
| SPTBN1 | 6711 | 1194 | CGAGUGCAAUGAAACCAAA | D-018149-04 |
| SPTBN1 | 6711 | 1195 | CGGAAGAGAUCGCCAAUUA | D-018149-01 |
| SPTBN1 | 6711 | 1196 | CUUAUGUGGUGACUUAUUA | D-018149-03 |
| SRP46 | 10929 | 1197 | CAAAUCGAGCUCUGCGCGA | D-012323-03 |
| SRP46 | 10929 | 1198 | CACUACAGCUCAUCUGGUU | D-012323-01 |
| SRP46 | 10929 | 1199 | GAAUCUCGCUACGGCGGAU | D-012323-02 |
| SRP46 | 10929 | 1200 | CGAUCUCGCUAUAGGGGUU | D-012323-04 |
| SSB | 6741 | 1201 | GAACAUUGCAUAAAGCAUU | D-006877-02 |
| SSB | 6741 | 1202 | GCUAAGAAAUUUGUAGAGA | D-006877-04 |
| SSB | 6741 | 1203 | AGAUAAAGGUCAAGUACUA | D-006877-03 |
| SSB | 6741 | 1204 | GAAAUGAAAUCUCUAGAAG | D-006877-01 |
| ST3GAL5 | 8869 | 1205 | CAAUGGCGCUGUUAUUUGA | D-011546-01 |
| ST3GAL5 | 8869 | 1206 | GUGCACCAGUUGAGGGAUA | D-011546-02 |
| ST3GAL5 | 8869 | 1207 | GACCAUGCAUAAUGUGACA | D-011546-03 |
| ST3GAL5 | 8869 | 1208 | CGGAAGUUCUCCAGUAAAG | D-011546-04 |
| ST6GALNAC5 | 81849 | 1209 | GGACGGAUACCUCGGAGUG | D-014685-02 |
| ST6GALNAC5 | 81849 | 1210 | GGCAAAGACAGGAAGAUAU | D-014685-03 |
| ST6GALNAC5 | 81849 | 1211 | GAUCAAUGUUUAUGGCAUG | D-014685-01 |
| ST6GALNAC5 | 81849 | 1212 | GGGCACGGACAUUCAAUAU | D-014685-04 |
| STAC2 | 342667 | 1213 | CAAGAUCGGCGACCGGGUU | D-027277-04 |
| STAC2 | 342667 | 1214 | AAACAGGGCUUGCGAUGUA | D-027277-01 |
| STAC2 | 342667 | 1215 | CGACAAGGAGCCUGAGUGA | D-027277-02 |
| STAC2 | 342667 | 1216 | GCAAGGAUGCUGACGGCUU | D-027277-03 |
| STARD3NL | 83930 | 1217 | CCUCUUAUUCGUAACAUUA | D-018591-02 |
| STARD3NL | 83930 | 1218 | AGAGAGGGCAGCACUUAUA | D-018591-04 |
| STARD3NL | 83930 | 1219 | CAGGAGGACUUUCUGUUUG | D-018591-01 |
| STARD3NL | 83930 | 1220 | GCAUUGAGAACACAUUAGA | D-018591-03 |
| STT3A | 3703 | 1221 | GACAAUAACACAUGGAAUA | D-017073-02 |
| STT3A | 3703 | 1222 | CCACAUACAUGAAGAAUCU | D-017073-03 |
| STT3A | 3703 | 1223 | UAAAGGACCUGGAUAAUCG | D-017073-04 |
| STT3A | 3703 | 1224 | GCAGUAGGAUCAUAUUUGA | D-017073-01 |
| STX5A | 6811 | 1225 | GAGCUAACAUAUAUCAUCA | D-017768-02 |
| STX5A | 6811 | 1226 | AGUCGAAACUGGCUUCUAU | D-017768-04 |
| STX5A | 6811 | 1227 | GCAAGUCCCUCUUUGAUGA | D-017768-01 |
| STX5A | 6811 | 1228 | GAGCCCAGCUGGACGUUGA | D-017768-03 |
| SUI1 | 10209 | 1229 | UCGCUGAUGAUUACGAUAA | D-015804-03 |
| SUI1 | 10209 | 1230 | UAAUUGAGCAUCCGGAAUA | D-015804-01 |
| SUI1 | 10209 | 1231 | GUGAAGGCGUUUAAGAAAA | D-015804-04 |
| SUI1 | 10209 | 1232 | UAAUUCAGCUACAGGGUGA | D-015804-02 |
| SUV420H1 | 51111 | 1233 | GAGGCAAGUUGUCUAAUGA | D-013366-02 |
| SUV420H1 | 51111 | 1234 | GAGAGGAGGUCGAACAGAU | D-013366-03 |
| SUV420H1 | 51111 | 1235 | GAGGAGAACAUGCUACUUA | D-013366-01 |
| SUV420H1 | 51111 | 1236 | UAGCAAAUAUGGACUCAGA | D-013366-04 |
| TAOK1 | 57551 | 1237 | CCAAGUAUCUCGUCACAAA | D-004846-02 |
| TAOK1 | 57551 | 1238 | GAACAGACCCGGAAAUUAG | D-004846-04 |
| TAOK1 | 57551 | 1239 | GGUCACACAUGUCUUAUAC | D-004846-05 |
| TAOK1 | 57551 | 1240 | GAACAAAUGUCUGGCUAUA | D-004846-01 |
| TCEB3 | 6924 | 1241 | AGAAAGAGGUGUCACAGAA | D-005143-03 |
| TCEB3 | 6924 | 1242 | GCAGCACUGUUUCCUAUGA | D-005143-04 |
| TCEB3 | 6924 | 1243 | GUAAAUAGCUUGCGAAAAC | D-005143-05 |
| TCEB3 | 6924 | 1244 | GAAAGGUGCCUGAUGUGUU | D-005143-06 |
| TFAM | 7019 | 1245 | CGGAGUGGCAGGUAUAUAA | D-019734-01 |
| TFAM | 7019 | 1246 | UCUUCUACGUCGCACAAUA | D-019734-03 |
| TFAM | 7019 | 1247 | GAAGAAUUGCCCAGCGUUG | D-019734-02 |
| TFAM | 7019 | 1248 | CCAAGAAGCUAAGGGUGAU | D-019734-04 |
| TFAP4 | 7023 | 1249 | GGAUUCCAGUCCCUCAAGA | D-009504-01 |
| TFAP4 | 7023 | 1250 | GAAGGUGCCCUCUUUGCAA | D-009504-02 |
| TFAP4 | 7023 | 1251 | GCCCACAUGUACCCGGAAA | D-009504-04 |
| TFAP4 | 7023 | 1252 | GCAGACAGCCGAGUACAUC | D-009504-03 |
| TFDP2 | 7029 | 1253 | GGAUAGAACGGAUAAAGCA | D-003328-09 |
| TFDP2 | 7029 | 1254 | CGAAAUCCCUGGUGCCAAA | D-003328-07 |
| TFDP2 | 7029 | 1255 | CACAGGACCUUCUUGGUUA | D-003328-06 |
| TFDP2 | 7029 | 1256 | UGAGAUCCAUGAUGACAUA | D-003328-08 |
| TFE3 | 7030 | 1257 | GGCAGCAGGUGAAACAGUA | D-009363-04 |
| TFE3 | 7030 | 1258 | GCUCAAGCCUCCCAAUAUC | D-009363-05 |
| TFE3 | 7030 | 1259 | CGCAGGCGAUUCAACAUUA | D-009363-03 |
| TFE3 | 7030 | 1260 | GGAAUCUGCUUGAUGUGUA | D-009363-01 |
| THAP3 | 90326 | 1261 | AAACAUGGACACUGCACUU | D-031883-03 |
| THAP3 | 90326 | 1262 | GCAAGAACCUAAAGCACAA | D-031883-01 |
| THAP3 | 90326 | 1263 | CCACGGUGUUCGCCUUUCA | D-031883-02 |
| THAP3 | 90326 | 1264 | AGGAAUGGGUGCUGAACAU | D-031883-04 |
| THOC2 | 57187 | 1265 | GGAGAGACGUGUUCAAUAU | D-025006-01 |
| THOC2 | 57187 | 1266 | CAGCAUAGAUAUCGUCUGU | D-025006-03 |
| THOC2 | 57187 | 1267 | GAAAUAAGGCUGAUCAAUU | D-025006-02 |
| THOC2 | 57187 | 1268 | AAAGAACGCCGAAGUCUGA | D-025006-04 |
| TIAM2 | 26230 | 1269 | GAACUUCAGGCGUCACAUA | D-008434-05 |
| TIAM2 | 26230 | 1270 | UAAGAGAGCCGUCAUACUG | D-008434-08 |
| TIAM2 | 26230 | 1271 | GUGUAAGGAUCGCCUGGUA | D-008434-07 |
| TIAM2 | 26230 | 1272 | CGACCUAAAUUCUGUUCUA | D-008434-06 |
| TIMM8A | 1678 | 1273 | GAACAGACCCAGAAAUCCA | D-010342-01 |
| TIMM8A | 1678 | 1274 | UGGACAAGCCUGGGCCAAA | D-010342-02 |
| TIMM8A | 1678 | 1275 | GUUGAGCGCUUCAUUGAUA | D-010342-03 |
| TIMM8A | 1678 | 1276 | ACUGAACUUUGUUGGGAGA | D-010342-04 |
| TM9SF2 | 9375 | 1277 | GGAAAGCGCCCAUCUGAAA | D-010221-04 |
| TM9SF2 | 9375 | 1278 | GAAUUUGGCUGGAAACUUG | D-010221-01 |
| TM9SF2 | 9375 | 1279 | GCACAAAGAUAUUGCUAGA | D-010221-02 |
| TM9SF2 | 9375 | 1280 | CCUAUUGGCUGUUACAUUA | D-010221-03 |
| TMED2 | 10959 | 1281 | GAACAAGCUAGAAGAAAUG | D-008074-03 |
| TMED2 | 10959 | 1282 | GACAAGAUAUGGAAACAGA | D-008074-01 |
| TMED2 | 10959 | 1283 | UCUACUACCUGAAGAGAUU | D-008074-04 |
| TMED2 | 10959 | 1284 | GGAUGGAACAUACAAAUUU | D-008074-02 |
| TMEM132C | 92293 | 1285 | CAAUAACCGUGCUAGAUGA | D-027086-02 |
| TMEM132C | 92293 | 1286 | GCGCUGUGACUACAUCUUU | D-027086-04 |
| TMEM132C | 92293 | 1287 | GACAGGAGCAGCAGUUUAU | D-027086-03 |
| TMEM132C | 92293 | 1288 | GCAGAUGAACUUUGAAAUA | D-027086-01 |
| TMEM163 | 81615 | 1289 | GGAGGACCGAGGCUUACUA | D-014673-02 |
| TMEM163 | 81615 | 1290 | CUACGAGAUGUUUGAGUGA | D-014673-04 |
| TMEM163 | 81615 | 1291 | GCGGAAGUGUUCAAGCAUG | D-014673-01 |
| TMEM163 | 81615 | 1292 | CGAUUGUCCUGUGGCGUUA | D-014673-03 |
| TMEM181 | 57583 | 1293 | GAACCACGAUGUACAUUCA | D-024897-01 |
| TMEM181 | 57583 | 1294 | CGGAUGAUGAUGUGAUUUA | D-024897-03 |
| TMEM181 | 57583 | 1295 | GAGUUGAUACCGGAAAUUU | D-024897-04 |
| TMEM181 | 57583 | 1296 | GCCCAGAGUUGCCACUAAA | D-024897-02 |
| TMTC1 | 83857 | 1297 | GAACAUGGGUGGCAUCCAA | D-016838-03 |
| TMTC1 | 83857 | 1298 | GAACAGCUCUCAAGUUGUA | D-016838-02 |
| TMTC1 | 83857 | 1299 | GCAAAGAUGUACUAUCAGA | D-016838-04 |
| TMTC1 | 83857 | 1300 | GGAAGAAGCUAUCACCUUA | D-016838-01 |
| TNPO3 | 23534 | 1301 | GAGGGUAUCAGACCUGGUA | D-019949-04 |
| TNPO3 | 23534 | 1302 | GCAGUGAUAUUUAGGCAUA | D-019949-01 |
| TNPO3 | 23534 | 1303 | GGAGAUCCUUACAGUGUUA | D-019949-02 |
| TNPO3 | 23534 | 1304 | GAAGGGAUGUGUGCAAACA | D-019949-03 |
| TOMM70A | 9868 | 1305 | CAACAAAGCUAUUAACCUG | D-021243-02 |
| TOMM70A | 9868 | 1306 | GCCCAUCUGUAUUCACUUU | D-021243-03 |
| TOMM70A | 9868 | 1307 | CGAAGGCUAUGCACUAUAC | D-021243-04 |
| TOMM70A | 9868 | 1308 | GCAAAGAAAUACGGAUUAA | D-021243-01 |
| TOR2A | 27433 | 1309 | GUUCAGCUCUACAGCCUUA | D-015292-01 |
| TOR2A | 27433 | 1310 | GCUACAAGAAGGAUCUGAA | D-015292-02 |
| TOR2A | 27433 | 1311 | CGGGACCAAUUACCGCAAA | D-015292-03 |
| TOR2A | 27433 | 1312 | UGUCAUUCCGCCUGGUUGA | D-015292-04 |
| TORC2 | 200186 | 1313 | CGACUACCAUCUGCACUUA | D-018947-02 |
| TORC2 | 200186 | 1314 | CUAAGAAGCUAUCCUCAUC | D-018947-03 |
| TORC2 | 200186 | 1315 | ACAAGGAGCUCUCAUUAUG | D-018947-01 |
| TORC2 | 200186 | 1316 | UGACAGCUCUCCCUAUAGU | D-018947-04 |
| TORC3 | 64784 | 1317 | GGACGGACUCAACAUGUUA | D-014210-01 |
| TORC3 | 64784 | 1318 | GAAGCCAACUUUCCUUUCU | D-014210-03 |
| TORC3 | 64784 | 1319 | CAACGCAUCUGCUCUUCAC | D-014210-04 |
| TORC3 | 64784 | 1320 | GGAAUAGUGUGAACAACAU | D-014210-02 |
| TRAPPC1 | 58485 | 1321 | GUUACAAACUCCAUUACUA | D-013781-01 |
| TRAPPC1 | 58485 | 1322 | CCACAACCUGUACCUGUUU | D-013781-04 |
| TRAPPC1 | 58485 | 1323 | GGAUCAAAGUUGUCAUGAA | D-013781-03 |
| TRAPPC1 | 58485 | 1324 | CGACUGGACUCCUAUGUUC | D-013781-02 |
| TREM5 | 124599 | 1325 | GGAUGGGAGACCUACAUUA | D-017772-03 |
| TREM5 | 124599 | 1326 | GCAGAUGUUUACUGGUGUG | D-017772-04 |
| TREM5 | 124599 | 1327 | CUAAAGACAUGGCCACUUA | D-017772-01 |
| TREM5 | 124599 | 1328 | GGGAACAGCCUAUCUACAU | D-017772-02 |
| TRIM55 | 84675 | 1329 | GAAUUCAGUUUAUGGAUGA | D-007092-02 |
| TRIM55 | 84675 | 1330 | GAAGUUUGAUUACCUGUAU | D-007092-01 |
| TRIM55 | 84675 | 1331 | GCGCAUCUCUGAAUUACAA | D-007092-03 |
| TRIM55 | 84675 | 1332 | GAAAUGUGCCAGUGAUAUU | D-007092-04 |
| TRIM58 | 25893 | 1333 | GAUUGGAGUUUGAGAAGCA | D-013985-03 |
| TRIM58 | 25893 | 1334 | GAAAGUCCUCGCUGCAUUG | D-013985-01 |
| TRIM58 | 25893 | 1335 | GGAAAGAGUUGGAGGACGC | D-013985-04 |
| TRIM58 | 25893 | 1336 | CUAUGAAGCCGGUGAAAUU | D-013985-02 |
| TRMT5 | 57570 | 1337 | CCACAGAUCUCUAAAUACA | D-021968-02 |
| TRMT5 | 57570 | 1338 | GUGCAUCACGUUUCAGAUU | D-021968-04 |
| TRMT5 | 57570 | 1339 | GACAAUAUGUACCGAAAUU | D-021968-03 |
| TRMT5 | 57570 | 1340 | GGAAAGAAAUAGUCAGUAA | D-021968-01 |
| TUBAL3 | 79861 | 1341 | GAGCAUUUCUGCACUGGUA | D-009010-01 |
| TUBAL3 | 79861 | 1342 | GCACACAAAUGCAUCUUUC | D-009010-02 |
| TUBAL3 | 79861 | 1343 | UAUGAUAUAUGCCAUCGUA | D-009010-04 |
| TUBAL3 | 79861 | 1344 | GAAUGUAGACCUAAUUGAA | D-009010-03 |
| UBQLN4 | 56893 | 1345 | CGACUUUGCUGCUCAGAUG | D-021178-03 |
| UBQLN4 | 56893 | 1346 | CAAUAACCCUGAACUCAUG | D-021178-02 |
| UBQLN4 | 56893 | 1347 | GGUCAGGGAUGUUCAAUAG | D-021178-01 |
| UBQLN4 | 56893 | 1348 | GAGCCUCGGUCAAGGAGUU | D-021178-04 |
| USP26 | 83844 | 1349 | CCACAAAGCUGGAGGUAAA | D-006075-02 |
| USP26 | 83844 | 1350 | CCACACAUUGGAUCAGAUA | D-006075-05 |
| USP26 | 83844 | 1351 | GCACAAGACUUCCGUUGGA | D-006075-04 |
| USP26 | 83844 | 1352 | GAAGAUACCUCACUUUGUC | D-006075-03 |
| USP6 | 9098 | 1353 | GAACCUGAUUGACGGGAUC | D-006096-09 |
| USP6 | 9098 | 1354 | CAACGGACCUGGAUAUAGG | D-006096-05 |
| USP6 | 9098 | 1355 | GAGCGGAAGGACAUACUUA | D-006096-08 |
| USP6 | 9098 | 1356 | GCGGAGAGGUUCACAACAA | D-006096-07 |
| VDRIP | 29079 | 1357 | UGAAAUUGGCACUUAAUCA | D-020687-04 |
| VDRIP | 29079 | 1358 | GAGAAGAGAGACAGUGAUA | D-020687-01 |
| VDRIP | 29079 | 1359 | UGAACAAUCCUUCCACUAA | D-020687-03 |
| VDRIP | 29079 | 1360 | CUAGGGAACUUAUAGAAAU | D-020687-02 |
| VPRBP | 9730 | 1361 | UCACAGAGUAUCUUAGAGA | D-021119-03 |
| VPRBP | 9730 | 1362 | GGACGACAAUAAUGAGAAC | D-021119-04 |
| VPRBP | 9730 | 1363 | GGAGGGAAUUGUCGAGAAU | D-021119-02 |
| VPRBP | 9730 | 1364 | GAUGGCGGAUGCUUUGAUA | D-021119-01 |
| VPS33B | 26276 | 1365 | UCACAGAUAUGACUAAGGA | D-007261-04 |
| VPS33B | 26276 | 1366 | GGAGAGGCAUGGACAUUAA | D-007261-01 |
| VPS33B | 26276 | 1367 | CAAGAUGGCAUAUGAAUUG | D-007261-02 |
| VPS33B | 26276 | 1368 | AAACAGCGCUCGCCUUAUG | D-007261-03 |
| VPS53 | 55275 | 1369 | GCGCCGACCUCUUUGUCUA | D-017048-04 |
| VPS53 | 55275 | 1370 | GCAAUUAGAUCACGCCAAA | D-017048-02 |
| VPS53 | 55275 | 1371 | AGAAGUACCUCCGAGAAUA | D-017048-03 |
| VPS53 | 55275 | 1372 | GAAAGGAGAUUUAGAUCAA | D-017048-01 |
| WBSCR17 | 64409 | 1373 | GAUCCGCGCUCGCAUUGAG | D-013019-03 |
| WBSCR17 | 64409 | 1374 | ACAAUAAUACCGUUGCUUA | D-013019-01 |
| WBSCR17 | 64409 | 1375 | UAAGAACUCCAUCAAGUAG | D-013019-04 |
| WBSCR17 | 64409 | 1376 | GAUUACAAGUCUCAUGUGU | D-013019-02 |
| WDTC1 | 23038 | 1377 | GUGCACGACCUGACAGUAA | D-016542-04 |
| WDTC1 | 23038 | 1378 | GACAUCCGCAUGAUCCAUA | D-016542-03 |
| WDTC1 | 23038 | 1379 | CACCAUACCUGGAGCGUGU | D-016542-02 |
| WDTC1 | 23038 | 1380 | CUAGAGACCUCAUCCGUAG | D-016542-01 |
| WNT1 | 7471 | 1381 | CCACGAACCUGCUUACAGA | D-003937-01 |
| WNT1 | 7471 | 1382 | GCGUUUAUCUUCGCUAUCA | D-003937-02 |
| WNT1 | 7471 | 1383 | CAAACAGCGGCGUCUGAUA | D-003937-04 |
| WNT1 | 7471 | 1384 | UCAGAAGGUUCCAUCGAAU | D-003937-03 |
| XKR4 | 114786 | 1385 | GUACGAAACCACUUUAUAA | D-025942-01 |
| XKR4 | 114786 | 1386 | CGACCGCGAUCAGAAAUUC | D-025942-03 |
| XKR4 | 114786 | 1387 | GCAGGCUAUUCAUUUACUA | D-025942-02 |
| XKR4 | 114786 | 1388 | CCGCAAAGGCAAGCAUCUA | D-025942-04 |
| YTHDC2 | 64848 | 1389 | GGACUAGGAGGAGUAUUUA | D-014220-04 |
| YTHDC2 | 64848 | 1390 | CAGCAUAGUUUACUUGGUA | D-014220-02 |
| YTHDC2 | 64848 | 1391 | GCAAAUAGAUACCUAACUG | D-014220-01 |
| YTHDC2 | 64848 | 1392 | GCAGGCAUGUAUCCUAAUU | D-014220-03 |
| ZBTB2 | 57621 | 1393 | CGACCCGGUUCGAUUAGAA | D-014129-03 |
| ZBTB2 | 57621 | 1394 | CAGGUGAAUCGGACAAAUA | D-014129-02 |
| ZBTB2 | 57621 | 1395 | GAUCAUCAGUUGAGACAAG | D-014129-01 |
| ZBTB2 | 57621 | 1396 | AGACGAAGGGCGAUCCAUU | D-014129-04 |
| ZDHHC11 | 79844 | 1397 | GCAAAUGGACAAAGGAGUU | D-014447-04 |
| ZDHHC11 | 79844 | 1398 | GGAAAUACAUUGCCUACGU | D-014447-01 |
| ZDHHC11 | 79844 | 1399 | GAAGAUGUCAAGAAUAUGA | D-014447-02 |
| ZDHHC11 | 79844 | 1400 | CCAAGAAGAUGACCACCUU | D-014447-03 |
| ZIM2 | 23619 | 1401 | GGACUUCAAACACUUAGGA | D-031995-04 |
| ZIM2 | 23619 | 1402 | UGACUACGUUGGAGAGAGA | D-031995-03 |
| ZIM2 | 23619 | 1403 | GAGGAGGAAUCAUAUGCAA | D-031995-02 |
| ZIM2 | 23619 | 1404 | GCAAGUAGCCCUUAGGAGA | D-031995-01 |
| ZNF182 | 7569 | 1405 | ACAGAAGCUUGAUCUAAUU | D-024670-04 |
| ZNF182 | 7569 | 1406 | CGAUAAACACGAAUCAUUU | D-024670-03 |
| ZNF182 | 7569 | 1407 | CAAAGUUCAUGGCACAUUA | D-024670-01 |
| ZNF182 | 7569 | 1408 | CAAGAGUGCUCGUGACUGU | D-024670-02 |
| ZNF271 | 10778 | 1409 | GCACAUGUACUGAUCUUAU | D-015671-01 |
| ZNF271 | 10778 | 1410 | GCAGGAAGGCUUUCAGUCA | D-015671-03 |
| ZNF271 | 10778 | 1411 | GAGAAAACCUUUAGUGUGU | D-015671-02 |
| ZNF271 | 10778 | 1412 | GUUCUGAUCUCAUUAACCA | D-015671-04 |
| ZNF331 | 55422 | 1413 | GGCCUUUACUCGAGUCAAU | D-021386-04 |
| ZNF331 | 55422 | 1414 | GUAAAUCCCUUGGCCGUAA | D-021386-01 |
| ZNF331 | 55422 | 1415 | GGAGGUAUGUCAAUCAGAU | D-021386-03 |
| ZNF331 | 55422 | 1416 | CGACGUAGCCAUAGACUUU | D-021386-02 |
| ZNF354A | 6940 | 1417 | GGAUGUGGCUGUGCUGUUU | D-007685-04 |
| ZNF354A | 6940 | 1418 | GGAAUGUACCUUGGGAUUU | D-007685-05 |
| ZNF354A | 6940 | 1419 | GAAUGUACCUUGGGAUUUG | D-007685-03 |
| ZNF354A | 6940 | 1420 | AAAGGGAAGUUUUCAGAUA | D-007685-06 |
| ZNF436 | 80818 | 1421 | GGUCAGAUCUAAUUAAACA | D-014640-01 |
| ZNF436 | 80818 | 1422 | CAUCCGCUAUCAUAUAUGU | D-014640-03 |
| ZNF436 | 80818 | 1423 | GGAAAUGUUGUCUCACUAG | D-014640-04 |
| ZNF436 | 80818 | 1424 | GGAGUGAGAACGAGGUAAA | D-014640-02 |
| ZNF512B | 57473 | 1425 | CACCAAACCCAUUACGGUA | D-013934-02 |
| ZNF512B | 57473 | 1426 | GGUGAAGUGUCCAAACUCA | D-013934-01 |
| ZNF512B | 57473 | 1427 | GCAUCUACGGGCUCAAGUA | D-013934-03 |
| ZNF512B | 57473 | 1428 | GGACAAGGCCCGAGUUCAC | D-013934-04 |
| ZNF536 | 9745 | 1429 | CAAGUAAGCUCGACCCUUU | D-020506-01 |
| ZNF536 | 9745 | 1430 | CCACGUGGACCCUGCAUUU | D-020506-04 |
| ZNF536 | 9745 | 1431 | CUACAGUUCUGAUGGCUUA | D-020506-02 |
| ZNF536 | 9745 | 1432 | GGACAUCCCAUCACCUUAA | D-020506-03 |
| ZNF556 | 80032 | 1433 | GAACAUAGCGUUAAAGACA | D-014533-01 |
| ZNF556 | 80032 | 1434 | CGCAAGAAUUGUUGUACUA | D-014533-03 |
| ZNF556 | 80032 | 1435 | CCACAGAUGUCAAAUCACA | D-014533-02 |
| ZNF556 | 80032 | 1436 | GCGCACAUGUGAUGAUGCA | D-014533-04 |
| ZNF720 | 124411 | 1437 | CAAGAGAAGUCUGCCAAAU | D-022814-02 |
| ZNF720 | 124411 | 1438 | AAUUGUAGUUCACGCCUUA | D-022814-03 |
| ZNF720 | 124411 | 1439 | ACUCAAGGCCUCUUAAGAA | D-022814-04 |
| ZNF720 | 124411 | 1440 | GGUCGUACCUAACUAAACA | D-022814-01 |
| ZNF785 | 146540 | 1441 | CAAGGACACUCUGACCCGA | D-018331-04 |
| ZNF785 | 146540 | 1442 | GCGCUGGGAUUUUCAGUUC | D-018331-03 |
| ZNF785 | 146540 | 1443 | GCAAGAGUCGCUUCACUUA | D-018331-01 |
| ZNF785 | 146540 | 1444 | GCACUUUCCAGAUAUAUUU | D-018331-02 |
| ZNF791 | 163049 | 1445 | GAUACGAGCUAUUUGAGAA | D-015752-02 |
| ZNF791 | 163049 | 1446 | UGAGAAUGCACAAUCGAUA | D-015752-04 |
| ZNF791 | 163049 | 1447 | GAAGAAGACUGCCGGAGUA | D-015752-01 |
| ZNF791 | 163049 | 1448 | GGGAAGACCCGAAUGUUGA | D-015752-03 |
| ZNRD1 | 30834 | 1449 | CAUCAACGUUCGGGACUUU | D-017359-02 |
| ZNRD1 | 30834 | 1450 | GUCAUGAAGGAAUGGCAUA | D-017359-03 |
| ZNRD1 | 30834 | 1451 | GGACCUGGAUUUCUGUUCA | D-017359-01 |
| ZNRD1 | 30834 | 1452 | GGGAAGGUUGUGAAGACUU | D-017359-04 |
| ZZEF1 | 23140 | 1453 | CGAAACACCCGUAUAACAA | D-031841-03 |
| ZZEF1 | 23140 | 1454 | AUAACUAGCUGCUGUUCUA | D-031841-02 |
| ZZEF1 | 23140 | 1455 | GUAUCGCACUCCAGAUUUA | D-031841-01 |
| ZZEF1 | 23140 | 1456 | GAAGAGGAUUUUGGGAUUA | D-031841-04 |

TABLE 4

| | | siRNAs | Merck or | T cell | | |
|---|---|---|---|---|---|---|
| Gene | Gene ID | scoring | Novartis Hit | Expression | Presumed Activity | Transmembrane |
| ADAM10 | 102 | 3 | | Y | Protease | Y |
| DDX49 | 54555 | 2 | | Y | Helicase | |
| DDX55 | 57696 | 3 | | Y | Helicase | |
| DMXL1 | 1657 | 2 | Novartis | Y | Protein interaction (WD40 domains) | |
| DUSP16 | 80824 | 2 | Novartis | Y | Phosphatase | |
| GPN3 | 51184 | 3 | | Y | ATP binding | |
| HERC3 | 8916 | 3 | | Y | ubiquitin-protein ligase | |
| PIP5K1C | 23396 | 3 | | Y | Kinase | |
| RNF170 | 81790 | 3 | | Y | ubiquitin-protein ligase | |
| RNF26 | 79102 | 1 | Merck | Y | ubiquitin-protein ligase | |
| TM9SF3 | 56889 | 3 | | Y | Cargo transport | Y |
| TMEM181 | 57583 | 3 | | Y | G protein coupled receptor | Y |
| TNPO3 | 23534 | 4 | Novartis | Y | Nuclear importer | |
| TRIM55 | 84675 | 2 | Novartis | Y | ubiquitin-protein ligase | |

1-6. (canceled)

7. A method for treating or preventing HIV infection in a cell comprising downmodulating one or more of the HIV-dependency factors (HDFs) in the cell to thereby treat or prevent HIV infection in the cell.

8. The method of claim 7, wherein the one or more HDFs are selected from the group consisting of ADDAM10, DDX49, DDX55, DMXL1, DUSP16, GPN3, HERC3, PIP5K1C, RNF170, RNF26, RM9SF3, TMEM181, TNP03, TRIM55, and combinations thereof.

9. The method of claim 7, wherein downmodulating the one or more HDFs comprises contacting the cell with an agent that downmodulates the HDFs.

10. The method of claim 9, wherein the agent inhibits HDF gene expression, protein synthesis, HDF function or HDF activity, or combinations thereof.

11. A method for treating or preventing HIV infection in a subject comprising downmodulating one or more of the HIV-dependency factors (HDFs) in cells of the subject, to thereby treat or prevent HIV infection in the subject.

12. The method of claim 11, wherein the one or more HDFs are selected from the group consisting of ADDAM10, DDX49, DDX55, DMXL1, DUSP16, GPN3, HERC3, PIP5K1C, RNF170, RNF26, RM9SF3, TMEM181, TNPO3, TRIM55, and combinations thereof.

13. The method of claim 11, which further comprises selecting a subject diagnosed with or at risk for HIV infection, prior to downmodulating.

14. The method of claim 11, wherein downmodulating the HDFs comprises administering an agent that downmodulates the HDF to the subject such that the agent contacts HIV host cells of the subject.

15. A small inhibitory nucleic acid sequence that downmodulates an HIV-dependency factor (HDF).

16. The small inhibitory nucleic acid sequence of claim 15, wherein the HDF is selected from the group consisting of ADDAM10, DDX49, DDX55, DMXL1, DUSP16, GPN3, HERC3, PIP5K1C, RNF170, RNF26, RM9SF3, TMEM181, TNPO3, and TRIM55.

17. The small inhibitory nucleic acid sequence of claim 15 that is an RNAi.

18. The small inhibitory nucleic acid sequence of claim 17, wherein the RNAi comprises the nucleic acid sequence of an siRNA listed in Table 3.
